SEQUENCE LISTING (219 sequences)

SEQ ID NO: 1 - DNA, 26 bp, Artificial Sequence
  source: /note=“Description of Artificial Sequence: Synthetic oligonucleotide”
taactataacggtcctaaggtagcga

SEQ ID NO: 2 - DNA, 2820 bp, Artificial Sequence
  source: /note=“Description of Artificial Sequence: Synthetic polynucleotide”
ggccggcctgactaactaaactagtgcgattaacattagcacagagagcgttttgccgagtcccatgtcgtcggcaagcacgccgccgttgggccggccgtttttttctctgttaatcatccactgtatgccttttttctgatgcgccaatagattgggcgtttgatgttcaaagttgggatcgtcgttgtcgggcgcttggtcaaaaaattcttgcaattgcaatttgtaattatccattgtggcgacggctattctcttcgattaaactatggttattgcgcgctcgtaatctaagatatcacatttaacacggacattatctaatcaacgtgattgacgacgagttcatttattgtatcttcaataatttgttcgtaaactggtaatgttgttacttgtatcgcaccaagttttctaacatacacacgaacatgtttgtagcttgtttctgaaaaattttgcacaaaatacggcaacccgtccgcgtacatgttagcttgtagcgtgcgcgtgtacacgaccaatttgagggccacgacgtttttaaaaagcgtgtaattcatgtacgtgtaacgcgactagttgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggcc

SEQ ID NO: 3 - DNA, 2405 bp, Artificial Sequence
  source: /note=“Description of Artificial Sequence: Synthetic polynucleotide”
ggccggcctgactaactaaactagtgtatactgattgtgagcgctcacaatttattttacaaagattcagaaatacgcatcacttacaacaagggggactttgaaattttgcatttgaggttgccgggacctttaattcaacccaacacaatatattatagttaaataagaattattatcaaatcatttgtatattaattaaaatactatactgtaaattacattttatttacaatcactgattgtgagcgctcacaattcccatggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaagctagcacgcgtagctgatgcatagcatgcggtaccgggagatgggggaggctaactgaaacacggaaggagacaataccggaaggaacccgcgctatgacggcaataaaaagacagaataaaacgcacgggtgttgggtcgtttgttctctagattagttagtcaggccggatggataattacaaattgcaattgcaagaattttttgaccaagcgcccgacaacgacgatcccaactttgaacatcaaacgcccaatctattggcgcatcagaaaaaaggcatacagtggatgattaacagagaaaaaaacggccggcc

SEQ ID NO: 4 - DNA, 15 bp, Artificial Sequence
  source: /note=“Description of Artificial Sequence: Synthetic oligonucleotide”
atgaacaaagattaa

SEQ ID NO: 5 - DNA, 18 bp, Artificial Sequence
  source: /note=“Description of Artificial Sequence: Synthetic oligonucleotide”
atgcctcaatgctactag

SEQ ID NO: 6 - DNA, 1128 bp, Artificial Sequence
  source: /note=“Description of Artificial Sequence: Synthetic polynucleotide”
atgacgcaacctaagaagaagaggaaggttcccgggcaagtgactatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgtcaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 7 - DNA, 1128 bp, Artificial Sequence
  source: /note=“Description of Artificial Sequence: Synthetic polynucleotide”
atgacccagcccaaaaaaaagagaaaagtccctggtcaggtaactatgaagccggtcaccttgtacgacgtcgctgaatacgcaggtgtgtcctatcaaacagtgagtcgtgtggtcaaccaggccagccacgttagtgcaaaaacccgtgagaaagtggaggcggcaatggccgagcttaattacataccaaaccgtgttgcacaacagcttgcaggcaagcaatccctgctcattggcgttgcaactagtagcctggctcttcatgcgccttcccagattgtggccgctattaagagtagagcagatcagttgggagcatccgttgtcgtcagcatggttgaaaggagtggcgttgaggcttgcaaagctgcggtccataacctgctggcacaaagagtgagcggactcatcatcaactacccactggatgatcaagacgcgattgccgtcgaggctgcatgtacgaacgttccagctctttttcttgacgtgtcagaccaaacacccataaattctattatttttagtcacgaggacggtacccgcttgggtgttgaacacttggtagctttgggacatcagcagatcgcattgttggctggtccacttagtagcgtgagtgcaagacttagactggcaggttggcacaagtacttgacccgtaatcaaatacaacctatagctgagcgcgaaggtgactggagcgctatgagcggtttccagcaaacgatgcagatgctgaacgagggaattgtacccaccgctatgttggtcgctaacgaccaaatggccttgggcgccatgagagctattactgaaagcggcttgcgcgtgggcgcagatatctctgtcgtaggttacgatgatacggaagactctagctgctatatccccccaagcacgacgatcaagcaggatttccgtctccttggacagaccagtgtagacaggctccttcaactgagccagggccaagctgtgaagggcaaccaactcttgccagtgtcgttggtgaagcgcaagaccaccctggctcccaacacgcaaactgcttctcccagggctttggctgattccctgatgcagctcgcccgccaagtcagtagacttgaatcaggccagtga

SEQ ID NO: 8 - DNA, 20 bp, Artificial Sequence
  source: /note=“Description of Artificial Sequence: Synthetic oligonucleotide”
gattgtgagcgctcacaatt

SEQ ID NO: 9 - DNA, 2401 bp, Artificial Sequence
  source: /note=“Description of Artificial Sequence: Synthetic polynucleotide”
tgtgttgggttgaattaaaggtccgtaaatagggccggccgttaacatcatggagataattaaaatgataaccatctcgcaaataaataagtattttactgttttcgtaacagttttgtaataaaaaaacctataaatattccggattattcataccgtcccaccatcgggcgcggttaaccttaagaataaaaatgccggggttttacgagattgtgattaaggtccccagcgaccttgacgagcatctgcccggcatttctgacagctttgtgaactgggtggccgagaaggaatgggagttgccgccagattctgacatggatctgaatctgattgagcaggcacccctgaccgtggccgagaagctgcagcgcgactttctgacggaatggcgccgtgtgagtaaggccccggaggcccttttctttgtgcaatttgagaagggagagagctacttccacatgcacgtgctcgtggaaaccaccggggtgaaatccatggttttgggacgtttcctgagtcagattcgcgaaaaactgattcagagaatttaccgcgggatcgagccgactttgccaaactggttcgcggtcacaaagaccagaaatggcgccggaggcgggaacaaggtggtggatgagtgctacatccccaattacttgctccccaaaacccagcctgagctccagtgggcgtggactaatatggaacagtatttaagcgcctgtttgaatctcacggagcgtaaacggttggtggcgcagcatctgacgcacgtgtcgcagacgcaggagcagaacaaagagaatcagaatcccaattctgatgcgccggtgatcagatcaaaaacttcagccaggtacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaactcgaggaatcaagcttgcatgcctgcaggtcgactctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggatctgatcactgcttgagcctag

SEQ ID NO: 10 - DNA, 1432 bp, Artificial Sequence
  source: /note=“Description of Artificial Sequence: Synthetic polynucleotide”
agacacggtaaccgccgctaactacgctagcgtatcatggagataattaaaatgataaccatctcgcaaataaataagtattttactgttttcgtaacagttttgtaataaaaaaacctataaatattccggattattcataccgtcccaccatcgggcgcggttaaccttaagaataaaaatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaacaatcaaattatcgcaagctagcttgattgaaatgttcaaagaccagtttgacaat

SEQ ID NO: 11 - DNA, 15 bp, Artificial Sequence
  source: /note=“Description of Artificial Sequence: Synthetic oligonucleotide”
taaaacaaagattaa

SEQ ID NO: 12 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttagcttacttacttaattaacctacaacatgaacaaagattaaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 13 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttaacctacaacatgaacaaagattaaattagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 14 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaagcttacttacttacttagcttacttacttacttaacctacaacatgaacaaagattaaattagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 15 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaagcttacttacttacttaacctacaacatgaacaaagattaaattagcttacttacttacttagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 16 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaaacctacaacatgaacaaagattaaattagcttacttacttacttagcttacttacttacttagcttacttacttacttagcttacttacttaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 17 - DNA, 2070 bp, Artificial Sequence
  source: /note=“Description of Artificial Sequence: Synthetic polynucleotide”
atcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttagcttacttacttaattaacctacaactaaaacaaagattaaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 18 - DNA, 49 bp, Artificial Sequence
  source: /note=“Description of Artificial Sequence: Synthetic primer”
gattaaggccttaattaggctgcggcccgggccgagcgcgcagccttaa

SEQ ID NO: 19 - DNA, 3270 bp, Artificial Sequence
  source: /note=“Description of Artificial Sequence: Synthetic polynucleotide”
tggcagggaacaaacgacccaacacccgtgcgttttattctgtctttttattgccgtcatagcgcgggttccttccggtattgtctccttccgtgtttcagttagcctcccccatctcccggtacacgcggttagaggcggttgctagcggcgcgccgcgcctcggtgatgacggtgaaaacctctataacgcgaagtaatcttttcggttttaaagaaaaagggcagggtggtgacaccttgcccttttttgccggattattacggcaaatcacactccagccaactctcagggaccgcttctggcgcaggaaaccacgcaaaacgccactcaccgtttaagctacgcagttgttgactcggacggtcagtgcgtgcctcttcactattgcgccacgaggcaaagggagggtgcgctgccaaacggttaagctgcgtaactccagggttctcccagtcacggcgttgtaaaaccaccgctaaagaattgccggggaagttgaccagccgcgtaacctggcaaaatcggttacggttgagtaataaatggatgccctgcgtaagcggggcattggacggggtgatcatggtcatctagtatttctcctctttctctagtaaacagtactatgtgattataccagccccctcactacatgtcaagaataaactgccaaagaaacaaataggggttccgcgcacggcgcgccttaacaggatacagtctacgtcgagtgattgtaaataaaatgtaatttacagtatagtattttaattaatatacaaatgatttgataataattcttatttaactataatatattgtgttgggttgaattaaaggtccgtatactccggaatattaatagatcatggagataattaaaatgataaccatctcgcaaataaataagtattttactgttttcgtaacagttttgtaataaaaaaacctataaatcgcggatctgccgccaagcttacttacttacttagcttacttacttacttagcttacttacttacttagcttacttacttaattaacctacaacatgaacaaagattaaattatggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaactcgaggaatcaagcttgcatgcctgcaggtcgactctagagcctgcagtctcgacaagcttgtcgagaagtactagaggatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgcctggatctgatcactgc

SEQ ID NO: 20 - DNA, 687 bp, Artificial Sequence
  source: /note=“Description of Artificial Sequence: Synthetic polynucleotide”
cagtgatcagatccaggattaaggccttaattaggctgcggcgcgccgtgcgcggaacccctatttgtttctggaaagcgggcagtgagcgcaacgcaattaatgtgagttagctcactcattaggcaccccaggctttacactttatgcttccggctcgtatgttgtgtggaattgcgagcggataacaactagagaaagaggagaaatactagatgaccatgatcaccccgtccaatgccccgcttacgcagggcatccatttattactcaaccgtaaccgattttgccaggttacgcggctggtcaacttccccggcaattctttagcggtggttttacaacgccgtgactgggagaaccctggagttacgcagcttaaccgtttggcagcgcaccctccctttgcctcgtggcgcaatagtgaagaggcacgcactgaccgtccgagtcaacaactgcgtagcttaaacggtgagtggcgttttgcgtggtttcctgcgccagaagcggtccctgagagttggctggagtgtgatttgccgtaataatccggcaaaaaagggcaaggtgtcaccaccctgccctttttctttaaaaccgaaaagattacttcgcgttatagaggttttcaccgtcatcaccgaggcgcgccgagcgcgcagccttaatcctgccatagccact

SEQ ID NO: 21 - RNA, 11 nt, Artificial Sequence
  source: /note=“Description of Artificial Sequence: Synthetic oligonucleotide”
cgguguauggc

SEQ ID NO: 22 - RNA, 11 nt, Artificial Sequence
  source: /note=“Description of Artificial Sequence: Synthetic oligonucleotide”
cgguguaugag

SEQ ID NO: 23 - RNA, 11 nt, Artificial Sequence
  source: /note=“Description of Artificial Sequence: Synthetic oligonucleotide”
uaguggaugag

SEQ ID NO: 24 - RNA, 11 nt, Artificial Sequence
  source: /note=“Description of Artificial Sequence: Synthetic oligonucleotide”
uauuggaugag

SEQ ID NO: 25 - RNA, 11 nt, Artificial Sequence
  source: /note=“Description of Artificial Sequence: Synthetic oligonucleotide”
caguggaugag

SEQ ID NO: 26 - RNA, 11 nt, Artificial Sequence
  source: /note=“Description of Artificial Sequence: Synthetic oligonucleotide”
cauuggaugag

SEQ ID NO: 27 - RNA, 11 nt, Artificial Sequence
  source: /note=“Description of Artificial Sequence: Synthetic oligonucleotide”
ugguggaugag

SEQ ID NO: 28 - RNA, 11 nt, Artificial Sequence
  source: /note=“Description of Artificial Sequence: Synthetic oligonucleotide”
aauuggaugag

SEQ ID NO: 29 - RNA, 11 nt, Artificial Sequence
  source: /note=“Description of Artificial Sequence: Synthetic oligonucleotide”
aaguggaugag

SEQ ID NO: 30 - RNA, 11 nt, Artificial Sequence
  source: /note=“Description of Artificial Sequence: Synthetic oligonucleotide”
ccguggaugag

SEQ ID NO: 31 - RNA, 11 nt, Artificial Sequence
  source: /note=“Description of Artificial Sequence: Synthetic oligonucleotide”
ggguggaugag

SEQ ID NO: 32 - DNA, 11 bp, Artificial Sequence
  source: /note=“Description of Artificial Sequence: Synthetic oligonucleotide”
cggtgtatggc

SEQ ID NO: 33 - DNA, 11 bp, Artificial Sequence
  source: /note=“Description of Artificial Sequence: Synthetic oligonucleotide”
cggtgtatgag

SEQ ID NO: 34 - DNA, 11 bp, Artificial Sequence
  source: /note=“Description of Artificial Sequence: Synthetic oligonucleotide”
tagtggatgag

SEQ ID NO: 35 - DNA, 11 bp, Artificial Sequence
  source: /note=“Description of Artificial Sequence: Synthetic oligonucleotide”
tattggatgag

SEQ ID NO: 36 - DNA, 11 bp, Artificial Sequence
  source: /note=“Description of Artificial Sequence: Synthetic oligonucleotide”
cagtggatgag

SEQ ID NO: 37 - DNA, 11 bp, Artificial Sequence
  source: /note=“Description of Artificial Sequence: Synthetic oligonucleotide”
cattggatgag

SEQ ID NO: 38 - DNA, 11 bp, Artificial Sequence
  source: /note=“Description of Artificial Sequence: Synthetic oligonucleotide”
tggtggatgag

SEQ ID NO: 39 - DNA, 11 bp, Artificial Sequence
  source: /note=“Description of Artificial Sequence: Synthetic oligonucleotide”
aattggatgag

SEQ ID NO: 40 - DNA, 11 bp, Artificial Sequence
  source: /note=“Description of Artificial Sequence: Synthetic oligonucleotide”
aagtggatgag

SEQ ID NO: 41 - DNA, 11 bp, Artificial Sequence
  source: /note=“Description of Artificial Sequence: Synthetic oligonucleotide”
ccgtggatgag

SEQ ID NO: 42 - DNA, 11 bp, Artificial Sequence
  source: /note=“Description of Artificial Sequence: Synthetic oligonucleotide”
gggtggatgag

SEQ ID NO: 43 - DNA, 2211 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 44 - DNA, 2217 bp, Artificial Sequence
  source: /note=“Description of Artificial Sequence: Synthetic polynucleotide”
cggtgtatggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 45 - DNA, 2217 bp, Artificial Sequence
  source: /note=“Description of Artificial Sequence: Synthetic polynucleotide”
cggtgtatgagtgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 46 - protein, 736 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 47 - protein, 736 aa, Artificial Sequence
  source: /note=“Description of Artificial Sequence: Synthetic polypeptide”
MSADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 48 - protein, 736 aa, Artificial Sequence; identical to SEQ ID NO: 46
  source: /note=“Description of Artificial Sequence: Synthetic polypeptide”

SEQ ID NO: 49 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 50 - DNA, 2238 bp, Artificial Sequence
  source: /note=“Description of Artificial Sequence: Synthetic polynucleotide”
cggtgtatggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 51 - DNA, 2238 bp, Artificial Sequence
  source: /note=“Description of Artificial Sequence: Synthetic polynucleotide”
cggtgtatgagtgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 52 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 53 - protein, 743 aa, Artificial Sequence
  source: /note=“Description of Artificial Sequence: Synthetic polypeptide”
MSADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 54 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSDGTLAVPFKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 55 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaggactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 56 - protein, 743 aa, Artificial Sequence; identical to SEQ ID NO: 52
  source: /note=“Description of Artificial Sequence: Synthetic polypeptide”

SEQ ID NO: 57 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 58 - DNA, 2238 bp, Artificial Sequence
  source: /note=“Description of Artificial Sequence: Synthetic polynucleotide”
cggtgtatggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 59 - DNA, 2238 bp, Artificial Sequence
  source: /note=“Description of Artificial Sequence: Synthetic polynucleotide”
cggtgtatgagtgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 60 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 61 - protein, 743 aa, Artificial Sequence
  source: /note=“Description of Artificial Sequence: Synthetic polypeptide”
MSADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSPLNGAVHLYAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 62 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgtgattctccgaagggttggcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 63 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgtgattctccgaagggttggcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 64 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARDSPKGWQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 65 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttattctacggatgtgaggatgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 66 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAYSTDVRMQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 67 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacagattgttatgaattcgttgaaggctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 68 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQIVMNSLKAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - DNA, 2232 bp, Artificial Sequence
  source: /note=“Description of Artificial Sequence: Synthetic polynucleotide”
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgggagagtcctcgtgggctgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 70 - protein, 743 aa, Artificial Sequence
  source: /note=“Description of Artificial Sequence: Synthetic polypeptide”
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQARESPRGLQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - protein, 736 aa, Artificial Sequence
  source: /note=“Description of Artificial Sequence: Synthetic polypeptide”
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 72 - DNA, 2211 bp, Artificial Sequence
  source: /note=“Description of Artificial Sequence: Synthetic polynucleotide”
atggctgccgatggttatcttccagattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 73 - RNA, 11 nt, Artificial Sequence
  source: /note=“Description of Artificial Sequence: Synthetic oligonucleotide”
uaguguauguc

SEQ ID NO: 74 - RNA, 11 nt, Artificial Sequence
  source: /note=“Description of Artificial Sequence: Synthetic oligonucleotide”
uaguuuauguc

SEQ ID NO: 75 - RNA, 11 nt, Artificial Sequence
  source: /note=“Description of Artificial Sequence: Synthetic oligonucleotide”
uguuuuauguc

SEQ ID NO: 76 - RNA, 11 nt, Artificial Sequence
  source: /note=“Description of Artificial Sequence: Synthetic oligonucleotide”
ucuuuuauguc

SEQ ID NO: 77 - RNA, 11 nt, Artificial Sequence
  source: /note=“Description of Artificial Sequence: Synthetic oligonucleotide”
uaguguauggg

SEQ ID NO: 78 - RNA, 11 nt, Artificial Sequence
  source: /note=“Description of Artificial Sequence: Synthetic oligonucleotide”
uaguuuauggg

SEQ ID NO: 79 - RNA, 11 nt, Artificial Sequence
  source: /note=“Description of Artificial Sequence: Synthetic oligonucleotide”
uguuuuauggg

SEQ ID NO: 80 - RNA, 11 nt, Artificial Sequence
  source: /note=“Description of Artificial Sequence: Synthetic oligonucleotide”
ucuuuuauggg

SEQ ID NO: 81 - RNA, 11 nt, Artificial Sequence
  source: /note=“Description of Artificial Sequence: Synthetic oligonucleotide”
uaguguauggc

SEQ ID NO: 82 - RNA, 11 nt, Artificial Sequence
  source: /note=“Description of Artificial Sequence: Synthetic oligonucleotide”
uaguuuauggc

SEQ ID NO: 83 - RNA, 11 nt, Artificial Sequence
  source: /note=“Description of Artificial Sequence: Synthetic oligonucleotide”
uguuuuauggc

SEQ ID NO: 84 - RNA, 11 nt, Artificial Sequence
  source: /note=“Description of Artificial Sequence: Synthetic oligonucleotide”
ucuuuuauggc

SEQ ID NO: 85 - RNA, 11 nt, Artificial Sequence
  source: /note=“Description of Artificial Sequence: Synthetic oligonucleotide”
cauuguauguc

SEQ ID NO: 86 - RNA, 11 nt, Artificial Sequence
  source: /note=“Description of Artificial Sequence: Synthetic oligonucleotide”
ucguuuaugga

SEQ ID NO: 87 - RNA, 11 nt, Artificial Sequence
  source: /note=“Description of Artificial Sequence: Synthetic oligonucleotide”
caguuuauggu

SEQ ID NO: 88 - RNA, 11 nt, Artificial Sequence
  source: /note=“Description of Artificial Sequence: Synthetic oligonucleotide”
cauuguauggu

SEQ ID NO: 89 - RNA, 11 nt, Artificial Sequence
  source: /note=“Description of Artificial Sequence: Synthetic oligonucleotide”
uaguguaugcu

SEQ ID NO: 90 - RNA, 11 nt, Artificial Sequence
  source: /note=“Description of Artificial Sequence: Synthetic oligonucleotide”
cauuguaugcu

SEQ ID NO: 91 - RNA, 11 nt, Artificial Sequence
  source: /note=“Description of Artificial Sequence: Synthetic oligonucleotide”
ucuuuuauguc

SEQ ID NO: 92 - RNA, 11 nt, Artificial Sequence
  source: /note=“Description of Artificial Sequence: Synthetic oligonucleotide”
uguuuuauguc

SEQ ID NO: 93 - RNA, 11 nt, Artificial Sequence
  source: /note=“Description of Artificial Sequence: Synthetic oligonucleotide”
uaguuuauguc

SEQ ID NO: 94 - RNA, 11 nt, Artificial Sequence
  source: /note=“Description of Artificial Sequence: Synthetic oligonucleotide”
uaguguauguc

SEQ ID NO: 95 - RNA, 13 nt, Artificial Sequence
  source: /note=“Description of Artificial Sequence: Synthetic oligonucleotide”
cgacaugaauguc

SEQ ID NO: 96 - RNA, 13 nt, Artificial Sequence
  source: /note=“Description of Artificial Sequence: Synthetic oligonucleotide”
agggauggauguc

SEQ ID NO: 97 - RNA, 13 nt, Artificial Sequence
  source: /note=“Description of Artificial Sequence: Synthetic oligonucleotide”
cuugauggauguc

SEQ ID NO: 98 - RNA, 13 nt, Artificial Sequence
  source: /note=“Description of Artificial Sequence: Synthetic oligonucleotide”
cgugauguauguc

SEQ ID NO: 99 - RNA, 13 nt, Artificial Sequence
  source: /note=“Description of Artificial Sequence: Synthetic oligonucleotide”
ggcgauggauguc

SEQ ID NO: 100 - RNA, 13 nt, Artificial Sequence
  source: /note=“Description of Artificial Sequence: Synthetic oligonucleotide”
cgugauggauguc

SEQ ID NO: 101 - RNA, 13 nt, Artificial Sequence
  source: /note=“Description of Artificial Sequence: Synthetic oligonucleotide”
cguaaugcauguc

SEQ ID NO: 102 - RNA, 13 nt, Artificial Sequence
  source: /note=“Description of Artificial Sequence: Synthetic oligonucleotide”
gaacaugaauguc

SEQ ID NO: 103 - RNA, 13 nt, Artificial Sequence
  source: /note=“Description of Artificial Sequence: Synthetic oligonucleotide”
ggacaugcauguc

SEQ ID NO: 104 - RNA, 13 nt, Artificial Sequence
  source: /note=“Description of Artificial Sequence: Synthetic oligonucleotide”
aagaaugaauguc

SEQ ID NO: 105 - RNA, 13 nt, Artificial Sequence
  source: /note=“Description of Artificial Sequence: Synthetic oligonucleotide”
cccgauggauguc

SEQ ID NO: 106 - RNA, 13 nt, Artificial Sequence
  source: /note=“Description of Artificial Sequence: Synthetic oligonucleotide”
gccgauguauguc

SEQ ID NO: 107 - RNA, 13 nt, Artificial Sequence
  source: /note=“Description of Artificial Sequence: Synthetic oligonucleotide”
cgggaugaauguc

SEQ ID NO: 108 - RNA, 14 nt, Artificial Sequence
  source: /note=“Description of Artificial Sequence: Synthetic oligonucleotide”
gaccaugcauggcu

SEQ ID NO: 109 - RNA, 13 nt, Artificial Sequence
  source: /note=“Description of Artificial Sequence: Synthetic oligonucleotide”
aauuaugcauggc

SEQ ID NO: 110 - RNA, 13 nt, Artificial Sequence
  source: /note=“Description of Artificial Sequence: Synthetic oligonucleotide”
agacauguauggc

SEQ ID NO: 111 - RNA, 13 nt, Artificial Sequence
  source: /note=“Description of Artificial Sequence: Synthetic oligonucleotide”
aagaauguauggc

SEQ ID NO: 112 - RNA, 14 nt, Artificial Sequence
  source: /note=“Description of Artificial Sequence: Synthetic oligonucleotide”
gaccaugcauggcu

SEQ ID NO: 113 - RNA, 13 nt, Artificial Sequence
  source: /note=“Description of Artificial Sequence: Synthetic oligonucleotide”
cgcgauggauggc

SEQ ID NO: 114 - RNA, 13 nt, Artificial Sequence
  source: /note=“Description of Artificial Sequence: Synthetic oligonucleotide”
agcgauggauggc

SEQ ID NO: 115 - RNA, 13 nt, Artificial Sequence
  source: /note=“Description of Artificial Sequence: Synthetic oligonucleotide”
gaucaugcauggc

SEQ ID NO: 116 - RNA, 13 nt, Artificial Sequence
  source: /note=“Description of Artificial Sequence: Synthetic oligonucleotide”
cguaauggauggc

SEQ ID NO: 117 - RNA, 13 nt, Artificial Sequence
  source: /note=“Description of Artificial Sequence: Synthetic oligonucleotide”
aacgauguauggc

SEQ ID NO: 118 - DNA, 11 bp, Artificial Sequence
  source: /note=“Description of Artificial Sequence: Synthetic oligonucleotide”
tagtgtatgtc

SEQ ID NO: 119 - DNA, 11 bp, Artificial Sequence
  source: /note=“Description of Artificial Sequence: Synthetic oligonucleotide”
tagtttatgtc

SEQ ID NO: 120 - DNA, 11 bp, Artificial Sequence
  source: /note=“Description of Artificial Sequence: Synthetic oligonucleotide”
tgttttatgtc

SEQ ID NO: 121 - DNA, 11 bp, Artificial Sequence
  source: /note=“Description of Artificial Sequence: Synthetic oligonucleotide”
tcttttatgtc

SEQ ID NO: 122 - DNA, 11 bp, Artificial Sequence
  source: /note=“Description of Artificial Sequence: Synthetic oligonucleotide”
tagtgtatggg

SEQ ID NO: 123 - DNA, 11 bp, Artificial Sequence
  source: /note=“Description of Artificial Sequence: Synthetic oligonucleotide”
tagtttatggg

SEQ ID NO: 124 - DNA, 11 bp, Artificial Sequence
  source: /note=“Description of Artificial Sequence: Synthetic oligonucleotide”
tgttttatggg

SEQ ID NO: 125 - DNA, 11 bp, Artificial Sequence
  source: /note=“Description of Artificial Sequence: Synthetic oligonucleotide”
tcttttatggg

SEQ ID NO: 126 - DNA, 11 bp, Artificial Sequence
  source: /note=“Description of Artificial Sequence: Synthetic oligonucleotide”
tagtgtatggc

SEQ ID NO: 127 - DNA, 11 bp, Artificial Sequence
  source: /note=“Description of Artificial Sequence: Synthetic oligonucleotide”
tagtttatggc

SEQ ID NO: 128 - DNA, 11 bp, Artificial Sequence
  source: /note=“Description of Artificial Sequence: Synthetic oligonucleotide”
tgttttatggc

SEQ ID NO: 129 - DNA, 11 bp, Artificial Sequence
  source: /note=“Description of Artificial Sequence: Synthetic oligonucleotide”
tcttttatggc

SEQ ID NO: 130 - DNA, 11 bp, Artificial Sequence
  source: /note=“Description of Artificial Sequence: Synthetic oligonucleotide”
cattgtatgtc

SEQ ID NO: 131 - DNA, 11 bp, Artificial Sequence
  source: /note=“Description of Artificial Sequence: Synthetic oligonucleotide”
tcgtttatgga

SEQ ID NO: 132 - DNA, 11 bp, Artificial Sequence
  source: /note=“Description of Artificial Sequence: Synthetic oligonucleotide”
cagtttatggt

SEQ ID NO: 133 - DNA, 11 bp, Artificial Sequence
  source: /note=“Description of Artificial Sequence: Synthetic oligonucleotide”
cattgtatggt

SEQ ID NO: 134 - DNA, 11 bp, Artificial Sequence
  source: /note=“Description of Artificial Sequence: Synthetic oligonucleotide”
tagtgtatgct

SEQ ID NO: 135 - DNA, 11 bp, Artificial Sequence
  source: /note=“Description of Artificial Sequence: Synthetic oligonucleotide”
cattgtatgct

SEQ ID NO: 136 - DNA, 11 bp, Artificial Sequence
  source: /note=“Description of Artificial Sequence: Synthetic oligonucleotide”
tcttttatgtc

SEQ ID NO: 137 - DNA, 11 bp, Artificial Sequence
  source: /note=“Description of Artificial Sequence: Synthetic oligonucleotide”
tgttttatgtc

SEQ ID NO: 138 - DNA, 11 bp, Artificial Sequence
  source: /note=“Description of Artificial Sequence: Synthetic oligonucleotide”
tagtttatgtc

SEQ ID NO: 139 - DNA, 11 bp, Artificial Sequence
  source: /note=“Description of Artificial Sequence: Synthetic oligonucleotide”
tagtgtatgtc

SEQ ID NO: 140 - DNA, 13 bp, Artificial Sequence
  source: /note=“Description of Artificial Sequence: Synthetic oligonucleotide”
cgacatgaatgtc

SEQ ID NO: 141 - DNA, 13 bp, Artificial Sequence
  source: /note=“Description of Artificial Sequence: Synthetic oligonucleotide”
agggatggatgtc

SEQ ID NO: 142 - DNA, 13 bp, Artificial Sequence
  source: /note=“Description of Artificial Sequence: Synthetic oligonucleotide”
cttgatggatgtc

SEQ ID NO: 143 - DNA, 13 bp, Artificial Sequence
  source: /note=“Description of Artificial Sequence: Synthetic oligonucleotide”
cgtgatgtatgtc

SEQ ID NO: 144 - DNA, 13 bp, Artificial Sequence
  source: /note=“Description of Artificial Sequence: Synthetic oligonucleotide”
ggcgatggatgtc

SEQ ID NO: 145 - DNA, 13 bp, Artificial Sequence
  source: /note=“Description of Artificial Sequence: Synthetic oligonucleotide”
cgtgatggatgtc

SEQ ID NO: 146 - DNA, 13 bp, Artificial Sequence
  source: /note=“Description of Artificial Sequence: Synthetic oligonucleotide”
cgtaatgcatgtc

SEQ ID NO: 147 - DNA, 13 bp, Artificial Sequence
  source: /note=“Description of Artificial Sequence: Synthetic oligonucleotide”
gaacatgaatgtc

SEQ ID NO: 148 - DNA, 13 bp, Artificial Sequence
  source: /note=“Description of Artificial Sequence: Synthetic oligonucleotide”
ggacatgcatgtc

SEQ ID NO: 149 - DNA, 13 bp, Artificial Sequence
  source: /note=“Description of Artificial Sequence: Synthetic oligonucleotide”
aagaatgaatgtc

SEQ ID NO: 150 - DNA, 13 bp, Artificial Sequence
  source: /note=“Description of Artificial Sequence: Synthetic oligonucleotide”
cccgatggatgtc

SEQ ID NO: 151 - DNA, 13 bp, Artificial Sequence
  source: /note=“Description of Artificial Sequence: Synthetic oligonucleotide”
gccgatgtatgtc

SEQ ID NO: 152 - DNA, 13 bp, Artificial Sequence
  source: /note=“Description of Artificial Sequence: Synthetic oligonucleotide”
cgggatgaatgtc

SEQ ID NO: 153 - DNA, 14 bp, Artificial Sequence
  source: /note=“Description of Artificial Sequence: Synthetic oligonucleotide”
gaccatgcatggct

SEQ ID NO: 154 - DNA, 13 bp, Artificial Sequence
  source: /note=“Description of Artificial Sequence: Synthetic oligonucleotide”
aattatgcatggc

SEQ ID NO: 155 - DNA, 13 bp, Artificial Sequence
  source: /note=“Description of Artificial Sequence: Synthetic oligonucleotide”
agacatgtatggc

SEQ ID NO: 156 - DNA, 13 bp, Artificial Sequence
  source: /note=“Description of Artificial Sequence: Synthetic oligonucleotide”
aagaatgtatggc

SEQ ID NO: 157 - DNA, 14 bp, Artificial Sequence
  source: /note=“Description of Artificial Sequence: Synthetic oligonucleotide”
gaccatgcatggct

SEQ ID NO: 158 - DNA, 13 bp, Artificial Sequence
  source: /note=“Description of Artificial Sequence: Synthetic oligonucleotide”
cgcgatggatggc

SEQ ID NO: 159 - DNA, 13 bp, Artificial Sequence
  source: /note=“Description of Artificial Sequence: Synthetic oligonucleotide”
agcgatggatggc

SEQ ID NO: 160 - DNA, 13 bp, Artificial Sequence
  source: /note=“Description of Artificial Sequence: Synthetic oligonucleotide”
gatcatgcatggc

SEQ ID NO: 161 - DNA, 13 bp, Artificial Sequence
  source: /note=“Description of Artificial Sequence: Synthetic oligonucleotide”
cgtaatggatggc

SEQ ID NO: 162 - DNA, 13 bp, Artificial Sequence
  source: /note=“Description of Artificial Sequence: Synthetic oligonucleotide”
aacgatgtatggc

SEQ ID NO: 163 - DNA, 11 bp, Artificial Sequence
  source: /note=“Description of Artificial Sequence: Synthetic oligonucleotide”
ggcaacacggc

SEQ ID NO: 164 - DNA, 504 bp, Artificial Sequence
  source: /note=“Description of Artificial Sequence: Synthetic polynucleotide”
tgaaactaacttacaagttggctagtttgttaaaatacgcgctgcgcttgactcgggaatacaaagaaaacattattccacactttgatcacttgactcgattgcgcgatttaatcgacggcttgattaaaagcgaggatgtacaacgttttaatcgcactaatcgcaatgatttaatttcggcttgcttgcaaatcaacgttcggacgtacttgcccaacgccacgatagatttgcgcaaacaacccaactgtatatattttcgaatttgccaatattgccacttggaggccgacgtgccttcgcccgacgatcattcggtgtacagatacttgtgcgtcgcgtgcggcacgccgctggtcatcgaccacccgctcgacgtgttcggccacacggaggaaggcgtcaacgaactgctcgaggtgcagcgagtcaacgcgggcggggagttgtaggcgtcataactatttattaaataagataatttaaaaaatcgccgttaat

SEQ ID NO: 165 - DNA, 29 bp, Artificial Sequence
  source: /note=“Description of Artificial Sequence: Synthetic oligonucleotide”
ataagataatttaaaaaatcgccgttaat

SEQ ID NO: 166 - DNA, 65 bp, Artificial Sequence
  source: /note=“Description of Artificial Sequence: Synthetic oligonucleotide”
gggcggggagttgtaggcgtcataactatttattaaataagataatttaaaaaatcgccgttaat

SEQ ID NO: 167 - DNA, 92 bp, Artificial Sequence
  source: /note=“Description of Artificial Sequence: Synthetic oligonucleotide”
atcatggagataattaaaatgataaccatctcgcaaataaataagtattttactgttttcgtaacagttttgtaataaaaaaacctataaat

SEQ ID NO: 168 - protein, 724 aa, Artificial Sequence
  source: /note=“Description of Artificial Sequence: Synthetic polypeptide”
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 169 - DNA, 2175 bp, Artificial Sequence
  source: /note=“Description of Artificial Sequence: Synthetic polynucleotide”
atgtcttttgttgatcaccctccagattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctctactactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 170-199 - skipped (30 entries)

SEQ ID NO: 200 - DNA, 101 bp, Artificial Sequence
  source: /note=“Description of Artificial Sequence: Synthetic polynucleotide”
gacctttaattcaacccaacacaatatattatagttaaataagaattattatcaaatcatttgtatattaattaaaatactatactgtaaattacatttta

SEQ ID NO: 201 - DNA, 1866 bp, Artificial Sequence
  source: /note=“Description of Artificial Sequence: Synthetic polynucleotide”
atggcggggttttacgagattgtgattaaggtccccagcgaccttgacgagcatctgcccggcatttctgacagctttgtgaactgggtggccgagaaggagtgggagttgccgccagattctgacttggatctgaatctgattgagcaggcacccctgaccgtggccgagaagctgcagcgcgactttctgacggagtggcgccgtgtgagtaaggccccggaggcccttttctttgtgcaatttgagaagggagagagctacttccacttacacgtgctcgtggaaaccaccggggtgaaatccttagttttgggacgtttcctgagtcagattcgcgaaaaactgattcagagaatttaccgcgggatcgagccgactttgccaaactggttcgcggtcacaaagaccagaaacggcgccggaggcgggaacaaggtggtggacgagtgctacatccccaattacttgctccccaaaacccagcctgagctccagtgggcgtggactaatttagaacagtatttaagcgcctgtttgaatctcacggagcgtaaacggttggtggcgcagcatctgacgcacgtgtcgcagacgcaggagcagaacaaagagaatcagaatcccaattctgacgcgccggtgatcagatcaaaaacttcagccagatacatggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 202 - protein, 621 aa, Artificial Sequence
  source: /note=“Description of Artificial Sequence: Synthetic polypeptide”
MAGFYEIVIKVPSDLDEHLPGISDSFVNWVAEKEWELPPDSDLDLNLIEQAPLTVAEKLQRDFLTEWRRVSKAPEALFFVQFEKGESYFHLHVLVETTGVKSLVLGRFLSQIREKLIQRIYRGIEPTLPNWFAVTKTRNGAGGGNKVVDECYIPNYLLPKTQPELQWAWTNLEQYLSACLNLTERKRLVAQHLTHVSQTQEQNKENQNPNSDAPVIRSKTSARYMELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 203 - DNA, 1194 bp, Artificial Sequence
  source: /note=“Description of Artificial Sequence: Synthetic polynucleotide”
atggagctggtcgggtggctcgtggacaaggggattacctcggagaagcagtggatccaggaggaccaggcctcatacatctccttcaatgcggcctccaactcgcggtcccaaatcaaggctgccttggacaatgcgggaaagattatgagcctgactaaaaccgcccccgactacctggtgggccagcagcccgtggaggacatttccagcaatcggatttataaaattttggaactaaacgggtacgatccccaatatgcggcttccgtctttctgggatgggccacgaaaaagttcggcaagaggaacaccatctggctgtttgggcctgcaactaccgggaagaccaacatcgcggaggccatagcccacactgtgcccttctacgggtgcgtaaactggaccaatgagaactttcccttcaacgactgtgtcgacaagatggtgatctggtgggaggaggggaagatgaccgccaaggtcgtggagtcggccaaagccattctcggaggaagcaaggtgcgcgtggaccagaaatgcaagtcctcggcccagatagacccgactcccgtgatcgtcacctccaacaccaacatgtgcgccgtgattgacgggaactcaacgaccttcgaacaccagcagccgttgcaagaccggatgttcaaatttgaactcacccgccgtctggatcatgactttgggaaggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataa

SEQ ID NO: 204 - protein, 397 aa, Artificial Sequence
  source: /note=“Description of Artificial Sequence: Synthetic polypeptide”
MELVGWLVDKGITSEKQWIQEDQASYISFNAASNSRSQIKAALDNAGKIMSLTKTAPDYLVGQQPVEDISSNRIYKILELNGYDPQYAASVFLGWATKKFGKRNTIWLFGPATTGKTNIAEAIAHTVPFYGCVNWTNENFPFNDCVDKMVIWWEEGKMTAKVVESAKAILGGSKVRVDQKCKSSAQIDPTPVIVTSNTNMCAVIDGNSTTFEHQQPLQDRMFKFELTRRLDHDFGKVTKQEVKDFFRWAKDHVVEVEHEFYVKKGGAKKRPAPSDADISEPKRVRESVAQPSTSDAEASINYADRYQNKCSRHVGMNLMLFPCRQCERMNQNSNICFTHGQKDCLECFPVSESQPVSVVKKAYQKLCYIHHIMGKVPDACTACDLVNVDLDDCIFEQ

SEQ ID NO: 205 - DNA, 2211 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 206 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgatgggactttggcggtgccttttaaggcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 207 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtccgcttaatggtgccgtccatctttatgctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 208 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgtgattctccgaagggttggcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 209 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggcttattctacggatgtgaggatgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 210 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacagattgttatgaattcgttgaaggctcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 211 - DNA, 2232 bp, Artificial Sequence
  source: /note=“Description of Artificial Sequence: Synthetic polynucleotide”
acggctgccgacggttatctacccgattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcacaggctcgggagagtcctcgtgggctgcaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgccgatcctccaacggccttcaacaaggacaagctgaactctttcatcacccagtattctactggccaagtcagcgtggagatcgagtgggagctgcagaaggaaaacagcaagcggtggaacccggagatccagtacacttccaactattacaagtctaataatgttgaatttgctgttaatactgaaggtgtatatagtgaaccccgccccattggcaccagatacctgactcgtaatctgtaa

SEQ ID NO: 212 - DNA, 2211 bp, Artificial Sequence
  source: /note=“Description of Artificial Sequence: Synthetic polynucleotide”
acggctgccgacggttatctacccgattggctcgaggacaacctctctgagggcattcgcgagtggtgggacttgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 213 - DNA, 2175 bp, Artificial Sequence
  source: /note=“Description of Artificial Sequence: Synthetic polynucleotide”
acgagttttgttgatcacccacccgattggttggaagaagttggtgaaggtcttcgcgagtttttgggccttgaagcgggcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 214-216 - skipped (3 entries)

SEQ ID NO: 217 - DNA, 166 bp, Artificial Sequence
  source: /note=“Description of Artificial Sequence: Synthetic polynucleotide”
aaattatcgcaagataaggcgcacgttgattgggtcacccgagtgtacgttgataaagtcacgtgggcacccaacgcgttgataagcatcggtatataagggcctacagtgttctggtaaatcagttgcactgtgctcttcacaggaacactacaagacctacaag

SEQ ID NO: 218 - protein, 204 aa, Artificial Sequence
  source: /note=“Description of Artificial Sequence: Synthetic polypeptide”
METQTQYLTPSLSDSHQQPPLVWELIRWLQAVAHQWQTITRAPTEWVIPREIGIAIPHGWATESSPPAPEPGPCPPTTTTSTNKFPANQEPRTTITTLATAPLGGILTSTDSTATFHHVTGKDSSTTTGDSDPRDSTSSSLTFKSKRSRRMTVRRRLPITLPARFRCLLTRSTSSRTSSARRIKDASRRSQQTSSWCHSMDTSP

SEQ ID NO: 219 - DNA, 648 bp, Artificial Sequence
  source: /note=“Description of Artificial Sequence: Synthetic polynucleotide”
atggagacacagacgcagtacctgacgccatcattgtctgatagccatcagcagcctccgcttgtatgggaacttatacgttggctccaggccgtcgcccatcaatggcagacgatcactagggctcctaccgaatgggtaatccccagagagattggtatcgccatcccccatggctgggctaccgaatcatctcctcccgcccctgagccaggtccttgtcctccgacaactacaacttcgacgaataaattcccggctaaccaagagccacgtactaccatcacaactttggcaacagctccactgggtggaatactcacaagcacagactcgactgctacgttccaccatgtgacaggtaaggatagtagtacaacgaccggcgactctgatccaagggactctacgtcatcgtctttgacttttaagtcgaaaagaagccgtcgtatgactgttcgtcgcaggcttcccattacccttcccgctaggttcagatgcctccttacacgcagtaccagttccagaacttcatcggctaggagaataaaggatgcttctagacgcagccaacaaactagttcatggtgccattccatggacactagcccaagcggagactataaggatgatgacgacaagtaataa

(19) United States
(12) Patent Application Publication
(10) Pub. No.: US 2024/0141377 A1
(43) Pub. Date: May 2, 2024

(54) CONTROLLED EXPRESSION OF VIRAL PROTEINS

(71) Applicant: VOYAGER THERAPEUTICS, INC., Cambridge, MA (US)

(72) Inventors: Christopher NGUYEN, Cambridge, MA (US); Jeffrey Morley SLACK, Cambridge, MA (US); Peter SLADE, Milton, MA (US); Ryan Joseph NISTLER, Boston, MA (US)

(21) Appl. No.: 18/279,354

(22) PCT Filed: Mar. 3, 2022
(86) PCT No.: PCT/US2022/018687

§ 371 Date: Aug. 29, 2023

Related U.S. Application Data
(60) Provisional application No. 63/238,049, filed on Aug. 27, 2021.
(60) Provisional application No. 63/186,408, filed on May 10, 2021.
(60) Provisional application No. 63/155,916, filed on Mar. 3, 2021.

(51) Int. Cl. C12N 15/86; C07K 14/005

(52) U.S. Cl. CPC C12N 15/86; C07K 14/005; C12N 2710/14044; C12N 2750/14122; C12N 2750/14143; C12N 2750/14151

(57) ABSTRACT

The present disclosure describes methods and systems for use in the production of adeno-associated virus (AAV) particles, including recombinant adeno-associated virus (rAAV) particles. In certain embodiments, the production process and system use Spodoptera frugiperda insect cells (such as Sf9 or Sf21) as viral production cells (VPCs).

### RELATED APPLICATIONS

This application claims priority to U.S. Provisional Application 63/155,916 filed on Mar. 3, 2021, U.S. Provisional Application 63/186,408 filed on May 10, 2021, and U.S. Provisional Application 63/238,049 filed on Aug. 27, 2021, the entire contents of which are hereby incorporated by reference.

### REFERENCE TO THE SEQUENCE LISTING

The present application is being filed along with a Sequence Listing in electronic format. The Sequence Listing is provided as a file entitled V2071-707702_SL.txt, created on Aug. 27, 2021, which is 40,305 bytes in size. The information in the electronic format of the sequence listing is incorporated herein by reference in its entirety.

### FIELD OF THE DISCLOSURE

The present disclosure describes methods and systems for use in the production of adeno-associated virus (AAV) particles, including recombinant adeno-associated virus (rAAV) particles. In certain embodiments, the production process and system use Spodoptera frugiperda insect cells (such as Sf9 or Sf21) as viral production cells (VPCs). In certain embodiments, the production process and system use AAV expression constructs, e.g., Baculoviral Expression Vectors (BEVs) and/or Baculoviral Infected Insect Cells (BIICs), in the production of AAV particles (e.g., rAAVs). In certain embodiments, the production process and system allow for the controlled expression of AAV nonstructural (e.g., replication) proteins, such as Rep78 and Rep52.

### BACKGROUND

AAVs have emerged as one of the most widely studied and utilized viral vectors for gene transfer to mammalian cells. See, e.g., Tratschin et al., Mol. Cell Biol., 5(11):3251-3260 (1985) and Grimm et al., Hum. Gene Ther., 10(15):2445-2450 (1999), the contents of which are each incorporated herein by reference in their entireties. AAV vectors, e.g., AAV particles, are promising candidates for therapeutic gene delivery. The design and production of improved AAV particles for this purpose is an active field of study.

There remains a need for improved systems and methods for producing AAV structural (e.g., capsid) proteins and AAV capsids, AAV nonstructural (e.g., replication) proteins, and corresponding AAV vectors (e.g., rAAV particles).

### SUMMARY

The present disclosure pertains at least in part, to compositions and methods for the production of AAV particles and expression of AAV capsid proteins (e.g., VP1, VP2, and/or VP3) and replication proteins (e.g., Rep52 and/or Rep78). The present disclosure also describes AAV expression constructs (e.g., bacmids) and AAV production systems and methods of using the same for the production of recombinant adeno-associated viral (rAAV) particles. In some embodiments, an AAV expression construct described herein demonstrates improved properties over previous AAV expression constructs including improved passage stability, increased AAV viral titers, improved capsid protein ratios, improved capsid quality, and improved AAV capsid potency (e.g., increased transduction efficiency), for AAV capsid proteins of different AAV serotypes, including but not limited to AAV9 capsid proteins and variants thereof.

Accordingly, in some aspects, the present disclosure provides an AAV expression construct comprising (i) at least two Rep-coding regions, each comprising a nucleotide sequence encoding a Rep protein independently chosen from Rep52, Rep40, Rep68, or Rep78 protein, e.g., a Rep52 protein and a Rep78 protein; and (ii) a VP-coding region comprising a nucleotide sequence encoding at least one, two, or three VP proteins, chosen from a VP1 protein, a VP2 protein, a VP3 protein, or a combination thereof, wherein the at least two Rep-coding regions each comprise a different nucleotide sequence and/or is present in different location; wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94; optionally wherein the AAV expression construct is stably maintained for at least 5-10 passages, e.g., at least 5, 6, 7, 8, 9, or 10 passages, in a host cell (e.g., an insect cell). In some embodiments, the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein. In some embodiments, the AAV expression construct comprises a second VP-coding region. In some embodiments, the second VP-coding region comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein). In some embodiments, the second VP-coding region is operably linked to a ctx promoter. In some embodiments, the AAV expression construct comprises a modified Kozak sequence. In some embodiments, the modified Kozak sequence is present at the 5′ end of the VP-coding region.

In another aspect, the present disclosure provides an AAV expression construct comprising a variant baculovirus genome comprising: (i) a first Rep-coding region which is present in the v-cath locus of the variant baculovirus genome; (ii) a second Rep-coding which region is present in the egt locus of the variant baculovirus genome; and (iii) a VP-coding region which is present in the v-cath locus of the variant baculovirus genome.

In another aspect, the present disclosure provides an AAV expression construct comprising a variant baculovirus genome comprising: (i) a first Rep-coding region which is present in the v-cath locus of the variant baculovirus genome and is operably linked to a polh promoter; (ii) a second Rep-coding region which is present in the egt locus of the variant baculovirus genome and is operably linked to a polh promoter; and (iii) a VP-coding region which is present in the v-cath locus of the variant baculovirus genome and is operably linked to a p10 promoter.

In yet another aspect, the present disclosure provides an AAV expression construct comprising a variant baculovirus genome comprising: (i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein); (ii) a second Rep-coding region, which is present in the egt locus of the variant baculovirus genome and comprises a nucleotide sequence encoding a Rep52 protein but not a Rep78 protein; and (iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

In yet another aspect, the present disclosure provides an AAV expression construct comprising a variant baculovirus genome comprising: (i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter; (ii) a second Rep-coding region is present in the egt locus of the variant baculovirus genome and comprises a nucleotide sequence encoding a Rep52 protein but not a Rep78 protein, wherein the second Rep-coding region is operably linked to a polh promoter; and (iii) a VP-coding region which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; optionally wherein the VP-coding region is present in the reverse orientation relative to the first Rep-coding region.

In yet another aspect, the present disclosure provides an AAV expression construct comprising a variant baculovirus genome comprising: (i) a first Rep-coding region, which is present in the v-cath locus of the variant baculovirus genome; (ii) a second Rep-coding region, which is present in the egt locus of the variant baculovirus genome; (iii) a VP-coding region, which is present in the v-cath locus of the variant baculovirus genome; and (iv) a second VP-coding region, which is present in the SOD locus of the variant baculovirus genome.

In yet another aspect, the present disclosure provides an AAV expression construct comprising a variant baculovirus genome comprising: (i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein); (ii) a second Rep-coding region, which is present in the egt locus of the variant baculovirus genome and comprises a nucleotide sequence encoding a Rep52 protein but not a Rep78 protein; (iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and (iv) a second VP-coding region, which is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).

In another aspect, the present disclosure provides an AAV expression construct comprising a variant baculovirus genome comprising: (i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter; (ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter; (iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and (iv) a second VP-coding region, which is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), wherein the second VP-coding region is operably linked to a ctx promoter; optionally wherein, the VP-coding region is present in the reverse orientation relative to the first Rep-coding region.

In another aspect, the present disclosure provides an AAV expression construct comprising a variant baculovirus genome comprising: (i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter; (ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter; and (iii) a VP-coding region, which is present in the v-cath locus of the variant baculovirus genome and is operably linked to a p10 promoter, wherein the VP region comprises: (a) a modified Kozak sequence which is present at the 5′ end of the VP-coding region (e.g., at the start of the VP-coding region), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

In another aspect, the present disclosure provides an AAV payload expression construct comprising a payload coding region comprising a nucleotide sequence encoding a payload wherein the AAV expression construct comprises at least a portion of a baculovirus genome, e.g., variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

In yet another aspect, the present disclosure provides a cell comprising an AAV expression construct described herein and/or an AAV payload construct described herein. In some embodiments, the cell is an insect cell.

In yet another aspect, the present disclosure provides a VP1 protein encoded by an AAV expression construct described herein. In another aspect, the present disclosure provides a VP2 protein encoded by an AAV expression construct described herein. In yet another aspect, the present disclosure provides a VP3 protein encoded by an AAV expression construct described herein. In yet another aspect, the present disclosure provides a Rep78 protein encoded by an AAV expression construct described herein. In yet another aspect, the present disclosure provides a Rep52 protein encoded by an AAV expression construct described herein. In yet another aspect, the present disclosure provides an AAV capsid protein encoded by an AAV expression construct described herein.

In yet another aspect, the present disclosure provides an AAV production system comprising an AAV expression construct described herein and an AAV payload construct described herein. In some embodiments, the AAV production system comprises a viral production cell comprising the AAV expression construct and AAV payload construct.

In yet another aspect, the present disclosure provides a method of producing one, two, three, four, or all of a Rep78 protein, a Rep52 protein, a VP1 protein, a VP protein, and/or a VP3 protein, the method comprising: (i) providing a cell comprising an AAV expression construct described herein; (ii) incubating the cell under conditions suitable to produce the one, two, three, four, or all of the Rep78 protein, the Rep52 protein, the VP1 protein, the VP protein, and/or the VP3 protein.

In yet another aspect, the present disclosure provides a method of producing an AAV particle, the method comprising: (i) providing a cell comprising an AAV expression construct described herein and an AAV payload construct described herein; (ii) incubating the cell under conditions suitable to produce the AAV particle; thereby producing the AAV particle.

In certain embodiments, the present disclosure presents AAV expression constructs for use in controlling the expression of AAV nonstructural (e.g., replication) proteins, such as Rep78 and Rep52, during the production of recombinant adeno-associated viral (rAAV) particles. In certain embodiments, the present disclosure presents AAV expression constructs which comprise: a first Rep-coding region comprising a first open reading frame (ORF) which comprises a start codon and a nucleotide sequence encoding one or more AAV Rep proteins selected from Rep78 and Rep52; and a second Rep-coding region comprising a second ORF which comprises a start codon and a nucleotide sequence encoding one or more AAV Rep proteins selected from Rep78 and Rep52. In certain embodiments, the first Rep-coding region comprises a nucleotide sequence encoding Rep78 only. In certain embodiments, the second Rep-coding region comprises a nucleotide sequence encoding Rep52 only.

In certain embodiments, at least a portion of the first Rep-coding region is codon optimized from a reference Rep-coding nucleotide sequence. In certain embodiments, the first Rep-coding region is codon optimized for an insect cell; optionally a Spodoptera frugiperda insect cell. In certain embodiments, at least a portion of the second Rep-coding region is codon optimized from a reference Rep-coding nucleotide sequence. In certain embodiments, the second Rep-coding region is codon optimized for an insect cell; optionally a Spodoptera frugiperda insect cell.

In certain embodiments, the first Rep-coding region comprises one or more expression control regions which comprise one or more promoter sequences. In certain embodiments, the expression control region of the first Rep-coding region comprises at least one promoter sequence selected from: polh, ΔIE-1, p10, Δp10, and variations or derivatives thereof. In certain embodiments, the expression control region of the first Rep-coding region comprises at least one polh promoter. In certain embodiments, the first Rep-coding region comprises a polh promoter, and the first ORF comprises a nucleotide sequence encoding Rep78 only.

In certain embodiments, the second Rep-coding region comprises one or more expression control regions which comprise one or more promoter sequences. In certain embodiments, the expression control region of the second Rep-coding region comprises at least one promoter sequence selected from: polh, ΔIE-1, p10, Δp10, and variations or derivatives thereof. In certain embodiments, the expression control region of the second Rep-coding region comprises at least one polh promoter. In certain embodiments, the second Rep-coding region comprises a polh promoter, and the second ORF comprises a nucleotide sequence encoding Rep52 only.

In certain embodiments, the first Rep-coding region comprises one or more expression-modifier sequences 5′ of the first ORF. In certain embodiments, the first Rep-coding region comprises one or more expression-modifier sequences 5′ of the first ORF, wherein the one or more expression-modifier sequences decreases translation initiation at the start codon of the first ORF. In certain embodiments, the first Rep-coding region comprises between 3-100 nucleotides between the expression-modifier sequence and the start codon of the first ORF. In certain embodiments, the first Rep-coding region comprises between 3-25 nucleotides or between 3-10 nucleotides between the expression-modifier sequence and the start codon of the first ORF. In certain embodiments, the first Rep-coding region comprises 3 nucleotides between the expression-modifier sequence and the start codon of the first ORF.

In certain embodiments, the second Rep-coding region comprises one or more expression-modifier sequences 5′ of the second ORF. In certain embodiments, the second Rep-coding region comprises one or more expression-modifier sequences 5′ of the second ORF, wherein the one or more expression-modifier sequences decreases translation initiation at the start codon of the second ORF. In certain embodiments, the second Rep-coding region comprises between 3-100 nucleotides between the expression-modifier sequence and the start codon of the second ORF. In certain embodiments, the second Rep-coding region comprises between 3-25 nucleotides or between 3-10 nucleotides between the expression-modifier sequence and the start codon of the second ORF. In certain embodiments, the second Rep-coding region comprises 3 nucleotides between the expression-modifier sequence and the start codon of the second ORF.

In certain embodiments, the one or more expression-modifier sequences comprises a minicistron sequence. In certain embodiments, the minicistron insertion sequence is from a baculovirus gene. In certain embodiments, the minicistron insertion sequence is from a baculovirus gp64 gene. In certain embodiments, the minicistron insertion sequence comprises SEQ ID NO: 4. In certain embodiments, the minicistron insertion sequence comprises SEQ ID NO: 5.

In certain embodiments, the AAV expression construct comprises a recombinant baculovirus genome (i.e., bacmid). In certain embodiments, the first Rep-coding region is located in a first location of the baculovirus genome, and the second Rep-coding region is located in a second location of the baculovirus genome which is different from the first location of the baculovirus genome. In certain embodiments, the first Rep-coding region is located in the Tn7/polh gene region of the baculovirus genome. In certain embodiments, the first Rep-coding region is located in the egt gene region of the baculovirus genome. In certain embodiments, the first Rep-coding region is located in the v-cath gene region of the baculovirus genome. In certain embodiments, the second Rep-coding region is located in the Tn7/polh gene region of the baculovirus genome. In certain embodiments, the second Rep-coding region is located in the egt gene region of the baculovirus genome. In certain embodiments, the second Rep-coding region is located in the v-cath gene region of the baculovirus genome.

In certain embodiments, the first Rep-coding region is located in the Tn7/polh gene region of the baculovirus genome, and the second Rep-coding region is located in the egt gene region of the baculovirus genome. In certain embodiments, the second Rep-coding region is located in the Tn7/polh gene region of the baculovirus genome, and the first Rep-coding region is located in the egt gene region of the baculovirus genome.

In certain embodiments, the first Rep-coding region is located in the v-cath gene region of the baculovirus genome, and the second Rep-coding region is located in the egt gene region of the baculovirus genome. In certain embodiments, the second Rep-coding region is located in the v-cath gene region of the baculovirus genome, and the first Rep-coding region is located in the egt gene region of the baculovirus genome.

In certain embodiments, the AAV expression construct comprises a VP-coding region comprising a first open reading frame (ORF) which comprises a start codon and a nucleotide sequence encoding one or more AAV VP proteins selected from VP1, VP2, VP3, or a combination thereof. In certain embodiments, the VP-coding region is located in the v-cath gene region of the baculovirus genome. In certain embodiments, the AAV expression construct comprises a VP-coding region located in the v-cath gene region of the baculovirus genome and at least one Rep-coding region located in the v-cath gene region of the baculovirus genome. In certain embodiments, the first Rep-coding region comprises a nucleotide sequence encoding Rep78 only, and is located in v-catch gene region of the baculovirus genome. In certain embodiments, the first Rep-coding region comprises a nucleotide sequence encoding Rep78 only, and is located in v-catch gene region of the baculovirus genome, and the second Rep-coding region comprises a nucleotide sequence encoding Rep52 only not located in the v-cath gene region of the baculovirus genome (e.g., in the egt gene region of the baculovirus genome). In certain embodiments, the second Rep-coding region comprises a nucleotide sequence encoding Rep52 only, and is located in v-catch gene region of the baculovirus genome, and the first Rep-coding region comprises a nucleotide sequence encoding Rep78 only not located in the v-cath gene region of the baculovirus genome (e.g., in the egt gene region of the baculovirus genome).

In certain embodiments, the AAV expression construct comprises: (i) a VP-coding region located in the v-cath gene region of the baculovirus genome; (ii) a first Rep-coding region comprising a nucleotide sequence encoding Rep78 only located in v-catch gene region of the baculovirus genome; and (iii) a second Rep-coding region comprising a nucleotide sequence encoding Rep52 only not located in the v-cath gene region of the baculovirus genome (e.g., in the egt gene region of the baculovirus genome). In certain embodiments, the AAV expression construct comprises: (i) a VP-coding region located in the v-cath gene region of the baculovirus genome; (ii) a second Rep-coding region comprising a nucleotide sequence encoding Rep52 only located in v-catch gene region of the baculovirus genome; and (iii) a first Rep-coding region comprising a nucleotide sequence encoding Rep78 only not located in the v-cath gene region of the baculovirus genome (e.g., in the egt gene region of the baculovirus genome).

In certain embodiments, the present disclosure presents an AAV viral production system comprising an AAV expression construct of the present disclosure, and an AAV payload construct which comprises a transgene payload. In certain embodiments, the AAV viral production system comprises an AAV viral production cell which comprises the AAV expression construct and the AAV payload construct. In certain embodiments, the AAV viral production cell is an insect cell. In certain embodiments, the AAV viral production cell is a Sf9 cell or a Sf21 cell.

In certain embodiments, the present disclosure presents methods of expressing AAV Rep78 and Rep52 proteins in an AAV viral production cell. In certain embodiments, the present disclosure presents methods of expressing AAV Rep78 and Rep52 proteins in an AAV viral production cell, comprising: (i) providing an AAV expression construct of the present disclosure; (ii) transfecting the AAV expression construct into an AAV viral production cell; (iii) and exposing the AAV viral production cell to conditions which allow the AAV viral production cell to process the Rep-coding regions into corresponding AAV Rep78 and Rep52 proteins. In certain embodiments, the AAV viral production cell is an insect cell. In certain embodiments, the AAV viral production cell is a Sf9 cell or a Sf21 cell. In certain embodiments, the present disclosure presents Rep78 proteins produced by a method of the present disclosure. In certain embodiments, the present disclosure presents Rep52 proteins produced by a method of the present disclosure.

In certain embodiments, the present disclosure presents method for producing recombinant adeno-associated virus (rAAV) particles in an AAV viral production cell. In certain embodiments, the present disclosure presents method for producing recombinant adeno-associated virus (rAAV) particles in an AAV viral production cell, comprising: (i) providing an AAV viral production system of the present disclosure which comprises an AAV expression construct and an AAV payload construct comprising a nucleotide sequence encoding a transgene payload, wherein the AAV expression construct comprises one or more VP-coding regions which comprise one or more nucleotide sequences encoding VP1, VP2 and VP3 capsid proteins, and one or more nucleotide sequences encoding Rep78 and Rep52 proteins; (ii) transfecting the AAV viral production system into an AAV viral production cell; and (iii) exposing the AAV viral production cell to conditions which allow the AAV viral production cell to process the AAV expression construct and the AAV payload construct into rAAV particles. In certain embodiments, the method further comprises (iv) collecting the rAAV particles from the AAV viral production cell. In certain embodiments, the AAV viral production cell is an insect cell. In certain embodiments, the AAV viral production cell is a Sf9 cell or a Sf21 cell.

In certain embodiments, the present disclosure presents recombinant adeno-associated virus (rAAV) particles produced by methods of the present disclosure. In certain embodiments, the present disclosure presents pharmaceutical compositions comprising rAAV particles of the present disclosure and a pharmaceutically acceptable excipient.

Those skilled in the art will recognize or be able to ascertain using no more than routine experimentation, many equivalents to the specific embodiments of the invention described herein. Such equivalents are intended to be encompassed by the following enumerated embodiments.

### Enumerated Embodiments

1. An AAV expression construct comprising:
(i) at least two Rep-coding regions, each comprising a nucleotide sequence encoding a Rep protein independently chosen from Rep52, Rep40, Rep68, or Rep78 protein, e.g., a Rep52 protein and a Rep78 protein; and
(ii) a VP-coding region comprising a nucleotide sequence encoding at least one, two, or three VP proteins, chosen from a VP1 protein, a VP2 protein, a VP3 protein, or a combination thereof,
wherein the at least two Rep-coding regions each comprise a different nucleotide sequence and/or is present in different location;
wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94;
optionally wherein the AAV expression construct is stably maintained for at least 5-10 passages, e.g., at least 5, 6, 7, 8, 9, or 10 passages, in a host cell (e.g., an insect cell).
   2. The AAV expression construct of embodiment 1, wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   3. The AAV expression construct of embodiment 1 or 2, wherein the variant baculovirus genome comprises a nucleotide sequence or a portion thereof from a baculovirus genome selected from Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV).
   4. The AAV expression construct of any one of embodiments 1-3, wherein the variant baculovirus genome comprises a nucleotide sequence or a portion thereof from the AcMNPV (e.g., AcMNPV E2) baculovirus genome.
   5. The AAV expression construct of any one of embodiments 1-4, wherein the disruption results in inactivation of the non-essential gene (e.g., auxiliary and/or per os infectivity factor gene) or the regulatory region of the non-essential gene (e.g., promoter modification or insertion of heterologous DNA adjacent to non-essential gene).
   6. The AAV expression construct of any one of embodiments 1-5, wherein the disruption of the at least two non-essential genes is or comprises an insertion, deletion, substitution, or mutation (e.g., frame-shift mutation).
   7. The AAV expression construct of any one of embodiments 1-6, wherein the disruption of one or both of the at least two non-essential genes is present in the regulatory region of the non-essential gene (e.g., a promoter modification or insertion of heterologous DNA adjacent to non-essential gene).
   8. The AAV expression construct of any one of embodiments 1-7, wherein the variant baculovirus genome comprises a disruption of at least three, four, five, six, seven, eight, nine, or ten non-essential genes (e.g., auxiliary and/or per os infectivity factor genes), wherein the at least three, four, five, six, seven, eight, nine, or ten non-essential genes are independently chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   9. The AAV expression construct of any one of embodiments 1-8, wherein the at least two non-essential genes comprise:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.
   10. The AAV expression construct of any one of embodiments 1-9, wherein the disruption comprises a deletion of a chiA gene, a v-cath gene, a p26 gene, a p10 gene, and/or a p74 gene, or a portion thereof.
   11. The AAV expression construct of any one of embodiments 1-10, wherein the disruption comprises an insertion of a heterologous sequence in the non-essential gene or adjacent region.
   12. The AAV expression construct of any one of embodiments 1-11, wherein the disruption comprises a one or more mutations in the non-essential gene or adjacent region.
   13. The AAV expression construct of any one of embodiments 1-12, wherein one or both of the at least two non-essential genes are present near (e.g., downstream or upstream) of a homologous repeat region 5 (hr5).
   14. The AAV expression construct of any one of embodiments 1-13, wherein the at least two Rep-coding regions each comprise a different nucleotide sequence and is present in different locations in the variant baculovirus genome.
   15. The AAV expression construct of any one of embodiments 1-14, wherein the at least two Rep-coding regions comprise a first Rep-coding region and a second Rep-coding region.
   16. The AAV expression construct of embodiment 15, wherein the first Rep-coding region comprises a first a first open reading frame (ORF) comprising a start codon and a nucleotide sequence encoding a Rep78 protein and the second Rep-coding region comprises a second ORF comprising a start codon and a nucleotide sequence encoding a Rep52 protein.
   17. The AAV expression construct of embodiment 15 or 16, wherein the first Rep-coding region, the second Rep-coding region, or both comprises an ATG start codon (e.g., a canonical start codon).
   18. The AAV expression construct of embodiment 15 or 16, wherein the first Rep-coding region, the second Rep-coding region, or both comprises an ACG start codon, a CTG start codon, a TTG start codon, or a GTG start codon (e.g., a non-canonical start codon).
   19. The AAV expression construct of any one of embodiments 15-18, wherein the first Rep-coding region comprises a nucleotide sequence encoding a Rep78 protein.
   20. The AAV expression construct of any one of embodiments 15-19, wherein the first Rep-coding region comprises a nucleotide sequence encoding primarily a Rep78 protein, e.g., at least 50%, 60%, 70%, 80%, 90% or more Rep78 protein relative to a Rep52 protein.
   21. The AAV expression construct of any one of embodiments 15-20, wherein the first Rep-coding region comprises a nucleotide sequence encoding a Rep78 protein only.
   22. The AAV expression construct of any one of embodiments 15-19, wherein the first Rep-coding region comprises a nucleotide sequence encoding a Rep78 protein but not a Rep52 protein.
   23. The AAV expression construct of any one of embodiments 15-22, wherein the second Rep-coding comprises a nucleotide sequence encoding a Rep52 protein.
   24. The AAV expression construct of any one of embodiments 15-23, wherein the second Rep-coding comprises a nucleotide sequence encoding primarily a Rep52 protein, e.g., at least 50%, 60%, 70%, 80%, 90% or more Rep52 protein relative to a Rep78 protein.
   25. The AAV expression construct of any one of embodiments 15-24, wherein the second Rep-coding comprises a nucleotide sequence encoding a Rep52 protein only.
   26. The AAV expression construct of any one of embodiments 15-23, wherein the second Rep-coding comprises a nucleotide sequence encoding a Rep52 protein but not a Rep78 protein.
   27. The AAV expression construct of any one of embodiments 15-26, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) the second Rep-coding region comprises a nucleotide sequence encoding a Rep52 protein but not a Rep78 protein.
   28. The AAV expression construct of any one of embodiments 15-27, wherein the first Rep-coding region comprises the nucleotide sequence of SEQ ID NO: 201, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 201; or a nucleotide sequence having at least 10, 20, 50, 100, 150, 200, 250, 300, 350, 400, or 450 but no more than 500 modifications (e.g., substitutions) relative to SEQ ID NO: 201.
   29. The AAV expression construct of any one of embodiments 15-28, wherein the first Rep-coding region encodes the amino acid sequence of SEQ ID NO: 202; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 202; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 202.
   30. The AAV expression construct of any one of embodiments 15-29, wherein the second Rep-coding region comprises the nucleotide sequence of SEQ ID NO: 203, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 203; or a nucleotide sequence having at least 10, 20, 50, 100, 150, 200, 250, 300, 350, 400, or 450 but no more than 500 modifications (e.g., substitutions) relative to SEQ ID NO: 203.
   31. The AAV expression construct of any one of embodiments 15-30, wherein the second Rep-coding region encodes the amino acid sequence of SEQ ID NO: 204; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 204; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 204.
   32. The AAV expression construct of any one of embodiments 15-31, wherein the nucleotide sequence of the first Rep-coding region, the second Rep-coding region, or both are codon optimized for an insect cell, optionally a Spodoptera frugiperda insect cell (e.g., an Sf9 insect cell).
   33. The AAV expression construct of any one of embodiments 15-32, wherein the nucleotide sequence of the first Rep-coding region is operably linked to a first promoter.
   34. The AAV expression construct of any one of embodiments 15-33, wherein the nucleotide sequence of the second Rep-coding region is operably linked to a second promoter.
   35. The AAV expression construct of embodiment 33 or 34, wherein the first promoter, the second promoter, or both the first promoter and the second promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   36. The AAV expression construct of any one of embodiments 33-35, wherein the first promoter, the second promoter, or both the first promoter and the second promoter is chosen from a polyhedrin (polh) promoter, a p10 promoter, a conotoxin (ctx) promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   37. The AAV expression construct of embodiment 33-36, wherein the first promoter and the second promoter are the same.
   38. The AAV expression construct of embodiment 33-36, wherein the first promoter and the second promoter are different.
   39. The AAV expression construct of embodiment 33-37, the first promoter and the second promoter are each a polh promoter.
   40. The AAV expression construct of embodiment 39, wherein the polh promoter comprises the nucleotide sequence of SEQ ID NO: 167; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167; or a nucleotide sequence comprising at least one, two, three, four, five, six, or seven, but no more than ten modifications (e.g., substitutions) relative to SEQ ID NO: 167.
   41. The AAV expression construct of any one of embodiments 15-40, wherein the first Rep-coding region or the second Rep-coding region comprises an expression-modifier sequence which decreases transcription initiation of the first Rep-coding region.
   42. The AAV expression construct of embodiment 41, wherein the expression-modifier sequence comprises a minicistron sequence.
   43. The AAV expression construct of any one of embodiments 15-42, the first Rep-coding region comprises a minicistron sequence, optionally wherein the minicistron sequence is present at the 5′ end of the first Rep-coding region.
   44. The AAV expression construct of any one of embodiments 41-43, the first Rep-coding region comprises between 3-100 nucleotides between the expression-modifier sequence and the start codon of the first ORF; optionally between 3-25 nucleotides, between 3-10 nucleotides, or 3 nucleotides between the expression-modifier sequence and the start codon of the first ORF.
   45. The AAV expression construct of any one of embodiments 42-44, wherein the minicistron sequence is from a baculovirus gene; optionally a baculovirus gp64 gene.
   46. The AAV expression construct of any one of embodiments 42-45, wherein the minicistron sequence comprises SEQ ID NO: 4 or SEQ ID NO: 5; a nucleotide sequence at least 70%, 75%, 80%, 85%, 90%, 95%, 96%, 97%, 98%, or 99% identical to SEQ ID NO: 4 or 5; a nucleotide sequence comprising one, two, or three modifications (e.g., substitutions), but no more than four modifications (e.g., substitutions) relative to SEQ ID NO: 4 or 5; or a nucleotide sequence comprising one, two, or three, but no more than four different nucleotides relative to SEQ ID NO: 4 or 5.
   47. The AAV expression construct of any one of embodiments 15-46, which comprises in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   48. The AAV expression construct of any one of embodiments 15-46, which comprises in 5′ to 3′ order: a polh promoter, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein).
   49. The AAV expression construct of any one of embodiments 15-48, which comprises in 5′ to 3′ order: a polh promoter and the second Rep-coding region comprising a nucleotide sequence encoding a Rep52 protein but not a Rep78 protein.
   50. The AAV expression construct of any one of embodiments 15-49, which comprises:
(i) in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   51. The AAV expression construct of any one of embodiments 15-49, wherein:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.
   52. The AAV expression construct of any one of embodiments 15-51, wherein:
(i) the first Rep-coding region is present in first location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94; and
(ii) the second Rep-coding region is present in a second location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94;
wherein the first locus and the second locus are different.
   53. The AAV expression construct of embodiment 15-52, wherein the first Rep-coding region is present in v-cath locus and the second Rep-coding region is present in the egt locus.
   54. The AAV expression construct of embodiment 15-52, wherein the first Rep-coding region is present in Tn7/polh locus and the second Rep-coding region is present in the egt locus.
   55. The AAV expression construct of any one of embodiments 15-53, wherein the first Rep-coding region is present in the v-cath locus of the variant baculovirus genome and is operably linked to a polh promoter, and the second Rep-coding region is present in the egt locus of the variant baculovirus genome and is operably linked to a polh promoter.
   56. The AAV expression construct of any one of embodiments 15-53 or 55, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) the second Rep-coding region comprises a nucleotide sequence encoding a Rep52 protein but not a Rep78 protein, wherein the second Rep-coding region is present in the egt locus of the variant baculovirus genome.
   57. The AAV expression construct of any one of embodiments 15-53 or 55-56, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome and is operably linked to a polh promoter; and
(ii) the second Rep-coding region comprises a nucleotide sequence encoding a Rep52 protein but not a Rep78 protein, wherein the second Rep-coding region is present in the egt locus of the variant baculovirus genome and is operably linked to a polh promoter.
   58. The AAV expression construct of any one of embodiments 15-53 or 55-57, which comprises:
(i) in 5′ to 3′ order a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   59. The AAV expression construct of any one of embodiments 15-53 or 55-58, which comprises:
(i) in 5′ to 3′ order: a polh promoter and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) in 5′ to 3′ order: a polh promoter and the second Rep-coding region comprising a nucleotide sequence encoding a Rep52 protein but not a Rep78 protein, wherein the second Rep-coding region is present in the egt locus of the variant baculovirus genome.
   60. The AAV expression construct of any one of embodiments 1 or 3-59, wherein the VP-coding region comprises a nucleotide sequence encoding:
(i) primarily a VP1 protein, e.g., at least 50%, 60%, 70%, 80%, 90% or more VP1 protein relative to a VP2 protein and/or a VP3 protein;
(ii) a VP1 protein only;
(iii) a VP1 protein, but not a VP2 protein or a VP3 protein;
(iv) primarily a VP2 protein, e.g., at least about 50%, 60%, 70%, 80%, 90% or more VP2 protein relative to a VP1 protein and/or a VP3 protein;
(v) a VP2 protein only;
(vi) a VP2 protein, but not a VP1 protein or a VP3 protein;
(vii) a VP3 protein only;
(viii) a VP3 protein, but not a VP1 protein or a VP2 protein;
(ix) a VP1 protein and a VP2 protein, but not a VP3 protein;
(x) a VP1 protein and a VP3 protein, but not a VP2 protein;
(xi) a VP2 protein and a VP3 protein, but not a VP1 protein;
(xii) a VP1 protein, a VP2 protein, and a VP3 protein.
   61. The AAV expression construct of any one of embodiments 1-60, wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   62. The AAV expression construct of any one of embodiments 1-61, wherein the VP-coding region comprises a single polycistronic ORF encoding a VP1 protein, a VP2 protein, and a VP3 protein.
   63. The AAV expression construct of any one of embodiments 1-62, wherein the ORF encoding the VP1 protein comprises an ACG start codon, the ORF encoding the VP2 protein comprises an ACG start codon, and the ORF encoding the VP3 protein comprises an ATG start codon.
   64. The AAV expression construct of any one of embodiments 1-62, wherein the ORF encoding the VP1 protein comprises an ATG start codon, the ORF encoding the VP2 protein comprises an ACG start codon, and the ORF encoding the VP3 protein comprises an ATG start codon.
   65. The AAV expression construct of any one of embodiments 1-64, wherein the VP-coding region encodes an AAV1 capsid protein, an AAV2 capsid protein, an AAV3 capsid protein, an AAV4 capsid protein, an AAVS capsid protein, an AAV6 capsid protein, an AAV8 capsid protein, an AAV9 capsid protein, an AAVrh10 capsid protein or a variant of any of the aforesaid capsid proteins.
   66. The AAV expression construct of any one of embodiments 1-65, wherein the VP-coding region encodes an AAVS capsid protein or variant thereof, or an AAV9 capsid protein or variant thereof.
   67. The AAV expression construct of any one of embodiments 1-66, wherein the VP-coding region encodes a VP1 protein comprising the amino acid sequence of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences.
   68. The AAV expression construct of any one of embodiments 1-67, wherein the VP-coding region encodes a VP2 protein e.g., a fragment or a portion, of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences, optionally wherein the VP2 protein comprises amino acids 138-736 or SEQ ID NOs: 71 or 46-48; amino acids 138-743 of SEQ ID NOs: 52, 53, 54, 56, 60, 61, 64, 66, 68; or amino acids 137-724 of SEQ ID NO: 168.
   69. The AAV expression construct of any one of embodiments 1-68, wherein the VP-coding region encodes a VP3 protein e.g., a fragment or a portion, of any of SEQ ID NOs: 46, 47, 48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences, optionally wherein the VP3 protein comprises amino acids 203-736 of SEQ ID NOs: 71 or 46-48; amino acids 203-743 of SEQ ID NOs: 52, 53, 54, 56, 60, 61, 64, 66, 68; or amino acids 193-724 of SEQ ID NO: 168.
   70. The AAV expression construct of any one of embodiments 1-69, wherein the VP-coding region comprises the nucleotide sequence of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences.
   71. The AAV expression construct of any one of embodiments 1-70, wherein the VP-coding region comprises a nucleotide sequence encoding a VP2 protein e.g., a fragment or a portion, of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences, optionally wherein the nucleotide sequence encoding the VP2 protein comprises nucleotides 412-2211 of SEQ ID NOs: 43-45, 72, 205, or 212; nucleotides 412-2232 of SEQ ID NOs: 49-51, 57-59, 62, 63, 65, 67, 69, 72, or 206-211; or nucleotides 409-2175 of SEQ ID NO: 169 or 213.
   72. The AAV expression construct of any one of embodiments 1-71, wherein the VP-coding region comprises a nucleotide sequence encoding a VP3 protein e.g., a fragment or a portion, of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences, optionally wherein the nucleotide sequence encoding the VP3 protein comprises nucleotides 607-2211 of SEQ ID NOs: 43-45, 72, 205, or 212; nucleotides 607-2232 of SEQ ID NOs: 49-51, 57-59, 62, 63, 65, 67, 69, 72, or 206-211; or nucleotides 577-2175 of SEQ ID NO: 169 or 213.
   73. The AAV expression construct of any one of embodiments 1-72, wherein the nucleotide sequence of the VP-coding region is codon optimized for an insect cell, optionally a Spodoptera frugiperda insect cell (e.g., an Sf9 insect cell).
   74. The AAV expression construct of any one of embodiments 1-73, wherein nucleotide sequence of the VP-coding region is operably linked to a promoter.
   75. The AAV expression construct of embodiment 74, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   76. The AAV expression construct of embodiment 74 or 75, wherein the promoter is chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   77. The AAV expression of any one of embodiments 74-76, wherein the promoter is a p10 promoter.
   78. The AAV expression construct of embodiment 77, wherein the p10 promoter comprises the nucleotide sequence of SEQ ID NO: 200; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 200; or a nucleotide sequence comprising at least one, two, three, four, five, six, or seven, but no more than ten modifications (e.g., substitutions) relative to SEQ ID NO: 200.
   79. The AAV expression construct of any one embodiments 1-78, which comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   80. The AAV expression construct of any one of embodiments 15-79, wherein the VP-coding region is present in a different location in the variant baculovirus genome than the location of the first Rep-coding region, the second Rep-coding region or both.
   81. The AAV expression construct of any one of embodiments 15-79, wherein the VP-coding region is present in the same location of the variant baculovirus genome as the first Rep-coding region.
   82. The AAV expression construct of any one of embodiments 15-81, wherein the VP-coding region is present in the reverse orientation relative to the first Rep-coding region.
   83. The AAV expression construct of any one of embodiments 15-79, 81, or 82, wherein the VP-coding region is present in the same location of the variant baculovirus genome as the first Rep-coding region and is present in the reverse orientation relative to the first Rep-coding region.
   84. The AAV expression construct of any one of embodiments 1-83, wherein the VP-coding region is present in a location in variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   85. The AAV expression construct of any one of embodiments 1-84, wherein the VP-coding region is present in the v-cath gene locus of the variant baculovirus genome.
   86. The AAV expression construct of any one of embodiments 1-84, wherein the VP-coding region is present in the Tn7/polh gene locus of the variant baculovirus genome.
   87. The AAV expression construct of any one of embodiments 1-85, wherein the VP-coding region is present in the v-cath gene locus of the variant baculovirus genome and wherein the VP-coding region is operably linked to a p10 promoter.
   88. The AAV expression construct of any one of embodiments 1-85 or 87, wherein the VP-coding region is present in the v-cath gene locus of the variant baculovirus genome and wherein the VP-coding region comprises a single polycistronic ORF encoding a VP1 protein, a VP2 protein, and a VP3 protein.
   89. The AAV expression construct of any one of embodiments 1-85, 87, or 88, wherein the VP-coding region is present in the v-cath gene locus of the variant baculovirus genome and wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   90. The AAV expression construct of any one of embodiments 1-85 or 87-89, wherein the VP-coding region is present in the v-cath gene locus of the variant baculovirus genome and wherein the VP-coding region:
(i) comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(ii) is operably linked to a p10 promoter.
   91. The AAV expression construct of any one of embodiments 1-85 or 87-90, which comprises in 5′ to 3′ order, a p10 promoter and the VP-coding region comprising a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, wherein the VP-coding region is present in the v-cath gene locus of the variant baculovirus genome.
   92. The AAV expression construct of any one of embodiments 1-85 or 87-91, wherein the VP-coding region is present in the v-cath gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon; and
(ii) is operably linked to a p10 promoter.
   93. The AAV expression construct of any one of embodiments 1-85 or 87-92, wherein the VP-coding region is present in the v-cath gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon; and
(ii) is operably linked to a p10 promoter.
   94. The AAV expression construct of any one of embodiments 1-85 or 87-93, which comprises in 5′ to 3′ order a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ACG start codon, the ORF encoding the VP2 protein comprises an ACG start codon, and the ORF encoding the VP3 protein comprises an ATG start codon, wherein the VP-coding region is present in the v-cath gene locus of the variant baculovirus genome; and.
   95. The AAV expression construct of any one of embodiments 1-85 or 87-93, which comprises in 5′ to 3′ order, a p10 promoter; and the VP-coding region comprising a nucleotide sequence comprising a single polycistronic ORF encoding a VP1 protein, a VP2 protein, and a VP3 protein, wherein the ORF encoding the VP1 protein comprises an ATG start codon, the ORF encoding the VP2 protein comprises an ACG start codon, and the ORF encoding the VP3 protein comprises an ATG start codon, wherein the VP-coding region is present in the v-cath gene locus of the variant baculovirus genome.
   96. The AAV expression construct of any one of embodiments 1-95, which further comprises a second VP-coding region.
   97. The AAV expression construct of embodiment 96, wherein the second VP-coding region comprises a nucleotide sequence encoding a VP1 protein.
   98. The AAV expression construct of embodiment 96 or 97, wherein the second VP-coding region comprises a nucleotide sequence encoding primarily a VP1 protein, e.g., at least 50%, 60%, 70%, 80%, 90% or more VP1 protein relative to a VP2 protein and/or a VP3 protein.
   99. The AAV expression construct of any one of embodiments 96-98, wherein the second VP-coding region comprises a nucleotide sequence encoding a VP1 protein only.
   100. The AAV expression construct of embodiment 96 or 97, wherein the second VP-coding region comprises a nucleotide sequence encoding a VP1 protein but not a VP2 protein or a VP3 protein.
   101. The AAV expression construct of any one of embodiments 96-99, wherein the second VP-coding region comprises a single ORF, comprising a start codon and a nucleotide sequence encoding primarily a VP1 protein, e.g., at least 50%, 60%, 70%, 80%, 90% or more VP1 protein relative to a VP2 protein and/or a VP3 protein.
   102. The AAV expression construct of embodiment 96-99 or 101, wherein the second VP-coding region comprises a single ORF, comprising a start codon and a nucleotide sequence encoding a VP1 protein.
   103. The AAV expression construct of embodiment 96, 97, or 99, wherein the second VP-coding region comprises a single ORF, comprising a start codon and a nucleotide sequence encoding a VP1 protein, but not a VP2 protein or a VP3 protein.
   104. The AAV expression construct of embodiment 101-103, wherein the ORF comprises an ATG start codon (e.g., a canonical start codon).
   105. The AAV expression construct of embodiment 101-103, wherein the ORF comprises an ACG start codon, a CTG start codon, a TTG start codon, or GTG start codon (e.g., a non-canonical start codon).
   106. The AAV expression construct of any one of embodiments 96-105, wherein the second VP-coding region encodes an AAV1 capsid protein, an AAV2 capsid protein, an AAV3 capsid protein, an AAV4 capsid protein, an AAVS capsid protein, an AAV6 capsid protein, an AAV8 capsid protein, an AAV9 capsid protein, an AAVrh10 capsid protein or a variant of any of the aforesaid capsid proteins.
   107. The AAV expression construct of any one of embodiments 96-106, wherein the second VP-coding region encodes an AAVS capsid protein or variant thereof, or an AAV9 capsid protein or variant thereof.
   108. The AAV expression construct of any one of embodiments 96-107, wherein the second VP-coding region encodes a VP1 protein comprising the amino acid sequence of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences.
   109. The AAV expression construct of embodiment 96-108, wherein the second VP-coding region comprises the nucleotide sequence of SEQ ID NO: 43, 49, 57, 62, 63, 65, 67, 69, 72, 169, or 205-213, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 43, 49, 57, 62, 63, 65, 67, 69, 72, 169, or 205-213; or a nucleotide sequence having at least 10, 20, 50, 100, 150, 200, 250, 300, 350, 400, or 450 but no more than 500 modifications (e.g., substitutions) relative to SEQ ID NO: 43, 49, 57, 62, 63, 65, 67, 69, 72, 169, or 205-213.
   110. The AAV expression construct of any one of embodiments 96-109, wherein the nucleotide sequence of the second VP-coding region is codon optimized for an insect cell, optionally a Spodoptera frugiperda insect cell (e.g., an Sf9 insect cell).
   111. The AAV expression construct of any one of embodiments 96-110, wherein nucleotide sequence of the second VP-coding region is operably linked to a promoter.
   112. The AAV expression construct of embodiment 111, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
   113. The AAV expression construct of embodiment 111 or 112, wherein the promoter is chosen from a polh promoter, a p10 promoter, a ctx promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
   114. The AAV expression of any one of embodiments 111-113, wherein the promoter is a ctx promoter.
   115. The AAV expression of embodiment 113 or 114, wherein the ctx promoter comprises the promoter region of the ctx gene (e.g., AcORF3) and the 5′ UTR of the ctx gene.
   116. The AAV expression construct of embodiment 113-115, wherein the ctx promoter comprises the nucleotide sequence of any of SEQ ID NOs: 164-166; a nucleotide sequence at least 70%, 75%, 80%, 85%, 90%, 95%, 96%, 97%, 98%, or 99% identical to any of SEQ ID NOs: 164-166; a nucleotide sequence comprising at least one, two, three, four, five, six, or seven, but no more than ten different nucleotides relative to any of SEQ ID NOs: 164-166; or a nucleotide sequence comprising at least one, two, three, four, five, six, or seven, but no more than ten modifications (e.g., substitutions) relative to any of SEQ ID NOs: 164-166.
   117. The AAV expression construct of embodiment 113-116, wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   118. The AAV expression construct of any one of embodiments 96-117, which comprises in 5′ to 3′ order: a ctx promoter and the second VP-coding region comprising a nucleotide sequence encoding primarily a VP1 protein, e.g., at least 50%, 60%, 70%, 80%, 90% or more VP1 protein relative to a VP2 protein and or a VP3 protein (e.g., but not a VP2 protein or a VP3 protein).
   119. The AAV expression construct of any one of embodiments 96-118, which comprises in 5′ to 3′ order, a ctx promoter and the second VP-coding region comprising a nucleotide sequence encoding a VP1 protein only.
   120. The AAV expression construct of any one of embodiments 96-119, which comprises in 5′ to 3′ order, a ctx promoter and the second VP-coding region comprising a nucleotide sequence encoding a VP1 protein but not a VP2 protein or a VP3 protein.
   121. The AAV expression construct of any one of embodiments 96-120, wherein the second VP-coding region is present at a different location in the variant baculovirus genome than one, two, or all of the first Rep-coding region, the second Rep-coding region, or the VP-coding region.
   122. The AAV expression construct of any one of embodiments 96-121, wherein the second VP-coding region is present at a location in the variant baculovirus genome chosen from chiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
   123. The AAV expression construct of any one of embodiments 96-122, wherein the second VP-coding region is present in the SOD gene locus of the variant baculovirus genome.
   124. The AAV expression construct of any one of embodiments 96-123, wherein the second VP-coding region is present in the SOD gene locus of the variant baculovirus genome and is operably linked to a ctx promoter, optionally wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   125. The AAV expression construct of any one of embodiments 96-124, wherein the second VP-coding region is present in the SOD gene locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   126. The AAV expression construct of any one of embodiments 96-125, wherein the second VP-coding region is present in the SOD gene locus of the variant baculovirus genome and wherein the second VP-coding region:
(i) comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); and
(ii) is operably linked to a ctx promoter, optionally wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.
   127. The AAV expression construct of any one of embodiments 96-126, which comprises in 5′ to 3′ order, a ctx promoter; and the second VP-coding region comprising a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); wherein the second VP-coding region is present in the SOD gene locus of the variant baculovirus genome.
   128. The AAV expression construct of any one of embodiments 96-127, which comprises in 5′ to 3′ order, a ctx promoter; and a single ORF, comprising a start codon and a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein); wherein the second VP-coding region is present in the SOD gene locus of the variant baculovirus genome.
   129. The AAV expression construct of any one of embodiments 1-128, which further comprises a modified Kozak sequence.
   130. The AAV expression construct of embodiment 129, wherein the modified Kozak sequence is capable of modulating expression, e.g., increasing expression, of a protein encoded by a gene that is immediately downstream of the modified Kozak sequence.
   131. The AAV expression construct of embodiment 129 or 130, wherein the modified Kozak sequence comprises a start codon for the translation of a protein encoded by a gene that is immediately downstream of the modified Kozak sequence.
   132. The AAV expression construct of any one of embodiments 129-131, wherein the modified Kozak comprises the nucleotide sequence of sequence of any one of SEQ ID NOs: 32-42, or a nucleotide sequence comprising no more than one, two, or three different nucleotides relative to SEQ ID NOs: 32-42.
   133. The AAV expression construct of any one of embodiments 129-132, wherein nucleotide sequence encoding the modified Kozak comprises the nucleotide sequence of any one of SEQ ID NOs: 21-31, or a nucleotide sequence comprising no more than one, two, or three different nucleotides relative to SEQ ID NOs: 21-31.
   134. The AAV expression construct of any one of embodiments 129-131, wherein the modified Kozak sequence comprises the nucleotide sequence of any one of SEQ ID NOs: 118-162, or a nucleotide sequence comprising no more than one, two, or three different nucleotides relative to SEQ ID NOs: 118-162.
   135. The AAV expression construct of any one of embodiments 129-131 or 134, wherein the nucleotide sequence encoding the modified Kozak sequence comprises the nucleotide sequence of any one of SEQ ID NOs: 73-117, or a nucleotide sequence comprising no more than one, two, or three different nucleotides relative to SEQ ID NOs: 73-117.
   136. The AAV expression construct of any one of embodiments 129-133, wherein the modified Kozak sequence comprises the nucleotide sequence of SEQ ID NO: 33, or a nucleotide sequence comprising no more than one, two, or three different nucleotides relative to SEQ ID NO: 33.
   137. The AAV expression construct of any one of embodiments 129-133 or 136, wherein nucleotide sequence encoding the modified Kozak sequence comprises the nucleotide sequence of SEQ ID NO: 22, or a nucleotide sequence comprising no more than one, two, or three different nucleotides relative to SEQ ID NO: 22.
   138. The AAV expression construct of any one of embodiments 129-133, wherein the modified Kozak sequence comprises the nucleotide sequence of SEQ ID NO: 32, or a nucleotide sequence comprising no more than one, two, or three different nucleotides relative to SEQ ID NO: 32.
   139. The AAV expression construct of any one of embodiments 129-133 or 138, wherein nucleotide sequence encoding the modified Kozak sequence comprises the nucleotide sequence of SEQ ID NO: 21, or a nucleotide sequence comprising no more than one, two, or three different nucleotides relative to SEQ ID NO: 21.
   140. The AAV expression construct of any one of embodiments 129-139, wherein the modified Kozak sequence is present at the 5′ end of the VP-coding region, e.g., at the start of the VP-coding region encoding the VP1 protein (e.g., the ORF encoding the VP1 protein).
   141. The AAV expression construct of any one of embodiments 129-140, wherein the modified Kozak sequence comprises the start codon of the ORF encoding the VP1 protein.
   142. The AAV expression construct of any one of embodiments 129-141, wherein the start codon comprises an ATG.
   143. The AAV expression construct of any one of embodiments 129-133 or 136-142, wherein the modified Kozak sequence and the VP-coding region comprises the nucleotide sequence of SEQ ID NO: 44, 45, 50, 51, 58, or 59, or a nucleotide sequence at least 70%, 75%, 80%, 85%, 90%, 95%, 96%, 97%, 98%, or 99% identical to the aforesaid sequences.
   144. The AAV expression construct of any one of embodiments 129-133, 136, 137, 140-143, wherein the VP-coding region comprising a modified Kozak sequence encodes a VP1 protein comprising the amino acid sequence of SEQ ID NOs: 47, 53, or 61, or an amino acid sequence at least 70%, 75%, 80%, 85%, 90%, 95%, 96%, 97%, 98%, or 99% identical to the aforesaid sequences.
   145. The AAV expression construct of any one of embodiments 129-133 or 138-142, wherein the VP-coding region comprising a modified Kozak sequence encodes a VP1 protein comprising the amino acid sequence of SEQ ID NOs: 46, 52, 54, 60, 64, 66, 68, 70, 71, or 168, or an amino acid sequence at least 70%, 75%, 80%, 85%, 90%, 95%, 96%, 97%, 98%, or 99% identical to the aforesaid sequences.
   146. The AAV expression construct of any one of embodiments 129-145, which comprises in 5′ to 3′ order, a p10 promoter, and a VP-coding region comprising a modified Kozak sequence and a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein;
wherein the modified Kozak sequence is present at the 5′ end of the VP-coding region, e.g., at the start of the VP-coding region encoding the VP1 protein (e.g., the ORF encoding the VP1 protein);
optionally wherein, the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   147. The AAV expression construct of any one of embodiments 15-53, 55-85, or 86-146, wherein:
(i) the first Rep-coding region is present in the v-cath locus of the variant baculovirus genome;
(ii) the second Rep-coding region is present in the egt locus of the variant baculovirus genome;
and
(iii) the VP-coding region is present in the v-cath locus of the variant baculovirus genome.
   148. The AAV expression construct of any one of embodiments 15-53, 55-85, or 86-147, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   149. The AAV expression construct of any one of embodiments 15-53, 55-85, or 86-148, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter; and
(iii) the VP-coding region is present in the v-cath locus of the variant baculovirus genome and is operably linked to a p10 promoter.
   150. The AAV expression construct of any one of embodiments 15-53, 55-85, or 86-149, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, wherein the second Rep-coding region is operably linked to a polh promoter; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the first Rep-coding region.
   151. The AAV expression construct of any one of embodiments 96-150, wherein:
(i) the first Rep-coding region is present in the v-cath locus of the variant baculovirus genome;
(ii) the second Rep-coding region is present in the egt locus of the variant baculovirus genome;
(iii) the VP-coding region is present in the v-cath locus of the variant baculovirus genome; and
(iv) the second VP-coding region is present in the SOD locus of the variant baculovirus genome.
   152. The AAV expression construct of any one of embodiments 96-151, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(iv) the second VP-coding region is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   153. The AAV expression construct of any one of embodiments 96-152, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter;
(iii) the VP-coding region is present in the v-cath locus of the variant baculovirus genome and is operably linked to a p10 promoter; and
(iv) the second VP-coding region is present in the SOD locus of the variant and is operably linked to a ctx promoter.
   154. The AAV expression construct of any one of embodiments 96-153, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and
(iv) the second VP-coding region is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), and wherein the second VP-coding region is operably linked to a ctx promoter;
optionally wherein, the VP-coding region is present in the reverse orientation relative to the first Rep-coding region.
   155. The AAV expression construct of any one of embodiments 129-150, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   156. The AAV expression construct of any one of embodiments 129-150 or 155, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, which is present at the 5′ end of the VP-coding region, e.g., at the start of the VP-coding region encoding the VP1 protein (e.g., the ORF encoding the VP1 protein), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.
   157. The AAV expression construct of any one of embodiments 129-150, 155, or 156, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises:
   (a) a modified Kozak sequence (e.g., comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33), which is present at the 5′ end of the VP-coding region (e.g., at the start of the VP-coding region); and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      158. The AAV expression construct of any one of embodiments 129-150 or 155-157, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises in 5′ to 3′ order:
   (a) a modified Kozak sequence, optionally comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      159. An AAV expression construct comprising a variant baculovirus genome comprising:
(i) a first Rep-coding region which is present in the v-cath locus of the variant baculovirus genome;
(ii) a second Rep-coding which region is present in the egt locus of the variant baculovirus genome; and
(iii) a VP-coding region which is present in the v-cath locus of the variant baculovirus genome.
   160. An AAV expression construct comprising a variant baculovirus genome comprising:
(i) a first Rep-coding region which is present in the v-cath locus of the variant baculovirus genome and is operably linked to a polh promoter;
(ii) a second Rep-coding region which is present in the egt locus of the variant baculovirus genome and is operably linked to a polh promoter; and
(iii) a VP-coding region which is present in the v-cath locus of the variant baculovirus genome and is operably linked to a p10 promoter.
   161. An AAV expression construct comprising a variant baculovirus genome comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) a second Rep-coding region, which is present in the egt locus of the variant baculovirus genome and comprises a nucleotide sequence encoding a Rep52 protein but not a Rep78 protein; and
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
   162. An AAV expression construct comprising a variant baculovirus genome comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) a second Rep-coding region is present in the egt locus of the variant baculovirus genome and comprises a nucleotide sequence encoding a Rep52 protein but not a Rep78 protein, wherein the second Rep-coding region is operably linked to a polh promoter; and
(iii) a VP-coding region which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the first Rep-coding region.
   163. An AAV expression construct comprising a variant baculovirus genome comprising:
(i) a first Rep-coding region, which is present in the v-cath locus of the variant baculovirus genome;
(ii) a second Rep-coding region, which is present in the egt locus of the variant baculovirus genome;
(iii) a VP-coding region, which is present in the v-cath locus of the variant baculovirus genome; and
(iv) a second VP-coding region, which is present in the SOD locus of the variant baculovirus genome.
   164. An AAV expression construct comprising a variant baculovirus genome comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) a second Rep-coding region, which is present in the egt locus of the variant baculovirus genome and comprises a nucleotide sequence encoding a Rep52 protein but not a Rep78 protein;
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(iv) a second VP-coding region, which is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).
   165. An AAV expression construct comprising a variant baculovirus genome comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter;
(iii) a VP-coding region, which is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and
(iv) a second VP-coding region, which is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), wherein the second VP-coding region is operably linked to a ctx promoter;
optionally wherein, the VP-coding region is present in the reverse orientation relative to the first Rep-coding region.
   166. An AAV expression construct comprising a variant baculovirus genome comprising:
(i) a first Rep-coding region, which is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) a second Rep-coding region, which is present in the egt locus of the variant baculovirus genome and comprises a nucleotide sequence encoding a Rep52 protein but not a Rep78 protein, and wherein the second Rep-coding region is operably linked to a polh promoter; and
(iii) a VP-coding region, which is present in the v-cath locus of the variant baculovirus genome and is operably linked to a p10 promoter, wherein the VP region comprises:
   (a) a modified Kozak sequence which is present at the 5′ end of the VP-coding region (e.g., at the start of the VP-coding region), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
   (b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.
      167. The AAV expression construct of any one of the preceding embodiments, further comprising a nucleotide sequence encoding an assembly-activating protein (AAP).
      168. The AAV expression construct of embodiment 167, wherein the encoded AAP protein is an AAV2 AAP protein.
      169. The AAV expression construct of embodiment 167 or 168, wherein the encoded AAP protein comprises the amino acid sequence of SEQ ID NO: 218; an amino acid sequence comprising at least 70%, 75%, 80%, 85%, 90%, 95%, 96%, 97%, 98%, 99% sequence identity to SEQ ID NO: 218; an amino acid sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 218; or an amino acid sequence comprising at least one, two, three, four, five, six or seven, but no more than 30, 20, or 10 different amino acids relative to SEQ ID NO: 218.
      170. The AAV expression construct of any one of embodiments 167-169, wherein the nucleotide sequence encoding the AAP protein comprises the nucleotide sequence of SEQ ID NO: 219; a nucleotide sequence comprising at least 70%, 75%, 80%, 85%, 90%, 95%, 96%, 97%, 98%, 99% sequence identity to SEQ ID NO: 219; a nucleotide sequence comprising at least one, two, three, four, five, six or seven modifications, e.g., substitutions (e.g., conservative substitutions), insertions, or deletions, but no more than 30, 20, or 10 modifications, e.g., substitutions (e.g., conservative substitutions), insertions, or deletions, relative to SEQ ID NO: 219; or a nucleotide sequence comprising at least one, two, three, four, five, six or seven, but no more than 30, 20, or 10 different nucleotides relative to SEQ ID NO: 219.
      171. The AAV expression construct of any one of embodiments 167-170, wherein the nucleotide sequence encoding the AAP protein is operably linked to a promoter.
      172. The AAV expression construct of embodiment 171, wherein the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof.
      173. The AAV expression construct of embodiment 171 or 172, wherein the promoter is chosen from a polh promoter, a p10 promoter, a ctx promoter, a gp64 promoter an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.
      174. The AAV expression construct of any one of embodiments 171-173, wherein the promoter is a gp64 promoter, optionally wherein the promoter is gp64 promoter from a OpMNPV baculovirus genome.
      175. The AAV expression construct of embodiment 173 or 174, wherein the gp64 promoter comprises the nucleotide sequence of SEQ ID NO: 217; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 217; or a nucleotide sequence comprising at least one, two, three, four, five, six, or seven, but no more than ten modifications (e.g., substitutions) relative to SEQ ID NO: 217.
      176. The AAV expression construct of any one of embodiments 167-175, wherein the nucleotide sequence encoding the AAP protein is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
      177. The AAV expression construct of any one of embodiments 167-176, wherein the nucleotide sequence encoding the AAP protein is present in the p26, p10, and p74 gene loci.
      178. The AAV expression construct of any one of embodiments 167-177, wherein the nucleotide sequence encoding the AAP protein is present in the p26, p10, and p74 gene loci, and is operably linked to a gp64 promoter.
      179. The AAV expression construct of any one of embodiments 1-178, which further comprises a payload coding region comprising a nucleotide sequence encoding a payload.
      180. The AAV expression construct of any one of the preceding embodiments, which is stably maintained for at least 5-10 passages, e.g., at least 5, 6, 7, 8, 9, or 10 passages, in a host cell (e.g., an insect cell), e.g., when measured by an assay described herein, e.g., Western blot assay, a qPCR assay, or a SEAP assay, e.g., as described in Examples 5-8.
      181. The AAV expression construct of any one of embodiments 1-180, which is capable of producing higher AAV titers relative to a reference, e.g., an AAV expression construct comprising overlapping VP coding regions and a bicistronic Rep78/52 coding region (e.g., a Bac-to-Bac expression construct as described in Example 8), when measured by an assay, e.g., a SEAP assay or qPCR assay, e.g., as described in Example 7 or 8.
      182. The AAV expression construct of any one of embodiments 96-181, which is capable of producing improved VP ratios (e.g., ratios of VP3:VP2:VP1), relative to a reference, e.g., an AAV expression construct comprising a single VP-coding region having an ACG start codon, when measured by an assay, e.g., a Western blot assay or qPCR assay, e.g., as described in Example 7.
      183. The AAV expression construct of any one of embodiments 96-128, which is capable of producing more potent AAV capsid (e.g., AAV capsid with increased transduction efficiency), relative to a reference, e.g., an AAV expression construct comprising a single VP-coding region having an ACG start codon, when measured by an assay, e.g., a Western blot assay or qPCR assay, e.g., as described in Example 7.
      184. An AAV payload expression construct comprising a payload coding region comprising a nucleotide sequence encoding a payload wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.
      185. The AAV payload construct of embodiment 184, wherein the payload coding region is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.
      186. The AAV expression construct of any one of embodiments 179-183, or the AAV payload construct of embodiment 184 or 185, wherein the payload coding region comprises a start codon and a nucleotide sequence encoding the payload.
      187. The AAV expression construct of any one of embodiments 179-183 or 186, or the AAV payload construct of any one of embodiments 184-186, wherein the payload coding region is present in the v-cath locus of the variant baculovirus genome.
      188. The AAV expression construct of any one of embodiments 179-183, 186, or 187, or the AAV payload construct of any one of embodiments 184-187, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.
      189. A cell comprising the AAV expression construct of any one of embodiments 179-183 or 186-188, and/or the AAV payload construct of any one of embodiments 184-188.
      190. The cell of embodiment 189, which is an insect cell, optionally wherein the insect cell is an Sf9 cell or an Sf21 cell.
      191. A VP1 protein encoded by the AAV expression constructs of any one of embodiments 1-183.
      192. A VP2 protein encoded by the AAV expression constructs of any one of embodiments 1-183.
      193. A VP3 protein encoded by the AAV expression constructs of any one of embodiments 1-183.
      194. An AAV capsid protein encoded by the AAV expression construct of any one of embodiments 1-183, optionally wherein the AAV capsid protein is an AAV9 capsid or variant thereof or an AAV5 capsid or variant thereof.
      195. A Rep-78 protein encoded by the AAV expression constructs of any one of embodiments 1-183.
      196. A Rep-52 protein encoded by the AAV expression constructs of any one of embodiments 1-183.
      197. An AAV viral production system comprising the AAV expression construct of any one of embodiments 1-183, and the AAV payload expression construct of embodiment 184-188.
      198. The AAV viral production system of embodiment 197, which further comprises a viral production cell, which comprises the AAV expression construct and the AAV payload expression construct.
      199. The AAV viral production system of embodiment 197 or 198, where the viral production cell is an insect cell (e.g., an Sf9 cell or an Sf21 cell).
      200. A method of producing one, two, three, four, or all of a Rep78 protein, a Rep52 protein, a VP1 protein, a VP protein, and/or a VP3 protein, the method comprising:
(i) providing a cell comprising the AAV expression construct of any one of embodiments 1-183;
(ii) incubating the cell under conditions suitable to produce the one, two, three, four, or all of the Rep78 protein, the Rep52 protein, the VP1 protein, the VP protein, and/or the VP3 protein.
   201. The method of embodiment 200, further comprising, prior to step (i), introducing the AAV expression construct into the cell.
   202. A method of producing an AAV particle, the method comprising:
(i) providing a cell comprising the AAV expression construct of any one of embodiments 1-183 and the AAV payload construct of any one of embodiments 184-188, or a cell comprising the AAV production system of embodiments 197-199;
(ii) incubating the cell under conditions suitable to produce the AAV particle;
thereby producing the AAV particle.
   203. The method of embodiment 202, further comprising, prior to step (i), introducing the AAV expression construct and the AAV payload construct into the cell.
   204. The method of any one of embodiments 200-203, wherein the cell is an insect cell, e.g., an Sf9 cell or an Sf21 cell.
   205. An AAV particle made by the method of any one of embodiments 200-204.
   206. A pharmaceutical composition comprising the AAV particle of embodiment 205, and a pharmaceutically acceptable excipient.
   207. A nucleic acid comprising a nucleotide sequence comprising a modified Kozak sequence and a VP-coding region, wherein the modified Kozak sequence comprises the nucleotide sequence of any one of SEQ ID NOs: 32-42, or a nucleotide sequence comprising no more than one, two, or three different nucleotides relative to SEQ ID NOs: 32-42.
   208. The nucleic acid of embodiment 207, wherein the modified Kozak sequence is capable of modulating expression, e.g., increasing expression, of a protein encoded by a gene that is immediately downstream of the modified Kozak sequence.
   209. The nucleic acid of embodiment 207 or 208, wherein the modified Kozak sequence comprises a start codon for the translation of a protein encoded by a gene that is immediately downstream of the modified Kozak sequence.
   210. The nucleic acid of any one of embodiments 207-209, wherein the modified Kozak sequence comprises the nucleotide sequence of SEQ ID NO: 33, or a nucleotide sequence comprising no more than one, two, or three different nucleotides relative to SEQ ID NO: 33.
   211. The nucleic acid of any one of embodiments 207-210, wherein nucleotide sequence encoding the modified Kozak sequence comprises the nucleotide sequence of SEQ ID NO: 22, or a nucleotide sequence comprising no more than one, two, or three different nucleotides relative to SEQ ID NO: 22.
   212. The nucleic acid of embodiment 207-209, wherein the modified Kozak sequence comprises the nucleotide sequence of SEQ ID NO: 32, or a nucleotide sequence comprising no more than one, two, or three different nucleotides relative to SEQ ID NO: 32.
   213. The nucleic acid of any one of embodiments 207-209 or 212, wherein nucleotide sequence encoding the modified Kozak sequence comprises the nucleotide sequence of SEQ ID NO: 21, or a nucleotide sequence comprising no more than one, two, or three different nucleotides relative to SEQ ID NO: 21.
   214. The nucleic acid of any one of embodiments 207-213, wherein the modified Kozak sequence is present at the 5′ end of the VP-coding region, e.g., at the start of the VP-coding region encoding the VP1 protein (e.g., the ORF encoding the VP1 protein).
   215. The nucleic acid of any one of embodiments 207-2014, wherein the modified Kozak sequence comprises the start codon of the ORF encoding the VP1 protein.
   216. The nucleic acid of any one of embodiments 207-215, wherein the start codon comprises an ATG.
   217. The nucleic acid of any one of embodiments 207-216, which comprises the nucleotide sequence of SEQ ID NO: 44, 45, 50, 51, 58, or 59, or a nucleotide sequence at least 70%, 75%, 80%, 85%, 90%, 95%, 96%, 97%, 98%, or 99% identical to the aforesaid sequences.
   218. The nucleic acid of any one of embodiments 207-217, which encodes a VP1 protein comprising the amino acid sequence of SEQ ID NOs: 47, 53, or 61, or an amino acid sequence at least 70%, 75%, 80%, 85%, 90%, 95%, 96%, 97%, 98%, or 99% identical to the aforesaid sequences.
   219. The nucleic acid of any one of embodiments 207-218, which encodes a VP1 protein comprising the amino acid sequence of SEQ ID NOs: 46, 52, 54, 60, 64, 66, 68, 70, 71, or 168, or an amino acid sequence at least 70%, 75%, 80%, 85%, 90%, 95%, 96%, 97%, 98%, or 99% identical to the aforesaid sequences.
   220. The nucleic acid of any one of embodiments 207-219, which is operably linked to a p10 promoter. 221. An AAV expression construct comprising the nucleic acid of any one of embodiments 207-220.
   222. An AAV expression construct comprising: a first Rep-coding region comprising a first open reading frame (ORF) which comprises a start codon and a nucleotide sequence encoding one or more AAV Rep proteins selected from Rep78 and Rep52; and a second Rep-coding region comprising a second ORF which comprises a start codon and a nucleotide sequence encoding one or more AAV Rep proteins selected from Rep78 and Rep52.
   223. The AAV expression construct of embodiment 222, wherein the first Rep-coding region comprises a nucleotide sequence encoding Rep78 only.
   224. The AAV expression construct of embodiment 222 or 223, wherein the second Rep-coding region comprises a nucleotide sequence encoding Rep52 only.
   225. The AAV expression construct of any one of embodiments 222-224, wherein at least a portion of the first Rep-coding region is codon optimized from a reference Rep-coding nucleotide sequence.
   226. The AAV expression construct of embodiment 225, wherein the first Rep-coding region is codon optimized for an insect cell; optionally a Spodoptera frugiperda insect cell.
   227. The AAV expression construct of any one of embodiments 222-226, wherein at least a portion of the second Rep-coding region is codon optimized from a reference Rep-coding nucleotide sequence.
   228. The AAV expression construct of embodiment 227, wherein the second Rep-coding region is codon optimized for an insect cell; optionally a Spodoptera frugiperda insect cell.
   229. The AAV expression construct of any one of embodiments 222-228, wherein the first Rep-coding region comprises one or more expression control regions which comprise one or more promoter sequences.
   230. The AAV expression construct of embodiment 229, wherein the expression control region of the first Rep-coding region comprises at least one promoter sequence selected from: polh, ΔIE-1, p10, Δp10, and variations or derivatives thereof; optionally wherein the expression control region of the first Rep-coding region comprises at least one polh promoter.
   231. The AAV expression construct of embodiment 222, wherein the first Rep-coding region comprises a polh promoter, and wherein the first ORF comprises a nucleotide sequence encoding Rep78 only.
   232. The AAV expression construct of any one of embodiments 222-231, wherein the second Rep-coding region comprises one or more expression control regions which comprise one or more promoter sequences.
   233. The AAV expression construct of embodiment 232, wherein the expression control region of the second Rep-coding region comprises at least one promoter sequence selected from: polh, ΔIE-1, p10, Δp10, and variations or derivatives thereof; optionally wherein the expression control region of the second Rep-coding region comprises at least one polh promoter.
   234. The AAV expression construct of embodiment 222 or embodiment 231, wherein the second Rep-coding region comprises a polh promoter, and wherein the second ORF comprises a nucleotide sequence encoding Rep52 only.
   235. The AAV expression construct of any one of embodiments 1-234, wherein the first Rep-coding region comprises one or more expression-modifier sequences 5′ of the first ORF, wherein the one or more expression-modifier sequences decreases transcription initiation at the start codon of the first ORF.
   236. The AAV expression construct of embodiment 235, wherein the first Rep-coding region comprises between 3-100 nucleotides between the expression-modifier sequence and the start codon of the first ORF; optionally between 3-25 nucleotides, between 3-10 nucleotides, or 3 nucleotides between the expression-modifier sequence and the start codon of the first ORF.
   237. The AAV expression construct of any one of embodiments 1-236, wherein the second Rep-coding region comprises one or more expression-modifier sequences 5′ of the second ORF, wherein the one or more expression-modifier sequences decreases transcription initiation at the start codon of the second ORF.
   238. The AAV expression construct of embodiment 237, wherein the second Rep-coding region comprises between 3-100 nucleotides between the expression-modifier sequence and the start codon of the second ORF; optionally between 3-25 nucleotides, between 3-10 nucleotides, or 3 nucleotides between the expression-modifier sequence and the start codon of the second ORF.
   239. The AAV expression construct of any one of embodiments 235-238, wherein the one or more expression-modifier sequences comprises a minicistron sequence.
   240. The AAV expression construct of embodiment 239, wherein the minicistron insertion sequence is from a baculovirus gene; optionally a baculovirus gp64 gene.
   241. The AAV expression construct of embodiment 239, wherein the minicistron insertion sequence comprises SEQ ID NO: 4.
   242. The AAV expression construct of embodiment 239, wherein the minicistron insertion sequence comprises SEQ ID NO: 5.
   243. The AAV expression construct of any one of embodiments 1-242, wherein the AAV expression construct comprises a recombinant baculovirus genome (i.e., bacmid).
   244. The AAV expression construct of embodiment 243, wherein the first Rep-coding region is located in a first location of the baculovirus genome, and the second Rep-coding region is located in a second location of the baculovirus genome which is different from the first location of the baculovirus genome.
   245. The AAV expression construct of embodiment 243 or 244, wherein the first Rep-coding region is located in the Tn7/polh gene region or the egt gene region of the baculovirus genome; optionally wherein the first Rep-coding region is located in the Tn7/polh gene region of the baculovirus genome.
   246. The AAV expression construct of embodiment 243 or 244, wherein the first Rep-coding region is located in the v-cath gene region or the egt gene region of the baculovirus genome; optionally wherein the first Rep-coding region is located in v-catch gene region of the baculovirus genome.
   247. The AAV expression construct of any one of embodiments 243-246, wherein the second Rep-coding region is located in the Tn7/polh gene region or the egt gene region of the baculovirus genome; optionally wherein the second Rep-coding region is located in the egt gene region of the baculovirus genome.
   248. The AAV expression construct of any one of embodiments 222-247, wherein the AAV expression construct comprises a VP-coding region comprising a first open reading frame (ORF) which comprises a start codon and a nucleotide sequence encoding one or more AAV VP proteins selected from VP1, VP2, VP3, or a combination thereof.
   249. The AAV expression construct of embodiment 248, wherein the VP-coding region is located in the Tn7/polh gene region or the v-cath gene region of the baculovirus genome; optionally wherein the VP-coding region is located in the v-cath gene region of the baculovirus genome.
   250. The AAV expression construct of any one of embodiments 248-249, wherein the AAV expression construct comprises a VP-coding region located in the v-cath gene region of the baculovirus genome and at least one Rep-coding region located in the v-cath gene region of the baculovirus genome.
   251. The AAV expression construct of embodiment 250, wherein the first Rep-coding region comprises a nucleotide sequence encoding Rep78 only, and is located in v-catch gene region of the baculovirus genome; and the second Rep-coding region comprises a nucleotide sequence encoding Rep52 only is not located in the v-cath gene region of the baculovirus genome; optionally wherein the second Rep-coding region is located in the egt gene region of the baculovirus genome.
   252. The AAV expression construct of embodiment 250, wherein the second Rep-coding region comprises a nucleotide sequence encoding Rep52 only, and is located in v-catch gene region of the baculovirus genome; and the first Rep-coding region comprises a nucleotide sequence encoding Rep78 only is not located in the v-cath gene region of the baculovirus genome; optionally wherein the first Rep-coding region is located in the egt gene region of the baculovirus genome.
   253. An AAV payload construct comprising a payload region comprising a first open reading frame (ORF) which comprises a start codon and a nucleotide sequence encoding at least one payload polynucleotide, wherein the payload region is located in the v-cath gene region of the baculovirus genome.
   254. An AAV viral production system comprising an AAV expression construct and an AAV payload construct which comprises a payload polynucleotide; wherein the AAV expression construct is an AAV expression construct of any one of embodiments 222-252.
   255. An AAV viral production system comprising an AAV expression construct and an AAV payload construct which comprises a payload polynucleotide; wherein the AAV payload construct is an AAV payload construct of embodiment 254.
   256. The AAV viral production system of embodiment 34, wherein the AAV viral production system comprises an AAV viral production cell which comprises the AAV expression construct and the AAV payload construct.
   257. The AAV viral production system of embodiment 35, wherein the AAV viral production cell is an insect cell; optionally a Sf9 cell or a Sf21 cell.
   258. A method of expressing AAV Rep78 and Rep52 proteins in an AAV viral production cell, the method comprising: (i) providing an AAV expression construct of any one of embodiments 1-257; (ii) transfecting the AAV expression construct into an AAV viral production cell; (iii) and exposing the AAV viral production cell to conditions which allow the AAV viral production cell to process the Rep-coding regions into corresponding AAV Rep78 and Rep52 proteins.
   259. The method of embodiment 258, wherein the AAV viral production cell is an insect cell; optionally a Sf9 cell or a Sf21 cell.
   260. An Rep78 protein and/or Rep52 protein produced by the method of embodiment 258 or embodiment 259.
   261. A method of producing recombinant adeno-associated virus (rAAV) particle in an AAV viral production cell, the method comprising: (i) providing an AAV viral production system of embodiment 254 or 255, wherein the AAV expression construct comprises one or more VP-coding regions which comprise one or more nucleotide sequences encoding VP1, VP2 and VP3 capsid proteins; (ii) transfecting the AAV viral production system into an AAV viral production cell; (iii) exposing the AAV viral production cell to conditions which allow the AAV viral production cell to process the AAV expression construct and the AAV payload construct into rAAV particles; and, optionally, (iv) collecting the rAAV particles from the AAV viral production cell.
   262. The method of embodiment 261, wherein the AAV viral production cell is an insect cell; optionally a Sf9 cell or a Sf21 cell.
   263. A recombinant adeno-associated virus (rAAV) particle produced by the method of embodiment 261 or embodiment 262.
   264. A pharmaceutical composition comprising the rAAV particle of embodiment 263 and a pharmaceutically acceptable excipient.

### BRIEF DESCRIPTION OF THE DRAWINGS

The foregoing and other objects, features and advantages will be apparent from the following description of particular embodiments of the present disclosure, as illustrated in the accompanying figures. The figures are not necessarily to scale or comprehensive, with emphasis instead being placed upon illustrating the principles of various embodiments of the present disclosure.

FIG. 1A presents a gel column showing Western blot analysis results from split Rep78/Rep52 studies of the present disclosure. FIG. 1B presents qPCR analysis results from split Rep78/Rep52 studies of the present disclosure.

FIG. 2 presents a gel column showing Western blot analysis results from polh_MC_Rep78-only studies of the present disclosure.

FIG. 3A presents a gel column showing Western blot analysis results from split Rep78/Rep52 studies of the present disclosure. FIG. 3B presents ddPCR titer results for clarified lysate samples from split Rep78/Rep52 studies of the present disclosure. FIG. 3C presents ddPCR titer results for AFB-purified samples from split Rep78/Rep52 studies of the present disclosure. FIG. 3D presents AUC % full capsid analysis results of AFB-purified samples from split Rep78/Rep52 studies of the present disclosure. FIG. 3E presents rAAV potency analysis results by HTT knockdown relative to a reference, of AFB-purified samples from split Rep78/Rep52 studies of the present disclosure

FIG. 4 presents a gel column showing Western blot analysis results from related to AB189 capsid insert testing.

FIG. 5A presents a gel column showing Western blot analysis results from related BIIC passage stability testing for Bacmid AA965 and Bacmid AB189; FIG. 5B, FIG. 5C, and FIG. 5D present corresponding Western blot band dosimetry measurements for Western VP1 (FIG. 5B), Western VP3 (FIG. 5C), and Western Rep78 (FIG. 5D).

FIG. 6A and FIG. 6B present graphs for qPCR titer results related to BIIC passage stability testing for Bacmid AA965 and Bacmid AB189, with FIG. 6A showing log scaling and FIG. 6B showing linear scaling.

FIG. 7 is a schematic showing the AAV expression construct, Bacmid AB189-VP1_{ACG}, for producing AAV replication (Rep52 and Rep78) and capsid proteins (VP1, VP2, and VP3). Bacmid AB189 comprises a Rep78 coding region with a minicistron upstream under the control of the polyhedrin (polh) promoter in the v-cath baculovirus gene locus; a Rep52 coding region under the control of the polh promoter in the egt baculovirus gene locus; and overlapping VP 1, 2, and 3 coding regions (expressed from a single polycistronic ORF) under the control of the p10 promoter also in the v-cath baculovirus gene locus.

FIG. 8 is a schematic showing the AAV expression construct, Bacmid AB189-VP1_{ACG}CTX VP1 for producing AAV replication (Rep52 and Rep78) and capsid proteins (VP1, VP2, and VP3). Bacmid AB189-VP1_{ACG}CTX VP1 comprises a Rep78 coding region with a minicistron upstream under the control of the polyhedrin (polh) promoter in the v-cath baculovirus gene locus; a Rep52 coding region under the control of the polh promoter in the egt baculovirus gene locus; overlapping VP 1, 2, and 3 coding (expressed from a single polycistronic ORF) regions under the control of the p10 promoter also in the v-cath baculovirus gene locus; and a second VP-coding region encoding primarily VP1 present in the SOD baculovirus gene locus and is under the control of the CTX promoter (e.g., a CTX promoter comprising the nucleotide sequence of SEQ ID NO: 164).

FIG. 9 provides a Western blot showing the relative levels of VP1, 2, and 3 proteins of an AAV9.v1 capsid produced by Bacmid AB189-VP1_{ACG} comprising a single copy of VP1 in a polycistronic ORF with overlapping VP1, VP2, and VP3 coding regions (left side of gel, top construct) and Bacmid AB189-VP1_{ACG}-CTX VP1 comprising two copies of VP1, including one copy of VP1 in a polycistronic ORF with overlapping VP1, VP2, and VP3 coding regions and a second copy of VP1 under the control of the CTX promoter (e.g., a CTX promoter comprising the nucleotide sequence of SEQ ID NO: 164) (right side of Western blot and bottom construct).

FIG. 10A provides a schematic of Bacmid AB189 comprising a modified Kozak sequence for initiating of translation of VP1. FIG. 10B provides a Western blot showing production of VP1, VP2, and VP3 proteins from Bacmid AB189 encoding an AAV9.v2 or AAV9.v5 capsid variant with or without the VPlaug13 (SEQ ID NO: 21 (RNA) or 32 (DNA)) or VPlaug14 (SEQ ID NO: 22 (RNA) or SEQ ID NO: 33 (DNA)) modified Kozak sequences.

FIG. 11 is a graph depicting the AAV viral titer in vg/mL over the hours post-infection of Sf9 cells with Bacmid AB189-modified Kozak-VPlaug13-AAV9.v2, Bacmid AB189-modified Kozak-VPlaug14-AAV9.v2, or Bacmid AB189-AAV9.v2_{ACG} control.

FIG. 12 is a graph depicting SEAP activity per vg for the AAV expression constructs indicated on the X axis, which are from left to right, AB189-AAV9.v1ACG sample 1 (S1), AB189-AAV9.v1ACG sample 2 (S2), AB189-AAV9.v1ACG-CTX VP1 S1, AB189-AAV9.v1ACG-CTX VP1 S2, AB189-modified Kozak-VP1aug13-AAV9 S1, AB189-modified Kozak-VPlaug13-AAV9 S2, AB189-modified Kozak-VP1aug14-AAV9 S1, and AB189-modified Kozak-VP1aug14-AAV9 S2.

FIG. 13A provides a schematic of the bac-to-bac construct (top) and Bacmid AB189-VP1_{ACG} (bottom). FIG. 13B is a graph depicting the rAAV1 viral titers over the Rep/Cap BIIC passage produced by Bac-bac or Bacmid AB189-VP1_{ACG}. FIG. 13C is Western blot showing AAV Cap and Rep proteins produced by Bac-bac (left) or Bacmid AB189-VP1_{ACG} (right).

FIG. 14 provides a graph depicting transducing units/μL over bacmid passage for bac-to-bac or Bacmid AB189-VP1_{ACG} encoding an AAV1 capsid protein.

### DETAILED DESCRIPTION

Baculovirus expression systems are a widely used tool in recombinant protein production. Their high scalability and productivity have been further extended to the production of recombinant adeno-associated virus (rAAV). However, baculovirus-based rAAV production is hindered by several factors including passage stability, complexity, and the number of protein products needed to support rAAV replication, and the generally low-throughput and bespoke nature of techniques used to modify large viral genomes.

Described herein are compositions, e.g., AAV expression constructs, and methods for the production of AAV particles and the expression of AAV capsid proteins (e.g., VP1, VP2, and/or VP3) and replication proteins (e.g., Rep52 and/or Rep78). In some embodiments, an AAV expression construct described herein demonstrates improved properties over previous AAV expression constructs including improved passage stability, increased AAV viral titers, improved capsid protein ratios, improved capsid quality, and improved AAV capsid potency (e.g., increased transduction efficiency), for AAV capsid proteins of different AAV serotypes, including but not limited to AAV9 capsid proteins and variants thereof. Without wishing to be bound by theory, the compositions and methods described herein allow for more efficient production of AAV-based gene therapies.

### I. Adeno-Associated Viruses (AAVs)

### Overview

Adeno-associated viruses (AAV) are small non-enveloped icosahedral capsid viruses of the Parvoviridae family characterized by a single stranded DNA viral genome. Parvoviridae family viruses consist of two subfamilies Parvovirinae, which infect vertebrates, and Densovirinae, which infect invertebrates. The Parvoviridae family includes the Dependovirus genus which includes AAV, capable of replication in vertebrate hosts including, but not limited to, human, primate, bovine, canine, equine, and ovine species.

The parvoviruses and other members of the Parvoviridae family are generally described in Kenneth I. Berns, “Parvoviridae: The Viruses and Their Replication,” Chapter 69 in Fields Virology (3d Ed. 1996), the contents of which are incorporated by reference in their entirety.

AAV have proven to be useful as a biological tool due to their relatively simple structure, their ability to infect a wide range of cells (including quiescent and dividing cells) without integration into the host genome and without replicating, and their relatively benign immunogenic profile. The genome of the virus may be manipulated to contain a minimum of components for the assembly of a functional recombinant virus, or viral particle, which is loaded with or engineered to target a particular tissue and express or deliver a desired payload.

### AAV Viral Genomes

The wild-type AAV viral genome is a linear, single-stranded DNA (ssDNA) molecule approximately 5,000 nucleotides (nt) in length. Inverted terminal repeats (ITRs) traditionally cap the viral genome at both the 5′ and the 3′ end, providing origins of replication for the viral genome. While not wishing to be bound by theory, an AAV viral genome typically comprises two ITR sequences. These ITRs have a characteristic T-shaped hairpin structure defined by a self-complementary region (145 nt in wild-type AAV) at the 5′ and 3′ ends of the ssDNA which form an energetically stable double stranded region. The double stranded hairpin structures comprise multiple functions comprising, but not limited to, acting as an origin for DNA replication by functioning as primers for the endogenous DNA polymerase complex of the host viral replication cell.

The wild-type AAV viral genome further comprises nucleotide sequences for two open reading frames, one for the four non-structural Rep proteins (Rep78, Rep68, Rep52, Rep40, encoded by Rep genes) and one for the three capsid, or structural, proteins (VP1, VP2, VP3, encoded by capsid genes or Cap genes). The Rep proteins are important for replication and packaging, while the capsid proteins are assembled to create the protein shell of the AAV, or AAV capsid. Alternative splicing and alternate initiation codons and promoters result in the generation of four different Rep proteins from a single open reading frame and the generation of three capsid proteins from a single open reading frame. Though it varies by AAV serotype, as a non-limiting example, for AAV9/hu.14 (SEQ ID NO: 123 of U.S. Pat. No. 7,906,111, the content of which is incorporated herein by reference in its entirety as related to AAV9/hu.14) VP1 refers to amino acids 1-736, VP2 refers to amino acids 138-736, and VP3 refers to amino acids 203-736. In other words, VP1 is the full-length capsid sequence, while VP2 and VP3 are shorter components of the whole. As a result, changes in the sequence in the VP3 region, are also changes to VP1 and VP2, however, the percent difference as compared to the parent sequence will be greatest for VP3 since it is the shortest sequence of the three. Though described here in relation to the amino acid sequence, the nucleic acid sequence encoding these proteins can be similarly described. Together, the three capsid proteins assemble to create the AAV capsid protein. While not wishing to be bound by theory, the AAV capsid protein typically comprises a molar ratio of 1:1:10 of VP1:VP2:VP3. As used herein, an “AAV serotype” is defined primarily by the AAV capsid. In some instances, the ITRs are also specifically described by the AAV serotype (e.g., AAV2/9).

For use as a biological tool, the wild-type AAV viral genome can be modified to replace the rep/cap sequences with a nucleic acid sequence comprising a payload region with at least one ITR region. Typically, in recombinant AAV viral genomes there are two ITR regions. The rep/cap sequences can be provided in trans during production to generate AAV particles.

In addition to the encoded heterologous payload, AAV vectors may comprise the viral genome, in whole or in part, of any naturally occurring and/or recombinant AAV serotype nucleotide sequence or variant. AAV variants may have sequences of significant homology at the nucleic acid (genome or capsid) and amino acid levels (capsids), to produce constructs which are generally physical and functional equivalents, replicate by similar mechanisms, and assemble by similar mechanisms. See Chiorini et al., J. Vir. 71: 6823-33(1997); Srivastava et al., J. Vir. 45:555-64 (1983); Chiorini et al., J. Vir. 73:1309-1319 (1999); Rutledge et al., J. Vir. 72:309-319 (1998); and Wu et al., J. Vir. 74: 8635-47 (2000), the contents of each of which are incorporated herein by reference in their entireties as related to AAV variants and equivalents, insofar as they do not conflict with the present disclosure.

In certain embodiments, AAV particles, viral genomes and/or payloads of the present disclosure, and the methods of their use, may be as described in WO2017189963, the content of which is incorporated herein by reference in its entirety as related to AAV particles, viral genomes and/or payloads.

AAV particles of the present disclosure may be formulated in any of the gene therapy formulations of the disclosure comprising any variations of such formulations apparent to those skilled in the art. The reference to “AAV particles”, “AAV particle formulations” and “formulated AAV particles” in the present application refers to the AAV particles which may be formulated and those which are formulated without limiting either.

In certain embodiments, AAV particles of the present disclosure are recombinant AAV (rAAV) viral particles which are replication defective, lacking sequences encoding functional Rep and Cap proteins within their viral genome. These defective AAV particles may lack most or all parental coding sequences and essentially carry only one or two AAV ITR sequences and the nucleic acid of interest (i.e., payload) for delivery to a cell, a tissue, an organ or an organism.

In certain embodiments, the viral genome of the AAV particles of the present disclosure comprises at least one control element which provides for the replication, transcription and translation of a coding sequence encoded therein. Not all of the control elements need always be present as long as the coding sequence is capable of being replicated, transcribed and/or translated in an appropriate host cell. Non-limiting examples of expression control elements comprise sequences for transcription initiation and/or termination, promoter and/or enhancer sequences, efficient RNA processing signals such as splicing and polyadenylation signals, sequences that stabilize cytoplasmic mRNA, sequences that enhance translation efficacy (e.g., Kozak consensus sequence), sequences that enhance protein stability, and/or sequences that enhance protein processing and/or secretion.

According to the present disclosure, AAV particles for use in therapeutics and/or diagnostics comprise a virus that has been distilled or reduced to the minimum components necessary for transduction of a nucleic acid payload or cargo of interest. In this manner, AAV particles are engineered as vehicles for specific delivery while lacking the deleterious replication and/or integration features found in wild-type viruses.

AAV particles of the present disclosure may be produced recombinantly and may be based on adeno-associated virus (AAV) parent or reference sequences. As used herein, a “vector” is any molecule or moiety which transports, transduces, or otherwise acts as a carrier of a heterologous molecule such as the nucleic acids described herein.

In addition to single stranded AAV viral genomes (e.g., ssAAVs), the present disclosure also provides for self-complementary AAV (scAAVs) viral genomes. scAAV viral genomes contain DNA strands which anneal together to form double stranded DNA. By skipping second strand synthesis, scAAVs allow for rapid expression in the cell.

In certain embodiments, the AAV viral genome of the present disclosure is a scAAV. In certain embodiments, the AAV viral genome of the present disclosure is a ssAAV.

Methods for producing and/or modifying AAV particles are disclosed in the art, such as pseudotyped AAV particles (PCT Patent Publication Nos. WO200028004; WO200123001; WO2004112727; WO 2005005610 and WO 2005072364, the contents of each of which are incorporated herein by reference in their entireties as related to producing and/or modifying AAV particles, insofar as they do not conflict with the present disclosure).

AAV particles may be modified to enhance the efficiency of delivery. Such modified AAV particles can be packaged efficiently and be used to successfully infect the target cells at high frequency and with minimal toxicity. In certain embodiments the capsids of the AAV particles are engineered according to the methods described in US Publication Number US 20130195801, the content of which is incorporated herein by reference in its entirety as related to modifying AAV particles to enhance the efficiency of delivery.

In certain embodiments, the AAV particles comprise a payload construct and/or region encoding a polypeptide or protein of the present disclosure, and may be introduced into mammalian cells. In certain embodiments, the AAV particles comprise a payload construct and/or region encoding a polypeptide or protein of the present disclosure, and may be introduced into insect cells.

In certain embodiments, the AAV particles of the present disclosure comprise a viral genome with at least one ITR region and a payload region. In certain embodiments, the viral genome has two ITRs. These two ITRs flank the payload region at the 5′ and 3′ ends. The ITRs function as origins of replication comprising recognition sites for replication. ITRs comprise sequence regions which can be complementary and symmetrically arranged. ITRs incorporated into viral genomes of the present disclosure may be comprised of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome comprising two ITRs. In certain embodiments, the ITRs are of the same serotype as one another. In another embodiment, the ITRs are of different serotypes. Non-limiting examples comprise zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length.

In certain embodiments, each ITR may be 141 nucleotides in length. In certain embodiments, each ITR may be 130 nucleotides in length. In certain embodiments, each ITR may be 119 nucleotides in length.

### Viral Genome Size

In certain embodiments, the AAV particle which includes a payload described herein may be single stranded or double stranded viral genome. The size of the viral genome may be small, medium, large or the maximum size. Additionally, the viral genome may include a promoter and a polyA tail.

In certain embodiments, the viral genome which includes a payload described herein may be a small single stranded viral genome. A small single stranded viral genome may be 2.1 to 3.5 kb in size such as about 2.1, 2.2, 2.3, 2.4, 2.5, 2.6, 2.7, 2.8, 2.9, 3.0, 3.1, 3.2, 3.3, 3.4, and 3.5 kb in size. As a non-limiting example, the small single stranded viral genome may be 3.2 kb in size. As another non-limiting example, the small single stranded viral genome may be 2.2 kb in size. Additionally, the viral genome may include a promoter and a polyA tail.

In certain embodiments, the viral genome which includes a payload described herein may be a small double stranded viral genome. A small double stranded viral genome may be 1.3 to 1.7 kb in size such as about 1.3, 1.4, 1.5, 1.6, and 1.7 kb in size. As a non-limiting example, the small double stranded viral genome may be 1.6 kb in size. Additionally, the viral genome may include a promoter and a polyA tail.

In certain embodiments, the viral genome which includes a payload described herein e.g., polynucleotide, siRNA or dsRNA, may be a medium single stranded viral genome. A medium single stranded viral genome may be 3.6 to 4.3 kb in size such as about 3.6, 3.7, 3.8, 3.9, 4.0, 4.1, 4.2 and 4.3 kb in size. As a non-limiting example, the medium single stranded viral genome may be 4.0 kb in size. Additionally, the viral genome may include a promoter and a polyA tail.

In certain embodiments, the viral genome which includes a payload described herein may be a medium double stranded viral genome. A medium double stranded viral genome may be 1.8 to 2.1 kb in size such as about 1.8, 1.9, 2.0, and 2.1 kb in size. As a non-limiting example, the medium double stranded viral genome may be 2.0 kb in size. Additionally, the viral genome may include a promoter and a polyA tail.

In certain embodiments, the viral genome which includes a payload described herein may be a large single stranded viral genome. A large single stranded viral genome may be 4.4 to 6.0 kb in size such as about 4.4, 4.5, 4.6, 4.7, 4.8, 4.9, 5.0, 5.1, 5.2, 5.3, 5.4, 5.5, 5.6, 5.7, 5.8, 5.9 and 6.0 kb in size. As a non-limiting example, the large single stranded viral genome may be 4.7 kb in size. As another non-limiting example, the large single stranded viral genome may be 4.8 kb in size. As yet another non-limiting example, the large single stranded viral genome may be 6.0 kb in size. Additionally, the viral genome may include a promoter and a polyA tail.

In certain embodiments, the viral genome which includes a payload described herein may be a large double stranded viral genome. A large double stranded viral genome may be 2.2 to 3.0 kb in size such as about 2.2, 2.3, 2.4, 2.5, 2.6, 2.7, 2.8, 2.9 and 3.0 kb in size. As a non-limiting example, the large double stranded viral genome may be 2.4 kb in size. Additionally, the viral genome may include a promoter and a polyA tail.

In certain embodiments, an viral genome of the present disclosure can include at least one filler region. In certain embodiments, an viral genome of the present disclosure can include at least one multiple cloning site (MCS) region. In certain embodiments, an viral genome of the present disclosure can include at least one promoter region. In certain embodiments, an viral genome of the present disclosure can include at least one exon region. In certain embodiments, an viral genome of the present disclosure can include at least one intron region.

### Viral Genome Regions: Inverted Terminal Repeats (ITRs)

The AAV particles of the present disclosure include a viral genome with at least one Inverted Terminal Repeat (ITR) region and a payload region. In certain embodiments, the viral genome has two ITRs. These two ITRs flank the payload region at the 5′ and 3′ ends. The ITRs function as origins of replication including recognition sites for replication. ITRs include sequence regions which can be complementary and symmetrically arranged. ITRs incorporated into viral genomes of the present disclosure may be included of naturally occurring polynucleotide sequences or recombinantly derived polynucleotide sequences.

The ITRs may be derived from the same serotype as the capsid, or a derivative thereof. The ITR may be of a different serotype than the capsid. In certain embodiments, the AAV particle has more than one ITR. In a non-limiting example, the AAV particle has a viral genome including two ITRs. In certain embodiments, the ITRs are of the same serotype as one another. In another embodiment, the ITRs are of different serotypes. Non-limiting examples include zero, one or both of the ITRs having the same serotype as the capsid. In certain embodiments both ITRs of the viral genome of the AAV particle are AAV2 ITRs.

Independently, each ITR may be about 100 to about 150 nucleotides in length. An ITR may be about 100-105 nucleotides in length, 106-110 nucleotides in length, 111-115 nucleotides in length, 116-120 nucleotides in length, 121-125 nucleotides in length, 126-130 nucleotides in length, 131-135 nucleotides in length, 136-140 nucleotides in length, 141-145 nucleotides in length or 146-150 nucleotides in length. In certain embodiments, the ITRs are 140-142 nucleotides in length. Non-limiting examples of ITR length are 102, 130, 140, 141, 142, 145 nucleotides in length, and those having at least 95% identity thereto.

In certain embodiments, each ITR may be 141 nucleotides in length. In certain embodiments, each ITR may be 130 nucleotides in length. In certain embodiments, each ITR may be 119 nucleotides in length.

In certain embodiments, the AAV particles include two ITRs and one ITR is 141 nucleotides in length and the other ITR is 130 nucleotides in length. In certain embodiments, the AAV particles include two ITRs and both ITR are 141 nucleotides in length.

Independently, each ITR may be about 75 to about 175 nucleotides in length. The ITR may, independently, have a length such as, but not limited to, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, and 175 nucleotides. The length of the ITR for the viral genome may be 75-80, 75-85, 75-100, 80-85, 80-90, 80-105, 85-90, 85-95, 85-110, 90-95, 90-100, 90-115, 95-100, 95-105, 95-120, 100-105, 100-110, 100-125, 105-110, 105-115, 105-130, 110-115, 110-120, 110-135, 115-120, 115-125, 115-140, 120-125, 120-130, 120-145, 125-130, 125-135, 125-150, 130-135, 130-140, 130-155, 135-140, 135-145, 135-160, 140-145, 140-150, 140-165, 145-150, 145-155, 145-170, 150-155, 150-160, 150-175, 155-160, 155-165, 160-165, 160-170, 165-170, 165-175, and 170-175 nucleotides. As a non-limiting example, the viral genome comprises an ITR that is about 105 nucleotides in length. As a non-limiting example, the viral genome comprises an ITR that is about 141 nucleotides in length. As a non-limiting example, the viral genome comprises an ITR that is about 130 nucleotides in length. As a non-limiting example, the viral genome comprises an ITR that is about 105 nucleotides in length and 141 nucleotides in length. As a non-limiting example, the viral genome comprises an ITR that is about 105 nucleotides in length and 130 nucleotides in length. As a non-limiting example, the viral genome comprises an ITR that is about 130 nucleotides in length and 141 nucleotides in length.

### AAV Serotypes

AAV particles of the present disclosure may include or be derived from any natural or recombinant AAV serotype. According to the present disclosure, the AAV particles may utilize or be based on a serotype or include a peptide selected from any of the following: VOY101, VOY201, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B3 (PHP.B3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TMP, AAVPHP.B-TTP, AAVPHP.S/G2A12, AAVG2A15/G2A3 (G2A3), AAVG2B4 (G2B4), AAVG2B5 (G2B5), PHP.S, AAV1, AAV2, AAV2G9, AAV3, AAV3a, AAV3b, AAV3-3, AAV4, AAV4-4, AAV5, AAV6, AAV6.1, AAV6.2, AAV6.1.2, AAV7, AAV7.2, AAV8, AAV9, AAV9.11, AAV9.13, AAV9.16, AAV9.24, AAV9.45, AAV9.47, AAV9.61, AAV9.68, AAV9.84, AAV9.9, AAV10, AAV11, AAV12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAV1-7/rh.48, AAV1-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-11/rh.53, AAV4-8/r11.64, AAV4-9/rh.54, AAV4-19/rh.55, AAV5-3/rh.57, AAV5-22/rh.58, AAV7.3/hu.7, AAV16.8/hu.10, AAV16.12/hu.11, AAV29.3/bb.1, AAV29.5/bb.2, AAV106.1/hu.37, AAV114.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAV-DJ, AAV-DJ8, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu.12, AAVH6, AAVLK03, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.5R1, AAVcy.2, AAVcy.3, AAVcy.4, AAVcy.5, AAVCy.5R1, AAVCy.5R2, AAVCy.5R3, AAVCy.5R4, AAVcy.6, AAVhu.1, AAVhu.2, AAVhu.3, AAVhu.4, AAVhu.5, AAVhu.6, AAVhu.7, AAVhu.9, AAVhu.10, AAVhu.11, AAVhu.13, AAVhu.15, AAVhu.16, AAVhu.17, AAVhu.18, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu.14/9, AAVhu.t 19, AAVrh.2, AAVrh.2R, AAVrh.8, AAVrh.8R, AAVrh.10, AAVrh.12, AAVrh.13, AAVrh.13R, AAVrh.14, AAVrh.17, AAVrh.18, AAVrh.19, AAVrh.20, AAVrh.21, AAVrh.22, AAVrh.23, AAVrh.24, AAVrh.25, AAVrh.31, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533A mutant, AAAV, BAAV, caprine AAV, bovine AAV, AAVhE1.1, AAVhEr1.5, AAVhER1.14, AAVhEr1.8, AAVhEr1.16, AAVhEr1.18, AAVhEr1.35, AAVhEr1.7, AAVhEr1.36, AAVhEr2.29, AAVhEr2.4, AAVhEr2.16, AAVhEr2.30, AAVhEr2.31, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), UPENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.10, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLv1-1, AAV Clv1-10, AAV CLv1-2, AAV CLv-12, AAV CLv1-3, AAV CLv-13, AAV CLv1-4, AAV Clv1-7, AAV Clv1-8, AAV Clv1-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M11, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-10, AAV CSp-11, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, AAVrh20, AAVrh32/33, AAVrh39, AAVrh46, AAVrh73, AAVrh74, AAVhu.26, or variants or derivatives thereof.

In some embodiments, an AAV expression construct described herein expresses an AAV capsid protein as provided in WO2021230987, WO2019028306, WO2019222329, WO2020077165, WO2020028751, WO2020223280, WO2019222444, WO2019222441, or WO2017100671, the contents of which are hereby incorporated by reference in their entirety. In some embodiments, an AAV expression construct described herein expresses an AAV capsid protein encoded by or comprising a sequence as provided in Table 7, or a sequence substantially identical (e.g., having at least about 70%, 75%, 80%, 85%, 90%, 92%, 95%, 97%, 98%, or 99% sequence identity) to any of the aforesaid sequences.

The AAV-DJ sequence may include two mutations: (1) R587Q where arginine (R; Arg) at amino acid 587 is changed to glutamine (Q; Gln) and (2) R590T where arginine (R; Arg) at amino acid 590 is changed to threonine (T; Thr). As another non-limiting example, may include three mutations: (1) K406R where lysine (K; Lys) at amino acid 406 is changed to arginine (R; Arg), (2) R587Q where arginine (R; Arg) at amino acid 587 is changed to glutamine (Q; Gln) and (3) R590T where arginine (R; Arg) at amino acid 590 is changed to threonine (T; Thr).

In certain embodiments, the AAV may be a serotype generated by the AAV9 capsid library with mutations in amino acids 390-627 (VP1 numbering) The serotype and corresponding nucleotide and amino acid substitutions may be, but is not limited to, AAV9.1 (G1594C; D532H), AAV6.2 (T1418A and T1436X; V473D and I479K), AAV9.3 (T1238A; F413Y), AAV9.4 (T1250C and A1617T; F417S), AAV9.5 (A1235G, A1314T, A1642G, C1760T; Q412R, T548A, A587V), AAV9.6 (T1231A; F411I), AAV9.9 (G1203A, G1785T; W595C), AAV9.10 (A1500G, T1676C; M559T), AAV9.11 (A1425T, A1702C, A1769T; T568P, Q590L), AAV9.13 (A1369C, A1720T; N457H, T574S), AAV9.14 (T1340A, T1362C, T1560C, G1713A; L447H), AAV9.16 (A1775T; Q592L), AAV9.24 (T1507C, T1521G; W503R), AAV9.26 (A1337G, A1769C; Y446C, Q590P), AAV9.33 (A1667C; D556A), AAV9.34 (A1534G, C1794T; N512D), AAV9.35 (A1289T, T1450A, C1494T, A1515T, C1794A, G1816A; Q430L, Y484N, N98K, V6061), AAV9.40 (A1694T, E565V), AAV9.41 (A1348T, T1362C; T450S), AAV9.44 (A1684C, A1701T, A1737G; N562H, K567N), AAV9.45 (A1492T, C1804T; N498Y, L602F), AAV9.46 (G1441C, T1525C, T1549G; G481R, W509R, L517V), 9.47 (G1241A, G1358A, A1669G, C1745T; S414N, G453D, K557E, T582I), AAV9.48 (C1445T, A1736T; P482L, Q579L), AAV9.50 (A1638T, C1683T, T1805A; Q546H, L602H), AAV9.53 (G1301A, A1405C, C1664T, G1811T; R134Q, S469R, A555V, G604V), AAV9.54 (C1531A, T1609A; L511I, L537M), AAV9.55 (T1605A; F535L), AAV9.58 (C1475T, C1579A; T492I, H527N), AAV.59 (T1336C; Y446H), AAV9.61 (A1493T; N498I), AAV9.64 (C1531A, A1617T; L511I), AAV9.65 (C1335T, T1530C, C1568A; A523D), AAV9.68 (C1510A; P504T), AAV9.80 (G1441A, G481R), AAV9.83 (C1402A, A1500T; P468T, E500D), AAV9.87 (T1464C, T1468C; S490P), AAV9.90 (A1196T; Y399F), AAV9.91 (T1316G, A1583T, C1782G, T1806C; L439R, K528I), AAV9.93 (A1273G, A1421G, A1638C, C1712T, G1732A, A1744T, A1832T; S425G, Q474R, Q546H, P571L, G578R, T582S, D611V), AAV9.94 (A1675T; M559L) and AAV9.95 (T1605A; F535L).

In any of the DNA and RNA sequences referenced and/or described herein, the single letter symbol has the following description: A for adenine; C for cytosine; G for guanine; T for thymine; U for Uracil; W for weak bases such as adenine or thymine; S for strong nucleotides such as cytosine and guanine; M for amino nucleotides such as adenine and cytosine; K for keto nucleotides such as guanine and thymine; R for purines adenine and guanine; Y for pyrimidine cytosine and thymine; B for any base that is not A (e.g., cytosine, guanine, and thymine); D for any base that is not C (e.g., adenine, guanine, and thymine); H for any base that is not G (e.g., adenine, cytosine, and thymine); V for any base that is not T (e.g., adenine, cytosine, and guanine); N for any nucleotide (which is not a gap); and Z is for zero.

In any of the amino acid sequences referenced and/or described herein, the single letter symbol has the following description: G (Gly) for Glycine; A (Ala) for Alanine; L (Leu) for Leucine; M (Met) for Methionine; F (Phe) for Phenylalanine; W (Trp) for Tryptophan; K (Lys) for Lysine; Q (Gln) for Glutamine; E (Glu) for Glutamic Acid; S (Ser) for Serine; P (Pro) for Proline; V (Val) for Valine; I (Ile) for Isoleucine; C (Cys) for Cysteine; Y (Tyr) for Tyrosine; H (His) for Histidine; R (Arg) for Arginine; N (Asn) for Asparagine; D (Asp) for Aspartic Acid; T (Thr) for Threonine; B (Asx) for Aspartic acid or Asparagine; J (Xle) for Leucine or Isoleucine; O (Pyl) for Pyrrolysine; U (Sec) for Selenocysteine; X (Xaa) for any amino acid; and Z (Glx) for Glutamine or Glutamic acid.

In certain embodiments, the AAV serotype may be, or may include a sequence, insert, modification or mutation as described in Patent Publications WO2015038958, WO2017100671, WO2016134375, WO2017083722, WO2017015102, WO2017058892, WO2017066764, U.S. Pat. Nos. 9,624,274, 9,475,845, US20160369298, US20170145405, the contents of which are herein incorporated by reference in their entirety.

In certain embodiments, the AAV may be a serotype generated by Cre-recombination-based AAV targeted evolution (CREATE) as described by Deverman et al., (Nature Biotechnology 34(2):204-209 (2016)), the contents of which are herein incorporated by reference in their entirety. In certain embodiments, the AAV serotype may be as described in Jackson et al (Frontiers in Molecular Neuroscience 9:154 (2016)), the contents of which are herein incorporated by reference in their entirety.

In certain embodiments, the AAV serotype is selected for use due to its tropism for cells of the central nervous system. In certain embodiments, the cells of the central nervous system are neurons. In another embodiment, the cells of the central nervous system are astrocytes.

In certain embodiments, the AAV serotype is selected for use due to its tropism for cells of the muscle(s).

In certain embodiments, the initiation codon for translation of the AAV VP1 capsid protein may be CTG, TTG, or GTG as described in U.S. Pat. No. 8,163,543, the contents of which are herein incorporated by reference in its entirety.

The present disclosure refers to structural capsid proteins (including VP1, VP2, and VP3) which are encoded by capsid (Cap) genes. These capsid proteins form an outer protein structural shell (i.e., capsid) of a viral vector such as AAV. VP capsid proteins synthesized from Cap polynucleotides generally include a methionine as the first amino acid in the peptide sequence (Met1), which is associated with the start codon (AUG or ATG) in the corresponding Cap nucleotide sequence. However, it is common for a first-methionine (Met1) residue or generally any first amino acid (AA1) to be cleaved off after or during polypeptide synthesis by protein processing enzymes such as Met-aminopeptidases. This “Met/AA-clipping” process often correlates with a corresponding acetylation of the second amino acid in the polypeptide sequence (e.g., alanine, valine, serine, threonine, etc.). Met-clipping commonly occurs with VP1 and VP3 capsid proteins but can also occur with VP2 capsid proteins.

Where the Met/AA-clipping is incomplete, a mixture of one or more (one, two or three) VP capsid proteins including the viral capsid may be produced, some of which may include a Met1/AA1 amino acid (Met+/AA+) and some of which may lack a Met1/AA1 amino acid as a result of Met/AA− clipping (Met−/AA−). For further discussion regarding Met/AA-clipping in capsid proteins, see Jin, et al. Direct Liquid Chromatography/Mass Spectrometry Analysis for Complete Characterization of Recombinant Adeno-Associated Virus Capsid Proteins. Hum Gene Ther Methods. 2017 Oct. 28(5):255-267; Hwang, et al. N-Terminal Acetylation of Cellular Proteins Creates Specific Degradation Signals. Science. 2010 Feb. 19. 327(5968): 973-977; the contents of which are each incorporated herein by reference in their entirety.

According to the present disclosure, references to capsid proteins is not limited to either clipped (Met−/AA−) or unclipped (Met+/AA+) and may, in context, refer to independent capsid proteins, viral capsids included of a mixture of capsid proteins, and/or polynucleotide sequences (or fragments thereof) which encode, describe, produce, or result in capsid proteins of the present disclosure. A direct reference to a “capsid protein” or “capsid polypeptide” (such as VP1, VP2 or VP2) may also include VP capsid proteins which include a Met1/AA1 amino acid (Met+/AA+) as well as corresponding VP capsid proteins which lack the Met1/AA1 amino acid as a result of Met/AA-clipping (Met−/AA−).

Further according to the present disclosure, a reference to a specific SEQ ID NO: (whether a protein or nucleic acid) which includes or encodes, respectively, one or more capsid proteins which include a Met1/AA1 amino acid (Met+/AA+) should be understood to teach the VP capsid proteins which lack the Met1/AA1 amino acid as upon review of the sequence, it is readily apparent any sequence which merely lacks the first listed amino acid (whether or not Met1/AA1).

As a non-limiting example, reference to a VP1 polypeptide sequence which is 736 amino acids in length and which includes a “Met1” amino acid (Met+) encoded by the AUG/ATG start codon may also be understood to teach a VP1 polypeptide sequence which is 735 amino acids in length and which does not include the “Met1” amino acid (Met−) of the 736 amino acid Met+ sequence. As a second non-limiting example, reference to a VP1 polypeptide sequence which is 736 amino acids in length and which includes an “AA1” amino acid (AA1+) encoded by any NNN initiator codon may also be understood to teach a VP1 polypeptide sequence which is 735 amino acids in length and which does not include the “AA1” amino acid (AA1−) of the 736 amino acid AA1+sequence.

References to viral capsids formed from VP capsid proteins (such as reference to specific AAV capsid serotypes), can incorporate VP capsid proteins which include a Met1/AA1 amino acid (Met+/AA1+), corresponding VP capsid proteins which lack the Met1/AA1 amino acid as a result of Met/AA1-clipping (Met−/AA1−), and combinations thereof (Met+/AA1+ and Met−/AA1−).

As a non-limiting example, an AAV capsid serotype can include VP1 (Met+/AA1+), VP1 (Met−/AA1−), or a combination of VP1 (Met+/AA1+) and VP1 (Met−/AA1−). An AAV capsid serotype can also include VP3 (Met+/AA1+), VP3 (Met−/AA1−), or a combination of VP3 (Met+/AA1+) and VP3 (Met−/AA1−); and can also include similar optional combinations of VP2 (Met+/AA1) and VP2 (Met−/AA1−).

### Payloads

AAV particles of the present disclosure can comprise, or be produced using, at least one payload construct which comprises at least one payload region. In certain embodiments, the payload region may be located within a viral genome, such as the viral genome of a payload construct. At the 5′ and/or the 3′ end of the payload region there may be at least one inverted terminal repeat (ITR). Within the payload region, there may be a promoter region, an intron region and a coding region.

In certain embodiments, the payload region of the AAV particle comprises one or more nucleic acid sequences encoding one or more payload, such as a payload polypeptide or polynucleotide. In certain embodiments, the payload region of the AAV particle comprises one or more nucleic acid sequences encoding one or more polypeptides or proteins of interest. In certain embodiments, the payload region of the AAV particle comprises one or more nucleic acid sequences encoding one or more modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the payload region can be included in a payload construct used for producing AAV particles. In certain embodiments, a payload construct of the present disclosure can be a bacmid, also known as a baculovirus plasmid or recombinant baculovirus genome. In certain embodiments, a payload construct of the present disclosure can be a baculovirus expression vector (BEV). In certain embodiments, a payload construct of the present disclosure can be a BIIC which includes a BEV. As used herein, the term “payloadBac” refers to a bacmid (such as a BEV) comprising a payload construct and/or payload region. Viral production cells (e.g., Sf9 cells) may be transfected with payloadBacs and/or with BIICs comprising payloadBacs.

In certain embodiments, the AAV particles of the present disclosure comprise one or more nucleic acid sequences encoding one or more payload, such as a payload polypeptide or polynucleotide, which are useful in the field of medicine for the treatment, prophylaxis, palliation, or amelioration of diseases and/or disorders, including neurological diseases and/or disorders. In certain embodiments, the AAV particles of the present disclosure are useful in the field of medicine for the treatment, prophylaxis, palliation or amelioration of Friedreich's ataxia, or any disease stemming from a loss or partial loss of frataxin protein. In certain embodiments, the AAV particles of the present disclosure are useful in the field of medicine for the treatment, prophylaxis, palliation, or amelioration of Parkinson's Disease. In certain embodiments, the AAV particles of the present disclosure are useful in the field of medicine for the treatment, prophylaxis, palliation, or amelioration of Amyotrophic lateral sclerosis. In certain embodiments, the AAV particles of the present disclosure are useful in the field of medicine for the treatment, prophylaxis, palliation, or amelioration of Huntington's Disease. In certain embodiments, the AAV particles of the present disclosure are useful in the field of medicine for the treatment, prophylaxis, palliation, or amelioration of Alzheimer's Disease.

### Payloads: Polypeptides and Variants

In certain embodiments, the payload region of the AAV particle comprises one or more nucleic acid sequences encoding a polypeptide or protein of interest. In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide, or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the AAV particle comprises a viral genome with a payload region comprising a nucleic acid sequence encoding a protein of interest (i.e., a payload protein, therapeutic protein).

Amino acid sequences encoded by payload regions of the viral genomes of the disclosure may be translated as a whole polypeptide, a plurality of polypeptides or fragments of polypeptides, which independently may be encoded by one or more nucleic acids, fragments of nucleic acids or variants of any of the aforementioned. In certain embodiments, polypeptides can include proteins, polypeptides, and peptides of any size, structure, or function. In some instances, the polypeptide encoded is smaller than about 50 amino acids (i.e., peptide). If the polypeptide is a peptide, it will be at least about 2, 3, 4, or at least 5 amino acid residues long. Thus, polypeptides include gene products, naturally occurring polypeptides, synthetic polypeptides, homologs, orthologs, paralogs, fragments and other equivalents, variants, and analogs of the foregoing. A polypeptide may be a single molecule or may be a multi-molecular complex such as a dimer, trimer, or tetramer. They may also include single chain or multichain polypeptides and may be associated or linked. The term polypeptide may also apply to amino acid polymers in which one or more amino acid residues are an artificial chemical analogue of a corresponding naturally occurring amino acid.

In certain embodiments, the polypeptide can be a polypeptide variant which differs in amino acid sequence from a native or reference sequence. The amino acid sequence variants may possess substitutions, deletions, and/or insertions at certain positions within the amino acid sequence, as compared to a native or reference sequence. Ordinarily, variants will possess at least about 50% identity (homology) to a native or reference sequence, and in certain embodiments, they will be at least about 80%, or at least about 90% identical (homologous) to a native or reference sequence.

In certain embodiments, the payload region of the AAV particle comprises one or more nucleic acid sequences encoding a polypeptide or protein of interest.

In certain embodiments, the AAV particle comprises a viral genome with a payload region comprising nucleic acid sequences encoding more than one polypeptide of interest. In certain embodiments, a viral genome encoding one or more polypeptides may be replicated and packaged into a viral particle. A target cell transduced with a viral particle comprising the viral genome may express each of the one or more polypeptides in the single target cell.

Where the AAV particle payload region encodes a polypeptide, the polypeptide may be a peptide, polypeptide, or protein. As a non-limiting example, the payload region may encode at least one therapeutic protein of interest. The AAV viral genomes encoding polypeptides described herein may be useful in the fields of human disease, viruses, infections veterinary applications and a variety of in vivo and in vitro settings.

In certain embodiments, administration of the formulated AAV particles (which comprise the viral genome) to a subject will increase the expression of a protein in a subject. In certain embodiments, the increase of the expression of the protein will reduce the effects and/or symptoms of a disease or ailment associated with the polypeptide encoded by the payload.

In certain embodiments, the formulated AAV particles of the present disclosure may be used to reduce the decline of functional capacity and activities of daily living as measured by a standard evaluation system such as, but not limited to, the total functional capacity (TFC) scale.

In certain embodiments, the AAV particle comprises a viral genome with a payload region comprising a nucleic acid sequence encoding a protein of interest (i.e., a payload protein, therapeutic protein).

In certain embodiments, the payload region comprises a nucleic acid sequence encoding a protein including but not limited to an antibody, Aromatic L-Amino Acid Decarboxylase (AADC), ApoE2, Frataxin, survival motor neuron (SMN) protein, glucocerebrosidase, N-sulfoglucosamine sulfohydrolase, N-acetyl-alpha-glucosaminidase, iduronate 2-sulfatase, alpha-L-iduronidase, palmitoyl-protein thioesterase 1, tripeptidyl peptidase 1, battenin, CLN5, CLN6 (linclin), MFSD8, CLN8, aspartoacylase (ASPA), progranulin (GRN), MeCP2, beta-galactosidase (GLB1) and/or gigaxonin (GAN).

In certain embodiments, the AAV particle includes a viral genome with a payload region comprising a nucleic acid sequence encoding AADC or any other payload known in the art for treating Parkinson's disease. As a non-limiting example, the payload may include a sequence such as NM_001082971.1 (GI: 132814447), NM_000790.3 (GI: 132814459), NM_001242886.1 (GI: 338968913), NM_001242887.1 (GI: 338968916), NM_001242888.1 (GI: 338968918), NM_001242889.1 (GI: 338968920), NM_001242890.1 (GI: 338968922) and fragment or variants thereof.

In certain embodiments, the AAV particle comprises a viral genome with a payload region comprising a nucleic acid sequence encoding frataxin or any other payload known in the art for treating Friedreich's Ataxia. As a non-limiting example, the payload may comprise a sequence such as NM_000144.4 (GI: 239787167), NM_181425.2 (GI: 239787185), NM_001161706.1 (GI: 239787197) and fragment or variants thereof.

In certain embodiments, the AAV particle comprises a viral genome with a payload region comprising a nucleic acid sequence encoding SMN or any other payload known in the art for treating spinal muscular atrophy (SMA). As a non-limiting example, the payload may comprise a sequence such as NM_001297715.1 (GI: 663070993), NM_000344.3 (GI: 196115055), NM_022874.2 (GI: 196115040) and fragment or variants thereof.

In certain embodiments, the AAV particle comprises a viral genome with a payload region comprising a nucleic acid sequence encoding any of the disease-associated proteins (and fragment or variants thereof) described in U. S. Patent publication No. 20180258424; the content of which is herein incorporated by reference in its entirety.

In certain embodiments, the AAV particle includes a viral genome with a payload region comprising a nucleic acid sequence encoding any of the disease-associated proteins (and fragment or variants thereof) described in any one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety

In certain embodiments, the formulated AAV particles of the present disclosure may be used to improve performance on any assessment used to measure symptoms of a neurodegenerative disorder/disease. Such assessments comprise, but are not limited to ADAS-cog (Alzheimer Disease Assessment Scale—cognitive), MMSE (Mini-Mental State Examination), GDS (Geriatric Depression Scale), FAQ (Functional Activities Questionnaire), ADL (Activities of Daily Living), GPCOG (General Practitioner Assessment of Cognition), Mini-Cog, AMTS (Abbreviated Mental Test Score), Clock-drawing test, 6-CIT (6-item Cognitive Impairment Test), TYM (Test Your Memory), MoCa (Montreal Cognitive Assessment), ACE-R (Addenbrookes Cognitive Assessment), MIS (Memory Impairment Screen), BADLS (Bristol Activities of Daily Living Scale), Barthel Index, Functional Independence Measure, Instrumental Activities of Daily Living, IQCODE (Informant Questionnaire on Cognitive Decline in the Elderly), Neuropsychiatric Inventory, The Cohen-Mansfield Agitation Inventory, BEHAVE-AD, EuroQol, Short Form-36 and/or MBR Caregiver Strain Instrument, or any of the other tests as described in Sheehan B Ther Adv Neurol Disord 5(6):349-358 (2012), the contents of which are herein incorporated by reference in their entirety.

In certain embodiments “variant mimics” are provided. As used herein, the term “variant mimic” is one which contains one or more amino acids which would mimic an activated sequence. For example, glutamate may serve as a mimic for phosphoro-threonine and/or phosphoro-serine. Alternatively, variant mimics may result in deactivation or in an inactivated product containing the mimic, e.g., phenylalanine may act as an inactivating substitution for tyrosine; or alanine may act as an inactivating substitution for serine.

In certain embodiments an “amino acid sequence variant” is provided. The term “amino acid sequence variant” refers to molecules with some differences in their amino acid sequences as compared to a native or starting sequence. The amino acid sequence variants may possess substitutions, deletions, and/or insertions at certain positions within the amino acid sequence. “Native” or “starting” sequence should not be confused with a wild type sequence. As used herein, a native or starting sequence is a relative term referring to an original molecule against which a comparison may be made. “Native” or “starting” sequences or molecules may represent the wild-type (that sequence found in nature) but do not have to be the wild-type sequence.

Ordinarily, variants will possess at least about 70% homology to a native sequence, and in certain embodiments, they will be at least about 80% or at least about 90% homologous to a native sequence. “Homology” as it applies to amino acid sequences is defined as the percentage of residues in the candidate amino acid sequence that are identical with the residues in the amino acid sequence of a second sequence after aligning the sequences and introducing gaps, if necessary, to achieve the maximum percent homology. Methods and computer programs for the alignment are well known in the art. It is understood that homology depends on a calculation of percent identity but may differ in value due to gaps and penalties introduced in the calculation.

By “homologs” as it applies to amino acid sequences is meant the corresponding sequence of other species having substantial identity to a second sequence of a second species.

“Analogs” is meant to comprise polypeptide variants which differ by one or more amino acid alterations, e.g., substitutions, additions or deletions of amino acid residues that still maintain the properties of the parent polypeptide.

Sequence tags or amino acids, such as one or more lysines, can be added to the peptide sequences of the disclosure (e.g., at the N-terminal or C-terminal ends). Sequence tags can be used for peptide purification or localization. Lysines can be used to increase peptide solubility or to allow for biotinylation. Alternatively, amino acid residues located at the carboxy and amino terminal regions of the amino acid sequence of a peptide or protein may optionally be deleted providing for truncated sequences. Certain amino acids (e.g., C-terminal or N-terminal residues) may alternatively be deleted depending on the use of the sequence, as for example, expression of the sequence as part of a larger sequence which is soluble or linked to a solid support.

In certain embodiments a “substitutional variant” is provided. “Substitutional variants” when referring to proteins are those that have at least one amino acid residue in a native or starting sequence removed and a different amino acid inserted in its place at the same position. The substitutions may be single, where only one amino acid in the molecule has been substituted, or they may be multiple, where two or more amino acids have been substituted in the same molecule.

As used herein the term “conservative amino acid substitution” refers to the substitution of an amino acid that is normally present in the sequence with a different amino acid of similar size, charge, or polarity. Examples of conservative substitutions comprise the substitution of a non-polar (hydrophobic) residue such as isoleucine, valine and leucine for another non-polar residue. Likewise, examples of conservative substitutions comprise the substitution of one polar (hydrophilic) residue for another such as between arginine and lysine, between glutamine and asparagine, and between glycine and serine. Additionally, the substitution of a basic residue such as lysine, arginine or histidine for another, or the substitution of one acidic residue such as aspartic acid or glutamic acid for another acidic residue are additional examples of conservative substitutions. Examples of non-conservative substitutions comprise the substitution of a non-polar (hydrophobic) amino acid residue such as isoleucine, valine, leucine, alanine, methionine for a polar (hydrophilic) residue such as cysteine, glutamine, glutamic acid or lysine and/or a polar residue for a non-polar residue.

In certain embodiments an “insertional variant” is provided. “Insertional variants” when referring to proteins are those with one or more amino acids inserted immediately adjacent to an amino acid at a particular position in a native or starting sequence “Immediately adjacent” to an amino acid means connected to either the alpha-carboxy or alpha-amino functional group of the amino acid.

In certain embodiments a “deletional variant” is provided. “Deletional variants” when referring to proteins, are those with one or more amino acids in the native or starting amino acid sequence removed. Ordinarily, deletional variants will have one or more amino acids deleted in a particular region of the molecule.

As used herein, the term “derivative” is used synonymously with the term “variant” and refers to a molecule that has been modified or changed in any way relative to a reference molecule or starting molecule. In certain embodiments, derivatives comprise native or starting proteins that have been modified with an organic proteinaceous or non-proteinaceous derivatizing agent, and post-translational modifications. Covalent modifications are traditionally introduced by reacting targeted amino acid residues of the protein with an organic derivatizing agent that is capable of reacting with selected side-chains or terminal residues, or by harnessing mechanisms of post-translational modifications that function in selected recombinant host cells. The resultant covalent derivatives are useful in programs directed at identifying residues important for biological activity, for immunoassays, or for the preparation of anti-protein antibodies for immunoaffinity purification of the recombinant glycoprotein. Such modifications are within the ordinary skill in the art and are performed without undue experimentation.

Certain post-translational modifications are the result of the action of recombinant host cells on the expressed polypeptide. Glutaminyl and asparaginyl residues are frequently post-translationally deamidated to the corresponding glutamyl and aspartyl residues. Alternatively, these residues are deamidated under mildly acidic conditions. Either form of these residues may be present in the proteins used in accordance with the present disclosure.

Other post-translational modifications comprise hydroxylation of proline and lysine, phosphorylation of hydroxyl groups of seryl or threonyl residues, methylation of the alpha-amino groups of lysine, arginine, and histidine side chains (T. E. Creighton, Proteins: Structure and Molecular Properties, W.H. Freeman & Co., San Francisco, pp. 79-86 (1983)).

“Features” when referring to proteins are defined as distinct amino acid sequence-based components of a molecule. Features of the proteins of the present disclosure comprise surface manifestations, local conformational shape, folds, loops, half-loops, domains, half-domains, sites, termini, or any combination thereof.

As used herein when referring to polynucleotides the term “loop” refers to a structural feature which may serve to reverse the direction of the backbone of a polynucleotide such that two regions at a distance of the polynucleotide are brought together spatially. Loops may be open or closed. Closed loops or “cyclic” loops may include 2, 3, 4, 5, 6, 7, 8, 9, 10 or more nucleotides.

As used herein the term “domain” refers to a motif of a polynucleotide having one or more identifiable structural or functional characteristics or properties (e.g., binding capacity, serving as a site for interactions).

As used herein the terms “site” as it pertains to polynucleotides is used synonymously with “nucleic acid residue” and/or “nucleotide.” A site represents a position within a polynucleotide that may be modified, manipulated, altered, derivatized or varied.

As used herein the terms “termini” or “terminus” refers to an extremity of a polynucleotide. Such extremity is not limited only to the first or final site of the polynucleotide but may include additional nucleotides in the terminal regions. The polynucleotides of the present disclosure may be characterized as having both a 5′ and a 3′ terminus.

Once any of the features have been identified or defined as a component of a molecule of the disclosure, any of several manipulations and/or modifications of these features may be performed by moving, swapping, inverting, deleting, randomizing, or duplicating. Furthermore, it is understood that manipulation of features may result in the same outcome as a modification to the molecules of the disclosure. For example, a manipulation which involves deleting a domain would result in the alteration of the length of a molecule just as modification of a nucleic acid to encode less than a full-length molecule would.

Modifications and manipulations can be accomplished by methods known in the art such as site directed mutagenesis. The resulting modified molecules may then be tested for activity using in vitro or in vivo assays such as those described herein, or any other suitable screening assay known in the art.

### Payloads: Modulatory Polynucleotides Targeting a Gene of Interest

The present disclosure comprises the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, 1 or 2 nucleotide overhangs at the 3′ end of each strand.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory, or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g., antisense strand RNA or antisense oligonucleotides). In many cases it requires higher concentration of the ss-siRNA to achieve the effective gene silencing potency of the corresponding duplex.

In certain embodiments, the siRNA molecules may be encoded in a modulatory polynucleotide which also comprises a molecular scaffold. As used herein a “molecular scaffold” is a framework or starting molecule that forms the sequence or structural basis against which to design or make a subsequent molecule.

In certain embodiments, the modulatory polynucleotide which comprises the payload (e.g., siRNA, miRNA or other RNAi agent described herein) comprises molecular scaffold which comprises a leading 5′ flanking sequence which may be of any length and may be derived in whole or in part from wild type microRNA sequence or be completely artificial. A 3′ flanking sequence may mirror the 5′ flanking sequence in size and origin. In certain embodiments, one or both of the 5′ and 3′ flanking sequences are absent.

In certain embodiments, the molecular scaffold may comprise one or more linkers known in the art. The linkers may separate regions or one molecular scaffold from another. As a non-limiting example, the molecular scaffold may be polycistronic.

In certain embodiments, the modulatory polynucleotide is designed using at least one of the following properties: loop variant, seed mismatch/bulge/wobble variant, stem mismatch, loop variant and basal stem mismatch variant, seed mismatch and basal stem mismatch variant, stem mismatch and basal stem mismatch variant, seed wobble and basal stem wobble variant, or a stem sequence variant.

In certain embodiments, the present disclosure presents the use of formulated AAV particles whose viral genomes encode modulatory polynucleotides, e.g., RNA or DNA molecules as therapeutic agents. Accordingly, the present disclosure provides viral genomes which encode polynucleotides which are processed into small double stranded RNA (dsRNA) molecules (small interfering RNA, siRNA, miRNA, pre-miRNA) targeting a gene of interest. The present disclosure also provides methods of their use for inhibiting gene expression and protein production of an allele of the gene of interest, for treating diseases, disorders, and/or conditions.

In certain embodiments, the AAV particle comprises a viral genome with a payload region comprising a nucleic acid sequence encoding or comprising one or more modulatory polynucleotides. In certain embodiments, the AAV particle comprises a viral genome with a payload region comprising a nucleic acid sequence encoding a modulatory polynucleotide of interest. In certain embodiments of the present disclosure, modulatory polynucleotides, e.g., RNA or DNA molecules, are presented as therapeutic agents. RNA interference mediated gene silencing can specifically inhibit targeted gene expression.

In certain embodiments, the payload region comprises a nucleic acid sequence encoding a modulatory polynucleotide which interferes with a target gene expression and/or a target protein production. In certain embodiments, the gene expression or protein production to be inhibited/modified may comprise but are not limited to superoxide dismutase 1 (SOD1), chromosome 9 open reading frame 72 (C9ORF72), TAR DNA binding protein (TARDBP), ataxin-3 (ATXN3), huntingtin (HTT), amyloid precursor protein (APP), apolipoprotein E (ApoE), microtubule-associated protein tau (MAPT), alpha-synuclein (SNCA), voltage-gated sodium channel alpha subunit 9 (SCN9A), and/or voltage-gated sodium channel alpha subunit 10 (SCN10A).

The present disclosure provides small interfering RNA (siRNA) duplexes (and modulatory polynucleotides encoding them) that target SOD1 mRNA to interfere with the gene expression and/or protein production of SOD1. The present disclosure also provides methods of their use for inhibiting gene expression and protein production of an allele of SOD1, for treating amyotrophic lateral sclerosis (ALS). In certain embodiments, the siRNA duplexes of the present disclosure may target SOD1 along any segment of the respective nucleotide sequence. In certain embodiments, the siRNA duplexes of the present disclosure may target SOD1 at the location of a SNP or variant within the nucleotide sequence.

The present disclosure provides small interfering RNA (siRNA) duplexes (and modulatory polynucleotides encoding them) that target HTT mRNA to interfere with the gene expression and/or protein production of HTT. The present disclosure also provides methods of their use for inhibiting gene expression and protein production of an allele of HTT, for treating Huntington's disease (HD). In certain embodiments, the siRNA duplexes of the present disclosure may target HTT along any segment of the respective nucleotide sequence. In certain embodiments, the siRNA duplexes of the present disclosure may target HTT at the location of a SNP or variant within the nucleotide sequence.

In certain embodiments, the AAV particle comprises a viral genome with a payload region comprising a nucleic acid sequence encoding any of the modulatory polynucleotides, RNAi molecules, siRNA molecules, dsRNA molecules, and/or RNA duplexes described in any one of the following International Publications: WO2016077687, WO2016077689, WO2018204786, WO2017201258, WO2017201248, WO2018204803, WO2018204797, WO2017189959, WO2017189963, WO2017189964, WO2015191508, WO2016094783, WO20160137949, WO2017075335; the contents of which are each herein incorporated by reference in their entirety.

In certain embodiments, a nucleic acid sequence encoding such siRNA molecules, or a single strand of the siRNA molecules, is inserted into adeno-associated viral vectors and introduced into cells, specifically cells in the central nervous system.

AAV particles have been investigated for siRNA delivery because of several unique features. Non-limiting examples of the features comprise (i) the ability to infect both dividing and non-dividing cells; (ii) a broad host range for infectivity, comprising human cells; (iii) wild-type AAV has not been associated with any disease and has not been shown to replicate in infected cells; (iv) the lack of cell-mediated immune response against the vector and (v) the non-integrative nature in a host chromosome thereby reducing potential for long-term expression. Moreover, infection with AAV particles has minimal influence on changing the pattern of cellular gene expression (Stilwell and Samulski et al., Biotechniques, 2003, 34, 148).

In certain embodiments, the encoded siRNA duplex of the present disclosure contains an antisense strand and a sense strand hybridized together forming a duplex structure, wherein the antisense strand is complementary to the nucleic acid sequence of the targeted gene of interest, and wherein the sense strand is homologous to the nucleic acid sequence of the targeted gene of interest. In other aspects, there are 0, for 2 nucleotide overhangs at the 3′ end of each strand.

According to the present disclosure, each strand of the siRNA duplex targeting the gene of interest can be about 19 to 25, 19 to 24 or 19 to 21 nucleotides in length, such as about 19 nucleotides, 20 nucleotides, 21 nucleotides, 22 nucleotides, 23 nucleotides, 24 nucleotides, or 25 nucleotides in length.

In certain embodiments, an siRNA or dsRNA comprises at least two sequences that are complementary to each other. The dsRNA comprises a sense strand having a first sequence and an antisense strand having a second sequence. The antisense strand comprises a nucleotide sequence that is substantially complementary to at least part of an mRNA encoding a gene of interest, and the region of complementarity is 30 nucleotides or less, and at least 15 nucleotides in length. Generally, the dsRNA is 19 to 25, 19 to 24 or 19 to 21 nucleotides in length. In certain embodiments, the dsRNA is from about 15 to about 25 nucleotides in length, and in certain embodiments the dsRNA is from about 25 to about 30 nucleotides in length.

The dsRNA encoded in an expression vector upon contacting with a cell expressing protein encoded by the gene of interest, inhibits the expression of protein encoded by the gene of interest by at least 10%, at least 20%, at least 25%, at least 30%, at least 35%, at least 40%, at least 50%, at least 60%, at least 70%, at least 80%, or more, when assayed by methods known in the art or a method as described herein.

According to the present disclosure, the siRNA molecules are designed and tested for their ability in reducing mRNA levels in cultured cells.

In certain embodiments, the siRNA molecules are designed and tested for their ability in reducing levels of the gene of interest in cultured cells.

The present disclosure also provides pharmaceutical compositions comprising at least one siRNA duplex targeting the gene of interest and a pharmaceutically acceptable carrier. In certain embodiments, the siRNA duplex is encoded by a viral genome in an AAV particle.

In certain embodiments, the present disclosure provides methods for inhibiting/silencing gene expression in a cell. In some aspects, the inhibition of gene expression refers to an inhibition by at least about 20%, such as by at least about 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%.

In certain embodiments, the encoded siRNA duplexes may be used to reduce the expression of protein or mRNA encoded by the gene of interest by at least about 20%, 30%, 40%, 50%, 60%, 70%, 80%, 85%, 90%, 95% and 100%, or at least 20-30%, 20-40%, 20-50%, 20-60%, 20-70%, 20-80%, 20-90%, 20-95%, 20-100%, 30-40%, 35-40%, 30-50%, 30-60%, 30-70%, 30-80%, 30-90%, 30-95%, 30-100%, 40-50%, 40-60%, 40-70%, 40-80%, 40-90%, 40-95%, 40-100%, 50-60%, 50-70%, 50-80%, 50-90%, 50-95%, 50-100%, 60-70%, 60-80%, 60-90%, 60-95%, 60-100%, 70-80%, 70-90%, 70-95%, 70-100%, 80-90%, 80-95%, 80-100%, 90-95%, 90-100% or 95-100%. As a non-limiting example, the expression of protein or mRNA may be reduced 50-90%. As a non-limiting example, the expression of protein or mRNA may be reduced 30-70%. As a non-limiting example, the expression of protein or mRNA may be reduced 40-70%.

In certain embodiments, the encoded siRNA duplexes may be used to reduce the expression of protein encoded by the gene of interest and/or transcribed mRNA in at least one region of the CNS. As a non-limiting example, the region is the neurons (e.g., cortical neurons).

In certain embodiments, the formulated AAV particles comprising such encoded siRNA molecules may be introduced directly into the central nervous system of the subject, for example, by infusion into the putamen.

In certain embodiments, the formulated AAV particles comprising such encoded siRNA molecules may be introduced directly into the central nervous system of the subject, for example, by infusion into the thalamus of a subject.

In certain embodiments, the formulated AAV particles comprising such encoded siRNA molecules may be introduced directly into the central nervous system of the subject, for example, by infusion into the white matter of a subject.

In certain embodiments, the formulated AAV particles comprising such encoded siRNA molecules may be introduced to the central nervous system of the subject, for example, by intravenous administration to a subject.

In certain embodiments, the pharmaceutical composition of the present disclosure is used as a solo therapy. In certain embodiments, the pharmaceutical composition of the present disclosure is used in combination therapy. The combination therapy may be in combination with one or more neuroprotective agents such as small molecule compounds, growth factors and hormones which have been tested for their neuroprotective effect on motor neuron degeneration.

The payloads of the formulated AAV particles of the present disclosure may encode one or more agents which are subject to RNA interference (RNAi) induced inhibition of gene expression. Provided herein are encoded siRNA duplexes or encoded dsRNA that target a gene of interest (referred to herein collectively as “siRNA molecules”). Such siRNA molecules, e.g., encoded siRNA duplexes, encoded dsRNA or encoded siRNA or dsRNA precursors can reduce or silence gene expression in cells, for example, astrocytes or microglia, cortical, hippocampal, entorhinal, thalamic, sensory, or motor neurons.

RNAi (also known as post-transcriptional gene silencing (PTGS), quelling, or co-suppression) is a post-transcriptional gene silencing process in which RNA molecules, in a sequence specific manner, inhibit gene expression, typically by causing the destruction of specific mRNA molecules. The active components of RNAi are short/small double stranded RNAs (dsRNAs), called small interfering RNAs (siRNAs), that typically contain 15-30 nucleotides (e.g., 19 to 25, 19 to 24 or 19-21 nucleotides) and 2-nucleotide 3′ overhangs and that match the nucleic acid sequence of the target gene. These short RNA species may be naturally produced in vivo by Dicer-mediated cleavage of larger dsRNAs and they are functional in mammalian cells.

In some embodiments, the modulatory polynucleotides of the viral genome may comprise at least one nucleic acid sequence encoding at least one siRNA molecule. The nucleic acid sequence may, independently if there is more than one, encode 1, 2, 3, 4, 5, 6, 7, 8, 9, or more than 9 siRNA molecules.

Naturally expressed small RNA molecules, known as microRNAs (miRNAs), elicit gene silencing by regulating the expression of mRNAs. The miRNAs containing RNA Induced Silencing Complex (RISC) targets mRNAs presenting a perfect sequence complementarity with nucleotides 2-7 in the 5′ region of the miRNA which is called the seed region, and other base pairs with its 3′ region. miRNA mediated down regulation of gene expression may be caused by cleavage of the target mRNAs, translational inhibition of the target mRNAs, or mRNA decay. miRNA targeting sequences are usually located in the 3′ UTR of the target mRNAs. A single miRNA may target more than 100 transcripts from various genes, and one mRNA may be targeted by different miRNAs.

siRNA duplexes or dsRNA targeting a specific mRNA may be designed as a payload of an AAV particle and introduced into cells for activating RNAi processes. Elbashir et al. demonstrated that 21-nucleotide siRNA duplexes (termed small interfering RNAs) were capable of effecting potent and specific gene knockdown without inducing immune response in mammalian cells (Elbashir S M et al., Nature, 2001, 411, 494-498). Since this initial report, post-transcriptional gene silencing by siRNAs quickly emerged as a powerful tool for genetic analysis in mammalian cells and has the potential to produce novel therapeutics.

The siRNA duplex comprised of a sense strand homologous to the target mRNA and an antisense strand that is complementary to the target mRNA offers much more advantage in terms of efficiency for target RNA destruction compared to the use of the single strand (ss)-siRNAs (e.g., antisense strand RNA or antisense oligonucleotides). In many cases it requires higher concentration of the ss-siRNA to achieve the effective gene silencing potency of the corresponding duplex.

Any of the foregoing molecules may be encoded by an AAV particle or viral genome.

Introduction into Cells

The encoded payload of the present disclosure may be introduced into cells by being encoded by the viral genome of an AAV particle. These AAV particles can be engineered and optimized to facilitate the entry into cells that are not readily amendable to transfection/transduction. Also, some synthetic viral vectors possess an ability to integrate the payload into the cell genome, thereby leading to stable payload expression and long-term therapeutic effect. In this manner, viral vectors are engineered as vehicles for specific delivery while lacking the deleterious replication and/or integration features found in wild-type virus.

In certain embodiments, the encoded payload is introduced into a cell by transfecting, infecting or transducing the cell with an AAV particle comprising nucleic acid sequences capable of producing the payload when processed in the cell. In certain embodiments, the payload is introduced into a cell by injecting into the cell or tissue an AAV particle comprising a nucleic acid sequence capable of producing the payload when processed in the cell.

Other methods for introducing AAV particles comprising the nucleic acid sequence for the payloads described herein may comprise photochemical internalization as described in U. S. Patent publication No. 20120264807, the content of which is incorporated herein by reference in its entirety as related to photochemical internalizations.

In certain embodiments, the formulations described herein may contain at least one AAV particle comprising the nucleic acid sequence encoding the payloads described herein. In certain embodiments, the payloads may target the gene of interest at one target site. In another embodiment, the formulation comprises a plurality of AAV particles, each AAV particle comprising a nucleic acid sequence encoding a payload targeting a gene of interest at a different target site. The gene of interest may be targeted at 2, 3, 4, 5 or more than 5 sites.

In certain embodiments, the AAV particles from any relevant species, such as, but not limited to, human, pig, dog, mouse, rat, or monkey may be introduced into cells.

In certain embodiments, the formulated AAV particles may be introduced into cells or tissues which are relevant to the disease to be treated. In certain embodiments, the formulated AAV particles may be introduced into cells which have a high level of endogenous expression of the target gene. In another embodiment, the formulated AAV particles may be introduced into cells which have a low level of endogenous expression of the target gene. In certain embodiments, the cells may be those which have a high efficiency of AAV transduction.

In certain embodiments, formulated AAV particles comprising a nucleic acid sequence encoding a payload of the present disclosure may be used to deliver the payload to the central nervous system (e.g., U.S. Pat. No. 6,180,613; the content of which is incorporated herein by reference in its entirety as related to the delivery and therapeutic use of siRNA molecules and AAV particles).

In certain embodiments, the formulated AAV particles comprising a nucleic acid sequence encoding a payload of the present disclosure may further comprise a modified capsid comprising peptides from non-viral origin. In other aspects, the AAV particle may contain a CNS specific chimeric capsid to facilitate the delivery of encoded siRNA duplexes into the brain and the spinal cord. For example, an alignment of cap nucleotide sequences from AAV variants exhibiting CNS tropism may be constructed to identify variable region (VR) sequence and structure.

In certain embodiments, AAV particle comprising the nucleic acid sequence for the siRNA molecules of the present disclosure may be formulated for CNS delivery. Agents that cross the brain blood barrier may be used. For example, some cell penetrating peptides that can target siRNA molecules to the brain blood barrier endothelium may be used to formulate the siRNA duplexes targeting the gene of interest.

In certain embodiments, the formulated AAV particle comprising a nucleic acid sequence encoding a payload of the present disclosure may be administered directly to the CNS. As a non-limiting example, the vector comprises a nucleic acid sequence encoding an siRNA molecule targeting the gene of interest. As a non-limiting example, the vector comprises a nucleic acid sequence encoding an polypeptide targeting a gene of interest.

In certain embodiments, the formulated AAV particle may be administered to a subject (e.g., to the CNS of a subject) in a therapeutically effective amount.

### II. AAV Production

### General Viral Production Process

Mammalian cells and/or insect cells are often used as viral production cells for the production of rAAV particles. In various embodiments, the methods and systems disclosed herein employ insect cells, e.g., Sf9 cells.

AAV production systems using mammalian or insect cells present a range of complications. There is continued need for methods and systems which allow for effective and efficient large scale (commercial) production of rAAV particles in mammalian and insect cells.

The details of one or more embodiments of the present disclosure are set forth in the accompanying description below. Other features, objects, and advantages of the present disclosure will be apparent from the description, drawings, and the claims. In the description, the singular forms also include the plural unless the context clearly dictates otherwise. Unless defined otherwise, all technical and scientific terms used herein have the same meaning as commonly understood by one of ordinary skill in the art to which this present disclosure belongs. In the case of conflict with disclosures incorporated by reference, the present express description will control.

In certain embodiments, the constructs, polynucleotides, polypeptides, vectors, serotypes, capsids formulations, or particles of the present disclosure may be, may comprise, may be modified by, may be used by, may be used for, may be used with, or may be produced with any sequence, element, construct, system, target or process described in one of the following International Publications: WO2016073693, WO2017023724, WO2018232055, WO2016077687, WO2016077689, WO2018204786, WO2017201258, WO2017201248, WO2018204803, WO2018204797, WO2017189959, WO2017189963, WO2017189964, WO2015191508, WO2016094783, WO2016137949, WO2017075335; the contents of which are each incorporated herein by reference in their entireties, insofar as they do not conflict with the present disclosure.

AAV production of the present disclosure comprises processes and methods for producing AAV particles and viral vectors which can contact a target cell to deliver a payload construct, e.g., a recombinant viral construct, which comprises a nucleotide encoding a payload molecule. In certain embodiments, the viral vectors are adeno-associated viral (AAV) vectors such as recombinant adeno-associated viral (rAAV) vectors. In certain embodiments, the AAV particles are adeno-associated viral (AAV) particles such as recombinant adeno-associated viral (rAAV) particles.

The present disclosure provides methods of producing AAV particles or viral vectors by (a) contacting a viral production cell with one or more viral expression constructs encoding at least one AAV capsid protein and/or at least one AAV replication protein, and one or more payload construct vectors, wherein said payload construct vector comprises a payload construct encoding a payload molecule selected from the group consisting of a transgene, a polynucleotide encoding protein, and a modulatory nucleic acid; (b) culturing said viral production cell under conditions such that at least one AAV particle or viral vector is produced, and (c) isolating said at least one AAV particle or viral vector.

In these methods a viral expression construct may encode at least one structural protein and/or at least one non-structural protein. The structural protein may comprise any of the native or wild type capsid proteins VP1, VP2, and/or VP3 or a chimeric protein. The non-structural protein may comprise any of the native or wild type Rep78, Rep68, Rep52 and/or Rep40 proteins or a chimeric protein.

In certain embodiments, an rAAV production method as disclosed herein comprises transient transfection, viral transduction and/or electroporation.

In certain embodiments, the viral production cell is selected from the group consisting of a mammalian cell and an insect cell. In certain embodiments, the insect cell comprises a Spodoptera frugiperda insect cell. In certain embodiments, the insect cell comprises an Sf9 insect cell. In certain embodiments, the insect cell comprises an Sf21 insect cell.

The payload construct vector of the present disclosure may comprise at least one inverted terminal repeat (ITR) and may comprise mammalian DNA.

Also provided are AAV particles and viral vectors produced according to the methods described herein.

The AAV particles of the present disclosure may be formulated as a pharmaceutical composition with one or more acceptable excipients.

In certain embodiments, an AAV particle or viral vector may be produced by a method described herein.

In certain embodiments, the AAV particles may be produced by contacting a viral production cell (e.g., an insect cell) with at least one viral expression construct encoding at least one capsid protein and at least one AAV replication protein, and at least one payload construct vector. In certain embodiments, separate viral expression constructs encoding the at least one capsid protein and the at least one AAV replication protein may be used. The viral production cell may be contacted by transient transfection, viral transduction and/or electroporation. The payload construct vector may comprise a payload construct encoding a payload molecule such as, but not limited to, a transgene, a polynucleotide encoding protein, and a modulatory nucleic acid. The viral production cell can be cultured under conditions such that at least one AAV particle or viral vector is produced, isolated (e.g., using temperature-induced lysis, mechanical lysis and/or chemical lysis) and/or purified (e.g., using filtration, chromatography and/or immunoaffinity purification). As a non-limiting example, the payload construct vector may comprise mammalian DNA.

In certain embodiments, the AAV particles are produced in an insect cell (e.g., Spodoptera frugiperda (Sf9) cell) using the method described herein. As a non-limiting example, the insect cell is contacted using viral transduction which may comprise baculoviral transduction.

In another embodiment, the AAV particles are produced in a mammalian cell using the method described herein. As a non-limiting example, the mammalian cell is contacted using transient transfection.

In certain embodiments, the viral expression construct may encode at least one structural protein and at least one non-structural protein. As a non-limiting example, the structural protein may comprise VP1, VP2, and/or VP3 capsid proteins. As another non-limiting example, the non-structural protein may comprise Rep78, Rep68, Rep52, and/or Rep40 replication proteins.

In certain embodiments, the AAV particle production method described herein produces greater than 10¹, greater than 10², greater than 10³, greater than 10⁴, or greater than 10⁵ AAV particles in a viral production cell.

In certain embodiments, a process of the present disclosure comprises production of viral particles in a viral production cell using a viral production system which comprises at least one viral expression construct and at least one payload construct. The at least one viral expression construct and at least one payload construct can be co-transfected (e.g., dual transfection, triple transfection) into a viral production cell. The transfection is completed using standard molecular biology techniques known and routinely performed by a person skilled in the art. The viral production cell provides the cellular machinery necessary for expression of the proteins and other biomaterials necessary for producing the AAV particles, comprising Rep proteins which replicate the payload construct and Cap proteins which assemble to form a capsid that encloses the replicated payload constructs. The resulting AAV particle is extracted from the viral production cells and processed into a pharmaceutical preparation for administration.

In certain embodiments, the process for production of viral particles utilizes seed cultures of viral production cells that comprise one or more baculoviruses (e.g., a Baculoviral Expression Vector (BEV) or baculovirus infected insect cells (BIICs) that have been transfected with a viral expression construct (e.g., comprised in an expressionBac) and a payload construct (e.g., comprised in a payloadBac)). In certain embodiments, the seed cultures are harvested, divided into aliquots and frozen, and may be used at a later time point to initiate an infection of a naïve population of production cells.

Large scale production of AAV particles may utilize a bioreactor. The use of a bioreactor allows for the precise measurement and/or control of variables that support the growth and activity of viral production cells such as mass, temperature, mixing conditions (impellor RPM or wave oscillation), CO₂ concentration, O₂ concentration, gas sparge rates and volumes, gas overlay rates and volumes, pH, Viable Cell Density (VCD), cell viability, cell diameter, and/or optical density (OD). In certain embodiments, the bioreactor is used for batch production in which the entire culture is harvested at an experimentally determined time point and AAV particles are purified. In another embodiment, the bioreactor is used for continuous production in which a portion of the culture is harvested at an experimentally determined time point for purification of AAV particles, and the remaining culture in the bioreactor is refreshed with additional growth media components.

In certain embodiments, AAV viral particles can be extracted from viral production cells in a process which comprises cell lysis, clarification, sterilization, and purification. Cell lysis comprises any process that disrupts the structure of the viral production cell, thereby releasing AAV particles. In certain embodiments cell lysis may comprise thermal shock, chemical, or mechanical lysis methods. In some embodiments, cell lysis is done chemically. Clarification of the lysed cells can comprise the gross purification of the mixture of lysed cells, media components, and AAV particles. In certain embodiments, clarification comprises centrifugation and/or filtration, comprising but not limited to depth end, tangential flow, and/or hollow fiber filtration.

The end result of viral production is a purified collection of AAV particles which comprise two components: (1) a payload construct (e.g., a recombinant viral genome construct) and (2) a viral capsid.

In certain embodiments, a viral production system or process of the present disclosure comprises steps for producing baculovirus infected insect cells (BIICs) using Viral Production Cells (VPC) and plasmid constructs. Viral Production Cells (VPCs) from a Cell Bank (CB) are thawed and expanded to provide a target working volume and VPC concentration. The resulting pool of VPCs is split into a Rep/Cap VPC pool and a Payload VPC pool. One or more Rep/Cap plasmid constructs (viral expression constructs) are processed into Rep/Cap Bacmid polynucleotides and transfected into the Rep/Cap VPC pool. One or more Payload plasmid constructs (payload constructs) are processed into Payload Bacmid polynucleotides and transfected into the Payload VPC pool. The two VPC pools are incubated to produce P1 Rep/Cap Baculoviral Expression Vectors (BEVs) and P1 Payload BEVs. The two BEV pools are expanded into a collection of Plaques, with a single Plaque being selected for Clonal Plaque (CP) Purification (also referred to as Single Plaque Expansion). The process can comprise a single CP Purification step or can comprise multiple CP Purification steps either in series or separated by other processing steps. The one-or-more CP Purification steps provide a CP Rep/Cap BEV pool and a CP Payload BEV pool. These two BEV pools can then be stored and used for future production steps, or they can be then transfected into VPCs to produce a Rep/Cap BIIC pool and a Payload BIIC pool.

In certain embodiments, a viral production system or process of the present disclosure comprises steps for producing AAV particles using Viral Production Cells (VPC) and baculovirus infected insect cells (BIICs). Viral Production Cells (VPCs) from a Cell Bank (CB) are thawed and expanded to provide a target working volume and VPC concentration. This expansion can include one or more small-volume expansion steps up to a working volume of 2000-5000 mL, followed by one or more large-volume expansion steps in large-scale bioreactors (e.g., Wave and/or N−1 bioreactors) up to a working volume of 25-500 L. The working volume of Viral Production Cells is seeded into a Production Bioreactor and can be further expanded to a working volume of 200-2500 L with a target VPC concentration for BIIC infection.

The working volume of VPCs in the Production Bioreactor is then co-infected with Rep/Cap BIICs and Payload BIICs, e.g., with a target VPC:BIIC ratio and a target BIIC:BIIC ratio. VCD infection can also utilize BEVs. The co-infected VPCs are incubated and expanded in the Production Bioreactor to produce a bulk harvest of AAV particles and VPCs.

In certain embodiments, a viral production system or process of the present disclosure comprises steps for producing a Drug Substance by processing, clarifying and purifying a bulk harvest of AAV particles and Viral Production Cells. A bulk harvest of AAV particles and VPCs (within a Production Bioreactor) are processed through cellular disruption and lysis (e.g., chemical lysis and/or mechanical lysis), followed by nuclease treatment of the lysis pool, thereby producing a crude lysate pool. The crude lysate pool is processed through one or more filtration and clarification steps, comprising depth filtration and/or microfiltration to provide a clarified lysate pool. The clarified lysate pool is processed through one or more chromatography and purification steps, comprising one or more affinity chromatography (AFC) steps and one or more ion-exchange chromatography (AEX or CEX) steps, either in series or alternating, to provide a purified product pool. The purified product pool is then optionally processed through nanofiltration, and then through tangential flow filtration (TFF). The TFF process comprises one or more diafiltration (DF) steps and one or more ultrafiltration (UF) steps, either in series or alternating. The product pool is further processed through viral retention filtration (VRF) and another filtration step to provide a drug substance pool. The drug substance pool can be further filtered, then aliquoted into vials for storage and treatment.

### Viral, e.g., AAV, Expression Constructs

The viral production system of the present disclosure comprises one or more viral expression constructs which can be transfected/transduced into a viral production cell (e.g., Sf9). In certain embodiments, a viral expression construct or a payload construct of the present disclosure can be a bacmid, also known as a baculovirus plasmid or recombinant baculovirus genome. In certain embodiments, a viral expression construct of the present disclosure can be a baculovirus expression vector (BEV). In certain embodiments, a viral expression construct of the present disclosure can be a BIIC which includes a BEV. Viral production cells (e.g., Sf9 cells) may be transfected with expressionBacs and/or with BIICs comprising expressionBacs.

In certain embodiments, the viral expression region comprises a protein-coding nucleotide sequence and at least one expression control sequence for expression in a viral production cell. In certain embodiments, the viral expression region comprises a protein-coding nucleotide sequence operably linked to least one expression control sequence for expression in a viral production cell. In certain embodiments, the viral expression construct contains parvoviral genes under control of one or more promoters. Parvoviral genes can comprise nucleotide sequences encoding non-structural AAV replication proteins, such as Rep genes which encode Rep52, Rep40, Rep68, or Rep78 proteins, e.g., a combination of Rep78 and Rep52. Parvoviral genes can comprise nucleotide sequences encoding structural AAV proteins, such as Cap genes which encode VP1, VP2, and VP3 proteins.

The viral production system of the present disclosure is not limited by the viral expression vector used to introduce the parvoviral functions into the virus replication cell. The presence of the viral expression construct in the virus replication cell need not be permanent. The viral expression constructs can be introduced by any means known, for example by chemical treatment of the cells, electroporation, or infection.

Viral expression constructs of the present disclosure may comprise any compound or formulation, biological or chemical, which facilitates transformation, transfection, or transduction of a cell with a nucleic acid. Exemplary biological viral expression constructs comprise plasmids, linear nucleic acid molecules, and recombinant viruses comprising baculovirus. Exemplary chemical vectors comprise lipid complexes. Viral expression constructs are used to incorporate nucleic acid sequences into virus replication cells in accordance with the present disclosure. (O'Reilly, David R., Lois K. Miller, and Verne A. Luckow. Baculovirus expression vectors: a laboratory manual. Oxford University Press, 1994.); Maniatis et al., eds. Molecular Cloning. CSH Laboratory, NY, N.Y. (1982); and Philiport and Scluber, eds. Liposomes as tools in Basic Research and Industry. CRC Press, Ann Arbor, Mich. (1995), the contents of which are each incorporated herein by reference in their entireties as related to viral expression constructs and uses thereof, insofar as they do not conflict with the present disclosure.

In certain embodiments, the viral expression construct is an AAV expression construct which comprises one or more nucleotide sequences encoding non-structural AAV replication proteins, structural AAV capsid proteins, or a combination thereof. In certain embodiments, the viral expression region is an AAV expression region of an expression construct which comprises one or more nucleotide sequences encoding non-structural AAV replication proteins, structural AAV capsid proteins, or a combination thereof.

In certain embodiments, the viral expression construct of the present disclosure may be a plasmid vector. In certain embodiments, the viral expression construct of the present disclosure may be a baculoviral construct.

The present disclosure is not limited by the number of viral expression constructs employed to produce AAV particles or viral vectors. In certain embodiments, one, two, three, four, five, six, or more viral expression constructs can be employed to produce AAV particles in viral production cells in accordance with the present disclosure. In one non-limiting example, five expression constructs may individually encode AAV VP1, AAV VP2, AAV VP3, Rep52, Rep78, and with an accompanying payload construct comprising a payload polynucleotide and at least one AAV ITR. In another embodiment, expression constructs may be employed to express, for example, Rep52 and Rep40, or Rep78 and Rep 68. Expression constructs may comprise any combination of VP1, VP2, VP3, Rep52/Rep40, and Rep78/Rep68 coding sequences.

In certain embodiments of the present disclosure, a viral expression construct may be used for the production of an AAV particles in insect cells. In certain embodiments, modifications may be made to the wild type AAV sequences of the capsid and/or rep genes, for example to improve attributes of the viral particle, such as increased infectivity or specificity, or to enhance production yields.

In certain embodiments, the viral expression construct may encode the components of a Parvoviral capsid with incorporated Gly-Ala repeat region, which may function as an immune evasion sequence, as described in US Patent Application 20110171262, the content of which is incorporated herein by reference in its entirety as related to Parvoviral capsid proteins.

In certain embodiments of the present disclosure, a viral expression construct may be used for the production of AAV particles in insect cells. In certain embodiments, modifications may be made to the wild type AAV sequences of the capsid and/or rep genes, for example to improve attributes of the viral particle, such as increased infectivity or specificity, or to enhance production yields from insect cells.

### VP-Coding Region

In certain embodiments, a viral expression construct can comprise a VP-coding region; a VP-coding region is a nucleotide sequence which comprises a VP nucleotide sequence encoding VP1, VP2, VP3, or a combination thereof. In certain embodiments, a viral expression construct can comprise a VP1-coding region; a VP1-coding region is a nucleotide sequence which comprises a VP1 nucleotide sequence encoding a VP1 protein. In certain embodiments, a viral expression construct can comprise a VP2-coding region; a VP2-coding region is a nucleotide sequence which comprises a VP2 nucleotide sequence encoding a VP2 protein. In certain embodiments, a viral expression construct can comprise a VP3-coding region; a VP3-coding region is a nucleotide sequence which comprises a VP3 nucleotide sequence encoding a VP3 protein.

In certain embodiments, a VP-coding region encodes one or more AAV capsid proteins of a specific AAV serotype. The AAV serotypes for VP-coding regions can be the same or different. In certain embodiments, a VP-coding region can be codon optimized. In certain embodiments, a VP-coding region or nucleotide sequence can be codon optimized for a mammal cell. In certain embodiments, a VP-coding region or nucleotide sequence can be codon optimized for an insect cell. In certain embodiments, a VP-coding region or nucleotide sequence can be codon optimized for a Spodoptera frugiperda cell. In certain embodiments, a VP-coding region or nucleotide sequence can be codon optimized for Sf9 or Sf21 cell lines.

In certain embodiments, the viral expression construct comprises a first VP-coding region which comprises a nucleotide sequence encoding one or more AAV capsid proteins selected from VP1, VP2, and VP3. In certain embodiments, the first VP-coding region comprises a nucleotide sequence encoding one or more AAV capsid proteins selected from VP2 and VP3. In certain embodiments, the first VP-coding region comprises a nucleotide sequence encoding VP1, VP2, and VP3 AAV capsid proteins. In certain embodiments, the first VP-coding region comprises a nucleotide sequence encoding VP2 and VP3 AAV capsid proteins. In certain embodiments, the first VP-coding region comprises a nucleotide sequence encoding only VP2 and VP3 AAV capsid proteins. In certain embodiments, the first VP-coding region comprises a nucleotide sequence encoding VP2 and VP3 AAV capsid proteins, but not VP1.

In certain embodiments, the nucleic acid construct comprises a second VP-coding region which comprises a nucleotide sequence encoding one or more AAV capsid proteins selected from VP1, VP2, and VP3. In certain embodiments, the second VP-coding region comprises a nucleotide sequence encoding VP1 AAV capsid proteins. In certain embodiments, the second VP-coding region comprises a nucleotide sequence encoding only VP1 AAV capsid proteins. In certain embodiments, the second VP-coding region comprises a nucleotide sequence encoding VP1 AAV capsid proteins, but not VP2 or VP3.

In certain embodiments, the viral expression construct is an engineered nucleic acid construct. In certain embodiments, the viral expression construct comprises a first nucleotide sequence which comprises the first VP-coding region and the second VP-coding region. In certain embodiments, the first nucleotide sequence comprises a first open reading frame (ORF) which comprises the first VP-coding region, and a second open reading frame (ORF) which comprises the second VP-coding region.

In certain embodiments, the viral expression construct comprises a first nucleotide sequence which comprises the first VP-coding region and a second nucleotide sequence which comprises the second VP-coding region. In certain embodiments, the first nucleotide sequence comprises a first open reading frame (ORF) which comprises the first VP-coding region, and the second nucleotide sequence comprises a second open reading frame (ORF) which comprises the second VP-coding region. In certain embodiments, the first open reading frame is different from the second open reading frame.

In certain embodiments, the viral expression construct comprises a first VP-coding region which comprises a nucleotide sequence encoding one or more AAV capsid proteins selected from VP1, VP2, and VP3; and a second VP-coding region which comprises a nucleotide sequence encoding one or more AAV capsid proteins selected from VP1, VP2, and VP3. In certain embodiments, the first VP-coding region comprises a nucleotide sequence encoding VP1, VP2, and VP3 AAV capsid proteins; and the second VP-coding region comprises a nucleotide sequence encoding only VP1 AAV capsid proteins. In certain embodiments, the first VP-coding region comprises a nucleotide sequence encoding VP1, VP2, and VP3 AAV capsid proteins; and the second VP-coding region comprises a nucleotide sequence encoding VP1 AAV capsid proteins, but not VP2 or VP3. In certain embodiments, the first VP-coding region comprises a nucleotide sequence encoding only VP2 and VP3 AAV capsid proteins; and the second VP-coding region comprises a nucleotide sequence encoding only VP1 AAV capsid proteins. In certain embodiments, the first VP-coding region comprises a nucleotide sequence encoding VP2 and VP3 AAV capsid proteins, but not VP1; and the second VP-coding region which comprises a nucleotide sequence encoding VP1 AAV capsid proteins, but not VP2 or VP3.

In certain embodiments, the first VP-coding region encodes AAV capsid proteins of an AAV serotype, e.g., AAV2, AAV9 or AAVPHPN. In certain embodiments, the second VP-coding region encodes AAV capsid proteins of an AAV serotype, e.g., AAV2, AAV9 or AAVPHPN. In certain embodiments, the AAV serotype of the first VP-coding region is the same as the AAV serotype of the second VP-coding region. In certain embodiments, the AAV serotype of the first VP-coding region is different from the AAV serotype of the second VP-coding region. In certain embodiments, a VP-coding region can be codon optimized for an insect cell. In certain embodiments, a VP-coding region can be codon optimized for a Spodoptera frugiperda cell.

In certain embodiments, the viral expression construct comprises: (i) a first nucleotide sequence which comprises a first expression control region comprising a first promoter sequence, and a first VP-coding region which comprises a nucleotide sequence encoding one or more AAV capsid proteins selected from VP1, VP2, and VP3; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the viral expression construct comprises: (i) a first nucleotide sequence which comprises a first expression control region comprising a first promoter sequence, and a first VP-coding region which comprises a nucleotide sequence encoding VP2 and VP3 AAV capsid proteins, but not VP1; and (ii) a second nucleotide sequence which comprises a second expression control region comprising a second promoter sequence, and a second VP-coding region which comprises a nucleotide sequence encoding VP1 AAV capsid proteins, but not VP2 or VP3. In certain embodiments, the nucleotide sequence of the second VP-coding region is codon optimized. In certain embodiments, the nucleotide sequence of the second VP-coding region is codon optimized for an insect cell, or more specifically for a Spodoptera frugiperda cell. In certain embodiments, the nucleotide sequence of the second VP-coding region is codon optimized codon optimized to have a nucleotide homology with the reference nucleotide sequence of less than 100%, less than 90%, or less than 80%.

In certain embodiments, the viral expression construct comprises: (i) a first nucleotide sequence which comprises a first expression control region comprising a first promoter sequence, a first start codon region which comprises a first start codon, a first VP-coding region which comprises a nucleotide sequence encoding one or more AAV capsid proteins selected from VP1, VP2, and VP3, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the nucleic acid construct comprises: (i) a first nucleotide sequence which comprises a first expression control region comprising a first promoter sequence, a first start codon region which comprises a first start codon, a first VP-coding region which comprises a nucleotide sequence encoding VP2 and VP3 AAV capsid proteins, but not VP1, and a first stop codon region which comprises a first stop codon; and (ii) a second nucleotide sequence which comprises a second expression control region comprising a second promoter sequence, a second start codon region which comprises a second start codon, a second VP-coding region which comprises a nucleotide sequence encoding VP1 AAV capsid proteins, but not VP2 or VP3, and a second stop codon region which comprises a second stop codon. In certain embodiments, the first start codon is ATG, the second start codon is ATG, or both the first and second start codons are ATG.

In certain embodiments, a nucleotide sequence encoding a VP1 capsid protein can be codon optimized. In certain embodiments, a nucleotide sequence encoding a VP1 capsid protein can be codon optimized for an insect cell. In certain embodiments, a nucleotide sequence encoding a VP2 capsid protein can be codon optimized. In certain embodiments, a nucleotide sequence encoding a VP2 capsid protein can be codon optimized for an insect cell. In certain embodiments, a nucleotide sequence encoding a VP3 capsid protein can be codon optimized. In certain embodiments, a nucleotide sequence encoding a VP3 capsid protein can be codon optimized for an insect cell.

In certain embodiments, a nucleotide sequence encoding a VP1 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP2 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In certain embodiments, a nucleotide sequence encoding a VP3 capsid protein can be codon optimized to have a nucleotide homology with the reference nucleotide sequence of less than 100%. In certain embodiments, the nucleotide homology between the codon-optimized VP1 nucleotide sequence and the reference VP1 nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

Structural VP proteins, VP1, VP2, and VP3 of a viral expression construct can be encoded in a single open reading frame regulated by utilization of both alternative splice acceptor and non-canonical translational initiation codons. VP1, VP2, and VP3 can be transcribed and translated from a single transcript in which both in-frame and/or out-of-frame start codons are engineered to control the VP1:VP2:VP3 ratio produced by the nucleotide transcript.

In certain embodiments, VP1 can be produced from a sequence which encodes for VP1 only. As used herein, the terms “only for VP1” or “VP1 only” refer to a nucleotide sequence or transcript which encodes primarily for VP1 capsid protein relative to non-VP1 capsid proteins (e.g., VP2 capsid proteins or VP3 capsid proteins). In some embodiments, the nucleotide sequence or transcript: (i) lacks a necessary element within the VP1 sequence such that transcription or translation of VP2 and VP3, as a full or partial sequence, from the VP1 sequence is reduced or inhibited (e.g., a deletion or mutation in one or more start codons within the VP1 sequence upstream of the VP2 or VP3 sequence); (ii) comprises an exogenous nucleic acid sequence or structure (e.g., one or more additional codons) within the VP1 sequence which prevents transcription or translation of VP2 and VP3 from the same sequence; and/or (iii) comprises a start codon for VP1 (e.g., ATG), such that VP1 is the primary VP protein produced by the nucleotide transcript.

In certain embodiments, VP2 can be produced from a sequence which encodes for VP2 only. As used herein, the terms “only for VP2” or “VP2 only” refer to a nucleotide sequence or transcript which encodes primarily for VP2 capsid protein relative to non-VP2 capsid proteins (e.g., VP1 capsid proteins or VP3 capsid proteins). In some embodiments, the nucleotide sequence or transcript: (i) is a truncated variant of a full VP capsid sequence (e.g., full VP1 capsid sequence) which encodes only VP2 capsid proteins; (ii) lacks a necessary element within the VP2 sequence such that transcription or translation of VP3, as a full or partial sequence, from the VP2 sequence is reduced or inhibited (e.g., a deletion or mutation in one or more start codons within the VP2 sequence upstream of the VP3 sequence); (iii) comprises an exogenous nucleic acid sequence or structure (e.g., one or more additional codons) within the VP2 sequence which prevents transcription or translation of VP3 from the same sequence; and/or (iv) comprises a start codon for VP2 (e.g., ATG), such that VP2 is the primary VP protein produced by the nucleotide transcript.

In certain embodiments, VP1 and VP2 can be produced from a sequence which encodes for VP1 and VP2 only. As used herein, the terms “only for VP1 and VP2” or “VP1 and VP2 only” refer to a nucleotide sequence or transcript which encodes primarily for VP1 and VP2 capsid proteins relative to non-VP1/VP2 capsid proteins (e.g., VP3 capsid proteins). In some embodiments, the nucleotide sequence or transcript: (i) lacks a necessary element within the VP1 and/or VP2 sequence such that transcription or translation of VP3, as a full or partial sequence, from the VP1/VP2 sequence is reduced or inhibited (e.g., a deletion or mutation in one or more start codons within the VP1/VP2 sequence upstream of the VP3 sequence); (ii) comprises an exogenous nucleic acid sequence or structure (e.g., one or more additional codons) within the VP1/VP2 sequence which prevents transcription or translation of VP3 from the same sequence; (iii) comprises start codons for VP1 (e.g., ATG) and/or VP2 (e.g., ATG), such that VP1 and VP2 are the primary VP proteins produced by the nucleotide transcript; and/or (iv) comprises VP1-only nucleotide transcript and a VP2-only nucleotide transcript connected by a linker, such as an IRES region.

In certain embodiments, VP3 can be produced from a sequence which encodes for VP3 only. As used herein, the terms “only for VP3” or “VP3 only” refers to a nucleotide sequence or transcript which encodes only VP3 capsid proteins relative to non-VP3 capsid proteins (e.g., VP1 capsid proteins or VP2 capsid proteins). In some embodiments, the nucleotide sequence or transcript: (i) is a truncated variant of a full VP capsid sequence (e.g., full VP1 capsid sequence) which encodes only VP3 capsid proteins; and/or (ii) comprise a start codon for VP3 (e.g., ATG), such that VP3 is the only VP protein produced from the nucleotide transcript.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence. The start codon can be ATG or a non-ATG codon (i.e., a suboptimal start codon where the start codon of the AAV VP1 capsid protein is a non-ATG). In certain embodiments, the viral expression construct used for AAV production may contain a nucleotide sequence encoding the AAV capsid proteins where the initiation codon of the AAV VP1 capsid protein is a non-ATG, i.e., a suboptimal initiation codon, allowing the expression of a modified ratio of the viral capsid proteins in the production system, to provide improved infectivity of the host cell. In a non-limiting example, a viral construct vector may contain a nucleic acid construct comprising a nucleotide sequence encoding AAV VP1, VP2, and VP3 capsid proteins, wherein the initiation codon for translation of the AAV VP1 capsid protein is CTG, TTG, or GTG, as described in U.S. Pat. No. 8,163,543, the content of which is incorporated herein by reference in its entirety as related to AAV capsid proteins and the production thereof.

In some embodiments, an AAV expression construct described herein comprises a VP-coding region. In some embodiments, the VP-coding region comprises a nucleotide sequence encoding: (i) primarily a VP1 protein, e.g., at least 50%, 60%, 70%, 80%, 90% or more VP1 protein relative to a VP2 protein and/or a VP3 protein; (ii) a VP1 protein only; (iii) a VP1 protein, but not a VP2 protein or a VP3 protein; (iv) primarily a VP2 protein, e.g., at least about 50%, 60%, 70%, 80%, 90% or more VP2 protein relative to a VP1 protein and/or a VP3 protein; (v) a VP2 protein only; (vi) a VP2 protein, but not a VP1 protein or a VP3 protein; (vii) a VP3 protein only; (viii) a VP3 protein, but not a VP1 protein or a VP2 protein; (ix) a VP1 protein and a VP2 protein, but not a VP3 protein; (x) a VP1 protein and a VP3 protein, but not a VP2 protein; (xi) a VP2 protein and a VP3 protein, but not a VP1 protein; (xii) a VP1 protein, a VP2 protein, and a VP3 protein.

In some embodiments, the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein. In some embodiments, the VP-coding region comprises a single polycistronic ORF encoding a VP1 protein, a VP2 protein, and a VP3 protein. In some embodiments, the ORF encoding the VP1 protein comprises an ACG start codon, the ORF encoding the VP2 protein comprises an ACG start codon, and the ORF encoding the VP3 protein comprises an ATG start codon. In some embodiments, the ORF encoding the VP1 protein comprises an ATG start codon, the ORF encoding the VP2 protein comprises an ACG start codon, and the ORF encoding the VP3 protein comprises an ATG start codon.

In some embodiments, the VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins. In some embodiments, the VP-coding region encodes an AAV5 capsid protein or variant thereof, or an AAV9 capsid protein or variant thereof. In some embodiments, the VP-coding region encodes a capsid protein as provided in WO2021230987, WO2019028306, WO2019222329, WO2020077165, WO2020028751, WO2020223280, WO2019222444, WO2019222441, or WO2017100671, the contents of which are hereby incorporated by reference in their entirety. In some embodiments, the VP-coding region encodes a capsid protein encoded by or comprising a sequence as provided in Table 7, or a sequence substantially identical (e.g., having at least about 70%, 75%, 80%, 85%, 90%, 92%, 95%, 97%, 98%, or 99% sequence identity) to any of the aforesaid sequences.

In some embodiments, the VP-coding encodes a VP1 protein comprising the amino acid sequence of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences. In some embodiments, the VP-coding region comprises the nucleotide sequence of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences.

In some embodiments, the VP-coding region encodes a VP2 protein e.g., a fragment or a portion, of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences. In some embodiments, the VP2 protein comprises amino acids 138-736 or SEQ ID NOs: 71 or 46-48; amino acids 138-743 of SEQ ID NOs: 52, 53, 54, 56, 60, 61, 64, 66, 68; or amino acids 137-724 of SEQ ID NO: 168. In some embodiments, the VP-coding region comprises a nucleotide sequence encoding a VP2 protein e.g., a fragment or a portion, of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences. In some embodiments, the nucleotide sequence encoding the VP2 protein comprises nucleotides 412-2211 of SEQ ID NOs: 43-45, 72, 205, or 212; nucleotides 412-2232 of SEQ ID NOs: 49-51, 57-59, 62, 63, 65, 67, 69, 72, or 206-211; or nucleotides 409-2175 of SEQ ID NO: 169 or 213. In some embodiments, the nucleotide sequence encoding the VP2 protein comprises nucleotides 418-2211 of SEQ ID NOs: 44 and 45 or nucleotides 418-2232 of SEQ ID NOs: 50, 51, 59, or 60.

In some embodiments, the VP-coding region encodes a VP3 protein e.g., a fragment or a portion, of any of SEQ ID NOs: 46, 47, 48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences. In some embodiments, the VP3 protein comprises amino acids 203-736 of SEQ ID NOs: 71 or 46-48; amino acids 203-743 of SEQ ID NOs: 52, 53, 54, 56, 60, 61, 64, 66, 68; or amino acids 193-724 of SEQ ID NO: 168. In some embodiments, the VP-coding region comprises a nucleotide sequence encoding a VP3 protein e.g., a fragment or a portion, of any of SEQ ID NOs: 43-45, 49-51, 57-59, 62, 63, 65, 67, 69, 72, 169, or 205-213, or a nucleotide sequence at least 70%, 75%, 80%, 85%, 90%, 95%, 96%, 97%, 98%, or 99% identical to any of the aforesaid nucleotide sequences. In some embodiments, the nucleotide sequence encoding the VP3 protein comprises nucleotides 607-2211 of SEQ ID NOs: 43-45, 72, 205, or 212; nucleotides 607-2232 of SEQ ID NOs: 49-51, 57-59, 62, 63, 65, 67, 69, 72, or 206-211; or nucleotides 577-2175 of SEQ ID NO: 169 or 213. In some embodiments, the nucleotide sequence encoding the VP3 protein comprises nucleotides 613-2211 of SEQ ID NOs: 44 and 45 or nucleotides 613-2232 of SEQ ID NOs: 50, 51, 59, or 60.

In some embodiments, any of the nucleotide sequences provided in Table 7 can comprises an ATG start codon (e.g., a non-canonical start codon). In some embodiments, any of the sequence a non-canonical start codon, e.g., ACG, CTG, TTG, and GTG. In some embodiments, any of the nucleotide sequences in Table 7 does not comprise a stop codon.

In some embodiments, the nucleotide sequence of the VP-coding region is codon optimized for an insect cell, optionally a Spodoptera frugiperda insect cell (e.g., an Sf9 insect cell).

TABLE 7
Exemplary full length capsid sequences

| | SEQ | |
|---|---|---|
| | ID | |
| Description | NO: | Sequence |
| AAV9 | 43 | ATGGCTGCCGATGGTTATCTTCCAGATTGGCTCGAGGACAACCTTAGTGAAGGAATTCG |
| (DNA v1) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 205 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCCCAAGCACAG |
| | | GCGCAGACCGGCTGGGTTCAAAACCAAGGAATACTTCCGGGTATGGTTTGGCAGGACAG |
| | | AGATGTGTACCTGCAAGGACCCATTTGGGCCAAAATTCCTCACACGGACGGCAACTTTC |
| | | ACCCTTCTCCGCTGATGGGAGGGTTTGGAATGAAGCACCCGCCTCCTCAGATCCTCATC |
| | | AAAAACACACCTGTACCTGCGGATCCTCCAACGGCCTTCAACAAGGACAAGCTGAACTC |
| | | TTTCATCACCCAGTATTCTACTGGCCAAGTCAGCGTGGAGATCGAGTGGGAGCTGCAGA |
| | | AGGAAAACAGCAAGCGCTGGAACCCGGAGATCCAGTACACTTCCAACTATTACAAGTCT |
| | | AATAATGTTGAATTTGCTGTTAATACTGAAGGTGTATATAGTGAACCCCGCCCCATTGG |
| | | CACCAGATACCTGACTCGTAATCTGTAA |
| AAV9 | 44 | cggtgtatggcTGCCGACGGTTATCTACCCGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug13 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGCCCAA |
| | | GCACAGGCGCAGACCGGCTGGGTTCAAAACCAAGGAATACTTCCGGGTATGGTTTGGCA |
| | | GGACAGAGATGTGTACCTGCAAGGACCCATTTGGGCCAAAATTCCTCACACGGACGGCA |
| | | ACTTTCACCCTTCTCCGCTGATGGGAGGGTTTGGAATGAAGCACCCGCCTCCTCAGATC |
| | | CTCATCAAAAACACACCTGTACCTGCGGATCCTCCAACGGCCTTCAACAAGGACAAGCT |
| | | GAACTCTTTCATCACCCAGTATTCTACTGGCCAAGTCAGCGTGGAGATCGAGTGGGAGC |
| | | TGCAGAAGGAAAACAGCAAGCGCTGGAACCCGGAGATCCAGTACACTTCCAACTATTAC |
| | | AAGTCTAATAATGTTGAATTTGCTGTTAATACTGAAGGTGTATATAGTGAACCCCGCCC |
| | | CATTGGCACCAGATACCTGACTCGTAATCTGTAA |
| AAV9 | 45 | cggtgtatgagTGCCGACGGTTATCTACCCGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug14 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGCCCAA |
| | | GCACAGGCGCAGACCGGCTGGGTTCAAAACCAAGGAATACTTCCGGGTATGGTTTGGCA |
| | | GGACAGAGATGTGTACCTGCAAGGACCCATTTGGGCCAAAATTCCTCACACGGACGGCA |
| | | ACTTTCACCCTTCTCCGCTGATGGGAGGGTTTGGAATGAAGCACCCGCCTCCTCAGATC |
| | | CTCATCAAAAACACACCTGTACCTGCGGATCCTCCAACGGCCTTCAACAAGGACAAGCT |
| | | GAACTCTTTCATCACCCAGTATTCTACTGGCCAAGTCAGCGTGGAGATCGAGTGGGAGC |
| | | TGCAGAAGGAAAACAGCAAGCGCTGGAACCCGGAGATCCAGTACACTTCCAACTATTAC |
| | | AAGTCTAATAATGTTGAATTTGCTGTTAATACTGAAGGTGTATATAGTGAACCCCGCCC |
| | | CATTGGCACCAGATACCTGACTCGTAATCTGTAA |
| AAV9 | 46 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9 | 47 | MSADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9.v1 | 48 | MAADGYLPDWLEDNLSEGIREWWALKPGAPQPKANQQHQDNARGLVLPGYKYLGPGNGL |
| | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAQ |
| | | AQTGWVQNQGILPGMVWQDRDVYLQGPIWAKIPHTDGNFHPSPLMGGFGMKHPPPQILI |
| | | KNTPVPADPPTAFNKDKLNSFITQYSTGQVSVEIEWELQKENSKRWNPEIQYTSNYYKS |
| | | NNVEFAVNTEGVYSEPRPIGTRYLTRNL |
| AAV9.v2 | 49 | ATGGCTGCCGATGGTTATCTTCCAGATTGGCTCGAGGACAACCTTAGTGAAGGAATTCG |
| (DNA v1) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v2 | 206 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v2 | 50 | cggtgtatggcTGCCGACGGTTATCTaCCcGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug13 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGATGGG |
| | | ACTTTGGCGGTGCCTTTTAAGGCACAGGCGCAGACCGGCTGGGTTCAAAACCAAGGAAT |
| | | ACTTCCGGGTATGGTTTGGCAGGACAGAGATGTGTACCTGCAAGGACCCATTTGGGCCA |
| | | AAATTCCTCACACGGACGGCAACTTTCACCCTTCTCCGCTGATGGGAGGGTTTGGAATG |
| | | AAGCACCCGCCTCCTCAGATCCTCATCAAAAACACACCTGTACCTGCGGATCCTCCAAC |
| | | GGCCTTCAACAAGGACAAGCTGAACTCTTTCATCACCCAGTATTCTACTGGCCAAGTCA |
| | | GCGTGGAGATCGAGTGGGAGCTGCAGAAGGAAAACAGCAAGCGCTGGAACCCGGAGATC |
| | | CAGTACACTTCCAACTATTACAAGTCTAATAATGTTGAATTTGCTGTTAATACTGAAGG |
| | | TGTATATAGTGAACCCCGCCCCATTGGCACCAGATACCTGACTCGTAATCTGTAA |
| AAV9.v2 | 51 | cggtgtatgagTGCCGAcGGTTATCTaCCcGATTGGCTCGAGGACAACCTTAGTGAAGG |
| (DNA | | AATTCGCGAGTGGTGGGCTTTGAAACCTGGAGCCCCTCAACCCAAGGCAAATCAACAAC |
| with | | ATCAAGACAACGCTCGAGGTCTTGTGCTTCCGGGTTACAAATACCTTGGACCCGGCAAC |
| VP1aug14 | | GGACTCGACAAGGGGGAGCCGGTCAACGCAGCAGACGCGGCGGCCCTCGAGCACGACAA |
| modified | | GGCCTACGACCAGCAGCTCAAGGCCGGAGACAACCCGTACCTCAAGTACAACCACGCCG |
| Kozak | | ACGCCGAGTTCCAGGAGCGGCTCAAAGAAGATACGTCTTTTGGGGGCAACCTCGGGCGA |
| underlined | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGGCCACAAACCACCAGAGTGATGGG |
| | | ACTTTGGCGGTGCCTTTTAAGGCACAGGCGCAGACCGGCTGGGTTCAAAACCAAGGAAT |
| | | ACTTCCGGGTATGGTTTGGCAGGACAGAGATGTGTACCTGCAAGGACCCATTTGGGCCA |
| | | AAATTCCTCACACGGACGGCAACTTTCACCCTTCTCCGCTGATGGGAGGGTTTGGAATG |
| | | AAGCACCCGCCTCCTCAGATCCTCATCAAAAACACACCTGTACCTGCGGATCCTCCAAC |
| | | GGCCTTCAACAAGGACAAGCTGAACTCTTTCATCACCCAGTATTCTACTGGCCAAGTCA |
| | | GCGTGGAGATCGAGTGGGAGCTGCAGAAGGAAAACAGCAAGCGCTGGAACCCGGAGATC |
| | | CAGTACACTTCCAACTATTACAAGTCTAATAATGTTGAATTTGCTGTTAATACTGAAGG |
| | | TGTATATAGTGAACCCCGCCCCATTGGCACCAGATACCTGACTCGTAATCTGTAA |
| AAV9.v2 | 52 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v2 | 53 | MSADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v3 | | ACGGCTGCCGACGGTTATCTACCCGATTGGCTCGAGGACAACCTTAGTGAAGGAATTCG |
| (DNA) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAGGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGATGGGACTTTG |
| | | GCGGTGCCTTTTAAGGCACAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCGGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGCTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v3 | 54 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSRTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v4 | 56 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRIWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSDGTL |
| | | AVPFKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v5 | 57 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtccgcttaatggt |
| | | gccgtccatctttatgctcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v5 | 207 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTCCGCTTAATGGT |
| | | GCCGTCCATCTTTATGCTCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v5 | 58 | cggtgtatggcTGCCGAcGGTTATCTaCCcGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug13 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGgccacaaaccaccagagtCCGCTT |
| | | AATGGTGCCGTCCATCTTTATGCTCAGGCGCAGaccggctggGTTCAAAACCAAGGAAT |
| | | ACTTCCGGGTATGGTTTGGCAGGACAGAGATGTGTACCTGCAAGGACCCATTTGGGCCA |
| | | AAATTCCTCACACGGACGGCAACTTTCACCCTTCTCCGCTGATGGGAGGGTTTGGAATG |
| | | AAGCACCCGCCTCCTCAGATCCTCATCAAAAACACACCTGTACCTGCCGATCCTCCAAC |
| | | GGCCTTCAACAAGGACAAGCTGAACTCTTTCATCACCCAGTATTCTACTGGCCAAGTCA |
| | | GCGTGGAGATCGAGTGGGAGCTGCAGAAGGAAAACAGCAAGCGgTGGAACCCGGAGATC |
| | | CAGTACACTTCCAACTATTACAAGTCTAATAATGTTGAATTTGCTGTTAATACTGAAGG |
| | | TGTATATAGTGAACCCCGCCCCATTGGCACCAGATACCTGACTCGTAATCTGTAA |
| AAV9.v5 | 59 | cggtgtatgagTGCCGACGGTTATCTaCCcGATTGGCTCGAGGACAACCTTAGTGAAGG |
| (DNA with | | AATTCGCGAGTGGTGGGCTTTGAAACCTGGAGCCCCTCAACCCAAGGCAAATCAACAAC |
| VP1aug14 | | ATCAAGACAACGCTCGAGGTCTTGTGCTTCCGGGTTACAAATACCTTGGACCCGGCAAC |
| modified | | GGACTCGACAAGGGGGAGCCGGTCAACGCAGCAGACGCGGCGGCCCTCGAGCACGACAA |
| Kozak | | GGCCTACGACCAGCAGCTCAAGGCCGGAGACAACCCGTACCTCAAGTACAACCACGCCG |
| underlined) | | ACGCCGAGTTCCAGGAGCGGCTCAAAGAAGATACGTCTTTTGGGGGCAACCTCGGGCGA |
| | | GCAGTCTTCCAGGCCAAAAAGAGGCTTCTTGAACCTCTTGGTCTGGTTGAGGAAGCGGC |
| | | TAAGACGGCTCCTGGAAAGAAGAGGCCTGTAGAGCAGTCTCCTCAGGAACCGGACTCCT |
| | | CCGCGGGTATTGGCAAATCGGGTGCACAGCCCGCTAAAAAGAGACTCAATTTCGGTCAG |
| | | ACTGGCGACACAGAGTCAGTCCCAGACCCTCAACCAATCGGAGAACCTCCCGCAGCCCC |
| | | CTCAGGTGTGGGATCTCTTACAATGGCTTCAGGTGGTGGCGCACCAGTGGCAGACAATA |
| | | ACGAAGGTGCCGATGGAGTGGGTAGTTCCTCGGGAAATTGGCATTGCGATTCCCAATGG |
| | | CTGGGGGACAGAGTCATCACCACCAGCACCCGAACCTGGGCCCTGCCCACCTACAACAA |
| | | TCACCTCTACAAGCAAATCTCCAACAGCACATCTGGAGGATCTTCAAATGACAACGCCT |
| | | ACTTCGGCTACAGCACCCCCTGGGGGTATTTTGACTTCAACAGATTCCACTGCCACTTC |
| | | TCACCACGTGACTGGCAGCGACTCATCAACAACAACTGGGGATTCCGGCCTAAGCGACT |
| | | CAACTTCAAGCTCTTCAACATTCAGGTCAAAGAGGTTACGGACAACAATGGAGTCAAGA |
| | | CCATCGCCAATAACCTTACCAGCACGGTCCAGGTCTTCACGGACTCAGACTATCAGCTC |
| | | CCGTACGTGCTCGGGTCGGCTCACGAGGGCTGCCTCCCGCCGTTCCCAGCGGACGTTTT |
| | | CATGATTCCTCAGTACGGGTATCTGACGCTTAATGATGGAAGCCAGGCCGTGGGTCGTT |
| | | CGTCCTTTTACTGCCTGGAATATTTCCCGTCGCAAATGCTAAGAACGGGTAACAACTTC |
| | | CAGTTCAGCTACGAGTTTGAGAACGTACCTTTCCATAGCAGCTACGCTCACAGCCAAAG |
| | | CCTGGACCGACTAATGAATCCACTCATCGACCAATACTTGTACTATCTCTCAAAGACTA |
| | | TTAACGGTTCTGGACAGAATCAACAAACGCTAAAATTCAGTGTGGCCGGACCCAGCAAC |
| | | ATGGCTGTCCAGGGAAGAAACTACATACCTGGACCCAGCTACCGACAACAACGTGTCTC |
| | | AACCACTGTGACTCAAAACAACAACAGCGAATTTGCTTGGCCTGGAGCTTCTTCTTGGG |
| | | CTCTCAATGGACGTAATAGCTTGATGAATCCTGGACCTGCTATGGCCAGCCACAAAGAA |
| | | GGAGAGGACCGTTTCTTTCCTTTGTCTGGATCTTTAATTTTTGGCAAACAAGGAACTGG |
| | | AAGAGACAACGTGGATGCGGACAAAGTCATGATAACCAACGAAGAAGAAATTAAAACTA |
| | | CTAACCCGGTAGCAACGGAGTCCTATGGACAAGTGgccacaaaccaccagagtCCGCTT |
| | | AATGGTGCCGTCCATCTTTATGCTCAGGCGCAGaccggctggGTTCAAAACCAAGGAAT |
| | | ACTTCCGGGTATGGTTTGGCAGGACAGAGATGTGTACCTGCAAGGACCCATTTGGGCCA |
| | | AAATTCCTCACACGGACGGCAACTTTCACCCTTCTCCGCTGATGGGAGGGTTTGGAATG |
| | | AAGCACCCGCCTCCTCAGATCCTCATCAAAAACACACCTGTACCTGCCGATCCTCCAAC |
| | | GGCCTTCAACAAGGACAAGCTGAACTCTTTCATCACCCAGTATTCTACTGGCCAAGTCA |
| | | GCGTGGAGATCGAGTGGGAGCTGCAGAAGGAAAACAGCAAGCGgTGGAACCCGGAGATC |
| | | CAGTACACTTCCAACTATTACAAGTCTAATAATGTTGAATTTGCTGTTAATACTGAAGG |
| | | TGTATATAGTGAACCCCGCCCCATTGGCACCAGATACCTGACTCGTAATCTGTAA |
| AAV9.v5 | 60 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSPLNG |
| | | AVHLYAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v5 | 61 | MSADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSPING |
| | | AVHLYAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v6 | 62 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgt |
| | | gattctccgaagggttggcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v6 | 208 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCACAGGCTCGT |
| | | GATTCTCCGAAGGGTTGGCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v6 | 63 | atggctgccgatggttatcttccagattggctcgaggacaaccttagtgaaggaattcg |
| (DNA v3) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgt |
| | | gattctccgaagggttggcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcggatcctccaacggcctt |
| | | caacaaggacaagctgaactctttcatcacccagtattctactggccaagtcagcgtgg |
| | | agatcgagtgggagctgcagaaggaaaacagcaagcgctggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v6 | 64 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAR |
| | | DSPKGWQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v7 | 65 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatottcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggcttat |
| | | tctacggatgtgaggatgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v7 | 209 | ACGGCTGCCGACGGTTATCTACCCGattggctcgaggacaaccttagtgaaggaattcg |
| (DNA v2) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggcttat |
| | | tctacggatgtgaggatgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v7 | 66 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAY |
| | | STDVRMQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v8 | 67 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacagattgtt |
| | | atgaattcgttgaaggctcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v8 | 210 | ACGGCTGCCGACGGTTATCTACCCGattggcTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGgccacaaaccaccagagtGCACAGATTGTT |
| | | ATGAATTCGTTGAAGGCTCAGGCGCAGaccggctggGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGgTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v8 | 68 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQIV |
| | | MNSLKAQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV9.v9 | 69 | atggctgccgatggttatcttccagattggctcgaggacaaccttagtgaaggaattcg |
| (DNA v1) | | cgagtggtgggctttgaaacctggagcccctcaacccaaggcaaatcaacaacatcaag |
| | | acaacgctcgaggtcttgtgcttccgggttacaaataccttggacccggcaacggactc |
| | | gacaagggggagccggtcaacgcagcagacgcggcggccctcgagcacgacaaggccta |
| | | cgaccagcagctcaaggccggagacaacccgtacctcaagtacaaccacgccgacgccg |
| | | agttccaggagcggctcaaagaagatacgtcttttgggggcaacctcgggcgagcagtc |
| | | ttccaggccaaaaagaggcttcttgaacctcttggtctggttgaggaagcggctaagac |
| | | ggctcctggaaagaagaggcctgtagagcagtctcctcaggaaccggactcctccgcgg |
| | | gtattggcaaatcgggtgcacagcccgctaaaaagagactcaatttcggtcagactggc |
| | | gacacagagtcagtcccagaccctcaaccaatcggagaacctcccgcagccccctcagg |
| | | tgtgggatctcttacaatggcttcaggtggtggcgcaccagtggcagacaataacgaag |
| | | gtgccgatggagtgggtagttcctcgggaaattggcattgcgattcccaatggctgggg |
| | | gacagagtcatcaccaccagcacccgaacctgggccctgcccacctacaacaatcacct |
| | | ctacaagcaaatctccaacagcacatctggaggatcttcaaatgacaacgcctacttcg |
| | | gctacagcaccccctgggggtattttgacttcaacagattccactgccacttctcacca |
| | | cgtgactggcagcgactcatcaacaacaactggggattccggcctaagcgactcaactt |
| | | caagctcttcaacattcaggtcaaagaggttacggacaacaatggagtcaagaccatcg |
| | | ccaataaccttaccagcacggtccaggtcttcacggactcagactatcagctcccgtac |
| | | gtgctcgggtcggctcacgagggctgcctcccgccgttcccagcggacgttttcatgat |
| | | tcctcagtacgggtatctgacgcttaatgatggaagccaggccgtgggtcgttcgtcct |
| | | tttactgcctggaatatttcccgtcgcaaatgctaagaacgggtaacaacttccagttc |
| | | agctacgagtttgagaacgtacctttccatagcagctacgctcacagccaaagcctgga |
| | | ccgactaatgaatccactcatcgaccaatacttgtactatctctcaaagactattaacg |
| | | gttctggacagaatcaacaaacgctaaaattcagtgtggccggacccagcaacatggct |
| | | gtccagggaagaaactacatacctggacccagctaccgacaacaacgtgtctcaaccac |
| | | tgtgactcaaaacaacaacagcgaatttgcttggcctggagcttcttcttgggctctca |
| | | atggacgtaatagcttgatgaatcctggacctgctatggccagccacaaagaaggagag |
| | | gaccgtttctttcctttgtctggatctttaatttttggcaaacaaggaactggaagaga |
| | | caacgtggatgcggacaaagtcatgataaccaacgaagaagaaattaaaactactaacc |
| | | cggtagcaacggagtcctatggacaagtggccacaaaccaccagagtgcacaggctcgg |
| | | gagagtcctcgtgggctgcaggcgcagaccggctgggttcaaaaccaaggaatacttcc |
| | | gggtatggtttggcaggacagagatgtgtacctgcaaggacccatttgggccaaaattc |
| | | ctcacacggacggcaactttcacccttctccgctgatgggagggtttggaatgaagcac |
| | | ccgcctcctcagatcctcatcaaaaacacacctgtacctgcCgatcctccaacggcctt |
| | | caacaaggacaagctgaactctttcatcacccagtattctactggccaagtcagcgtgg |
| | | agatcgagtgggagctgcagaaggaaaacagcaagcgGtggaacccggagatccagtac |
| | | acttccaactattacaagtctaataatgttgaatttgctgttaatactgaaggtgtata |
| | | tagtgaaccccgccccattggcaccagatacctgactcgtaatctgtaa |
| AAV9.v9 | 211 | ACGGCTGCCGACGGTTATCTACCCGATTGGCTCGAGGACAACCTTAGTGAAGGAATTCG |
| (DNA v2) | | CGAGTGGTGGGCTTTGAAACCTGGAGCCCCTCAACCCAAGGCAAATCAACAACATCAAG |
| | | ACAACGCTCGAGGTCTTGTGCTTCCGGGTTACAAATACCTTGGACCCGGCAACGGACTC |
| | | GACAAGGGGGAGCCGGTCAACGCAGCAGACGCGGCGGCCCTCGAGCACGACAAGGCCTA |
| | | CGACCAGCAGCTCAAGGCCGGAGACAACCCGTACCTCAAGTACAACCACGCCGACGCCG |
| | | AGTTCCAGGAGCGGCTCAAAGAAGATACGTCTTTTGGGGGCAACCTCGGGCGAGCAGTC |
| | | TTCCAGGCCAAAAAGAGGCTTCTTGAACCTCTTGGTCTGGTTGAGGAAGCGGCTAAGAC |
| | | GGCTCCTGGAAAGAAGAGGCCTGTAGAGCAGTCTCCTCAGGAACCGGACTCCTCCGCGG |
| | | GTATTGGCAAATCGGGTGCACAGCCCGCTAAAAAGAGACTCAATTTCGGTCAGACTGGC |
| | | GACACAGAGTCAGTCCCAGACCCTCAACCAATCGGAGAACCTCCCGCAGCCCCCTCAGG |
| | | TGTGGGATCTCTTACAATGGCTTCAGGTGGTGGCGCACCAGTGGCAGACAATAACGAAG |
| | | GTGCCGATGGAGTGGGTAGTTCCTCGGGAAATTGGCATTGCGATTCCCAATGGCTGGGG |
| | | GACAGAGTCATCACCACCAGCACCCGAACCTGGGCCCTGCCCACCTACAACAATCACCT |
| | | CTACAAGCAAATCTCCAACAGCACATCTGGAGGATCTTCAAATGACAACGCCTACTTCG |
| | | GCTACAGCACCCCCTGGGGGTATTTTGACTTCAACAGATTCCACTGCCACTTCTCACCA |
| | | CGTGACTGGCAGCGACTCATCAACAACAACTGGGGATTCCGGCCTAAGCGACTCAACTT |
| | | CAAGCTCTTCAACATTCAGGTCAAAGAGGTTACGGACAACAATGGAGTCAAGACCATCG |
| | | CCAATAACCTTACCAGCACGGTCCAGGTCTTCACGGACTCAGACTATCAGCTCCCGTAC |
| | | GTGCTCGGGTCGGCTCACGAGGGCTGCCTCCCGCCGTTCCCAGCGGACGTTTTCATGAT |
| | | TCCTCAGTACGGGTATCTGACGCTTAATGATGGAAGCCAGGCCGTGGGTCGTTCGTCCT |
| | | TTTACTGCCTGGAATATTTCCCGTCGCAAATGCTAAGAACGGGTAACAACTTCCAGTTC |
| | | AGCTACGAGTTTGAGAACGTACCTTTCCATAGCAGCTACGCTCACAGCCAAAGCCTGGA |
| | | CCGACTAATGAATCCACTCATCGACCAATACTTGTACTATCTCTCAAAGACTATTAACG |
| | | GTTCTGGACAGAATCAACAAACGCTAAAATTCAGTGTGGCCGGACCCAGCAACATGGCT |
| | | GTCCAGGGAAGAAACTACATACCTGGACCCAGCTACCGACAACAACGTGTCTCAACCAC |
| | | TGTGACTCAAAACAACAACAGCGAATTTGCTTGGCCTGGAGCTTCTTCTTGGGCTCTCA |
| | | ATGGACGTAATAGCTTGATGAATCCTGGACCTGCTATGGCCAGCCACAAAGAAGGAGAG |
| | | GACCGTTTCTTTCCTTTGTCTGGATCTTTAATTTTTGGCAAACAAGGAACTGGAAGAGA |
| | | CAACGTGGATGCGGACAAAGTCATGATAACCAACGAAGAAGAAATTAAAACTACTAACC |
| | | CGGTAGCAACGGAGTCCTATGGACAAGTGGCCACAAACCACCAGAGTGCACAGGCTCGG |
| | | GAGAGTCCTCGTGGGCTGCAGGCGCAGACCGGCTGGGTTCAAAACCAAGGAATACTTCC |
| | | GGGTATGGTTTGGCAGGACAGAGATGTGTACCTGCAAGGACCCATTTGGGCCAAAATTC |
| | | CTCACACGGACGGCAACTTTCACCCTTCTCCGCTGATGGGAGGGTTTGGAATGAAGCAC |
| | | CCGCCTCCTCAGATCCTCATCAAAAACACACCTGTACCTGCCGATCCTCCAACGGCCTT |
| | | CAACAAGGACAAGCTGAACTCTTTCATCACCCAGTATTCTACTGGCCAAGTCAGCGTGG |
| | | AGATCGAGTGGGAGCTGCAGAAGGAAAACAGCAAGCGGTGGAACCCGGAGATCCAGTAC |
| | | ACTTCCAACTATTACAAGTCTAATAATGTTGAATTTGCTGTTAATACTGAAGGTGTATA |
| | | TAGTGAACCCCGCCCCATTGGCACCAGATACCTGACTCGTAATCTGTAA |
| AAV9.v9 | 70 | MAADGYLPDWLEDNLSEGIREWWALKPGAPQPKANQQHQDNARGLVLPGYKYLGPGNGL |
| (amino acid) | | DKGEPVNAADAAALEHDKAYDQQLKAGDNPYLKYNHADAEFQERLKEDTSFGGNLGRAV |
| | | FQAKKRLLEPLGLVEEAAKTAPGKKRPVEQSPQEPDSSAGIGKSGAQPAKKRLNFGQTG |
| | | DTESVPDPQPIGEPPAAPSGVGSLTMASGGGAPVADNNEGADGVGSSSGNWHCDSQWLG |
| | | DRVITTSTRTWALPTYNNHLYKQISNSTSGGSSNDNAYFGYSTPWGYFDFNRFHCHFSP |
| | | RDWQRLINNNWGFRPKRLNFKLFNIQVKEVTDNNGVKTIANNLTSTVQVFTDSDYQLPY |
| | | VLGSAHEGCLPPFPADVFMIPQYGYLTLNDGSQAVGRSSFYCLEYFPSQMLRTGNNFQF |
| | | SYEFENVPFHSSYAHSQSLDRLMNPLIDQYLYYLSKTINGSGQNQQTLKFSVAGPSNMA |
| | | VQGRNYIPGPSYRQQRVSTTVTQNNNSEFAWPGASSWALNGRNSLMNPGPAMASHKEGE |
| | | DRFFPLSGSLIFGKQGTGRDNVDADKVMITNEEEIKTTNPVATESYGQVATNHQSAQAR |
| | | ESPRGLQAQTGWVQNQGILPGMVWQDRDVYLQGPIWAKIPHTDGNFHPSPLMGGFGMKH |
| | | PPPQILIKNTPVPADPPTAFNKDKLNSFITQYSTGQVSVEIEWELQKENSKRWNPEIQY |
| | | TSNYYKSNNVEFAVNTEGVYSEPRPIGTRYLTRNL |
| AAV1 | 72 | atggctgccgatggttatcttccagattggctcgaggacaacctctctgagggcattcg |
| (DNA v1) | | cgagtggtgggacttgaaacctggagccccgaagcccaaagccaaccagcaaaagcagg |
| | | acgacggccggggtctggtgcttcctggctacaagtacctcggacccttcaacggactc |
| | | gacaagggggagcccgtcaacgcggcggacgcagcggccctcgagcacgacaaggccta |
| | | cgaccagcagctcaaagcgggtgacaatccgtacctgcggtataaccacgccgacgccg |
| | | agtttcaggagcgtctgcaagaagatacgtcttttgggggcaacctcgggcgagcagtc |
| | | ttccaggccaagaagcgggttctcgaacctctcggtctggttgaggaaggcgctaagac |
| | | ggctcctggaaagaaacgtccggtagagcagtcgccacaagagccagactcctcctcgg |
| | | gcatcggcaagacaggccagcagcccgctaaaaagagactcaattttggtcagactggc |
| | | gactcagagtcagtccccgatccacaacctctcggagaacctccagcaacccccgctgc |
| | | tgtgggacctactacaatggcttcaggcggtggcgcaccaatggcagacaataacgaag |
| | | gcgccgacggagtgggtaatgcctcaggaaattggcattgcgattccacatggctgggc |
| | | gacagagtcatcaccaccagcacccgcacctgggccttgcccacctacaataaccacct |
| | | ctacaagcaaatctccagtgcttcaacgggggccagcaacgacaaccactacttcggct |
| | | acagcaccccctgggggtattttgatttcaacagattccactgccacttttcaccacgt |
| | | gactggcagcgactcatcaacaacaattggggattccggcccaagagactcaacttcaa |
| | | actcttcaacatccaagtcaaggaggtcacgacgaatgatggcgtcacaaccatcgcta |
| | | ataaccttaccagcacggttcaagtcttctcggactcggagtaccagcttccgtacgtc |
| | | ctcggctctgcgcaccagggctgcctccctccgttcccggcggacgtgttcatgattcc |
| | | gcaatacggctacctgacgctcaacaatggcagccaagccgtgggacgttcatcctttt |
| | | actgcctggaatatttcccttctcagatgctgagaacgggcaacaactttaccttcagc |
| | | tacacctttgaggaagtgcctttccacagcagctacgcgcacagccagagcctggaccg |
| | | gctgatgaatcctctcatcgaccaatacctgtattacctgaacagaactcaaaatcagt |
| | | ccggaagtgcccaaaacaaggacttgctgtttagccgtgggtctccagctggcatgtct |
| | | gttcagcccaaaaactggctacctggaccctgttatcggcagcagcgcgtttctaaaac |
| | | aaaaacagacaacaacaacagcaattttacctggactggtgcttcaaaatataacctca |
| | | atgggcgtgaatccatcatcaaccctggcactgctatggcctcacacaaagacgacgaa |
| | | gacaagttctttcccatgagcggtgtcatgatttttggaaaagagagcgccggagcttc |
| | | aaacactgcattggacaatgtcatgattacagacgaagaggaaattaaagccactaacc |
| | | ctgtggccaccgaaagatttgggaccgtggcagtcaatttccagagcagcagcacagac |
| | | cctgcgaccggagatgtgcatgctatgggagcattacctggcatggtgtggcaagatag |
| | | agacgtgtacctgcagggtcccatttgggccaaaattcctcacacagatggacactttc |
| | | acccgtctcctcttatgggggctttggactcaagaacccgcctcctcagatcctcatc |
| | | aaaaacacgcctgttcctgcgaatcctccggcggagttttcagctacaaagtttgcttc |
| | | attcatcacccaatactccacaggacaagtgagtgtggaaattgaatgggagctgcaga |
| | | aagaaaacagcaagcgctggaatcccgaagtgcagtacacatccaattatgcaaaatct |
| | | gccaacgttgattttactgtggacaacaatggactttatactgagcctcgccccattgg |
| | | cacccgttaccttacccgtcccctgtaa |
| AAV1 | 212 | ACGGCTGCCGACGGTTATCTACCCGATTGGCTCGAGGACAACCTCTCTGAGGGCATTCG |
| (DNA v2) | | CGAGTGGTGGGACTTGAAACCTGGAGCCCCGAAGCCCAAAGCCAACCAGCAAAAGCAGG |
| | | ACGACGGCCGGGGTCTGGTGCTTCCTGGCTACAAGTACCTCGGACCCTTCAACGGACTC |
| | | GACAAGGGGGAGCCCGTCAACGCGGCGGACGCAGCGGCCCTCGAGCACGACAAGGCCTA |
| | | CGACCAGCAGCTCAAAGCGGGTGACAATCCGTACCTGCGGTATAACCACGCCGACGCCG |
| | | AGTTTCAGGAGCGTCTGCAAGAAGATACGTCTTTTGGGGGCAACCTCGGGCGAGCAGTC |
| | | TTCCAGGCCAAGAAGCGGGTTCTCGAACCTCTCGGTCTGGTTGAGGAAGGCGCTAAGAC |
| | | GGCTCCTGGAAAGAAACGTCCGGTAGAGCAGTCGCCACAAGAGCCAGACTCCTCCTCGG |
| | | GCATCGGCAAGACAGGCCAGCAGCCCGCTAAAAAGAGACTCAATTTTGGTCAGACTGGC |
| | | GACTCAGAGTCAGTCCCCGATCCACAACCTCTCGGAGAACCTCCAGCAACCCCCGCTGC |
| | | TGTGGGACCTACTACAATGGCTTCAGGCGGTGGCGCACCAATGGCAGACAATAACGAAG |
| | | GCGCCGACGGAGTGGGTAATGCCTCAGGAAATTGGCATTGCGATTCCACATGGCTGGGC |
| | | GACAGAGTCATCACCACCAGCACCCGCACCTGGGCCTTGCCCACCTACAATAACCACCT |
| | | CTACAAGCAAATCTCCAGTGCTTCAACGGGGGCCAGCAACGACAACCACTACTTCGGCT |
| | | ACAGCACCCCCTGGGGGTATTTTGATTTCAACAGATTCCACTGCCACTTTTCACCACGT |
| | | GACTGGCAGCGACTCATCAACAACAATTGGGGATTCCGGCCCAAGAGACTCAACTTCAA |
| | | ACTCTTCAACATCCAAGTCAAGGAGGTCACGACGAATGATGGCGTCACAACCATCGCTA |
| | | ATAACCTTACCAGCACGGTTCAAGTCTTCTCGGACTCGGAGTACCAGCTTCCGTACGTC |
| | | CTCGGCTCTGCGCACCAGGGCTGCCTCCCTCCGTTCCCGGCGGACGTGTTCATGATTCC |
| | | GCAATACGGCTACCTGACGCTCAACAATGGCAGCCAAGCCGTGGGACGTTCATCCTTTT |
| | | ACTGCCTGGAATATTTCCCTTCTCAGATGCTGAGAACGGGCAACAACTTTACCTTCAGC |
| | | TACACCTTTGAGGAAGTGCCTTTCCACAGCAGCTACGCGCACAGCCAGAGCCTGGACCG |
| | | GCTGATGAATCCTCTCATCGACCAATACCTGTATTACCTGAACAGAACTCAAAATCAGT |
| | | CCGGAAGTGCCCAAAACAAGGACTTGCTGTTTAGCCGTGGGTCTCCAGCTGGCATGTCT |
| | | GTTCAGCCCAAAAACTGGCTACCTGGACCCTGTTATCGGCAGCAGCGCGTTTCTAAAAC |
| | | AAAAACAGACAACAACAACAGCAATTTTACCTGGACTGGTGCTTCAAAATATAACCTCA |
| | | ATGGGCGTGAATCCATCATCAACCCTGGCACTGCTATGGCCTCACACAAAGACGACGAA |
| | | GACAAGTTCTTTCCCATGAGCGGTGTCATGATTTTTGGAAAAGAGAGCGCCGGAGCTTC |
| | | AAACACTGCATTGGACAATGTCATGATTACAGACGAAGAGGAAATTAAAGCCACTAACC |
| | | CTGTGGCCACCGAAAGATTTGGGACCGTGGCAGTCAATTTCCAGAGCAGCAGCACAGAC |
| | | CCTGCGACCGGAGATGTGCATGCTATGGGAGCATTACCTGGCATGGTGTGGCAAGATAG |
| | | AGACGTGTACCTGCAGGGTCCCATTTGGGCCAAAATTCCTCACACAGATGGACACTTTC |
| | | ACCCGTCTCCTCTTATGGGCGGCTTTGGACTCAAGAACCCGCCTCCTCAGATCCTCATC |
| | | AAAAACACGCCTGTTCCTGCGAATCCTCCGGCGGAGTTTTCAGCTACAAAGTTTGCTTC |
| | | ATTCATCACCCAATACTCCACAGGACAAGTGAGTGTGGAAATTGAATGGGAGCTGCAGA |
| | | AAGAAAACAGCAAGCGCTGGAATCCCGAAGTGCAGTACACATCCAATTATGCAAAATCT |
| | | GCCAACGTTGATTTTACTGTGGACAACAATGGACTTTATACTGAGCCTCGCCCCATTGG |
| | | CACCCGTTACCTTACCCGTCCCCTGTAA |
| AAV1 | 71 | MAADGYLPDWLEDNLSEGIREWWDLKPGAPKPKANQQKQDDGRGLVLPGYKYLGPENGL |
| (amino acid) | | DKGEPVNAADAAALEHDKAYDQQLKAGDNPYLRYNHADAEFQERLQEDTSFGGNLGRAV |
| | | FQAKKRVLEPLGLVEEGAKTAPGKKRPVEQSPQEPDSSSGIGKTGQQPAKKRLNFGQTG |
| | | DSESVPDPQPLGEPPATPAAVGPTTMASGGGAPMADNNEGADGVGNASGNWHCDSTWLG |
| | | DRVITTSTRTWALPTYNNHLYKQISSASTGASNDNHYFGYSTPWGYFDFNRFHCHFSPR |
| | | DWQRLINNNWGFRPKRLNFKLFNIQVKEVTTNDGVTTIANNLTSTVQVFSDSEYQLPYV |
| | | LGSAHQGCLPPFPADVFMIPQYGYLTLNNGSQAVGRSSFYCLEYFPSQMLRTGNNFTFS |
| | | YTFEEVPFHSSYAHSQSLDRLMNPLIDQYLYYLNRTQNQSGSAQNKDLLFSRGSPAGMS |
| | | VQPKNWLPGPCYRQQRVSKTKTDNNNSNFTWTGASKYNLNGRESIINPGTAMASHKDDE |
| | | DKFFPMSGVMIFGKESAGASNTALDNVMITDEEEIKATNPVATERFGTVAVNFQSSSTD |
| | | PATGDVHAMGALPGMVWQDRDVYLQGPIWAKIPHTDGHFHPSPLMGGFGLKNPPPQILI |
| | | KNTPVPANPPAEFSATKFASFITQYSTGQVSVEIEWELQKENSKRWNPEVQYTSNYAKS |
| | | ANVDFTVDNNGLYTEPRPIGTRYLTRPL |
| AAV5 | 169 | atgtcttttgttgatcaccctccagattggttggaagaagttggtgaaggtcttcgcga |
| (DNA v1) | | gtttttgggccttgaagcgggcccaccgaaaccaaaacccaatcagcagcatcaagatc |
| | | aagcccgtggtcttgtgctgcctggttataactatctcggacccggaaacggtctcgat |
| | | cgaggagagcctgtcaacagggcagacgaggtcgcgcgagagcacgacatctcgtacaa |
| | | cgagcagcttgaggcgggagacaacccctacctcaagtacaaccacgcggacgccgagt |
| | | ttcaggagaagctcgccgacgacacatccttcgggggaaacctcggaaaggcagtcttt |
| | | caggccaagaaaagggttctcgaaccttttggcctggttgaagagggtgctaagacggc |
| | | ccctaccggaaagcggatagacgaccactttccaaaaagaaagaaggctcggaccgaag |
| | | aggactccaagccttccacctcgtcagacgccgaagctggacccagcggatcccagcag |
| | | ctgcaaatcccagcccaaccagcctcaagtttgggagctgatacaatgtctgcgggagg |
| | | tggcggcccattgggcgacaataaccaaggtgccgatggagtgggcaatgcctcgggag |
| | | attggcattgcgattccacgtggatgggggacagagtcgtcaccaagtccacccgaacc |
| | | tgggtgctgcccagctacaacaaccaccagtaccgagagatcaaaagcggctccgtcga |
| | | cggaagcaacgccaacgcctactttggatacagcaccccctgggggtactttgacttta |
| | | accgcttccacagccactggagcccccgagactggcaaagactcatcaacaactactgg |
| | | ggcttcagaccccggtccctcagagtcaaaatcttcaacattcaagtcaaagaggtcac |
| | | ggtgcaggactccaccaccaccatcgccaacaacctcacctccaccgtccaagtgttta |
| | | cggacgacgactaccagctgccctacgtcgtcggcaacgggaccgagggatgcctgccg |
| | | gccttccctccgcaggtctttacgctgccgcagtacggttacgcgacgctgaaccgcga |
| | | caacacagaaaatcccaccgagaggagcagcttcttctgcctagagtactttcccagca |
| | | agatgctgagaacgggcaacaactttgagtttacctacaactttgaggaggtgcccttc |
| | | cactccagcttcgctcccagtcagaacctgttcaagctggccaacccgctggtggacca |
| | | gtacttgtaccgcttcgtgagcacaaataacactggcggagtccagttcaacaagaacc |
| | | tggccgggagatacgccaacacctacaaaaactggttcccggggcccatgggccgaacc |
| | | cagggctggaacctgggctccggggtcaaccgcgccagtgtcagcgccttcgccacgac |
| | | caataggatggagctcgagggcgcgagttaccaggtgcccccgcagccgaacggcatga |
| | | ccaacaacctccagggcagcaacacctatgccctggagaacactatgatottcaacagc |
| | | cagccggcgaacccgggcaccaccgccacgtacctcgagggcaacatgctcatcaccag |
| | | cgagagcgagacgcagccggtgaaccgcgtggcgtacaacgtcggcgggcagatggcca |
| | | ccaacaaccagagctcTACTACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 213 | AcGagTTTTGTTGATCACCCaCCcGATTGGTTGGAAGAAGTTGGTGAAGGTCTTCGCGA |
| (DNA v2) | | GTTTTTGGGCCTTGAAGCGGGCCCACCGAAACCAAAACCCAATCAGCAGCATCAAGATC |
| | | AAGCCCGTGGTCTTGTGCTGCCTGGTTATAACTATCTCGGACCCGGAAACGGTCTCGAT |
| | | CGAGGAGAGCCTGTCAACAGGGCAGACGAGGTCGCGCGAGAGCACGACATCTCGTACAA |
| | | CGAGCAGCTTGAGGCGGGAGACAACCCCTACCTCAAGTACAACCACGCGGACGCCGAGT |
| | | TTCAGGAGAAGCTCGCCGACGACACATCCTTCGGGGGAAACCTCGGAAAGGCAGTCTTT |
| | | CAGGCCAAGAAAAGGGTTCTCGAACCTTTTGGCCTGGTTGAAGAGGGTGCTAAGACGGC |
| | | CCCTACCGGAAAGCGGATAGACGACCACTTTCCAAAAAGAAAGAAGGCCCGGACCGAAG |
| | | AGGACTCCAAGCCTTCCACCTCGTCAGACGCCGAAGCTGGACCCAGCGGATCCCAGCAG |
| | | CTGCAAATCCCAGCCCAACCAGCCTCAAGTTTGGGAGCTGATACAATGTCTGCGGGAGG |
| | | TGGCGGCCCATTGGGCGACAATAACCAAGGTGCCGATGGAGTGGGCAATGCCTCGGGAG |
| | | ATTGGCATTGCGATTCCACGTGGATGGGGGACAGAGTCGTCACCAAGTCCACCCGAACC |
| | | TGGGTGCTGCCCAGCTACAACAACCACCAGTACCGAGAGATCAAAAGCGGCTCCGTCGA |
| | | CGGAAGCAACGCCAACGCCTACTTTGGATACAGCACCCCCTGGGGGTACTTTGACTTTA |
| | | ACCGCTTCCACAGCCACTGGAGCCCCCGAGACTGGCAAAGACTCATCAACAACTACTGG |
| | | GGCTTCAGACCCCGGTCCCTCAGAGTCAAAATCTTCAACATTCAAGTCAAAGAGGTCAC |
| | | GGTGCAGGACTCCACCACCACCATCGCCAACAACCTCACCTCCACCGTCCAAGTGTTTA |
| | | CGGACGACGACTACCAGCTGCCCTACGTCGTCGGCAACGGGACCGAGGGATGCCTGCCG |
| | | GCCTTCCCTCCGCAGGTCTTTACGCTGCCGCAGTACGGTTACGCGACGCTGAACCGCGA |
| | | CAACACAGAAAATCCCACCGAGAGGAGCAGCTTCTTCTGCCTAGAGTACTTTCCCAGCA |
| | | AGATGCTGAGAACGGGCAACAACTTTGAGTTTACCTACAACTTTGAGGAGGTGCCCTTC |
| | | CACTCCAGCTTCGCTCCCAGTCAGAACCTCTTCAAGCTGGCCAACCCGCTGGTGGACCA |
| | | GTACTTGTACCGCTTCGTGAGCACAAATAACACTGGCGGAGTCCAGTTCAACAAGAACC |
| | | TGGCCGGGAGATACGCCAACACCTACAAAAACTGGTTCCCGGGGCCCATGGGCCGAACC |
| | | CAGGGCTGGAACCTGGGCTCCGGGGTCAACCGCGCCAGTGTCAGCGCCTTCGCCACGAC |
| | | CAATAGGATGGAGCTCGAGGGCGCGAGTTACCAGGTGCCCCCGCAGCCGAACGGCATGA |
| | | CCAACAACCTCCAGGGCAGCAACACCTATGCCCTGGAGAACACTATGATCTTCAACAGC |
| | | CAGCCGGCGAACCCGGGCACCACCGCCACGTACCTCGAGGGCAACATGCTCATCACCAG |
| | | CGAGAGCGAGACGCAGCCGGTGAACCGCGTGGCGTACAACGTCGGCGGGCAGATGGCCA |
| | | CCAACAACCAGAGCTCCACCACTGCCCCCGCGACCGGCACGTACAACCTCCAGGAAATC |
| | | GTGCCCGGCAGCGTGTGGATGGAGAGGGACGTGTACCTCCAAGGACCCATCTGGGCCAA |
| | | GATCCCAGAGACGGGGGCGCACTTTCACCCCTCTCCGGCCATGGGCGGATTCGGACTCA |
| | | AACACCCACCGCCCATGATGCTCATCAAGAACACGCCTGTGCCCGGAAATATCACCAGC |
| | | TTCTCGGACGTGCCCGTCAGCAGCTTCATCACCCAGTACAGCACCGGGCAGGTCACCGT |
| | | GGAGATGGAGTGGGAGCTCAAGAAGGAAAACTCCAAGAGGTGGAACCCAGAGATCCAGT |
| | | ACACAAACAACTACAACGACCCCCAGTTTGTGGACTTTGCCCCGGACAGCACCGGGGAA |
| | | TACAGAACCACCAGACCTATCGGAACCCGATACCTTACCCGACCCCTTTAA |
| AAV5 | 168 | MSFVDHPPDWLEEVGEGLREFLGLEAGPPKPKPNQQHQDQARGLVLPGYNYLGPGNGLD |
| (amino acid) | | RGEPVNRADEVAREHDISYNEQLEAGDNPYLKYNHADAEFQEKLADDTSFGGNLGKAVF |
| | | QAKKRVLEPFGLVEEGAKTAPTGKRIDDHFPKRKKARTEEDSKPSTSSDAEAGPSGSQQ |
| | | LQIPAQPASSLGADTMSAGGGGPLGDNNQGADGVGNASGDWHCDSTWMGDRVVTKSTRT |
| | | WVLPSYNNHQYREIKSGSVDGSNANAYFGYSTPWGYFDFNRFHSHWSPRDWQRLINNYW |
| | | GFRPRSLRVKIFNIQVKEVTVQDSTTTIANNLTSTVQVFTDDDYQLPYVVGNGTEGCLP |
| | | AFPPQVFTLPQYGYATLNRDNTENPTERSSFFCLEYFPSKMLRTGNNFEFTYNFEEVPF |
| | | HSSFAPSQNLFKLANPLVDQYLYRFVSTNNTGGVQFNKNLAGRYANTYKNWFPGPMGRT |
| | | QGWNLGSGVNRASVSAFATTNRMELEGASYQVPPQPNGMINNLQGSNTYALENTMIENS |
| | | QPANPGTTATYLEGNMLITSESETQPVNRVAYNVGGQMATNNQSSTTAPATGTYNLQEI |
| | | VPGSVWMERDVYLQGPIWAKIPETGAHFHPSPAMGGFGLKHPPPMMLIKNTPVPGNITS |
| | | FSDVPVSSFITQYSTGQVTVEMEWELKKENSKRWNPEIQYTNNYNDPQFVDFAPDSTGE |
| | | YRTTRPIGTRYLTRPL |

In some embodiments, the VP-coding region is operably linked to a promoter. In some embodiments, the promoter is a baculovirus major late promoter, a viral promoter, an insect viral promoter, a non-insect viral promoter, a vertebrate viral promoter, a chimeric promoter from one or more species including virus and non-virus elements, a synthetic promoter, or a variant thereof. In some embodiments, the promoter is chosen from a polh promoter, a p10 promoter, a Ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof. In some embodiments, the promoter is a p10 promoter. In some embodiments, the promoter comprises a nucleotide sequence provided in Table 17 or a nucleotide sequence at least 70%, 75%, 80%, 85%, 90%, 95%, 96%, 97%, 98%, or 99% identical thereto. In some embodiments, the promoter comprises the nucleotide sequence of SEQ ID NO: 200, or a nucleotide sequence at least 70%, 75%, 80%, 85%, 90%, 95%, 96%, 97%, 98%, or 99% identical thereto. In some embodiments, the promoter is a p10 promoter and comprises the nucleotide sequence of SEQ ID NO: 200, or a nucleotide sequence at least 70%, 75%, 80%, 85%, 90%, 95%, 96%, 97%, 98%, or 99% identical thereto.

TABLE 17
Exemplary Promoter Sequences

| | SEQ | |
|---|---|---|
| | ID | |
| Description | NO | Sequence |
| polh | 167 | ATCATGGAGATAATTAAAATGATAACCATCTCGCAAATAAATAAGTATTTTACTGTTTTC |
| Promoter | | GTAACAGTTTTGTAATAAAAAAACCTATAAAT |
| p10 | 200 | GACCTTTAATTCAACCCAACACAATATATTATAGTTAAATAAGAATTATTATCAAATCAT |
| promoter | | TTGTATATTAATTAAAATACTATACTGTAAATTACATTTTA |
| CTX | 164 | tgaaactaacttacaagTtggctagtttgttaaaatacgcgctgcgcttgactcgggaat |
| promoter | | acaaagaaaacattattccacactttgatcacttgactcgattgcgcgatttaatcgacg |
| | | gcTtgattaaaagcgaggatgtacaacgttttaatcgcactaatcgcaatgatttaattt |
| | | cggcttgcTtgcaaatcaacgttcggacgtacTtgcccaacgccacgatagatTtgcgca |
| | | aacaacccaactgtatatattttcgaatttgccaatattgccacttggaggccgacgtgc |
| | | cttcgcccgacgatcattcggtgtacagatacttgtgcgtcgcgtgcggcacgccgctgg |
| | | tcatcgaccacccgctcgacgtgttcggccacacggaggaaggcgtcaacgaactgctcg |
| | | aggtgcagcgagtcaacgcgggcggggagttgtaggcgtcataactatttattaaATAAG |
| | | ataatttaaaaaatcgccgttaat |
| gp64 | 217 | AAATTATCGCAAGATAAGGCGCACGTTGATTGGGTCACCCGAGTGTACGTTGATAAAGTC |
| promoter | | ACGTGGGCACCCAACGCGTTGATAAGCATCGGTATATAAGGGCCTACAGTGTTCTGGTAA |
| | | ATCAGTTGCACTGTGCTCTTCACAGGAACACTACAAGACCTACAAG |

### Rep-Coding Region

In certain embodiments, a viral expression construct can comprise a Rep52-coding region. A Rep52-coding region is a nucleotide sequence which comprises a Rep52 nucleotide sequence encoding a Rep52 protein. In certain embodiments, a viral expression construct can comprise a Rep78-coding region. A Rep78-coding region is a nucleotide sequence which comprises a Rep78 nucleotide sequence encoding a Rep78 protein. In certain embodiments, a viral expression construct can comprise a Rep40-coding region. A Rep40-coding region is a nucleotide sequence which comprises a Rep40 nucleotide sequence encoding a Rep40 protein. In certain embodiments, a viral expression construct can comprise a Rep68-coding region. A Rep68-coding region is a nucleotide sequence which comprises a Rep68 nucleotide sequence encoding a Rep68 protein.

In certain embodiments, Rep78 can be produced from a sequence which encodes for Rep78 only. As used herein, the terms “only for Rep78” or “Rep78 only” refer to a nucleotide sequence or transcript which encodes primarily for Rep78 protein relative to non-Rep78 replication proteins (e.g., Rep52 replication proteins). In some embodiments, the nucleotide sequence or transcript: (i) lacks a necessary element within the Rep78 sequence such that transcription or translation of Rep52, as a full or partial sequence, from the Rep78 sequence is reduced or inhibited (e.g., a deletion or mutation in one or more start codons within the Rep78 sequence upstream of the Rep52 sequence); (ii) comprises an exogenous nucleic acid sequence or structure (e.g., one or more additional codons) within the Rep78 sequence which prevents transcription or translation of Rep52 from the same sequence; and/or (iii) comprises a start codon for Rep78 (e.g., ATG), such that Rep78 is the primary Rep protein produced by the nucleotide transcript.

In certain embodiments, Rep52 can be produced from a sequence which encodes for Rep52 only. As used herein, the terms “only for Rep52” or “Rep52 only” refer to a nucleotide sequence or transcript which encodes primarily for Rep52 protein relative to non-Rep52 replication proteins (e.g., Rep78 replication proteins). In some embodiments, the nucleotide sequence or transcript: (i) is a truncated variant of a full Rep sequence (e.g., full Rep78 sequence) which encodes only Rep52 proteins; and/or (ii) comprises a start codon for Rep52 (e.g., ATG), such that Rep52 is the primary Rep protein produced by the nucleotide transcript.

In certain embodiments, the viral expression construct comprises a first nucleotide sequence which comprises: a Rep52-coding region which comprises a Rep52 sequence encoding a Rep52 protein, a Rep78-coding region which comprises a Rep78 sequence encoding a Rep78 protein, or a combination thereof. In certain embodiments, the first nucleotide sequence comprises both a Rep52-coding region and a Rep78-coding region. In certain embodiments, the first nucleotide sequence comprises a single open reading frame, consists essentially of a single open reading frame, or consists of a single open reading frame. In certain embodiments, the first nucleotide sequence comprises a first open reading frame which comprises a Rep52-coding region, and a second open reading frame which comprises a Rep78-coding region and which is different from the first open reading frame.

In some embodiments, a Rep-coding region in an AAV expression construct described herein comprises a nucleotide sequence in Table 16, or encodes a Rep protein comprising an amino acid sequence as provided in Table 16, or a sequence substantially identical (e.g., having at least 70%, 75%, 80%, 85%, 90%, 95%, 96%, 97%, 98%, or 99%) thereto. In some embodiments, the nucleotide sequence encoding the Rep52 comprises nucleotides 673-1866 of SEQ ID NO: 201. In some embodiments, the encoded Rep52 protein comprises amino acids 225-621 of SEQ ID NO: 202.

TABLE 16
Exemplary full length Rep sequences

| | SEQ | |
|---|---|---|
| | ID | |
| Description | NO: | Sequence |
| Rep78 | 201 | ATGGGGGGTTTTACGAGATTGTGATTAAGGTCCCCAGCGACCTTGACGAGCATCTGCC |
| (DNA) | | CGGCATTTCTGACAGCTTTGTGAACTGGGTGGCCGAGAAGGAGTGGGAGTTGCCGCCAG |
| | | ATTCTGACTTGGATCTGAATCTGATTGAGCAGGCACCCCTGACCGTGGCCGAGAAGCTG |
| | | CAGCGCGACTTTCTGACGGAGTGGCGCCGTGTGAGTAAGGCCCCGGAGGCCCTTTTCTT |
| | | TGTGCAATTTGAGAAGGGAGAGAGCTACTTCCACTTACACGTGCTCGTGGAAACCACCG |
| | | GGGTGAAATCCTTAGTTTTGGGACGTTTCCTGAGTCAGATTCGCGAAAAACTGATTCAG |
| | | AGAATTTACCGCGGGATCGAGCCGACTTTGCCAAACTGGTTCGCGGTCACAAAGACCAG |
| | | AAACGGCGCCGGAGGCGGGAACAAGGTGGTGGACGAGTGCTACATCCCCAATTACTTGC |
| | | TCCCCAAAACCCAGCCTGAGCTCCAGTGGGCGTGGACTAATTTAGAACAGTATTTAAGC |
| | | GCCTGTTTGAATCTCACGGAGCGTAAACGGTTGGTGGCGCAGCATCTGACGCACGTGTC |
| | | GCAGACGCAGGAGCAGAACAAAGAGAATCAGAATCCCAATTCTGACGCGCCGGTGATCA |
| | | GATCAAAAACTTCAGCCAGaTACATGGAGCTGGTCGGGTGGCTCGTGGACAAGGGGATT |
| | | ACCTCGGAGAAGCAGTGGATCCAGGAGGACCAGGCCTCATACATCTCCTTCAATGCGGC |
| | | CTCCAACTCGCGGTCCCAAATCAAGGCTGCCTTGGACAATGCGGGAAAGATTATGAGCC |
| | | TGACTAAAACCGCCCCCGACTACCTGGTGGGCCAGCAGCCCGTGGAGGACATTTCCAGC |
| | | AATCGGATTTATAAAATTTTGGAACTAAACGGGTACGATCCCCAATATGCGGCTTCCGT |
| | | CTTTCTGGGATGGGCCACGAAAAAGTTCGGCAAGAGGAACACCATCTGGCTGTTTGGGC |
| | | CTGCAACTACCGGGAAGACCAACATCGCGGAGGCCATAGCCCACACTGTGCCCTTCTAC |
| | | GGGTGCGTAAACTGGACCAATGAGAACTTTCCCTTCAACGACTGTGTCGACAAGATGGT |
| | | GATCTGGTGGGAGGAGGGGAAGATGACCGCCAAGGTCGTGGAGTCGGCCAAAGCCATTC |
| | | TCGGAGGAAGCAAGGTGCGCGTGGACCAGAAATGCAAGTCCTCGGCCCAGATAGACCCG |
| | | ACTCCCGTGATCGTCACCTCCAACACCAACATGTGCGCCGTGATTGACGGGAACTCAAC |
| | | GACCTTCGAACACCAGCAGCCGTTGCAAGACCGGATGTTCAAATTTGAACTCACCCGCC |
| | | GTCTGGATCATGACTTTGGGAAGGTCACCAAGCAGGAAGTCAAAGACTTTTTCCGGTGG |
| | | GCAAAGGATCACGTGGTTGAGGTGGAGCATGAATTCTACGTCAAAAAGGGTGGAGCCAA |
| | | GAAAAGACCCGCCCCCAGTGACGCAGATATAAGTGAGCCCAAACGGGTGCGCGAGTCAG |
| | | TTGCGCAGCCATCGACGTCAGACGCGGAAGCTTCGATCAACTACGCAGACAGGTACCAA |
| | | AACAAATGTTCTCGTCACGTGGGCATGAATCTGATGCTGTTTCCCTGCAGACAATGCGA |
| | | GAGAATGAATCAGAATTCAAATATCTGCTTCACTCACGGACAGAAAGACTGTTTAGAGT |
| | | GCTTTCCCGTGTCAGAATCTCAACCCGTTTCTGTCGTCAAAAAGGCGTATCAGAAACTG |
| | | TGCTACATTCATCATATCATGGGAAAGGTGCCAGACGCTTGCACTGCCTGCGATCTGGT |
| | | CAATGTGGATTTGGATGACTGCATCTTTGAACAATAA |
| Rep78 | 202 | MAGFYEIVIKVPSDLDEHLPGISDSFVNWVAEKEWELPPDSDLDLNLIEQAPLTVAEKL |
| (amino acid) | | QRDFLTEWRRVSKAPEALFFVQFEKGESYFHLHVLVETTGVKSLVLGRFLSQIREKLIQ |
| | | RIYRGIEPTLPNWFAVTKTRNGAGGGNKVVDECYIPNYLLPKTQPELQWAWINLEQYLS |
| | | ACLNLTERKRLVAQHLTHVSQTQEQNKENQNPNSDAPVIRSKTSARYMELVGWLVDKGI |
| | | TSEKQWIQEDQASYISFNAASNSRSQIKAALDNAGKIMSLIKTAPDYLVGQQPVEDISS |
| | | NRIYKILELNGYDPQYAASVFLGWATKKFGKRNTIWLFGPATTGKINIAEAIAHTVPFY |
| | | GCVNWTNENFPFNDCVDKMVIWWEEGKMTAKVVESAKAILGGSKVRVDQKCKSSAQIDP |
| | | TPVIVTSNTNMCAVIDGNSTTFEHQQPLQDRMFKFELTRRLDHDFGKVTKQEVKDFFRW |
| | | AKDHVVEVEHEFYVKKGGAKKRPAPSDADISEPKRVRESVAQPSTSDAEASINYADRYQ |
| | | NKCSRHVGMNLMLFPCRQCERMNQNSNICFTHGQKDCLECFPVSESQPVSVVKKAYQKL |
| | | CYIHHIMGKVPDACTACDLVNVDLDDCIFEQ |
| Rep52 | 203 | ATGGAGCTGGTCGGGTGGCTCGTGGACAAGGGGATTACCTCGGAGAAGCAGTGGATCCA |
| (DNA) | | GGAGGACCAGGCCTCATACATCTCCTTCAATGCGGCCTCCAACTCGCGGTCCCAAATCA |
| | | AGGCTGCCTTGGACAATGCGGGAAAGATTATGAGCCTGACTAAAACCGCCCCCGACTAC |
| | | CTGGTGGGCCAGCAGCCCGTGGAGGACATTTCCAGCAATCGGATTTATAAAATTTTGGA |
| | | ACTAAACGGGTACGATCCCCAATATGCGGCTTCCGTCTTTCTGGGATGGGCCACGAAAA |
| | | AGTTCGGCAAGAGGAACACCATCTGGCTGTTTGGGCCTGCAACTACCGGGAAGACCAAC |
| | | ATCGCGGAGGCCATAGCCCACACTGTGCCCTTCTACGGGTGCGTAAACTGGACCAATGA |
| | | GAACTTTCCCTTCAACGACTGTGTCGACAAGATGGTGATCTGGTGGGAGGAGGGGAAGA |
| | | TGACCGCCAAGGTCGTGGAGTCGGCCAAAGCCATTCTCGGAGGAAGCAAGGTGCGCGTG |
| | | GACCAGAAATGCAAGTCCTCGGCCCAGATAGACCCGACTCCCGTGATCGTCACCTCCAA |
| | | CACCAACATGTGCGCCGTGATTGACGGGAACTCAACGACCTTCGAACACCAGCAGCCGT |
| | | TGCAAGACCGGATGTTCAAATTTGAACTCACCCGCCGTCTGGATCATGACTTTGGGAAG |
| | | GTCACCAAGCAGGAAGTCAAAGACTTTTTCCGGTGGGCAAAGGATCACGTGGTTGAGGT |
| | | GGAGCATGAATTCTACGTCAAAAAGGGTGGAGCCAAGAAAAGACCCGCCCCCAGTGACG |
| | | CAGATATAAGTGAGCCCAAACGGGTGCGCGAGTCAGTTGCGCAGCCATCGACGTCAGAC |
| | | GCGGAAGCTTCGATCAACTACGCAGACAGgtaccaaaacaaatgttctcgtcacgtggg |
| | | catgaatctgatgctgtttccctgcagacaatgcgagagaatgaatcagaattcaaata |
| | | tctgcttcactcacggacagaaagactgtttagagtgctttcccgtgtcagaatctcaa |
| | | cccgtttctgtcgtcaaaaaggcgtatcagaaactgtgctacattcatcatatcatggg |
| | | aaaggtgccagacgcttgcactgcctgcgatctggtcaatgtggatttggatgactgca |
| | | tctttgaacaataa |
| Rep52 | 204 | MELVGWLVDKGITSEKQWIQEDQASYISFNAASNSRSQIKAALDNAGKIMSLIKTAPDY |
| (amino acid) | | LVGQQPVEDISSNRIYKILELNGYDPQYAASVFLGWATKKFGKRNTIWLFGPATTGKIN |
| | | IAEAIAHTVPFYGCVNWTNENFPFNDCVDKMVIWWEEGKMTAKVVESAKAILGGSKVRV |
| | | DQKCKSSAQIDPTPVIVTSNTNMCAVIDGNSTTFEHQQPLQDRMFKFELTRRLDHDFGK |
| | | VTKQEVKDFFRWAKDHVVEVEHEFYVKKGGAKKRPAPSDADISEPKRVRESVAQPSTSD |
| | | AEASINYADRYQNKCSRHVGMNLMLFPCRQCERMNQNSNICFTHGQKDCLECFPVSESQ |
| | | PVSVVKKAYQKLCYIHHIMGKVPDACTACDLVNVDLDDCIFEQ |

In certain embodiments, the viral expression construct comprises a Rep78-coding region in a first transcriptional cassette (e.g., ORF). In certain embodiments, the viral expression construct comprises a Rep52-coding region in a second transcriptional cassette (e.g., ORF), which is separate from the first (i.e., Rep78-coding) transcriptional cassette. In certain embodiments, the first (i.e., Rep78-coding) transcriptional cassette is at a first location of a baculovirus vector, and the second (i.e., Rep52-coding) transcriptional cassette is at a second location of the baculovirus vector. In certain embodiments, the first location and the second location are distal from each other (e.g., at least 5000 bp apart). In certain embodiments, the first location and the second location are at least 2000 bp apart, at least 2500 bp apart, at least 3000 bp apart, at least 3500 bp apart, at least 4000 bp apart, at least 4500 bp apart, at least 5000 bp apart, at least 5500 bp apart, at least 6000 bp apart, at least 6500 bp apart, at least 7000 bp apart, at least 7500 bp apart, at least 8000 bp apart, at least 8500 bp apart, at least 9000 bp apart, at least 9500 bp apart, at least 10000 bp apart, at least 10500 bp apart, at least 11000 bp apart, at least 11500 bp apart, at least 12000 bp apart, at least 12500 bp apart, at least 13000 bp apart, at least 13500 bp apart, at least 14000 bp apart, at least 14500 bp apart, at least 15000 bp apart, at least 15500 bp apart, at least 16000 bp apart, at least 16500 bp apart, at least 17000 bp apart, at least 17500 bp apart, at least 18000 bp apart, at least 18500 bp apart, at least 19000 bp apart, at least 19500 bp apart, at least 20000 bp apart, at least 20500 bp apart, at least 21000 bp apart, at least 21500 bp apart, at least 22000 bp apart, at least 22500 bp apart, at least 23000 bp apart, at least 23500 bp apart, at least 24000 bp apart, at least 24500 bp apart, or at least 25000 bp apart, within the baculovirus vector. In certain embodiments, the first (i.e., Rep78-coding) transcriptional cassette is in the polh gene location of the baculovirus vector. In certain embodiments, the first (i.e., Rep78-coding) transcriptional cassette is in the egt gene location of the baculovirus vector. In certain embodiments, the second (i.e., Rep52-coding) transcriptional cassette is in the polh gene location of the baculovirus vector. In certain embodiments, the second (i.e., Rep52-coding) transcriptional cassette is in the egt gene location of the baculovirus vector. In certain embodiments, the first (i.e., Rep78-coding) transcriptional cassette is in the polh gene location of the baculovirus vector, and the second (i.e., Rep52-coding) transcriptional cassette is in the egt gene location of the baculovirus vector. In certain embodiments, the first (i.e., Rep78-coding) transcriptional cassette is in the egt gene location of the baculovirus vector, and the second (i.e., Rep52-coding) transcriptional cassette is in the polh gene location of the baculovirus vector.

In certain embodiments, non-structural proteins, Rep52 and Rep78, of a viral expression construct can be encoded in a single open reading frame regulated by utilization of both alternative splice acceptor and non-canonical translational initiation codons.

Both Rep78 and Rep52 can be translated from a single transcript: Rep78 translation initiates at a first start codon (AUG or non-AUG) and Rep52 translation initiates from a Rep52 start codon (e.g., AUG) within the Rep78 sequence. Rep78 and Rep52 can also be translated from separate transcripts with independent start codons. The Rep52 initiation codons within the Rep78 sequence can be mutated, modified, or removed, such that processing of the modified Rep78 sequence will not produce Rep52 proteins.

In certain embodiments, the viral expression construct of the present disclosure may be a plasmid vector or a baculoviral construct that encodes the parvoviral rep proteins for expression in insect cells. In certain embodiments, a single coding sequence is used for the Rep78 and Rep52 proteins, wherein start codon for translation of the Rep78 protein is a suboptimal start codon, selected from the group consisting of ACG, TTG, CTG and GTG, that effects partial exon skipping upon expression in insect cells, as described in U.S. Pat. No. 8,512,981, the content of which is incorporated herein by reference in its entirety as related to the promotion of less abundant expression of Rep78 as compared to Rep52 to promote high vector yields.

In certain embodiments, the viral expression construct may be a plasmid vector or a baculoviral construct for the expression in insect cells that contains repeating codons with differential codon biases, for example to achieve improved ratios of Rep proteins, e.g., Rep78 and Rep52 thereby improving large scale (commercial) production of viral expression construct and/or payload construct vectors in insect cells, as taught in U.S. Pat. No. 8,697,417, the content of which is incorporated herein by reference in its entirety as related to AAV replication proteins and the production thereof.

In certain embodiment, improved ratios of rep proteins may be achieved using the method and constructs described in U.S. Pat. No. 8,642,314, the content of which is incorporated herein by reference in its entirety as related to AAV replications proteins and the production thereof.

In certain embodiments, the viral expression construct may encode mutant parvoviral Rep polypeptides which have one or more improved properties as compared with their corresponding wild-type Rep polypeptide, such as the preparation of higher virus titers for large scale production. Alternatively, they may be able to allow the production of better-quality viral particles or sustain more stable production of virus. In a non-limiting example, the viral expression construct may encode mutant Rep polypeptides with a mutated nuclear localization sequence or zinc finger domain, as described in Patent Application US 20130023034, the content of which is incorporated herein by reference in its entirety as related to AAV replications proteins and the production thereof.

In certain embodiments of the present disclosure, Rep52 or Rep78 is transcribed from the baculoviral derived polyhedron promoter (polh). Rep52 or Rep78 can also be transcribed from a weaker promoter, for example a deletion mutant of the IE-1 promoter, the ΔIE-1 promoter, has about 20% of the transcriptional activity of that IE-1 promoter. A promoter substantially homologous to the ΔIE-1 promoter may be used. In respect to promoters, a homology of at least 50%, 60%, 70%, 80%, 90% or more, is considered to be a substantially homologous promoter.

### Polynucleotide Inserts

In certain embodiments, a viral, e.g., AAV, expression construct or a payload construct of the present disclosure (e.g., bacmid) can be generated using molecular biology techniques (e.g., transposon donor/acceptor system). In certain embodiments, the polynucleotide incorporated into the bacmid can include an expression control sequence operably linked to a protein-coding nucleotide sequence. In certain embodiments, the polynucleotide incorporated into the bacmid can include an expression control sequence which includes a promoter, such as p10 or polh, and which is operably linked to a nucleotide sequence which encodes a structural AAV capsid protein (e.g., VP1, VP2, VP3 or a combination thereof). In certain embodiments, the polynucleotide incorporated into the bacmid can include an expression control sequence which includes a promoter, such as p10 or polh, and which is operably linked to a nucleotide sequence which encodes a non-structural AAV capsid protein (e.g., Rep78, Rep52, or a combination thereof).

In certain embodiments, the polynucleotide insert can be incorporated into the bacmid using the Gibson Assembly method, as described in Gibson et al. (2009) Nat. Methods 6, 343-345, and Gibson et al. (2010) Science 329, 52-56; the contents of which are each incorporated herein by reference in their entireties as related to the use of Gibson Assembly method for incorporating polynucleotide inserts into a bacmid. In certain embodiments, the polynucleotide insert can include one or more Gibson Assembly sequences at the 5′ end of the insert, at the 3′ end of the insert, or at both the 5′ end and 3′ end of the insert; such that the one or more Gibson Assembly sequences allow for the incorporation of the polynucleotide insert into a target location of bacmid. In certain embodiments, the Gibson Assembly method can include the use of NEBuilder Hifi optimized enzyme mix.

In certain embodiments, the polynucleotide insert can be incorporated into the bacmid at the location of a baculoviral gene. In certain embodiments, the polynucleotide insert can be incorporated into the bacmid at the location of a non-essential baculoviral gene. In certain embodiments, the polynucleotide insert can be incorporated into the bacmid by replacing a baculoviral gene or a portion of the baculoviral gene with the polynucleotide insert. In certain embodiments, the polynucleotide insert can be incorporated into the bacmid by replacing a baculoviral gene or a portion of the baculoviral gene with a fusion-polynucleotide which includes the polynucleotide insert and the baculoviral gene (or portion thereof) being replaced.

In certain embodiments, the polynucleotide can be incorporated into the bacmid at the location of a restriction endonuclease (REN) cleavage site (e.g., REN access point) associated with a baculoviral gene. In certain embodiments, the polynucleotide can be incorporated into the bacmid using one or more endonucleases (e.g., homing endonucleases). See, for example, Lihoradova et al., J Virol Methods, 140(1-2):59-65 (2007), the content of which is incorporated herein by reference in its entirety as related to the direct cloning of foreign DNA into baculovirus genomes.

In certain embodiments, the REN access point in the bacmid is FseI (corresponding with the global transactivator (gta) baculovirus gene) (ggccggcc). In certain embodiments, the REN access point in the bacmid is SdaI (corresponding with the DNA polymerase baculovirus gene) (cctgcagg). In certain embodiments, the REN access point in the bacmid is MauBI (corresponding with the lef-4 baculovirus gene) (cgcgcgcg). In certain embodiments, the REN access point in the bacmid is SbfI (corresponding with the gp64/gp67 baculovirus gene) (cctgcagg). In certain embodiments, the REN access point in the bacmid is I-CeuI (corresponding with the v-cath baculovirus gene) (SEQ ID NO: 1). In certain embodiments, the REN access point in the bacmid is AvrII (corresponding with the ecdysteroid UDP-glucosyltransferase (egt) baculovirus gene) (cctagg). In certain embodiments, the REN access point in the bacmid is NheI (gctagc). In certain embodiments, the REN access point in the bacmid is SpeI (actagt). In certain embodiments, the REN access point in the bacmid is BstZ17I (gtatac). In certain embodiments, the REN access point in the bacmid is NcoI (ccatgg). In certain embodiments, the REN access point in the bacmid is MluI (acgcgt).

Polynucleotides can be incorporated into these REN access points by: (i) providing a polynucleotide insert which has been engineered to include a target REN cleavage sequence (e.g., a polynucleotide insert engineered to include FseI REN sequences at both ends of the polynucleotide); (ii) proving a bacmid which includes the target REN access point for polynucleotide insertion (e.g., a variant of the AcMNPV bacmid bMON14272 which includes an FseI cleavage site (ii) digesting the REN-engineered polynucleotide with the appropriate REN enzyme (e.g., using FseI enzyme to digesting the REN-engineering polynucleotide which includes the FseI regions at both ends, to produce a polynucleotide-FseI insert); (iii) digesting the bacmid with the same REN enzyme to produce a single-cut bacmid at the REN access point (e.g., using FseI enzyme to produce a single-cut bacmid at the FseI location); and (iv) ligating the polynucleotide insert into the single-cut bacmid using an appropriate ligation enzyme, such as T4 ligase enzyme. The result is engineered bacmid DNA which includes the engineered polynucleotide insert at the target REN access point.

The insertion process can be repeated one or more times to incorporate other engineered polynucleotide inserts into the same bacmid at different REN access points (e.g., insertion of a first engineered polynucleotide insert at the AvrII REN access point in the egt, followed by insertion of a second engineered polynucleotide insert at the I-CeuI REN access point in the cath gene, and followed by insertion of a third engineered polynucleotide insert at the FseI REN access point in the gta gene).

In certain embodiments, the polynucleotide insert can be incorporated into the bacmid by splitting a baculoviral gene with the polynucleotide insert (e.g., the polynucleotide insert is incorporated into the middle of the gene, separating a 5′-portion of the gene from a 3′-portion of the bacmid gene). In certain embodiments, the polynucleotide insert can be incorporated into the bacmid by splitting a baculoviral gene with the fusion-polynucleotide which includes the polynucleotide insert and a portion of the baculoviral gene which was split. In certain embodiments, the 3′ end of the fusion-polynucleotide includes the 5′-portion of the gene that was split, such that the 5′-portion of the gene in the fusion-polynucleotide and the 3′-portion of the gene remaining in the bacmid form a full or functional portion of the baculoviral gene. In certain embodiments, the 5′ end of the fusion-polynucleotide includes the 3′-portion of the gene that was split, such that the 3′-portion of the gene in the fusion-polynucleotide and the 5′-portion of the gene remaining in the bacmid form a full or functional portion of the baculoviral gene. In certain embodiments, fusion-polynucleotides are engineered and produced to include components from the gta gene ORF (full/partial Ac-lef12 promoter, full/partial Ac-gta gene). Non-limiting examples of fusion polynucleotides of the present disclosure include the polynucleotides of SEQ ID NO: 2 and SEQ ID NO: 3.

In certain embodiments, restriction endonuclease (REN) cleavage can be used to remove one or more wild-type genes from a bacmid. In certain embodiments, restriction endonuclease (REN) cleavage can be used to remove one or more engineered polynucleotide insert which has been previously inserted into the bacmid. In certain embodiments, restriction endonuclease (REN) cleavage can be used to replace one or more engineered polynucleotide inserts with a different engineered polynucleotide insert which includes the same REN cleavage sequences (e.g., an engineered polynucleotide insert at the FseI REN access point can be replaced with a different engineered polynucleotide insert which includes FseI REN cleavage sequences).

### Expression Control

### Expression Control Regions

An AAV expression construct (e.g., expressionBac) of the present disclosure can comprise one or more expression control region encoded by expression control sequences. In certain embodiments, the expression control sequences are for expression in a viral production cell, such as an insect cell. In certain embodiments, the expression control sequences are operably linked to a protein-coding nucleotide sequence. In certain embodiments, the expression control sequences are operably linked to a VP coding nucleotide sequence or a Rep coding nucleotide sequence.

Herein, the terms “coding nucleotide sequence”, “protein-encoding gene” or “protein-coding nucleotide sequence” refer to a nucleotide sequence that encodes or is translated into a protein product, such as VP proteins or Rep proteins. Being operably linked indicates that the expression control sequence is positioned relative to the coding sequence such that it can promote the expression of the encoded gene product.

“Expression control sequence” refers to a nucleic acid sequence that regulates the expression of a nucleotide sequence to which it is operably linked. Thus, an expression control sequence can include promoters, enhancers, untranslated regions (UTRs), internal ribosome entry sites (IRES), transcription terminators, a start codon in front of a protein-encoding gene, splicing signal for introns, and stop codons. The term “expression control sequence” is intended to include, at a minimum, a sequence whose presence are designed to influence expression, and can also include additional advantageous components. For example, leader sequences and fusion partner sequences are expression control sequences. The term can also comprise the design of the nucleic acid sequence such that undesirable, potential initiation codons in and out of frame, are removed from the sequence. It can also comprise the design of the nucleic acid sequence such that undesirable potential splice sites are removed. It comprises sequences or polyadenylation sequences (pA) which direct the addition of a polyA tail, e.g., a string of adenine residues at the 3′-end of an mRNA, sequences referred to as polyA sequences. It also can be designed to enhance mRNA stability. Expression control sequences which affect the transcription and translation stability, e.g., promoters, as well as sequences which effect the translation, e.g., Kozak sequences, are known in insect cells. Expression control sequences can be of such nature as to modulate the nucleotide sequence to which it is operably linked such that lower expression levels or higher expression levels are achieved.

In certain embodiments, the expression control sequence can comprise one or more promoters. Promoters can comprise, but are not limited to, baculovirus major late promoters, insect virus promoters, non-insect virus promoters, vertebrate virus promoters, nuclear gene promoters, chimeric promoters from one or more species comprising virus and non-virus elements, and/or synthetic promoters. In certain embodiments, a promoter can be Ctx, Op-EI, EI, ΔEI, EI-1, pH, polh (polyhedrin), Δpolh, Dmhsp70, Hr 1, Hsp70, 4×Hsp27, EcRE+minimal, Hsp70, IE, IE-1, ΔIE-1, ΔIE, p10, Δp10 (modified variations or derivatives of p10), p5, p19, p35, p40, p6.9, and variations or derivatives thereof. In certain embodiments, the promoter is a ctx promoter. In certain embodiments, the promoter is a p10 promoter. In certain embodiments, the promoter is a polh promoter. In certain embodiments, a promoter can be selected from tissue-specific promoters, cell-type-specific promoters, cell-cycle-specific promoters, and variations or derivatives thereof. In certain embodiments, a promoter can be a CMV promoter, an alpha 1-antitrypsin (al-AT) promoter, a thyroid hormone-binding globulin promoter, a thyroxine-binding globulin (LPS) promoter, an HCR-ApoCII hybrid promoter, an HCR-hAAT hybrid promoter, an albumin promoter, an apolipoprotein E promoter, an α1-AT+EaIb promoter, a tumor-selective E2F promoter, a mononuclear blood IL-2 promoter, and variations or derivatives thereof. In certain embodiments, the promoter is a low-expression promoter sequence. In certain embodiments, the promoter is an enhanced-expression promoter sequence. In certain embodiments, the promoter can comprise Rep or Cap promoters as described in US Patent Application 20110136227, the content of which is incorporated herein by reference in its entirety as related to expression promoters.

In some embodiments, the AAV expression construct comprises a ctx promoter. In some embodiments, the CTX promoter comprises a sequence as provided in Table 8, or a sequence substantially identical (e.g., having at least about 70%, 75%, 80%, 85%, 90%, 92%, 95%, 97%, 98%, or 99% sequence identity) thereto.

TABLE 8
Exemplary CTX promoter sequences

| | SEQ | |
|---|---|---|
| | ID | |
| Description | NO: | Sequence |
| CTX | 164 | tgaaactaacttacaagTtggctagtttgttaaaatacgcgctgcgcttgactcgggaa |
| promoter | | tacaaagaaaacattattccacactttgatcacttgactcgattgcgcgatttaatcga |
| | | cggcTtgattaaaagcgaggatgtacaacgttttaatcgcactaatcgcaatgatttaa |
| | | tttcggcttgcTtgcaaatcaacgttcggacgtacTtgcccaacgccacgatagatTtg |
| | | cgcaaacaacccaactgtatatattttcgaatttgccaatattgccacttggaggccga |
| | | cgtgccttcgcccgacgatcattcggtgtacagatacttgtgcgtcgcgtgcggcacgc |
| | | cgctggtcatcgaccacccgctcgacgtgttcggccacacggaggaaggcgtcaacgaa |
| | | ctgctcgaggtgcagcgagtcaacgcgggcggggagttgtaggcgtcataactatttat |
| | | taaATAAGataatttaaaaaatcgccgttaat |
| CTX | 165 | ATAAGataatttaaaaaatcgccgttaat |
| promoter | | |
| CTX | 166 | gggcggggagttgtaggcgtcataactatttattaaATAAGataatttaaaaaatcgcc |
| promoter | | gttaat |

In certain embodiments, the expression control sequence can comprise one or more expression-modifier sequences, such as a minicistron insertion sequence. In certain embodiments, the expression control sequence can comprise one or more expression modifiers (e.g., minicistron insertion) which is upstream and functionally adjacent/near a start codon (e.g., VP1 start codon, Rep78 start codon). Without being bound by theory, insertion of an expression modifier (e.g., minicistron) upstream and functionally adjacent/near a start codon can result in scanning ribosomes being less competent to recognize, bind, and/or initiate translation at the target ORF start codon (e.g., Rep78 ATG start codon). This can result in decreased translation initiation at a target ORF start codon (e.g., Rep78 ATG start codon), and correspondingly result in increased initiation at downstream ORF start codons (e.g., Rep52 start codon within bicistronic sequence).

In certain embodiments, the expression modifier (e.g., minicistron insertion) is upstream of a target ORF start codon (e.g., Rep78 ATG start codon). In certain embodiments, the expression control sequence comprises one or more nucleotides between the expression modifier (e.g., minicistron insertion) and the target ORF start codon (e.g., Rep78 ATG start codon). In certain embodiments, the expression control sequence comprises between 1-100 nucleotides between the expression modifier and the target ORF start codon. In certain embodiments, the expression control sequence comprises between 3-100 nucleotides between the expression modifier and the target ORF start codon. In certain embodiments, the expression control sequence comprises between 3-75 nucleotides between the expression modifier and the target ORF start codon. In certain embodiments, the expression control sequence comprises between 3-50 nucleotides between the expression modifier and the target ORF start codon. In certain embodiments, the expression control sequence comprises between 3-25 nucleotides between the expression modifier and the target ORF start codon. In certain embodiments, the expression control sequence comprises between 3-15 nucleotides between the expression modifier and the target ORF start codon. In certain embodiments, the expression control sequence comprises between 3-10 nucleotides between the expression modifier and the target ORF start codon. In certain embodiments, the expression control sequence comprises between 3-6 nucleotides between the expression modifier and the target ORF start codon. In certain embodiments, the expression control sequence comprises 3 nucleotides between the expression modifier and the target ORF start codon.

In certain embodiments, the expression modifier is a minicistron insertion sequence (i.e., small open reading frame). In certain embodiments, the minicistron insertion sequence is from a baculovirus gene. In certain embodiments, the minicistron insertion sequence is from a baculovirus gp64 gene. In certain embodiments, the minicistron insertion sequence comprises SEQ ID NO: 4. In certain embodiments, the minicistron insertion sequence comprises SEQ ID NO: 5.

In certain embodiments, a viral expression construct can comprise the same promoter in all nucleotide sequences. In certain embodiments, a viral expression construct can comprise the same promoter in two or more nucleotide sequences. In certain embodiments, a viral expression construct can comprise a different promoter in two or more nucleotide sequences. In certain embodiments, a viral expression construct can comprise a different promoter in all nucleotide sequences.

In certain embodiments the viral expression construct encodes elements to improve expression in certain cell types. In a further embodiment, the expression construct may comprise polh and/or ΔIE-1 insect transcriptional promoters, CMV mammalian transcriptional promoter, and/or p10 insect specific promoters for expression of a desired gene in a mammalian or insect cell.

More than one expression control sequence can be operably linked to a given nucleotide sequence. For example, a promoter sequence, a translation initiation sequence, and a stop codon can be operably linked to a nucleotide sequence.

In certain embodiments, the viral expression construct can comprise one or more expression control sequence between protein-coding nucleotide sequences. In certain embodiments, an expression control region can comprise an IRES sequence region which comprises an IRES nucleotide sequence encoding an internal ribosome entry sight (IRES). The internal ribosome entry sight (IRES) can be selected from the group consisting or: FMDV-IRES from Foot-and-Mouth-Disease virus, EMCV-IRES from Encephalomyocarditis virus, and combinations thereof.

In certain embodiments, the viral expression construct is as described in PCT/US2019/054600 and/or U.S. Provisional Patent Application No. 62/741,855 the contents of which are each incorporated by reference in their entireties.

In certain embodiments, the viral expression construct may contain a nucleotide sequence which comprises a start codon region, such as a sequence encoding AAV capsid proteins which comprise one or more start codon regions. In certain embodiments, the start codon region can be within an expression control sequence.

In certain embodiments, the translational start site of eukaryotic mRNA can be controlled in part by a nucleotide sequence referred to as a Kozak sequence as described in Kozak, M Cell. 1986 Jan. 31; 44(2):283-92 and Kozak, M. J Cell Biol. 1989 February; 108(2):229-41 the contents of each of which are herein incorporated by reference in their entirety as related to Kozak sequences and uses thereof. Both naturally occurring and synthetic translational start sites of the Kozak form can be used in the production of polypeptides by molecular genetic techniques, Kozak, M. Mamm Genome. 1996 August; 7(8):563-74 the contents of which are herein incorporated by reference in their entirety as related to Kozak sequences and uses thereof. Splice sites are sequences on an mRNA which facilitate the removal of parts of the mRNA sequences after the transcription (formation) of the mRNA. Typically, the splicing occurs in the nucleus, prior to mRNA transport into a cell's cytoplasm.

In some embodiments, the AAV expression construct described herein comprises a modified Kozak sequence. In some embodiments, the modified Kozak sequence is present upstream of a VP coding region, e.g., a VP1, a VP2, or a VP3 coding region. In some embodiments, the modified Kozak sequence is present upstream of a Rep coding region, e.g., a Rep52 coding region or a Rep78 coding region. In some embodiments, the modified Kozak sequence comprises a sequence as provided in Table 9. In some embodiments, the modified Kozak sequence comprises a sequence as provided in Table 10. In some embodiments, the modified Kozak sequence comprises a sequence as described in Noderer, William L., et al. “Quantitative analysis of mammalian translation initiation sites by FACS-seq.” Molecular systems biology 10.8 (2014): 748; and Diaz de Arce et al. “Complete motif analysis of sequence requirements for translation initiation at non-AUG start codons” Nucleic Acids Res. 2018 January 46(2):985-994; and Kondratov et. al. “Direct Head-to-Head Evaluation of Recombinant Adeno-associated Viral Vectors Manufactured in Human versus Insect Cells” Molecular Therapy. 2017 Dec. 25(12):2661-2675, the contents of which are hereby incorporated by reference in their entirety. In some embodiments, the modified Kozak sequence comprises a sequence provided in US20200123572, WO2017181162, and WO2021222472, the contents of which are hereby incorporated by reference in their entirety.

TABLE 9
Exemplary Modified Kozak Sequences

| | | SEQ | | SEQ |
|---|---|---|---|---|
| | Sequence | ID | Sequence | ID |
| Name | (RNA) | NO: | (DNA) | NO: |
| VP1aug13 | cgguguAUGgc | 21 | cggtgtATGgc | 32 |
| VP1aug14 | cgguguAUGag | 22 | cggtgtATGag | 33 |
| VP1aug28 | uaguggAUGag | 23 | tagtggATGag | 34 |
| VP1aug29 | uauuggAUGag | 24 | tattggATGag | 35 |
| VP1aug30 | caguggAUGag | 25 | cagtggATGag | 36 |
| VP1aug31 | cauuggAUGag | 26 | cattggATGag | 37 |
| VP1aug32 | ugguggAUGag | 27 | tggtggATGag | 38 |
| VP1aug33 | aauuggAUGag | 28 | aattggATGag | 39 |
| VP1aug34 | aaguggAUGag | 29 | aagtggATGag | 40 |
| VP1aug35 | ccguggAUGag | 30 | ccgtggATGag | 41 |
| VP1aug36 | ggguggAUGag | 31 | gggtggATGag | 42 |

TABLE 10
Exemplary Modified Kozak Sequences

| | SEQ | | SEQ |
|---|---|---|---|
| | ID | | ID |
| Sequence (RNA) | NO: | Sequence (DNA) | NO: |
| UAGUGUAUGUC | 73 | TAGTGTATGTC | 118 |
| UAGUUUAUGUC | 74 | TAGTTTATGTC | 119 |
| UGUUUUAUGUC | 75 | TGTTTTATGTC | 120 |
| UCUUUUAUGUC | 76 | TCTTTTATGTC | 121 |
| UAGUGUAUGGG | 77 | TAGTGTATGGG | 122 |
| UAGUUUAUGGG | 78 | TAGTTTATGGG | 123 |
| UGUUUUAUGGG | 79 | TGTTTTATGGG | 124 |
| UCUUUUAUGGG | 80 | TCTTTTATGGG | 125 |
| UAGUGUAUGGC | 81 | TAGTGTATGGC | 126 |
| UAGUUUAUGGC | 82 | TAGTTTATGGC | 127 |
| UGUUUUAUGGC | 83 | TGTTTTATGGC | 128 |
| UCUUUUAUGGC | 84 | TCTTTTATGGC | 129 |
| CAUUGUAUGUC | 85 | CATTGTATGTC | 130 |
| UCGUUUAUGGA | 86 | TCGTTTATGGA | 131 |
| CAGUUUAUGGU | 87 | CAGTTTATGGT | 132 |
| CAUUGUAUGGU | 88 | CATTGTATGGT | 133 |
| UAGUGUAUGCU | 89 | TAGTGTATGCT | 134 |
| CAUUGUAUGCU | 90 | CATTGTATGCT | 135 |
| UCUUUUAUGUC | 91 | TCTTTTATGTC | 136 |
| UGUUUUAUGUC | 92 | TGTTTTATGTC | 137 |
| UAGUUUAUGUC | 93 | TAGTTTATGTC | 138 |
| UAGUGUAUGUC | 94 | TAGTGTATGTC | 139 |
| CGACAUGAAUGUC | 95 | CGACATGAATGTC | 140 |
| AGGGAUGGAUGUC | 96 | AGGGATGGATGTC | 141 |
| CUUGAUGGAUGUC | 97 | CTTGATGGATGTC | 142 |
| CGUGAUGUAUGUC | 98 | CGTGATGTATGTC | 143 |
| GGCGAUGGAUGUC | 99 | GGCGATGGATGTC | 144 |
| CGUGAUGGAUGUC | 100 | CGTGATGGATGTC | 145 |
| CGUAAUGCAUGUC | 101 | CGTAATGCATGTC | 146 |
| GAACAUGAAUGUC | 102 | GAACATGAATGTC | 147 |
| GGACAUGCAUGUC | 103 | GGACATGCATGTC | 148 |
| AAGAAUGAAUGUC | 104 | AAGAATGAATGTC | 149 |
| CCCGAUGGAUGUC | 105 | CCCGATGGATGTC | 150 |
| GCCGAUGUAUGUC | 106 | GCCGATGTATGTC | 151 |
| CGGGAUGAAUGUC | 107 | CGGGATGAATGTC | 152 |
| GACCAUGCAUGGCU | 108 | GACCATGCATGGCT | 153 |
| AAUUAUGCAUGGC | 109 | AATTATGCATGGC | 154 |
| AGACAUGUAUGGC | 110 | AGACATGTATGGC | 155 |
| AAGAAUGUAUGGC | 111 | AAGAATGTATGGC | 156 |
| GACCAUGCAUGGCU | 112 | GACCATGCATGGCT | 157 |
| CGCGAUGGAUGGC | 113 | CGCGATGGATGGC | 158 |
| AGCGAUGGAUGGC | 114 | AGCGATGGATGGC | 159 |
| GAUCAUGCAUGGC | 115 | GATCATGCATGGC | 160 |
| CGUAAUGGAUGGC | 116 | CGTAATGGATGGC | 161 |
| AACGAUGUAUGGC | 117 | AACGATGTATGGC | 162 |

In certain embodiments, the viral expression construct may contain a nucleotide sequence which comprises a stop codon region, such as a sequence encoding AAV capsid proteins which comprise one or more stop codon regions. In certain embodiments, the stop codon region can be within an expression control sequence.

In certain embodiments, the viral expression construct comprises one or more start codon regions which include a start codon. In certain embodiments, the viral expression construct comprises one or more stop codon regions which include a stop codon. In certain embodiments, the viral expression construct comprises one or more start codon regions and one or more stop codon regions. In certain embodiments, the start codon region and/or stop codon region can be within an expression control sequence.

In certain embodiments, the viral expression construct comprises one or more expression control regions which comprise an expression control sequence. In certain embodiments, the expression control region comprises one or more promoter sequences. In certain embodiments, the expression control region comprises one or more promoter sequences selected from the group consisting of: baculovirus major late promoters, insect virus promoters, non-insect virus promoters, vertebrate virus promoters, nuclear gene promoters, chimeric promoters from one or more species including virus and non-virus elements, synthetic promoters, and variations or derivatives thereof. In certain embodiments, the expression control region comprises one or more promoter sequences selected from the group consisting of: Ctx promoter, polh insect transcriptional promoters, ΔIE-1 insect transcriptional promoters, p10 insect specific promoters, Δp10 insect specific promoters (variations or derivatives of p10), CMV mammalian transcriptional promoter, and variations or derivatives thereof. In certain embodiments, the expression control region comprises one or more low-expression promoter sequences. In certain embodiments, the expression control region comprises one or more enhanced-expression promoter sequences.

In certain embodiments, an expression control region can comprise a 2A sequence region which comprises a 2A nucleotide sequence encoding a viral 2A peptide. The sequence allows for co-translation of multiple polypeptides within a single open reading frame (ORF). As the ORF is translated, glycine and proline residues with the 2A sequence prevent the formation of a normal peptide bond, which results in ribosomal “skipping” and “self-cleavage” within the polypeptide chain. The viral 2A peptide can be selected from the group consisting of: F2A from Foot-and-Mouth-Disease virus, T2A from Thosea asigna virus, E2A from Equine rhinitis A virus, P2A from porcine teschovirus-1, BmCPV2A from cytoplasmic polyhedrosis virus, BmIFV 2A from B. mori flacherie virus, and combinations thereof.

In some embodiments, the first and/or second nucleotide sequence comprises a start codon and/or stop codon and/or internal ribosome entry site (IRES). In certain embodiments, the IRES nucleotide sequence encodes an internal ribosome entry site (IRES) selected from the group consisting of: FMDV-IRES from Foot-and-Mouth-Disease virus, EMCV-IRES from Encephalomyocarditis virus, and combinations thereof.

The method of the present disclosure is not limited by the use of specific expression control sequences. However, when a certain stoichiometry of VP products are achieved (close to 1:1:10 for VP1, VP2, and VP3, respectively) and also when the levels of Rep52 or Rep40 (also referred to as the p19 Reps) are significantly higher than Rep78 or Rep68 (also referred to as the p5 Reps), improved yields of AAV in production cells (such as insect cells) may be obtained. In certain embodiments, the p5/p19 ratio is below 0.6 more, below 0.4, or below 0.3, but always at least 0.03. These ratios can be measured at the level of the protein or can be implicated from the relative levels of specific mRNAs.

In certain embodiments, AAV particles are produced in viral production cells (such as mammalian or insect cells) wherein all three VP proteins are expressed at a stoichiometry approaching, about or which is 1:1:10 (VP1:VP2:VP3).

In certain embodiments, AAV particles are produced in viral production cells (such as mammalian or insect cells) wherein all three VP proteins are expressed at a stoichiometry approaching, about or which is 2:2:10 (VP1:VP2:VP3).

In certain embodiments, AAV particles are produced in viral production cells (such as mammalian or insect cells) wherein all three VP proteins are expressed at a stoichiometry approaching, about or which is 2:0:10 (VP1:VP2:VP3).

In certain embodiments, AAV particles are produced in viral production cells (such as mammalian or insect cells) wherein all three VP proteins are expressed at a stoichiometry approaching, about or which is 1-2:0-2:10 (VP1:VP2:VP3).

In certain embodiments, AAV particles are produced in viral production cells (such as mammalian or insect cells) wherein all three VP proteins are expressed at a stoichiometry approaching, about or which is 1-2:1-2:10 (VP1:VP2:VP3).

In certain embodiments, AAV particles are produced in viral production cells (such as mammalian or insect cells) wherein all three VP proteins are expressed at a stoichiometry approaching, about or which is 2-3:0-3:10 (VP1:VP2:VP3).

In certain embodiments, AAV particles are produced in viral production cells (such as mammalian or insect cells) wherein all three VP proteins are expressed at a stoichiometry approaching, about or which is 2-3:2-3:10 (VP1:VP2:VP3).

In certain embodiments, AAV particles are produced in viral production cells (such as mammalian or insect cells) wherein all three VP proteins are expressed at a stoichiometry approaching, about or which is 3:3:10 (VP1:VP2:VP3).

In certain embodiments, AAV particles are produced in viral production cells (such as mammalian or insect cells) wherein all three VP proteins are expressed at a stoichiometry approaching, about or which is 3-5:0-5:10 (VP1:VP2:VP3).

In certain embodiments, AAV particles are produced in viral production cells (such as mammalian or insect cells) wherein all three VP proteins are expressed at a stoichiometry approaching, about or which is 3-5:3-5:10 (VP1:VP2:VP3).

In certain embodiments, the expression control regions are engineered to produce a VP1:VP2:VP3 ratio selected from the group consisting of: about or exactly 1:0:10; about or exactly 1:1:10; about or exactly 2:1:10; about or exactly 2:1:10; about or exactly 2:2:10; about or exactly 3:0:10; about or exactly 3:1:10; about or exactly 3:2:10; about or exactly 3:3:10; about or exactly 4:0:10; about or exactly 4:1:10; about or exactly 4:2:10; about or exactly 4:3:10; about or exactly 4:4:10; about or exactly 5:5:10; about or exactly 1-2:0-2:10; about or exactly 1-2:1-2:10; about or exactly 1-3:0-3:10; about or exactly 1-3:1-3:10; about or exactly 1-4:0-4:10; about or exactly 1-4:1-4:10; about or exactly 1-5:1-5:10; about or exactly 2-3:0-3:10; about or exactly 2-3:2-3:10; about or exactly 2-4:2-4:10; about or exactly 2-5:2-5:10; about or exactly 3-4:3-4:10; about or exactly 3-5:3-5:10; and about or exactly 4-5:4-5:10.

### Transcriptional Regulatory Systems

The present disclosure presents transcriptional regulatory systems which can be used to regulate the expression of a protein-coding nucleotide sequence. The present disclosure presents viral expression constructs which include a transcriptional regulatory system which can be used to regulate the expression of a protein-coding nucleotide sequence. The present disclosure presents expression control regions which include a transcriptional regulatory system which can be used to regulate the expression of a protein-coding nucleotide sequence (e.g., regulatable expression control region).

In certain embodiments, the transcriptional regulatory system is functional in increasing the expression of a protein-coding nucleotide sequence. In certain embodiments, the transcriptional regulatory system is functional in decreasing or silencing the expression of a protein-coding nucleotide sequence. In certain embodiments, the transcriptional regulatory system is functional in increasing, decreasing, or silencing the expression of a nucleotide sequence encoding one or more structural AAV capsid proteins (e.g., VP1, VP2, VP3, or a combination thereof). In certain embodiments, the transcriptional regulatory system is functional in increasing, decreasing, or silencing the expression of a nucleotide sequence encoding one or more non-structural AAV replication proteins (e.g., Rep78, Rep52, or a combination thereof). In certain embodiments, the transcriptional regulatory system is functional in increasing, decreasing or silencing the expression of a nucleotide sequence encoding one or more payload polypeptides.

In certain embodiments, the transcriptional regulatory system includes at least one regulator element and at least one regulator binding region. In certain embodiments, the regulator element can bind to the regulator binding region. In certain embodiments, the regulator element has a high affinity for binding to the regulator binding region. In certain embodiments, the regulator element is an inducible regulator element. In certain embodiments, the transcriptional regulatory system includes at least one regulator element, at least one regulator binding region, and at least one inducer element. In certain embodiments, the inducer element can reduce the affinity of the regulator element for binding to the regulator binding region. In certain embodiments, the regulator element has a high affinity for binding to the regulator binding region when the inducer element is not present or present at low concentrations, and a low affinity for binding to the regulator binding region when the inducer element is present or present at high concentrations. In certain embodiments, the inducer element binds to regulator element and causes a conformational change in the regulator element to reduce binding affinity to the regulator binding region.

In certain embodiments, the regulator element is a Lac repressor (LacR) protein, the regulator binding region is a Lac Operator (LacO) nucleotide sequence, and the inducer element is a LacR inducer element selected from Lactose, Allolactose and isopropyl-β-D-thiogalactose (IPTG). The LacR protein is a homotetrameric protein which binds to one or more Lac Operator (LacO) nucleotide sequences. The tetrameric LacR protein typically binds to two LacO sequences simultaneously (such as one LacO sequence on each side of a promoter) and constrains the promoter (e.g., p10 promoter) into a loop when acting on the LacO sequences. When this happens, transcription initiation of the promoter is reduced or fully repressed. Binding of LacR to LacO can controlled by the presence of an inducer element, such as the sugar allolactose. When allolactose binds to LacR, it causes LacR to conformationally change and to not bind to LacO nucleotide sequences. The synthetic analog of allolactose is isopropyl β-d-1-thiogalactopyranoside (IPTG). In certain embodiments, IPTG is preferred to allolactose because it is not metabolized and thus maintains stable induction of LacR after being added to cell cultures.

In certain embodiments, the regulator element is a Lac repressor (LacR) protein. LacR is typically a 360 amino acid protein with a molecular weight of 38 kDa which is typically encoded by the Lad gene. In certain embodiments, the regulator element is a Lac repressor (LacR) protein encoded by a LacR nucleotide sequence (i.e., Lad gene). In certain embodiments the LacR protein can be wt E. coli LacR from the Lad gene. In certain embodiments the LacR protein is an engineered LacR protein for expression in viral production cells, such as insect cells. Modifications to the Lad gene (and corresponding engineering LacR protein) can include: changing the translation initiation codon to ATG or a Kozak sequence (or modified Kozak sequence) which includes ATG; and the addition of an SV40 nuclear localization signal (NLS) to the N-terminus of LacR. In certain embodiments, the engineered LacR protein is encoded by a sequence which includes an NLS sequence, a linker sequence, and a modified Lad gene which includes a modified Kozak sequence and an ATG start codon. In certain embodiments, the engineered LacR protein is encoded by SEQ ID NO: 6. In certain embodiments, the engineered LacR protein is encoded by nucleotide sequence which has at least 70%, at least 75%, at least 80%, at least 85%, at least 90% or at least 95% identity to SEQ ID NO: 6.

In certain embodiments, the engineered LacR protein is codon optimized. In certain embodiments, the engineered LacR protein is codon optimized for insect cells. In certain embodiments, the engineered LacR protein is codon optimized for Spodoptera frugiperda insect cells. In certain embodiments, the engineered LacR protein is encoded by SEQ ID NO: 7. In certain embodiments, the engineered LacR protein is encoded by nucleotide sequence which has at least 70%, at least 75%, at least 80%, at least 85%, at least 90% or at least 95% identity to SEQ ID NO: 7.

In certain embodiments, the regulator binding region is a Lac Operator (LacO) nucleotide sequence (usually a 35 bp semipalindromic DNA element). In certain embodiments, the inducer element is a LacR inducer element, such as Lactose, Allolactose (intermediate metabolite of lactose), or isopropyl-β-D-thiogalactose (IPTG) (allolactose analogue). In certain embodiments, the LacR inducer element (e.g., IPTG) binds to LacR and causes a conformational change in LacR to reduce binding affinity to LacO.

In certain embodiments, the regulator element is a Tet repressor (TetR) protein or a tetracycline-controlled transactivator protein (tTA) (composed of TetR fused to strong transactivating domain of VP16 from Herpes simplex virus). In certain embodiments, the regulator element is a TetR protein encoded by a TetR nucleotide sequence. In certain embodiments, the regulator element is a tTA fusion protein encoded by a tTA nucleotide sequence. In certain embodiments, the regulator binding region is a Tet Operator (tetO) nucleotide sequence (usually a 19 bp DNA element) or a Tet Response Element (TRE) (which includes a series of two or more (e.g., seven) repeating tetO units). In certain embodiments, the inducer element is a TetR/tTA inducer element, such as tetracycline (Tet) or a tetracycline analog such as doxycycline (Dox). In certain embodiments, the regulator element includes a TetR protein or a tTA fusion protein, the regulator binding region includes at least one tetO nucleotide sequence (such as a TRE region which includes 2-7 repeating tetO units), and the inducer element is a TetR/tTA inducer element selected from tetracycline (Tet) or doxycycline (Dox). In certain embodiments, the TetR/tTA inducer element (e.g., Tet or Dox) binds to the TetR protein or TetR component of the tTA fusion protein, and causes conformational change in the TetR polypeptide to reduce binding affinity to tetO.

In certain embodiments, the transcriptional regulatory system can include one or more components as described in U.S. Pat. No. 6,133,027 (the contents of which are herein incorporated by reference in its entirety as related to transcriptional regulatory systems and components thereof), including specific regulator element, regulator binding regions, and inducer elements.

In certain embodiments, the transcriptional regulatory system includes at least one regulator binding region (e.g., regulator binding sequence) within the expression control region of a viral expression construct. In certain embodiments, the expression control region includes a promoter and at least one regulator binding region. In certain embodiments, the regulator binding region is 5-150 or 5-100 nucleotides from the promoter. In certain embodiments, the regulator binding region is between 5-10, 10-15, 15-20, 20-25, 25-30, 30-35, 35-40, 40-45, 45-50, 50-55, 55-60, 60-65, 65-70, 70-80, 80-90, 90-100, 100-110, 110-120, 120-130, 130-140 or 140-150 nucleotides from the promoter. In certain embodiments, the regulator binding region is placed in a region known to be not essential for promoter function. In certain embodiments, the regulator binding region is a Lac Operator (LacO) nucleotide sequence. In certain embodiments, the Lac Operator (LacO) nucleotide sequence is SEQ ID NO: 8. In certain embodiments, the Lac Operator (LacO) nucleotide sequence is a nucleotide sequence which has at least 75%, at least 80%, at least 85%, at least 90% or at least 95% identity to SEQ ID NO: 8. In certain embodiments, the regulator binding region includes at least one tetO nucleotide sequence (such as a TRE region which includes 2-7 repeating tetO units). In certain embodiments, the promoter is a p10 promoter. In certain embodiments, the promoter is a polh promoter. In certain embodiments, the regulator binding region is a Lac Operator (LacO) nucleotide sequence and the promoter is a p10 promoter.

In certain embodiments, the expression control region includes a promoter and 2-7 regulator binding regions. In certain embodiments, the expression control region includes a promoter and two regulator binding regions. In certain embodiments, the expression control region includes a promoter, and upstream regulator binding region which is upstream of the promoter, and a downstream regulator binding region which is downstream from the promoter. In certain embodiments, the two regulator binding regions have a space interval of 100-300 nucleotides between them (measured from the center nucleotide of each regulator binding region). In certain embodiments, the two regulator binding regions have a space interval of 150-300, 150-250, 150-225, or 150-210 nucleotides between them (measured from the center nucleotide of each regulator binding region). In certain embodiments, the two regulator binding regions have a space interval of 100-105, 105-110, 110-115, 115-120, 120-125, 125-130, 130-135, 135-140, 140-145, 145-150, 150-155, 155-160, 160-165, 165-170, 170-175, 175-180, 180-185, 185-190,190-195, 195-200, 200-205, 200-210, 200-215, 205-210, 205-215, 210-215, 215-220, 220-225, 225-230, 230-235, 235-240, 240-245, 245-250, 250-255, 255-260, 260-265, 265-270, 270-275, 275-280, 280-285, 285-290, 290-295 or 295-300 nucleotides from the promoter. In certain embodiments, the two regulator binding regions have a space interval of 112 nucleotides. In certain embodiments, the two regulator binding regions have a space interval of 148 nucleotides. In certain embodiments, the two regulator binding regions have a space interval of 152 nucleotides. In certain embodiments, the two regulator binding regions have a space interval of 200 nucleotides. In certain embodiments, the two regulator binding regions have a space interval of 208 nucleotides.

In certain embodiments, the transcriptional regulatory system includes a promoter, at least one regulator binding region within 100 nucleotides from the promoter and at least one regulator element that binds to the regulator binding region. In certain embodiments, the regulator element is functional in decreasing transcription from the promoter when the regulator element is bound to the regulator binding region within 100 nucleotides from the promoter. In certain embodiments, the regulator element is functional in silencing transcription from the promoter when the regulator element is bound to the regulator binding region within 100 nucleotides from the promoter. In certain embodiments, the regulator element is functional in decreasing or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter.

In certain embodiments, the expression control region includes a promoter, at least two regulator binding regions (i.e., regulator binding sequences) that are within 100 nucleotides from each end of the promoter region and with a space interval of 200-215 nucleotides (measured from the center nucleotide of each regulator binding sequence), and at least one regulator element that binds to the regulator binding region. In certain embodiments, the regulator element is functional in decreasing transcription from the promoter when the regulator element is bound to the regulator binding region within 100 nucleotides from the promoter. In certain embodiments, the regulator element is functional in silencing transcription from the promoter when the regulator element is bound to the regulator binding region within 100 nucleotides from the promoter. In certain embodiments, the regulator element is functional in decreasing or silencing the expression of a protein-coding nucleotide sequence from the promoter when the regulator element is bound to the regulator binding region within 100 nucleotides from the promoter. In certain embodiments, the regulator element is functional in decreasing or silencing the expression of a protein-coding nucleotide sequence from the promoter by interfering with RNA polymerase activity at the promoter, thereby inhibiting or reducing transcriptional elongation from the promoter.

The present disclosure presents a viral expression construct which includes a nucleotide sequence which encodes a regulator element. In certain embodiments, the viral expression construct includes: (i) a first region or open reading frame (ORF) which includes a protein-coding nucleotide sequence operably linked to an expression control sequence, wherein the expression control sequence includes a promoter and at least one regulator binding region within 100 nucleotides from the promoter; and (ii) a second region or ORF which includes a nucleotide sequence which encodes a regulator element; and wherein the regulator element encoded by the nucleotide sequence in the second region/ORF has a binding affinity for the at least one regulator binding region within the expression control sequence of the first region/ORF. In certain embodiments, the regulator element from the second region/ORF is functional in decreasing or silencing the expression of the protein-coding nucleotide sequence from the promoter in the first region/ORF when the regulator element is bound to the regulator binding region within the expression control sequence of the first region/ORF.

In certain embodiments, the transcriptional regulatory system is operable with a nucleotide sequence encoding one or more structural AAV capsid proteins (e.g., VP1, VP2, VP3, or a combination thereof), such that the concentration of the regulator element present in the transcriptional regulatory system is proportional to amount of the AAV capsid protein material produced by the expression of the protein-coding nucleotide sequence from a promoter. In certain embodiments, the transcriptional regulatory system is operable with a nucleotide sequence encoding VP1 only. In certain embodiments, the transcriptional regulatory system is operable with a nucleotide sequence encoding VP2 only. In certain embodiments, the transcriptional regulatory system is operable with a nucleotide sequence encoding VP3 only. In certain embodiments, a transcriptional regulatory system is engineered to provide a VP protein ratio (VP1:VP2:VP3) of about 1-2:1-2:10 when the viral expression construct is processed by a viral production cell. In certain embodiments, the transcriptional regulatory system is engineered to include a concentration of a regulator element which results in a VP protein ratio (VP1:VP2:VP3) of about 1-2:1-2:10 when the viral expression construct is processed by a viral production cell.

In certain embodiments, the transcriptional regulatory system is operable with a nucleotide sequence encoding one or more non-structural AAV replication proteins (e.g., Rep78, Rep52, or a combination thereof), such that the concentration of the regulator element present in the transcriptional regulatory system is proportional to amount of the AAV replication protein material produced by the expression of the protein-coding nucleotide sequence from a promoter. In certain embodiments, the transcriptional regulatory system is operable with a nucleotide sequence encoding Rep78 only. In certain embodiments, the transcriptional regulatory system is operable with a nucleotide sequence encoding Rep52 only. In certain embodiments, a transcriptional regulatory system is engineered to provide a ratio of p5 Rep proteins (Rep78 and Rep68) to p19 Rep proteins (Rep52 and Rep40) of about 1:1-10 when the viral expression construct is processed by a viral production cell. In certain embodiments, the transcriptional regulatory system is engineered to include a concentration of a regulator element which results in a ratio of p5 Rep proteins (Rep78 and Rep68) to p19 Rep proteins (Rep52 and Rep40) of about 1:1-10 when the viral expression construct is processed by a viral production cell.

In certain embodiments, the transcriptional regulatory system can include one or more regulatable elements presented in WO2016137949 or WO2017075335, the contents of each of which are herein incorporated by reference in their entireties insofar as they do not conflict with the present disclosure.

### Viral Production Cells and Vectors

### Insect Cells

Viral production of the present disclosure disclosed herein describes processes and methods for producing AAV particles or viral vector that contacts a target cell to deliver a payload construct, e.g., a recombinant AAV particle or viral construct, which comprises a nucleotide encoding a payload molecule. The viral production cell may be selected from any biological organism, comprising prokaryotic (e.g., bacterial) cells, and eukaryotic cells, comprising, insect cells, yeast cells and mammalian cells. In certain embodiments, the AAV particles or viral vectors of the present disclosure may be produced in a viral production cell that comprises an insect cell.

Growing conditions for insect cells in culture, and production of heterologous products in insect cells in culture are well-known in the art, see U.S. Pat. No. 6,204,059, the content of which is incorporated herein by reference in its entirety as related to the growth and use of insect cells in viral production.

Any insect cell which allows for replication of parvovirus and which can be maintained in culture can be used in accordance with the present disclosure. AAV viral production cells commonly used for production of recombinant AAV particles comprise, but is not limited to, Spodoptera frugiperda, comprising, but not limited to the Sf9 or Sf21 cell lines, Drosophila cell lines, or mosquito cell lines, such as Aedes albopictus derived cell lines. Use of insect cells for expression of heterologous proteins is well documented, as are methods of introducing nucleic acids, such as vectors, e.g., insect-cell compatible vectors, into such cells and methods of maintaining such cells in culture. See, for example, Methods in Molecular Biology, ed. Richard, Humana Press, N J (1995); O'Reilly et al., Baculovirus Expression Vectors, A Laboratory Manual, Oxford Univ. Press (1994); Samulski et al., J. Vir. 63:3822-8 (1989); Kajigaya et al., Proc. Nat'l. Acad. Sci. USA 88: 4646-50 (1991); Ruffing et al., J. Vir. 66:6922-30 (1992); Kimbauer et al., Vir. 219:37-44 (1996); Zhao et al., Vir. 272:382-93 (2000); and Samulski et al., U.S. Pat. No. 6,204,059, the contents of which are each incorporated herein by reference in their entireties as related to the use of insect cells in viral production, insofar as they do not conflict with the present disclosure.

In one embodiment, the AAV particles are made using the methods described in WO2015/191508, the content of which is incorporated herein by reference in its entirety.

In certain embodiments, insect host cell systems, in combination with baculoviral systems (e.g., as described by Luckow et al., Bio/Technology 6: 47 (1988)) may be used. In certain embodiments, an expression system for preparing chimeric peptide is Trichoplusia ni, Tn 5B1-4 insect cells/baculoviral system, which can be used for high levels of proteins, as described in U.S. Pat. No. 6,660,521, the content of which is incorporated herein by reference in its entirety.

Expansion, culturing, transfection, infection and storage of insect cells can be carried out in any cell culture media, cell transfection media or storage media known in the art, including Hyclone SFX Insect Cell Culture Media, Expression System ESF AF Insect Cell Culture Medium, ThermoFisher Sf90011 media, ThermoFisher Sf900111 media, or ThermoFisher Grace's Insect Media. Insect cell mixtures of the present disclosure can also include any of the formulation additives or elements described in the present disclosure, including (but not limited to) salts, acids, bases, buffers, surfactants (such as Poloxamer 188/Pluronic F-68), and other known culture media elements. Formulation additives can be incorporated gradually or as “spikes” (incorporation of large volumes in a short time).

### Baculovirus Production Systems

In certain embodiments, processes of the present disclosure can comprise production of AAV particles or viral vectors in a baculoviral system using a viral, e.g., AAV, expression construct and a payload construct vector (e.g., an AAV payload expression construct). In certain embodiments, the baculoviral system comprises Baculovirus expression vectors (BEVs) and/or baculovirus infected insect cells (BIICs). In certain embodiments, a viral expression construct, e.g., an AAV expression construct, or a payload construct, e.g., an AAV payload construct, of the present disclosure can be a bacmid, also known as a baculovirus plasmid or recombinant baculovirus genome. In certain embodiments, a VP-coding region encoding an AAV capsid protein (e.g., a VP1 protein, a VP2 protein, and/or a VP3 protein), a Rep-coding region encoding an AAV rep protein (e.g., a Rep52 protein, a Rep40 protein, a Rep68 protein, a Rep78 protein, or a combination thereof), and/or a payload coding region (e.g., encoding a payload described herein) incorporated into a bacmid by molecular biology techniques (e.g., transposon donor/acceptor system or Gibson Assembly) to generate an AAV expression construct described herein. Transfection of separate viral replication cell populations produces two or more groups (e.g., two, three) of AAV expression constructs, and one or more group which can comprise the payload construct (e.g., the baculovirus is a “Payload BEV” or “payloadBac”). The AAV expression construct described herein comprising a baculovirus genome, e.g., a variant baculovirus genome, may be used to for production of AAV particles or viral vector in a cell, e.g., a viral production cell.

In certain embodiments, the process comprises transfection of a single viral replication cell population to produce a single baculovirus (BEV) group which comprises both the viral expression construct and the payload construct. These baculoviruses may be used to infect a viral production cell for production of AAV particles or viral vector.

In certain embodiments, BEVs are produced using a Bacmid Transfection agent, such as Promega FuGENE HD, WFI water, or ThermoFisher Cellfectin II Reagent. In certain embodiments, BEVs are produced and expanded in viral production cells, such as an insect cell.

In certain embodiments, the method utilizes seed cultures of viral production cells that comprise one or more BEVs, comprising baculovirus infected insect cells (BIICs). The seed BIICs have been transfected/transduced/infected with an Expression BEV which comprises a viral expression construct, and also a Payload BEV which comprises a payload construct. In certain embodiments, the seed cultures are harvested, divided into aliquots and frozen, and may be used at a later time to initiate transfection/transduction/infection of a naïve population of production cells. In certain embodiments, a bank of seed BIICs is stored at −80° C. or in LN₂ vapor.

Baculoviruses are made of several essential proteins which are essential for the function and replication of the Baculovirus, such as replication proteins, envelope proteins and capsid proteins. The Baculovirus genome thus comprises several essential-gene nucleotide sequences encoding the essential proteins. As a non-limiting example, the genome can comprise an essential-gene region which comprises an essential-gene nucleotide sequence encoding an essential protein for the Baculovirus construct. The essential protein can comprise: GP64 baculovirus envelope protein, VP39 baculovirus capsid protein, or other similar essential proteins for the Baculovirus construct.

Baculovirus expression vectors (BEV) for producing AAV particles in insect cells, comprising but not limited to Spodoptera frugiperda (Sf9) cells, provide high titers of viral vector product. Recombinant baculovirus encoding the viral expression construct and payload construct initiates a productive infection of viral vector replicating cells. Infectious baculovirus particles released from the primary infection secondarily infect additional cells in the culture, exponentially infecting the entire cell culture population in a number of infection cycles that is a function of the initial multiplicity of infection, see Urabe, M. et al. J Virol. 2006 February; 80(4):1874-85, the content of which is incorporated herein by reference in its entirety as related to the production and use of BEVs and viral particles.

In certain embodiments, the production system of the present disclosure addresses baculovirus instability over multiple passages by utilizing a titerless infected-cells preservation and scale-up system. Small scale seed cultures of viral producing cells are transfected with viral expression constructs encoding the structural and/or non-structural components of the AAV particles. Baculovirus-infected viral producing cells are harvested into aliquots that may be cryopreserved in liquid nitrogen; the aliquots retain viability and infectivity for infection of large scale viral producing cell culture Wasilko D J et al. Protein Expr Purif. 2009 June; 65(2):122-32, the content of which is incorporated herein by reference in its entirety as related to the production and use of BEVs and viral particles.

A genetically stable baculovirus may be used to produce a source of the one or more of the components for producing AAV particles in invertebrate cells. In certain embodiments, defective baculovirus expression vectors may be maintained episomally in insect cells. In such an embodiment the corresponding bacmid vector is engineered with replication control elements, comprising but not limited to promoters, enhancers, and/or cell-cycle regulated replication elements.

In certain embodiments, a baculovirus, may be engineered with a marker for recombination into the chitinase/cathepsin locus. The chia/v-cath locus is non-essential for propagating baculovirus in tissue culture, and the V-cath (EC 3.4.22.50) is a cysteine endoprotease that is most active on Arg-Arg dipeptide containing substrates. The Arg-Arg dipeptide is present in densovirus and parvovirus capsid structural proteins but infrequently occurs in dependovirus VP1.

In certain embodiments, stable viral producing cells permissive for baculovirus infection are engineered with at least one stable integrated copy of any of the elements necessary for AAV replication and vector production comprising, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In certain embodiments, the AAV expression construct, e.g., the Baculovirus expression vectors (BEV), can be based on the Autographa californica multicapsid nucleopolyhedrosis virus (AcMNPV baculovirus, e.g., strain E2) or BmNPV baculovirus. In certain embodiments, a bacmid of the present disclosure is based on (e.g., engineered variant of) an AcMNPV bacmid such as bmon14272, vAce25ko or vAclef11KO.

In certain embodiments, the AAV expression construct, e.g., the BEV, comprises a disruption, e.g., alteration, in a v-cath proteinase gene, wherein the v-cath gene has been interrupted, mutated, partially deleted, or fully deleted (“v-cath modified BEV”). In certain embodiments, the BEVs lack the v-cath gene or comprise a mutationally inactivated version of the v-cath gene (“v-cath inactivated BEV”). In certain embodiments, the BEVs lack the v-cath gene. In certain embodiments, the BEVs comprise a mutationally inactivated version of the v-cath gene. In certain embodiments, the Baculovirus expression vectors (BEV) is a BEV in which the baculoviral chiA chitinase gene has been mutated, partially deleted, or fully deleted (“chiA modified BEV”). In certain embodiments, the BEVs lack the chiA gene or comprise a mutationally inactivated version of the chiA gene (“chiA inactivated BEV”). In certain embodiments, the BEVs lack the chiA gene. In certain embodiments, the BEVs comprise a mutationally inactivated version of the chiA gene. In certain embodiments, the Baculovirus expression vectors (BEV) is a BEV in which the baculoviral v-cath proteinase gene and/or the baculoviral chiA chitinase gene have been mutated, partially deleted, or fully deleted (“v-cath modified BEV”). In certain embodiments, the v-cath and/or chiA genes are mutated/deleted by homologous recombination. In certain embodiments, the v-cath and/or chiA genes are mutated/deleted by homologous recombination with regions mapping to the chiA C terminus and gp64 C terminus derived from AcMNPV strain C6 (rather than parental strain E2). In certain embodiments, the v-cath and/or chiA genes are mutated/deleted by homologous recombination, which results in several point mutations relative to strain E2 (i.e., in the vestigial chiA C-terminus). In certain embodiments, the v-cath and/or chiA genes are mutated/deleted by replacement with a 26-bp recognition site of homing endonuclease I-CeuI. In certain embodiments, the chiA gene is mutated/deleted such that a portion of the chiA C terminus is left to retain the promoter region of essential baculovirus gene lef7.

In certain embodiments, the v-cath and/or chiA genes are mutated/deleted by replacement with an AscI-flanked LacZa cassette (e.g., AscI-flanked codon-optimized LacZa cassette). In certain embodiments, the AscI-flanked LacZa cassette is inserted functionally downstream from a p10 promoter in the v-cath locus. In certain embodiments, the AscI-flanked LacZa cassette allows for blue/white colony phenotyping in colony screening steps. In certain embodiments, the AscI-flanked LacZa cassette can be digested with AscI, thereby resulting in DNA ends which are compatible with Gibson assembly of PacI-excised sequence inserts (e.g., PacI-excised transgene inserts from transgene plasmid constructs, or VP1/VP2/VP3 expression constructs).

In certain embodiments, the AAV expression construct, e.g., the BEV, can comprise a VP-coding region with a VP-coding sequence within the v-cath locus of the BEV. In certain embodiments, the BEV can comprise a VP-coding region in the v-cath locus which comprises a VP nucleotide sequence encoding VP1, VP2, VP3, or a combination thereof.

In certain embodiments, the Baculovirus expression vectors (BEV) can comprise a Rep-coding region with a Rep-coding sequence within the v-cath locus of the BEV. In certain embodiments, the BEV can comprise a Rep-coding region in the v-cath locus which comprises a Rep nucleotide sequence encoding Rep78, Rep52, or a combination thereof.

In certain embodiments, the AAV expression construct, e.g., the BEV), can comprise a VP-coding region with a VP-coding sequence within the v-cath locus of the BEV, and a Rep-coding region with a Rep-coding sequence within the v-cath locus of the BEV. In certain embodiments, the BEV can comprise a VP-coding region in the v-cath locus which comprises a VP nucleotide sequence encoding VP1, VP2, VP3, or a combination thereof, and a Rep-coding region in the v-cath locus which comprises a Rep-coding sequence encoding Rep78, Rep52, or a combination thereof.

In certain embodiments, the AAV expression construct, e.g., the BEV, can comprise: (1) a VP-coding region in the v-cath locus which comprises a VP nucleotide sequence encoding VP1, VP2, VP3, or a combination thereof; and (ii) a Rep-coding region in the v-cath locus which comprises a Rep-coding sequence encoding Rep78 or Rep52. In certain embodiments, the BEV can comprise: (1) a VP-coding region in the v-cath locus which comprises a VP nucleotide sequence encoding VP1, VP2, VP3, or a combination thereof; and (ii) a Rep-coding region in the v-cath locus which comprises a Rep-coding sequence encoding Rep78. In certain embodiments, the BEV can comprise: (1) a VP-coding region in the v-cath locus which comprises a VP nucleotide sequence encoding VP1, VP2, VP3, or a combination thereof; and (ii) a Rep-coding region in the v-cath locus which comprises a Rep-coding sequence encoding Rep52. In certain embodiments, the BEV can comprise: (1) a VP-coding region in the v-cath locus which comprises a VP nucleotide sequence encoding VP1, VP2, VP3, or a combination thereof; (ii) a Rep-coding region in the v-cath locus which comprises a Rep-coding sequence encoding Rep78; and (iii) a Rep-coding region in different locus (e.g., egt locus) which comprises a Rep-coding sequence encoding Rep52. In certain embodiments, the BEV can comprise: (1) a VP-coding region in the v-cath locus which comprises a VP nucleotide sequence encoding VP1, VP2, VP3, or a combination thereof; (ii) a Rep-coding region in the v-cath locus which comprises a Rep-coding sequence encoding Rep52; and (iii) a Rep-coding region in different locus (e.g., egt locus) which comprises a Rep-coding sequence encoding Rep78.

In certain embodiments, the AAV expression, e.g., the BEV, is a BEV in which baculovirus gene p26 is deleted or mutationally inactivated. In certain embodiments, the Baculovirus expression vectors (BEV) is a BEV in which baculovirus gene p10 is deleted or mutationally inactivated. In certain embodiments, the Baculovirus expression vectors (BEV) is a BEV in which baculovirus gene p74 is deleted or mutationally inactivated. In certain embodiments, the Baculovirus expression vectors (BEV) is a BEV in which baculovirus genes p26, p10, and/or p74 are deleted or mutationally inactivated. See, e.g., Hitchman et al, Cell biology and toxicology 26.1 (2010): 57-68; which is incorporated herein by reference in its entirety as related to the deletion, replacement, and/or mutational inactivation of p26, p10, and/or p74 genes in a baculovirus vector. In certain embodiments, the Baculovirus expression vectors (BEV) is a BEV in which baculovirus genes p26, p10, and/or p74 are deleted and replaced with an I-SceI-flanked chloramphenicol-resistance cassette. In certain embodiments, the chloramphenicol-resistance cassette is removed to provide a single I-SceI cut site.

In certain embodiments, the AAV expression, e.g., the BEV, comprises an AscI-flanked LacZa cassette (e.g., AscI-flanked codon-optimized LacZa cassette between the kanamycin resistance cassette and the mini-F replicon (e.g., polyhedrin locus) of the baculovirus vector (e.g., by replacing the native LacZa cassette, such as the native LacZa cassette in bMON14272). In certain embodiments, the AscI-flanked LacZa cassette allows for blue/white colony phenotyping in colony screening steps. In certain embodiments, the AscI-flanked LacZa cassette can be digested with AscI, thereby resulting in DNA ends which are compatible with Gibson assembly of PacI-excised sequence inserts (e.g., PacI-excised transgene inserts from transgene plasmid constructs). In certain embodiments, the AscI-flanked LacZa cassette is removed from the polyhedrin locus, and replaced with a single SrfI cut site.

In certain embodiments, the AAV expression construct, e.g., the BEV, is a BEV in which the SrfI site located in the ccdB ORF of the bacterial mini-F replicon is silently mutated (e.g., no amino acid change). In certain embodiments, the Baculovirus expression vectors (BEV) is a BEV in which AscI sites in the ac-arif-1 and ac-pkip-1 genes are silently mutated (e.g., no amino acid changes).

Viral production bacmids of the present disclosure can comprise deletion of certain baculoviral genes or loci.

The present disclosure presents methods for producing a baculovirus infected insect cell (BIIC), e.g., expression BIICs and/or payload BIICs. In certain embodiments, the present disclosure presents methods for producing a baculovirus infected insect cell (BIIC) which comprises the following steps: (a) introducing a volume of cell culture medium into a bioreactor; (b) introducing at least one viral production cell (VPC) into the bioreactor and expanding the number of VPCs in the bioreactor to a target VPC cell density; (c) introduction at least one Baculoviral Expression Vector (BEV) into the bioreactor, wherein the BEV comprises an AAV viral expression construct or an AAV payload construct; (d) incubating the mixture of VPCs and BEVs in the bioreactor under conditions which allow at least one BEV to infect at least one VPC to produce a baculovirus infected insect cell (BIIC); (e) incubating the bioreactor under conditions which allow the number of BIICs in the bioreactor to reach a target BIIC cell density; and (f) harvesting the BIICs from the bioreactor. In certain embodiments, the bioreactor has a volume of at least 5 L, 10 L, 20 L, 50 L, 100 L, or 200 L. In certain embodiments, the volume of cell culture medium (i.e., working volume) in the bioreactor is at least 5 L, 10 L, 20 L, 50 L, 100 L, or 200 L.

In certain embodiments, the VPC density at BEV introduction is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at BEV introduction is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the target VPC cell density at BEV introduction is 1.5-4.0×10⁶ cells/mL. In certain embodiments, the target VPC cell density at BEV introduction is 2.0-3.5×10⁶ cells/mL.

In certain embodiments, the BEVs are introduced into the bioreactor at a target Multiplicity of Infection (MOI) of BEVs to VPCs. In certain embodiments, the BEV MOI is 0.0005-0.003, or more specifically 0.001-0.002.

In certain embodiments, the BIICs are harvested from the bioreactor at a specific BIIC cell density. In certain embodiments, the BIICs harvested from the bioreactor have a specific BIIC cell density. In certain embodiments, the BIIC cell density at harvesting is 6.0-18.0×10⁶ cells/mL, 8.0-16.5×10⁶ cells/mL, 10.0-16.5×10⁶ cells/mL.

In certain embodiments, BIICs (expression BIICs, payload BIICs) are used to transfect viral production cells, e.g., Sf9 cells. In some embodiments, baculoviruses comprising bacmids such as BEVs are used to transfect viral production cells, e.g., Sf9 cells.

### Other

In certain embodiments expression hosts comprise, but are not limited to, bacterial species within the genera Escherichia, Bacillus, Pseudomonas, or Salmonella.

In certain embodiments, a host cell which comprises AAV rep and cap genes stably integrated within the cell's chromosomes, may be used for AAV particle production. In a non-limiting example, a host cell which has stably integrated in its chromosome at least two copies of an AAV rep gene and AAV cap gene may be used to produce the AAV particle according to the methods and constructs described in U.S. Pat. No. 7,238,526, the content of which is incorporated herein by reference in its entirety as related to the production of viral particles.

In certain embodiments, the AAV particle can be produced in a host cell stably transformed with a molecule comprising the nucleic acid sequences which permit the regulated expression of a rare restriction enzyme in the host cell, as described in US20030092161 and EP1183380, the contents of which are each incorporated herein by reference in their entireties as related to the production of viral particles, insofar as they do not conflict with the present disclosure.

In certain embodiments, production methods and cell lines to produce the AAV particle may comprise, but are not limited to those taught in PCT/US1996/010245, PCT/US1997/015716, PCT/US1997/015691, PCT/US1998/019479, PCT/US1998/019463, PCT/US2000/000415, PCT/US2000/040872, PCT/US2004/016614, PCT/US2007/010055, PCT/US1999/005870, PCT/US2000/004755, U.S. patent application Ser. Nos. 08/549,489, 08/462,014, 09/659,203, 10/246,447, 10/465,302, U.S. Pat. Nos. 6,281,010, 6,270,996, 6,261,551, 5,756,283, 6,428,988, 6,274,354, 6,943,019, 6,482,634, (Assigned to NIH: U.S. Pat. Nos. 7,238,526, 6,475,769), U.S. Pat. No. 6,365,394 (Assigned to NIH), U.S. Pat. Nos. 7,491,508, 7,291,498, 7,022,519, 6,485,966, 6,953,690, 6,258,595, EP2018421, EP1064393, EP1163354, EP835321, EP931158, EP950111, EP1015619, EP1183380, EP2018421, EP1226264, EP1636370, EP1163354, EP1064393, US20030032613, US20020102714, US20030073232, US20030040101 (Assigned to NIH), US20060003451, US20020090717, US20030092161, US20070231303, US20060211115, US20090275107, US2007004042, US20030119191, US20020019050, the contents of which are each incorporated herein by reference in their entireties, insofar as they do not conflict with the present disclosure.

### Viral Production Systems

### Large-Scale Production

In certain embodiments, AAV particle production may be modified to increase the scale of production. Large scale viral production methods according to the present disclosure may comprise any of the processes or processing steps taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference by reference in their entirety.

Methods of increasing AAV particle production scale typically comprise increasing the number of viral production cells. In certain embodiments, viral production cells comprise adherent cells. To increase the scale of AAV particle production by adherent viral production cells, larger cell culture surfaces are required. In certain embodiments, large-scale production methods comprise the use of roller bottles to increase cell culture surfaces. Other cell culture substrates with increased surface areas are known in the art. Examples of additional adherent cell culture products with increased surface areas comprise, but are not limited to iCELLis (Pall Corp, Port Washington, NY), CELLSTACK®, CELLCUBE® (Corning Corp., Corning, NY) and NUNC™ CELL FACTORY™ (Thermo Scientific, Waltham, MA.) In certain embodiments, large-scale adherent cell surfaces may comprise from about 1,000 cm² to about 100,000 cm².

In certain embodiments, large-scale viral production methods of the present disclosure may comprise the use of suspension cell cultures. Suspension cell culture can allow for significantly increased numbers of cells. Typically, the number of adherent cells that can be grown on about 10-50 cm² of surface area can be grown in about 1 cm³ volume in suspension.

In certain embodiments, large-scale cell cultures may comprise from about 1.0×10⁷ to about 9.9×10⁹ cells, from about 1.0×10⁸ to about 9.9×10¹⁰ cells, from about 1.0×10⁹ to about 9.9×10¹¹ cells, from about 1.0×10¹⁰ to about 9.9×10¹² cells, from about 1.0×10¹¹ to about 9.9×10¹³ cells, from about 1.0×10¹² to about 9.9×10¹⁴ cells, from about 1.0×10¹³ to about 9.9×10¹⁵ cells, from about 1.0×10¹⁴ to about 9.9×10¹⁶ cells, from about 1.0×10¹⁵ to about 9.9×10¹⁷ cells, or from about 1.0×10¹⁶ to about 9.9×10¹⁸ cells. In certain embodiments, large-scale cell cultures may comprise at least 1.0×10¹² AAV particles. In certain embodiments, large-scale cell cultures may comprise at least 1.0×10¹³ AAV particles. In certain embodiments, large-scale cell cultures may comprise at least 1.0×10¹⁴ AAV particles. In certain embodiments, large-scale cell cultures may comprise at least 1.0×10¹⁵ AAV particles. In certain embodiments, large-scale cell cultures may comprise at least 1.0×10¹⁶ AAV particles. In certain embodiments, large-scale cell cultures may comprise at least 1.0×10¹⁷ AAV particles. In certain embodiments, large-scale cell cultures may comprise at least 1.0×10¹⁸ AAV particles.

Transfection of replication cells in large-scale culture formats may be carried out according to any methods known in the art. For large-scale adherent cell cultures, transfection methods may comprise, but are not limited to the use of inorganic compounds (e.g., calcium phosphate,) organic compounds (e.g., polyethyleneimine (PEI)) or the use of non-chemical methods (e.g., electroporation). With cells grown in suspension, transfection methods may comprise, but are not limited to the use of inorganic compounds (e.g., calcium phosphate,) organic compounds (e.g., polyethyleneimine (PEI)) or the use of non-chemical methods (e.g., electroporation). In certain embodiments, transfection of large-scale suspension cultures may be carried out according to the section entitled “Transfection Procedure” described in Feng, L. et al., 2008. Biotechnol Appl Biochem. 50:121-32, the contents of which are herein incorporated by reference in their entirety. According to such embodiments, PEI-DNA complexes may be formed for introduction of plasmids to be transfected. In certain embodiments, cells being transfected with PEI-DNA complexes may be ‘shocked’ prior to transfection. This comprises lowering cell culture temperatures to 4° C. for a period of about 1 hour. In certain embodiments, cell cultures may be shocked for a period of from about 10 minutes to about 5 hours. In certain embodiments, cell cultures may be shocked at a temperature of from about 0° C. to about 20° C.

In certain embodiments, transfections may comprise one or more vectors for expression of an RNA effector molecule to reduce expression of nucleic acids from one or more payload construct. Such methods may enhance the production of AAV particles by reducing cellular resources wasted on expressing payload constructs. In certain embodiments, such methods may be carried according to those taught in US Publication No. US2014/0099666, the contents of which are herein incorporated by reference in their entirety.

### Bioreactors

In certain embodiments, suspension cell culture bioreactors may be used for large scale production of AAV particles. In certain embodiments, bioreactors comprise stirred tank reactors. Such reactors generally comprise a vessel, typically cylindrical in shape, with a stirrer (e.g., impeller.) In certain embodiments, such bioreactor vessels may be placed within a water jacket to control vessel temperature and/or to minimize effects from ambient temperature changes.

Bioreactor vessel volume may range in size from about 500 ml to about 2 L, from about 2 L to about 5 L, from about 5 L to about 20 L, from about 20 L to about 50 L, from about 50 L to about 100 L, from about 100 L to about 500 L, from about 500 L to about 2,000 L, from about 2,000 L to about 10,000 L, from about 10,000 L to about 20,000 L, from about 20,000 L to about 50,000 L, or more than 50,000 L. Vessel bottoms may be rounded or flat. In certain embodiments, animal cell cultures may be maintained in bioreactors with rounded vessel bottoms.

In certain embodiments, bioreactor vessels may be warmed through the use of a thermocirculator. Thermocirculators pump heated water around water jackets. In certain embodiments, heated water may be pumped through pipes (e.g., coiled pipes) that are present within bioreactor vessels. In certain embodiments, warm air may be circulated around bioreactors, comprising, but not limited to air space directly above culture medium. Additionally, pH and CO₂ levels may be maintained to optimize cell viability.

In certain embodiments, bioreactors may comprise hollow-fiber reactors. Hollow-fiber bioreactors may support the culture of both anchorage dependent and anchorage independent cells. Further bioreactors may comprise, but are not limited to, packed-bed or fixed-bed bioreactors. Such bioreactors may comprise vessels with glass beads for adherent cell attachment. Further packed-bed reactors may comprise ceramic beads.

In certain embodiments, viral particles are produced through the use of a disposable bioreactor. In certain embodiments, bioreactors may comprise GE WAVE bioreactor, a GE Xcellerax Bioreactor, a Sartorius Biostat Bioreactor, a ThermoFisher Hyclone Bioreactor, or a Pall Allegro Bioreactor.

In certain embodiments, AAV particle production in cell bioreactor cultures may be carried out according to the methods or systems taught in U.S. Pat. Nos. 5,064,764, 6,194,191, 6,566,118, 8,137,948 or US Patent Application No. US2011/0229971, the contents of each of which are herein incorporated by reference in their entirety.

### Expansion of Viral Production Cell (VPC) Mixtures

In certain embodiments, an AAV particle or viral vector of the present disclosure may be produced in a viral production cell (VPC), such as an insect cell. Production cells can be sourced from a Cell Bank (CB) and are often stored in frozen cell banks.

In certain embodiments, a viral production cell from a Cell Bank is provided in frozen form. The vial of frozen cells is thawed, typically until ice crystal dissipate. In certain embodiments, the frozen cells are thawed at a temperature between 10-50° C., 15-40° C., 20-30° C., 25-50° C., 30-45° C., 35-40° C., or 37-39° C. In certain embodiments, the frozen viral production cells are thawed using a heated water bath.

In certain embodiments, a thawed CB cell mixture will have a cell density of 1.0×10⁴-1.0×10⁹ cells/mL. In certain embodiments, the thawed CB cell mixture has a cell density of 1.0×10⁴-2.5×10⁴ cells/mL, 2.5×10⁴-5.0×10⁴ cells/mL, 5.0×10⁴-7.5×10⁴ cells/mL, 7.5×10⁴-1.0×10⁵ cells/mL, 1.0×10⁵-2.5×10⁵ cells/mL, 2.5×10⁵-5.0×10⁵ cells/mL, 5.0×10⁵-7.5×10⁵ cells/mL, 7.5×10⁵-1.0×10⁶ cells/mL, 1.0×10⁶-2.5×10⁶ cells/mL, 2.5×10⁶-5.0×10⁶ cells/mL, 5.0×10⁶-7.5×10⁶ cells/mL, 7.5×10⁶-1.0×10⁷ cells/mL, 1.0×10⁷-2.5×10⁷ cells/mL, 2.5×10⁷-5.0×10⁷ cells/mL, 5.0×10⁷-7.5×10⁷ cells/mL, 7.5×10⁷-1.0×10⁸ cells/mL, 1.0×10⁸-2.5×10⁸ cells/mL, 2.5×10⁸-5.0×10⁸ cells/mL, 5.0×10⁸-7.5×10⁸ cells/mL, or 7.5×10⁸-1.0×10⁹ cells/mL.

In certain embodiments, the volume of the CB cell mixture is expanded. This process is commonly referred to as a Seed Train, Seed Expansion, or CB Cellular Expansion. Cellular/Seed expansion can comprise successive steps of seeding and expanding a cell mixture through multiple expansion steps using successively larger working volumes. In certain embodiments, cellular expansion can comprise one, two, three, four, five, six, seven, or more than seven expansion steps. In certain embodiments, the working volume in the cellular expansion can comprise one or more of the following working volumes or working volume ranges: 5 mL, 10 mL, 20 mL, 5-20 mL, 25 mL, 30 mL, 40 mL, 50 mL, 20-50 mL, 75 mL, 100 mL, 125 mL, 150 mL, 175 mL, 200 mL, 50-200 mL, 250 mL, 300 mL, 400 mL, 500 mL, 750 mL, 1000 mL, 250-1000 mL, 1250 mL, 1500 mL, 1750 mL, 2000 mL, 1000-2000 mL, 2250 mL, 2500 mL, 2750 mL, 3000 mL, 2000-3000 mL, 3500 mL, 4000 mL, 4500 mL, 5000 mL, 3000-5000 mL, 5.5 L, 6.0 L, 7.0 L, 8.0 L, 9.0 L, 10.0 L, and 5.0-10.0 L.

In certain embodiments, a volume of cells from a first expanded cell mixture can be used to seed a second, separate Seed Train/Seed Expansion (instead of using thawed CB cell mixture). This process is commonly referred to as rolling inoculum. In certain embodiments, rolling inoculum is used in a series of two or more (e.g., two, three, four or five) separate Seed Trains/Seed Expansions.

In certain embodiments, large-volume cellular expansion can comprise the use of a bioreactor, such as a GE WAVE bioreactor, a GE Xcellerex Bioreactor, a Sartorius Biostat Bioreactor, a ThermoFisher Hyclone Bioreactor, or a Pall Allegro Bioreactor.

In certain embodiments, the cell density within a working volume is expanded to a target output cell density. In certain embodiments, the output cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, the output cell density of a working volume provides a seeding cell density for a larger, successive working volume. In certain embodiments, the seeding cell density of an expansion step is 1.0×10⁵-5.0×10⁵, 5.0×10⁵-1.0×10⁶, 1.0×10⁶-5.0×10⁶, 5.0×10⁶-1.0×10⁷, 1.0×10⁷-5.0×10⁷, 5.0×10⁷-1.0×10⁸, 5.0×10⁵, 6.0×10⁵, 7.0×10⁵, 8.0×10⁵, 9.0×10⁵, 1.0×10⁶, 2.0×10⁶, 3.0×10⁶, 4.0×10⁶, 5.0×10⁶, 6.0×10⁶, 7.0×10⁶, 8.0×10⁶, 9.0×10⁶, 1.0×10⁷, 2.0×10⁷, 3.0×10⁷, 4.0×10⁷, 5.0×10⁷, 6.0×10⁷, 7.0×10⁷, 8.0×10⁷, or 9.0×10⁷ cells/mL.

In certain embodiments, cellular expansion can last for 1-50 days. Each cellular expansion step or the total cellular expansion can last for 1-10 days, 1-5 days, 1-3 days, 2-3 days, 2-4 days, 2-5 days, 2-6 days, 3-4 days, 3-5 days, 3-6 days, 3-8 days, 4-5 days, 4-6 days, 4-8 days, 5-6 days, or 5-8 days. In certain embodiments, each cellular expansion step or the total cellular expansion can last for 1-100 generations, 1-1000 generations, 100-1000 generation, 100 generations or more, or 1000 generation or more.

In certain embodiments, infected or transfected production cells can be expanded in the same manner as CB cell mixtures, as set forth in the present disclosure.

### Infection of Viral Production Cells

In certain embodiments, AAV particles of the present disclosure are produced in a viral production cell (VPC), such as an insect cell, by infecting the VPC with a viral vector which comprises an AAV expression construct and/or a viral vector which comprises an AAV payload construct. In certain embodiments, the VPC is infected with an Expression BEV, which comprises an AAV expression construct and a Payload BEV which comprises an AAV payload construct.

In certain embodiments, AAV particles are produced by infecting a VPC with a viral vector which comprises both an AAV expression construct and an AAV payload construct. In certain embodiments, the VPC is infected with a single BEV which comprises both an AAV expression construct and an AAV payload construct.

In certain embodiments, VPCs (such as insect cells) are infected using Infection BIICs in an infection process which comprises the following steps: (i) A collection of VPCs are seeded into a Production Bioreactor; (ii) The seeded VPCs can optionally be expanded to a target working volume and cell density; (iii) Infection BIICs which comprise Expression BEVs and Infection BIICs which comprise Payload BEVs are injected into the Production Bioreactor, resulting in infected viral production cells; and (iv) incubation of the infected viral production cells to produce AAV particles within the viral production cells.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, the VPC density at infection is 1.0×10⁵-2.5×10⁵, 2.5×10⁵-5.0×10⁵, 5.0×10⁵-7.5×10⁵, 7.5×10⁵-1.0×10⁶, 1.0×10⁶-5.0×10⁶, 1.0×10⁶-2.0×10⁶, 1.5×10⁶-2.5×10⁶, 2.0×10⁶-3.0×10⁶, 2.5×10⁶-3.5×10⁶, 3.0×10⁶-3.4×10⁶, 3.0×10⁶-4.0×10⁶, 3.5×10⁶-4.5×10⁶, 4.0×10⁶-5.0×10⁶, 4.5×10⁶-5.5×10⁶, 5.0×10⁶-1.0×10⁷, 5.0×10⁶-6.0×10⁶, 5.5×10⁶-6.5×10⁶, 6.0×10⁶-7.0×10⁶, 6.5×10⁶-7.5×10⁶, 7.0×10⁶-8.0×10⁶, 7.5×10⁶-8.5×10⁶, 8.0×10⁶-9.0×10⁶, 8.5×10⁶-9.5×10⁶, 9.0×10⁶-1.0×10⁷, 9.5×10⁶-1.5×10⁷, 1.0×10⁷-5.0×10⁷, or 5.0×10⁷-1.0×10⁸ cells/mL. In certain embodiments, the VPC density at infection is 5.0×10⁵, 6.0×10⁵, 7.0×10⁵, 8.0×10⁵, 9.0×10⁵, 1.0×10⁶, 1.5×10⁶, 2.0×10⁶, 2.5×10⁶, 3.0×10⁶, 3.1×10⁶, 3.2×10⁶, 3.3×10⁶, 3.4×10⁶, 3.5×10⁶, 4.0×10⁶, 4.5×10⁶, 5.0×10⁶, 5.5×10⁶, 6.0×10⁶, 6.5×10⁶, 7.0×10⁶, 7.5×10⁶, 8.0×10⁶, 8.5×10⁶, 9.0×10⁶, 9.5×10⁶, 1.0×10⁷, 1.5×10⁷, 2.0×10⁷, 2.5×10⁷, 3.0×10⁷, 4.0×10⁷, 5.0×10⁷, 6.0×10⁷, 7.0×10⁷, 8.0×10⁷, or 9.0×10⁷ cells/mL. In certain embodiments, the VPC density at infection is 2.0-3.5×10⁶ cells/mL. In certain embodiments, the VPC density at infection is 3.5-5.0×10⁶ cells/mL. In certain embodiments, the VPC density at infection is 5.0-7.5×10⁶ cells/mL. In certain embodiments, the VPC density at infection is 5.0-10.0×10⁶ cells/mL.

In certain embodiments, Infection BIICs are combined with the VPCs in target ratios of VPC-to-BIIC. In certain embodiments, the VPC-to-BIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to BIIC volume). In certain embodiments, the VPC-to-BIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to BIIC volume). In certain embodiments, the VPC-to-BIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to BIIC cells). In certain embodiments, the VPC-to-BIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to BIIC cells).

In certain embodiments, Infection BIICs which comprise Expression BEVs are combined with the VPCs in target ratios of VPC-to-expressionBIIC. In certain embodiments, the VPC-to-expressionBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to expressionBIIC volume). In certain embodiments, the VPC-to-expressionBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to expressionBIIC volume). In certain embodiments, the VPC-to-expressionBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to expressionBIIC cells). In certain embodiments, the VPC-to-expressionBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to expressionBIIC cells).

In certain embodiments, Infection BIICs which comprise Payload BEVs are combined with the VPCs in target ratios of VPC-to-payloadBIIC. In certain embodiments, the VPC-to-payloadBIIC infection ratio (volume to volume) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC volume to payloadBIIC volume). In certain embodiments, the VPC-to-payloadBIIC infection ratio (volume to volume) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC volume to payloadBIIC volume). In certain embodiments, the VPC-to-payloadBIIC infection ratio (cell to cell) is between 1.0×10³-3.0×10³, 2.0×10³-4.0×10³, 3.0×10³-5.0×10³, 4.0×10³-6.0×10³, 5.0×10³-7.0×10³, 6.0×10³-8.0×10³, 7.0×10³-9.0×10³, 8.0×10³-1.0×10⁴, 9.0×10³-1.1×10⁴, 1.0×10³-5.0×10³, 5.0×10³-1.0×10⁴, 1.0×10⁴-3.0×10⁴, 2.0×10⁴-4.0×10⁴, 3.0×10⁴-5.0×10⁴, 4.0×10⁴-6.0×10⁴, 5.0×10⁴-7.0×10⁴, 6.0×10⁴-8.0×10⁴, 7.0×10⁴-9.0×10⁴, 8.0×10⁴-1.0×10⁵, 9.0×10⁴-1.1×10⁵, 1.0×10⁴-5.0×10⁴, 5.0×10⁴-1.0×10⁵, 1.0×10⁵-3.0×10⁵, 2.0×10⁵-4.0×10⁵, 3.0×10⁵-5.0×10⁵, 4.0×10⁵-6.0×10⁵, 5.0×10⁵-7.0×10⁵, 6.0×10⁵-8.0×10⁵, 7.0×10⁵-9.0×10⁵, 8.0×10⁵-1.0×10⁶, 9.0×10⁵-1.1×10⁶, 1.0×10⁵-5.0×10⁵, 5.0×10⁵-1.0×10⁶, 1.0×10⁶-3.0×10⁶, 2.0×10⁶-4.0×10⁶, 3.0×10⁶-5.0×10⁶, 4.0×10⁶-6.0×10⁶, 5.0×10⁶-7.0×10⁶, 6.0×10⁶-8.0×10⁶, 7.0×10⁶-9.0×10⁶, 8.0×10⁶-1.0×10⁷, 9.0×10⁶-1.1×10⁷, 1.0×10⁶-5.0×10⁶, or 5.0×10⁶-1.0×10⁷ (VPC cells to payloadBIIC cells). In certain embodiments, the VPC-to-payloadBIIC infection ratio (cell to cell) is about 1.0×10³, about 1.5×10³, about 2.0×10³, about 2.5×10³, about 3.0×10³, about 3.5×10³, about 4.0×10³, about 4.5×10³, about 5.0×10³, about 5.5×10³, about 6.0×10³, about 6.5×10³, about 7.0×10³, about 7.5×10³, about 8.0×10³, about 8.5×10³, about 9.0×10³, about 9.5×10³, about 1.0×10⁴, about 1.5×10⁴, about 2.0×10⁴, about 2.5×10⁴, about 3.0×10⁴, about 3.5×10⁴, about 4.0×10⁴, about 4.5×10⁴, about 5.0×10⁴, about 5.5×10⁴, about 6.0×10⁴, about 6.5×10⁴, about 7.0×10⁴, about 7.5×10⁴, about 8.0×10⁴, about 8.5×10⁴, about 9.0×10⁴, about 9.5×10⁴, about 1.0×10⁵, about 1.5×10⁵, about 2.0×10⁵, about 2.5×10⁵, about 3.0×10⁵, about 3.5×10⁵, about 4.0×10⁵, about 4.5×10⁵, about 5.0×10⁵, about 5.5×10⁵, about 6.0×10⁵, about 6.5×10⁵, about 7.0×10⁵, about 7.5×10⁵, about 8.0×10⁵, about 8.5×10⁵, about 9.0×10⁵, about 9.5×10⁵, about 1.0×10⁶, about 1.5×10⁶, about 2.0×10⁶, about 2.5×10⁶, about 3.0×10⁶, about 3.5×10⁶, about 4.0×10⁶, about 4.5×10⁶, about 5.0×10⁶, about 5.5×10⁶, about 6.0×10⁶, about 6.5×10⁶, about 7.0×10⁶, about 7.5×10⁶, about 8.0×10⁶, about 8.5×10⁶, about 9.0×10⁶, or about 9.5×10⁶ (VPC cells to payloadBIIC cells).

In certain embodiments, Infection BIICs which comprise Expression BEVs and Infection BIICs which comprise Payload BEVs are combined with the VPCs in target expressionBIIC-to-payloadBIIC ratios. In certain embodiments, the ratio of expressionBIICs to payloadBIICs is 10:1, 9:1, 8:1, 7:1, 6:1, 5:1, 4.5:1, 4:1, 3.5:1, 3:1, 2.5:1, 2:1, 1.5:1, 1:1, 1:1.5, 1:2, 1:2.5, 1:3, 1:3.5, 1:4, 1:4.5, 1:5, 1:5.5, 1:6, 1:6.5, 1:7, 1:7.5, 1:8, 1:9, or 1:10. In certain embodiments, the ratio of expressionBIICs to payloadBIICs is between 6.5-7.5:1, 6-7:1, 5.5-6.5:1, 5-6:1, 4.5-5.5:1, 4-5:1, 3.5-4.5:1, 3-4:1, 2.5-3.5:1, 2-3:1, 1.5-2.5:1, 1-2:1, 1-1.5:1, 1:1-1.5, 1:1-2, 1:1.5-2.5, 1:2-3, 1:2.5-3.5, 1:3-4, 1:3.5-4.5, 1:4-5, 1:4.5-5.5, 1:5-6, 1:5.5-6.5, 1:6-7, or 1:6.5-7.5.

In certain embodiments, infected Viral Production Cells are incubated under a certain Dissolved Oxygen (DO) Content (DO %). In certain embodiments, infected Viral Production Cells are incubated under a DO % between 10%-50%, 20%-40%, 10%-20%, 15%-25%, 20%-30%, 25%-35%, 30%-40%, 35%-45%, 40%-50%, 10%-15%, 15%-20%, 20%-25%, 25%-30%, 30%-35%, 35%-40%, 40%-45%, or 45%-50%. In certain embodiments, infected Viral Production Cells are incubated under a DO % of about 10%, about 15%, about 20%, about 25%, about 30%, about 35%, about 40%, about 45%, about 50%. In certain embodiments, infected Viral Production Cells are incubated under a DO % between 20%-30% or about 25%. In certain embodiments, infected Viral Production Cells are incubated under a DO % between 25%-35% or about 30%. In certain embodiments, infected Viral Production Cells are incubated under a DO % between 30%-40% or about 35%. In certain embodiments, infected Viral Production Cells are incubated under a DO % between 35%-45% or about 40%.

### Cell Lysis

Cells of the present disclosure, comprising, but not limited to viral production cells, may be subjected to cell lysis according to any methods known in the art. Cell lysis may be carried out to obtain one or more agents (e.g., viral particles) present within any cells of the disclosure. In certain embodiments, a bulk harvest of AAV particles and viral production cells is subjected to cell lysis according to the present disclosure.

In certain embodiments, cell lysis may be carried out according to any of the methods or systems presented in U.S. Pat. Nos. 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935, 7,968,333, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Cell lysis methods and systems may be chemical or mechanical. Chemical cell lysis typically comprises contacting one or more cells with one or more chemical lysis agent under chemical lysis conditions. Mechanical lysis typically comprises subjecting one or more cells to cell lysis carried out by mechanical force. Lysis can also be completed by allowing the cells to degrade after reaching ˜0% viability.

In certain embodiments, chemical lysis may be used to lyse cells. As used herein, the term “chemical lysis agent” refers to any agent that may aid in the disruption of a cell. In certain embodiments, lysis agents are introduced in solutions, termed lysis solutions or lysis buffers. As used herein, the term “chemical lysis solution” refers to a solution (typically aqueous) comprising one or more lysis agent. In addition to lysis agents, lysis solutions may comprise one or more buffering agents, solubilizing agents, surfactants, preservatives, cryoprotectants, enzymes, enzyme inhibitors and/or chelators. Lysis buffers are lysis solutions comprising one or more buffering agent. Additional components of lysis solutions may comprise one or more solubilizing agent. As used herein, the term “solubilizing agent” refers to a compound that enhances the solubility of one or more components of a solution and/or the solubility of one or more entities to which solutions are applied. In certain embodiments, solubilizing agents enhance protein solubility. In certain embodiments, solubilizing agents are selected based on their ability to enhance protein solubility while maintaining protein conformation and/or activity.

Exemplary lysis agents may comprise any of those described in U.S. Pat. Nos. 8,685,734, 7,901,921, 7,732,129, 7,223,585, 7,125,706, 8,236,495, 8,110,351, 7,419,956, 7,300,797, 6,699,706 and 6,143,567, the contents of each of which are herein incorporated by reference in their entirety. In certain embodiments, lysis agents may be selected from lysis salts, amphoteric agents, cationic agents, ionic detergents and non-ionic detergents. Lysis salts may comprise, but are not limited to, sodium chloride (NaCl) and potassium chloride (KCl.) Further lysis salts may comprise any of those described in U.S. Pat. Nos. 8,614,101, 7,326,555, 7,579,181, 7,048,920, 6,410,300, 6,436,394, 7,732,129, 7,510,875, 7,445,930, 6,726,907, 6,194,191, 7,125,706, 6,995,006, 6,676,935 and 7,968,333, the contents of each of which are herein incorporated by reference in their entirety.

In certain embodiments, cell lysates agents include amino acids such as arginine, or acidified amino acid mixtures such as arginine HCl. In certain embodiments, the cell lysate solution comprises a stabilizing additive. In certain embodiments, the stabilizing additive can comprise trehalose, glycine betaine, mannitol, potassium citrate, CuCl₂, proline, xylitol, NDSB 201, CTAB and K₂PO₄. In certain embodiments, the stabilizing additive can comprise amino acids such as arginine, or acidified amino acid mixtures such as arginine HCl. In certain embodiments, the stabilizing additive can comprise 0.1 M arginine or arginine HCl. In certain embodiments, the stabilizing additive can comprise 0.2 M arginine or arginine HCl. In certain embodiments, the stabilizing additive can comprise 0.25 M arginine or arginine HCl. In certain embodiments, the stabilizing additive can comprise 0.3 M arginine or arginine HCl. In certain embodiments, the stabilizing additive can comprise 0.4 M arginine or arginine HCl. In certain embodiments, the stabilizing additive can comprise 0.5 M arginine or arginine HCl. In certain embodiments, the stabilizing additive can comprise 0.6 M arginine or arginine HCl. In certain embodiments, the stabilizing additive can comprise 0.7 M arginine or arginine HCl. In certain embodiments, the stabilizing additive can comprise 0.8 M arginine or arginine HCl. In certain embodiments, the stabilizing additive can comprise 0.9 M arginine or arginine HCl. In certain embodiments, the stabilizing additive can comprise 1.0M arginine or arginine HCl.

Concentrations of salts may be increased or decreased to obtain an effective concentration for the rupture of cell membranes Amphoteric agents, as referred to herein, are compounds capable of reacting as an acid or a base Amphoteric agents may comprise, but are not limited to lysophosphatidylcholine, 3-((3-Cholamidopropyl) dimethylammonium)-1-propanesulfonate (CHAPS), ZWITTERGENT® and the like. Cationic agents may comprise, but are not limited to, cetyltrimethylammonium bromide (C (16) TAB) and Benzalkonium chloride. Lysis agents comprising detergents may comprise ionic detergents or non-ionic detergents.

Detergents may function to break apart or dissolve cell structures comprising, but not limited to cell membranes, cell walls, lipids, carbohydrates, lipoproteins and glycoproteins. Exemplary ionic detergents comprise any of those taught in U.S. Pat. Nos. 7,625,570 and 6,593,123 or US Publication No. US2014/0087361, the contents of each of which are herein incorporated by reference in their entirety. In certain embodiments, the lysis solution comprises one or more ionic detergents. Example of ionic detergents for use in a lysis solution comprise, but are not limited to, sodium dodecyl sulfate (SDS), cholate and deoxycholate. In certain embodiments, ionic detergents may be comprised in lysis solutions as a solubilizing agent. In certain embodiments, the lysis solution comprises one or more nonionic detergents. Non-ionic detergents for use in a lysis solution may comprise, but are not limited to, octylglucoside, digitonin, lubrol, C12E8, TWEEN®-20, TWEEN®-80, Triton X-100, Triton X-114, Brij-35, Brij-58, and Noniodet P-40. Non-ionic detergents are typically weaker lysis agents but may be comprised as solubilizing agents for solubilizing cellular and/or viral proteins. In certain embodiments, the lysis solution comprises one or more zwitterionic detergents. Zwitterionic detergents for use in a lysis solution may comprise, but are not limited to: Lauryl dimethylamine N-oxide (LDAO); N,N-Dimethyl-N-dodecylglycine betaine (Empigen® BB); 3-(N,N-Dimethylmyristylammonio) propanesulfonate (Zwittergent® 3-10); n-Dodecyl-N,N-dimethyl-3-ammonio-1-propanesulfonate (Zwittergent® 3-12); n-Tetradecyl-N,N-dimethyl-3-ammonio-1-propanesulfonate (Zwittergent® 3-14); 3-(N,N-Dimethyl palmitylammonio) propanesulfonate (Zwittergent® 3-16); 3-((3-cholamidopropyl) dimethylammonio)-1-propanesulfonate (CHAPS); and 3-([3-Cholamidopropyl] dimethylammonio)-2-hydroxy-1-propanesulfonate (CHAPSO).

In certain embodiments, the lysis solution comprises Triton X-100 (octyl phenol ethoxylate), such as 0.5% w/v of Triton X-100. In certain embodiments, the lysis solution comprises Lauryldimethylamine N-oxide (LDAO), such as 0.184% w/v (4×CMC) of LDAO. In certain embodiments, the lysis solution comprises a seed oil surfactant such as Ecosurf™ SA-9. In certain embodiments, the lysis solution comprises N,N-Dimethyl-N-dodecylglycine betaine (Empigen® BB). In certain embodiments, the lysis solution comprises a Zwittergent® detergent, such as Zwittergent® 3-12 (n-Dodecyl-N,N-dimethyl-3-ammonio-1-propanesulfonate), Zwittergent® 3-14 (n-Tetradecyl-N,N-dimethyl-3-ammonio-1-propanesulfonate), or Zwittergent® 3-16 (3-(N,N-Dimethyl palmitylammonio)propanesulfonate).

Further lysis agents may comprise enzymes and urea. In certain embodiments, one or more lysis agents may be combined in a lysis solution in order to enhance one or more of cell lysis and protein solubility. In certain embodiments, enzyme inhibitors may be comprised in lysis solutions in order to prevent proteolysis that may be triggered by cell membrane disruption.

In certain embodiments, the lysis solution comprises between 0.1-1.0% w/v, between 0.2-0.8% w/v, between 0.3-0.7% w/v, between 0.4-0.6% w/v, or about 0.5% w/v of a cell lysis agent (e.g., detergent). In certain embodiments, the lysis solution comprises between 0.3-0.35% w/v, between 0.35-0.4% w/v, between 0.4-0.45% w/v, between 0.45-0.5% w/v, between 0.5-0.55% w/v, between 0.55-0.6% w/v, between 0.6-0.65% w/v, or between 0.65-0.7% w/v of a cell lysis agent (e.g., detergent).

In certain embodiments, cell lysates generated from adherent cell cultures may be treated with one more nuclease, such as Benzonase nuclease (Grade I, 99% pure) or c-LEcta Denarase nuclease (formerly Sartorius Denarase). In certain embodiments, nuclease is added to lower the viscosity of the lysates caused by liberated DNA.

In certain embodiments, chemical lysis uses a single chemical lysis mixture. In certain embodiments, chemical lysis uses several lysis agents added in series to provide a final chemical lysis mixture.

In certain embodiments, a chemical lysis mixture comprises an acidified amino acid mixture (such as arginine HCl), a non-ionic detergent (such as Triton X-100), and a nuclease (such as Benzonase nuclease). In certain embodiments, the chemical lysis mixture can comprise an acid or base to provide a target lysis pH.

In certain embodiments, the lysis solution comprises 0.5% w/v Triton X-100 (octyl phenol ethoxylate) and 200 mM arginine hydrochloride. In certain embodiments, the lysis solution comprises 0.5% w/v Triton X-100 (octyl phenol ethoxylate) and 200 mM arginine hydrochloride, and lacks detectable nuclease. In certain embodiments, the lysis solution consists of 0.5% w/v Triton X-100 (octyl phenol ethoxylate) and 200 mM arginine hydrochloride.

In certain embodiments, chemical lysis is conducted under chemical lysis conditions. As used herein, the term “chemical lysis conditions” refers to any combination of environmental conditions (e.g., temperature, pressure, pH, etc.) in which targets cells can be lysed by a chemical lysis agent.

In certain embodiments, the lysis pH is between 3.0-3.5, 3.5-4.0, 4.0-4.5, 4.5-5.0, 5.0-5.5, 5.5-6.0, 6.0-6.5, 6.5-7.0, 7.0-7.5, or 7.5-8.0. In certain embodiments, the lysis pH is between 6.0-7.0, 6.5-7.0, 6.5-7.5, or 7.0-7.5.

In certain embodiments, the lysis temperature is between 15-35° C., between 20-30° C., between 25-39° C., between 20-21° C., between 20-22° C., between 21-22° C., between 21-23° C., between 22-23° C., between 22-24° C., between 23-24° C., between 23-25° C., between 24-25° C., between 24-26° C., between 25-26° C., between 25-27° C., between 26-27° C., between 26-28° C., between 27-28° C., between 27-29° C., between 28-29° C., between 28-30° C., between 29-30° C., between 29-31° C., between 30-31° C., between 30-32° C., between 31-32° C., or between 31-33° C.

In certain embodiments, the lysis solution comprises 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.). In certain embodiments, the lysis solution comprises 0.5% w/v Triton X-100 (octyl phenol ethoxylate) and 200 mM arginine hydrochloride, and lacks detectable nuclease, and lysis conditions comprise a duration of at least 4 hours (e.g., 4-6 hours, e.g., 4 hours) at 26° C.-28° C. (e.g., 27° C.). In certain embodiments, the lysis solution consists of 0.5% w/v Triton X-100 (octyl phenol ethoxylate) and 200 mM arginine hydrochloride, and lysis conditions comprise a duration of at least 4 hours (e.g., 4-6 hours, e.g., 4 hours) at 26° C.-28° C. (e.g., 27° C.).

In certain embodiments, mechanical cell lysis is carried out. Mechanical cell lysis methods may comprise the use of one or more lysis condition and/or one or more lysis force. As used herein, the term “lysis condition” refers to a state or circumstance that promotes cellular disruption. Lysis conditions may comprise certain temperatures, pressures, osmotic purity, salinity and the like. In certain embodiments, lysis conditions comprise increased or decreased temperatures. According to certain embodiments, lysis conditions comprise changes in temperature to promote cellular disruption. Cell lysis carried out according to such embodiments may comprise freeze-thaw lysis. As used herein, the term “freeze-thaw lysis” refers to cellular lysis in which a cell solution is subjected to one or more freeze-thaw cycle. According to freeze-thaw lysis methods, cells in solution are frozen to induce a mechanical disruption of cellular membranes caused by the formation and expansion of ice crystals. Cell solutions used according freeze-thaw lysis methods, may further comprise one or more lysis agents, solubilizing agents, buffering agents, cryoprotectants, surfactants, preservatives, enzymes, enzyme inhibitors and/or chelators. Once cell solutions subjected to freezing are thawed, such components may enhance the recovery of desired cellular products. In certain embodiments, one or more cryoprotectants are comprised in cell solutions undergoing freeze-thaw lysis. As used herein, the term “cryoprotectant” refers to an agent used to protect one or more substance from damage due to freezing. Cryoprotectants may comprise any of those taught in US Publication No. US2013/0323302 or U.S. Pat. Nos. 6,503,888, 6,180,613, 7,888,096, 7,091,030, the contents of each of which are herein incorporated by reference in their entirety. In certain embodiments, cryoprotectants may comprise, but are not limited to dimethyl sulfoxide, 1,2-propanediol, 2,3-butanediol, formamide, glycerol, ethylene glycol, 1,3-propanediol and n-dimethyl formamide, polyvinylpyrrolidone, hydroxyethyl starch, agarose, dextrans, inositol, glucose, hydroxyethylstarch, lactose, sorbitol, methyl glucose, sucrose, and urea. In certain embodiments, freeze-thaw lysis may be carried out according to any of the methods described in U.S. Pat. No. 7,704,721, the contents of which are herein incorporated by reference in their entirety.

As used herein, the term “lysis force” refers to a physical activity used to disrupt a cell. Lysis forces may comprise, but are not limited to mechanical forces, sonic forces, gravitational forces, optical forces, electrical forces and the like. Cell lysis carried out by mechanical force is referred to herein as “mechanical lysis.” Mechanical forces that may be used according to mechanical lysis may comprise high shear fluid forces. According to such methods of mechanical lysis, a microfluidizer may be used. Microfluidizers typically comprise an inlet reservoir where cell solutions may be applied. Cell solutions may then be pumped into an interaction chamber via a pump (e.g., high-pressure pump) at high speed and/or pressure to produce shear fluid forces. Resulting lysates may then be collected in one or more output reservoir. Pump speed and/or pressure may be adjusted to modulate cell lysis and enhance recovery of products (e.g., viral particles.) Other mechanical lysis methods may comprise physical disruption of cells by scraping.

Cell lysis methods may be selected based on the cell culture format of cells to be lysed. For example, with adherent cell cultures, some chemical and mechanical lysis methods may be used. Such mechanical lysis methods may comprise freeze-thaw lysis or scraping. In another example, chemical lysis of adherent cell cultures may be carried out through incubation with lysis solutions comprising surfactant, such as Triton-X-100.

In certain embodiments, a method for harvesting AAV particles without lysis may be used for efficient and scalable AAV particle production. In a non-limiting example, AAV particles may be produced by culturing an AAV particle lacking a heparin binding site, thereby allowing the AAV particle to pass into the supernatant, in a cell culture, collecting supernatant from the culture; and isolating the AAV particle from the supernatant, as described in US Patent Application 20090275107, the contents of which are incorporated herein by reference in their entirety.

### Clarification and Purification: General

Cell lysates comprising viral particles may be subjected to clarification and purification. Clarification generally refers to the initial steps taken in the purification of viral particles from cell lysates and serves to prepare lysates for further purification by removing larger, insoluble debris from a bulk lysis harvest. Viral production can comprise clarification steps at any point in the viral production process. Clarification steps may comprise, but are not limited to, centrifugation and filtration. During clarification, centrifugation may be carried out at low speeds to remove larger debris only. Similarly, filtration may be carried out using filters with larger pore sizes so that only larger debris is removed.

Purification generally refers to the final steps taken in the purification and concentration of viral particles from cell lysates by removing smaller debris from a clarified lysis harvest in preparing a final Pooled Drug Substance. Viral production can comprise purification steps at any point in the viral production process. Purification steps may comprise, but are not limited to, filtration and chromatography. Filtration may be carried out using filters with smaller pore sizes to remove smaller debris from the product or with larger pore sizes to retain larger debris from the product. Filtration may be used to alter the concentration and/or contents of a viral production pool or stream. Chromatography may be carried out to selectively separate target particles from a pool of impurities.

Large-scale production of high-concentration AAV formulations is complicated by the tendency for high concentrations of AAV particles to aggregate or agglomerate Small scale clarification and concentration systems, such as dialysis cassettes or spin centrifugation, are generally not sufficiently scalable for large-scale production. The present disclosure provides embodiments of a clarification, purification and concentration system for processing large volumes of high-concentration AAV production formulations. In certain embodiments, the large-volume clarification system comprises one or more of the following processing steps: Depth Filtration, Microfiltration (e.g., 0.2 μm Filtration), Affinity Chromatography, Ion Exchange Chromatography such as anion exchange chromatography (AEX) or cation exchange chromatography (CEX), a tangential flow filtration system (TFF), Nanofiltration (e.g., Virus Retentive Filtration (VRF)), Final Filtration (FF), and Fill Filtration.

Objectives of viral clarification and purification comprise high throughput processing of cell lysates and to optimize ultimate viral recovery. Advantages of comprising clarification and purification steps of the present disclosure comprise scalability for processing of larger volumes of lysate. In certain embodiments, clarification and purification may be carried out according to any of the methods or systems presented in U.S. Pat. Nos. 8,524,446, 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498, 7,491,508, US Publication Nos. US2013/0045186, US2011/0263027, US2011/0151434, US2003/0138772, and International Publication Nos. WO2002012455, WO1996039530, WO1998010088, WO1999014354, WO1999015685, WO1999047691, WO2000055342, WO2000075353 and WO2001023597, the contents of each of which are herein incorporated by reference in their entirety.

In certain embodiments, the compositions comprising at least one AAV particle may be isolated or purified using the methods or systems described in U.S. Pat. Nos. 6,146,874, 6,660,514, 8,283,151 or U.S. Pat. No. 8,524,446, the contents of which are herein incorporated by reference in their entirety.

### Clarification and Purification: Centrifugation

According to certain embodiments, cell lysates may be clarified by one or more centrifugation steps. Centrifugation may be used to pellet insoluble particles in the lysate. During clarification, centrifugation strength (which can be expressed in terms of gravitational units (g), which represents multiples of standard gravitational force) may be lower than in subsequent purification steps. In certain embodiments, centrifugation may be carried out on cell lysates at a gravitation force from about 200 g to about 800 g, from about 500 g to about 1500 g, from about 1000 g to about 5000 g, from about 1200 g to about 10000 g or from about 8000 g to about 15000 g. In certain embodiments, cell lysate centrifugation is carried out at 8000 g for 15 minutes. In certain embodiments, density gradient centrifugation may be carried out in order to partition particulates in the cell lysate by sedimentation rate. Gradients used according to methods or systems of the present disclosure may comprise, but are not limited to, cesium chloride gradients and iodixanol step gradients. In certain embodiments, centrifugation uses a decanter centrifuge system. In certain embodiments, centrifugation uses a disc-stack centrifuge system. In certain embodiments, centrifugation comprises ultracentrifugation, such two-cycle CsCl gradient ultracentrifugation or iodixanol discontinuous density gradient ultracentrifugation.

### Clarification and Purification: Filtration

In certain embodiments, one or more microfiltration, nanofiltration and/or ultrafiltration steps may be used during clarification, purification and/or sterilization. The one or more microfiltration, nanofiltration or ultrafiltration steps can comprise the use of a filtration system such as EMD Millipore Express SHC XL10 0.5/0.2 μm filter, EMD Millipore Express SHCXL6000 0.5/0.2 μm filter, EMD Millipore Express SHCXL150 filter, EMD Millipore Millipak Gamma Gold 0.22 μm filter (dual-in-line sterilizing grade filters), a Pall Supor EKV, 0.2 μm sterilizing-grade filter, Asahi Planova 35N, Asahi Planova 20N, Asahi Planova 75N, Asahi Planova BioEx, Millipore Viresolve NFR or a Sartorius Sartopore 2×LG, 0.8/0.2 μm.

In certain embodiments, one or more microfiltration steps may be used during clarification, purification and/or sterilization. Microfiltration utilizes microfiltration membranes with pore sizes typically between 0.1 μm and 10 μm. Microfiltration is generally used for general clarification, sterilization, and removal of microparticulates. In certain embodiments, microfiltration is used to remove aggregated clumps of viral particles. In certain embodiments, a production process or system of the present disclosure comprises at least one microfiltration step. The one or more microfiltration steps can comprise a Depth Filtration step with a Depth Filtration system, such as EMD Millipore Millistak⁺ POD filter (DOHC media series), Millipore MC0SP23CL3 filter (C0SP media series), or Sartorius Sartopore filter series. Microfiltration systems of the present disclosure can be pre-rinsed, packed, equilibrated, flushed, processed, eluted, washed, or cleaned with formulations known to those in the art, comprising AAV pharmaceutical, processing and storage formulations of the present disclosure. In certain embodiments, clarification comprises use of a C0SP media series filter. In some embodiments, the C0SP media series filter is effective to reduce or prevent 0.2-micron filter clogging.

In certain embodiments, one or more ultrafiltration steps may be used during clarification and purification. The ultrafiltration steps can be used for concentrating, formulating, desalting or dehydrating either processing and/or formulation solutions of the present disclosure. Ultrafiltration utilizes ultrafiltration membranes, with pore sizes typically between 0.001 and 0.1 μm. Ultrafiltration membranes can also be defined by their molecular weight cutoff (MWCO) and can have a range from 1 kD to 500 kD. Ultrafiltration is generally used for concentrating and formulating dissolved biomolecules such as proteins, peptides, plasmids, viral particles, nucleic acids, and carbohydrates. Ultrafiltration systems of the present disclosure can be pre-rinsed, packed, equilibrated, flushed, processed, eluted, washed, or cleaned with formulations known to those in the art, comprising AAV pharmaceutical, processing and storage formulations of the present disclosure.

In certain embodiments, one or more nanofiltration steps may be used during clarification and purification. Nanofiltration utilizes nanofiltration membranes, with pore sizes typically less than 100 nm. Nanofiltration is generally used for removal of unwanted endogenous viral impurities (e.g., baculovirus). In certain embodiments, nanofiltration can comprise viral removal filtration (VRF). VRF filters can have a filtration size typically between 15 nm and 100 nm. Examples of VRF filters comprise (but are not limited to): Planova 15N, Planova 20N, and Planova 35N (Asahi-Kasei Corp, Tokyo, Japan); and Viresolve NFP and Viresolve NFR (Millipore Corp, Billerica, MA, USA). Nanofiltration systems of the present disclosure can be pre-rinsed, packed, equilibrated, flushed, processed, eluted, washed, or cleaned with formulations known to those in the art, comprising AAV pharmaceutical, processing and storage formulations of the present disclosure. In certain embodiments, nanofiltration is used to remove aggregated clumps of viral particles.

In certain embodiments, one or more tangential flow filtration (TFF) (also known as cross-flow filtration) steps may be used during clarification and purification. Tangential flow filtration is a form of membrane filtration in which a feed stream (which comprises the target agent/particle to be clarified and concentrated) flows from a feed tank into a filtration module or cartridge. Within the TFF filtration module, the feed stream passes parallel to a membrane surface, such that one portion of the stream passes through the membrane (permeate/filtrate) while the remainder of the stream (retentate) is recirculated back through the filtration system and into the feed tank.

In certain embodiments, the TFF filtration module can be a flat plate module (stacked planar cassette), a spiral wound module (spiral-wound membrane layers), or a hollow fiber module (bundle of membrane tubes). Examples of TFF systems for use in the present disclosure comprise, but are not limited to: Spectrum mPES Hollow Fiber TFF system (0.5 mm fiber ID, 100 kDA MWCO) or Millipore Ultracel PLCTK system with Pellicon-3 cassette (0.57 m², 30 kDA MWCO).

New buffer materials can be added to the TFF feed tank as the feed stream is circulated through the TFF filtration system. In certain embodiments, buffer materials can be fully replenished as the flow stream circulates through the TFF filtration system. In this embodiment, buffer material is added to the stream in equal amounts to the buffer material lost in the permeate, resulting in a constant concentration. In certain embodiments, buffer materials can be reduced as the flow stream circulates through the filtration system. In this embodiment, a reduced amount of buffer material is added to the stream relative to the buffer material lost in the permeate, resulting in an increased concentration. In certain embodiments, buffer materials can be replaced as the flow stream circulates through the filtration system. In this embodiment, the buffer added to stream is different from buffer materials lost in the permeate, resulting in an eventual replacement of buffer material in the stream. TFF systems of the present disclosure can be pre-rinsed, packed, equilibrated, flushed, processed, eluted, washed or cleaned with formulations known to those in the art, comprising AAV pharmaceutical, processing and storage formulations of the present disclosure.

In certain embodiments, a TFF load pool can be spiked with an excipient or diluent prior to filtration. In certain embodiments, a TFF load pool is spiked with a high-salt mixture (such as sodium chloride or potassium chloride) prior to filtration. In certain embodiments, a TFF load pool is spiked with a high-sugar mixture (such as 50% w/v sucrose) prior to filtration.

The effectiveness of TFF processing can depend on several factors, comprising (but not limited to): shear stress from flow design, cross-flow rate, filtrate flow control, transmembrane pressure (TMP), membrane conditioning, membrane composition (e.g., hollow fiber construction) and design (e.g., surface area), system flow design, reservoir design, and mixing strategy. In certain embodiment, the filtration membrane can be exposed to pre-TFF membrane conditioning.

In certain embodiments, TFF processing can comprise one or more microfiltration stages. In certain embodiments, TFF processing can comprise one or more ultrafiltration stages. In certain embodiments, TFF processing can comprise one or more nanofiltration stages.

In certain embodiments, TFF processing can comprise one or more concentration stages, such as an ultrafiltration (UF) or microfiltration (MF) concentration stage. In the concentration stage, a reduced amount of buffer material is replaced as the stream circulates through the filtration system (relative to the amount of buffer material lost as permeate). The failure to completely replace all of the buffer material lost in the permeate results in an increased concentration of viral particles within the filtration stream. In certain embodiments, an increased amount of buffer material is replaced as the stream circulates through the filtration system. The incorporation of excess buffer material relative to the amount of buffer material lost in the permeate results in a decreased concentration of viral particles within the filtration stream.

In certain embodiments, TFF processing can comprise one or more diafiltration (DF) stages. The diafiltration stage comprises replacement of a first buffer material (such as a high salt material) within a second buffer material (such a low-salt or zero-salt material). In this embodiment, a second buffer is added to flow stream which is different from a first buffer material lost in the permeate, resulting in an eventual replacement of buffer material in the stream.

In certain embodiments, TFF processing can comprise multiple stages in series. In certain embodiments, a TFF processing process can comprise an ultrafiltration (UF) concentration stage followed by a diafiltration stage (DF). In certain embodiments, TFF comprising UF followed by DF results in increased rAAV recovery relative to TFF comprising DF followed by UF. In some embodiments, TFF comprising UF followed by DF results in about 70-80% recovery of rAAV.

In certain embodiments, a TFF processing can comprise a diafiltration stage followed by an ultrafiltration concentration stage. In certain embodiments, a TFF processing can comprise a first diafiltration stage, followed by an ultrafiltration concentration stage, followed by a second diafiltration stage. In certain embodiments, a TFF processing can comprise a first diafiltration stage which incorporates a high-salt-low-sugar buffer material into the flow stream, followed by an ultrafiltration/concentration stage which results in a high concentration of the viral material in the flow stream, followed by a second diafiltration stage which incorporates a low-salt-high-sugar or zero-salt-high-sugar buffer material into the flow stream. In certain embodiments, the salt can be sodium chloride, sodium phosphate, potassium chloride, potassium phosphate, or a combination thereof. In certain embodiments, the sugar can be sucrose, such as a 5% w/v sucrose mixture or a 7% w/v sucrose mixture.

In certain embodiments, the one or more TFF steps can comprise a formulation diafiltration step in which at least a portion of the liquid media of the viral production pool is replaced with a high-sucrose formulation buffer. In certain embodiments, the high-sucrose formulation buffer comprises between 6-8% w/v of a sugar or sugar substitute and between 90-100 mM of an alkali chloride salt. In certain embodiments, the high-sucrose formulation buffer comprises 7% w/v of sucrose and between 90-100 mM sodium chloride. In certain embodiments, the high-sucrose formulation buffer comprises 7% w/v of sucrose, 10 mM Sodium Phosphate, between 95-100 mM sodium chloride, and 0.001% (w/v) Poloxamer 188. In certain embodiments, the formulation diafiltration step is the final diafiltration step in the one or more TFF steps. In certain embodiments, the formulation diafiltration step is the only diafiltration step in the one or more TFF steps.

In certain embodiments, TFF processing can comprise multiple stages which occur contemporaneously. As a non-limiting example, a TFF clarification process can comprise an ultrafiltration stage which occurs contemporaneously with a concentration stage.

Methods of cell lysate clarification and purification by filtration are well understood in the art and may be carried out according to a variety of available methods comprising, but not limited to passive filtration and flow filtration. Filters used may comprise a variety of materials and pore sizes. For example, cell lysate filters may comprise pore sizes of from about 1 μM to about 5 μM, from about 0.5 μM to about 2 μM, from about 0.1 μM to about 1 μM, from about 0.05 μM to about 0.05 μM and from about 0.001 μM to about 0.1 μM. Exemplary pore sizes for cell lysate filters may comprise, but are not limited to, 2.0, 1.9, 1.8, 1.7, 1.6, 1.5, 1.4, 1.3, 1.2, 1.1, 1, 0.9, 0.8, 0.7, 0.6, 0.5, 0.4, 0.3, 0.2, 0.1, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.22, 0.21, 0.20, 0.19, 0.18, 0.17, 0.16, 0.15, 0.14, 0.13, 0.12, 0.11, 0.1, 0.09, 0.08, 0.07, 0.06, 0.05, 0.04, 0.03, 0.02, 0.01, 0.02, 0.019, 0.018, 0.017, 0.016, 0.015, 0.014, 0.013, 0.012, 0.011, 0.01, 0.009, 0.008, 0.007, 0.006, 0.005, 0.004, 0.003, 0.002, 0.001 and 0.001 μM. In certain embodiments, clarification may comprise filtration through a filter with 2.0 μM pore size to remove large debris, followed by passage through a filter with 0.45 μM pore size to remove intact cells.

Filter materials may be composed of a variety of materials. Such materials may comprise, but are not limited to, polymeric materials and metal materials (e.g., sintered metal and pored aluminum.) Exemplary materials may comprise, but are not limited to nylon, cellulose materials (e.g., cellulose acetate), polyvinylidene fluoride (PVDF), polyethersulfone, polyamide, polysulfone, polypropylene, and polyethylene terephthalate. In certain embodiments, filters useful for clarification of cell lysates may comprise, but are not limited to ULTIPLEAT PROFILE™ filters (Pall Corporation, Port Washington, NY), SUPOR™ membrane filters (Pall Corporation, Port Washington, NY).

In certain embodiments, flow filtration may be carried out to increase filtration speed and/or effectiveness. In certain embodiments, flow filtration may comprise vacuum filtration. According to such methods, a vacuum is created on the side of the filter opposite that of cell lysate to be filtered. In certain embodiments, cell lysates may be passed through filters by centrifugal forces. In certain embodiments, a pump is used to force cell lysate through clarification filters. Flow rate of cell lysate through one or more filters may be modulated by adjusting one of channel size and/or fluid pressure.

### Clarification and Purification: Chromatography

In certain embodiments, AAV particles in a formulation may be clarified and purified from cell lysates through one or more chromatography steps using one or more different methods of chromatography. Chromatography refers to any number of methods known in the art for selectively separating out one or more elements from a mixture. Such methods may comprise, but are not limited to, ion exchange chromatography (e.g., cation exchange chromatography and anion exchange chromatography), affinity chromatography (e.g., immunoaffinity chromatography, metal affinity chromatography, pseudo affinity chromatography such as Blue Sepharose resins), hydrophobic interaction chromatography (HIC), size-exclusion chromatography, and multimodal chromatography (MMC) (chromatographic methods that utilize more than one form of interaction between the stationary phase and analytes). In certain embodiments, methods or systems of viral chromatography may comprise any of those taught in U.S. Pat. Nos. 5,756,283, 6,258,595, 6,261,551, 6,270,996, 6,281,010, 6,365,394, 6,475,769, 6,482,634, 6,485,966, 6,943,019, 6,953,690, 7,022,519, 7,238,526, 7,291,498 and 7,491,508 or International Publication Nos. WO1996039530, WO1998010088, WO1999014354, WO1999015685, WO1999047691, WO2000055342, WO2000075353 and WO2001023597, the contents of each of which are herein incorporated by reference in their entirety.

Chromatography systems of the present disclosure can be pre-rinsed, packed, equilibrated, flushed, processed, eluted, washed, or cleaned with formulations known to those in the art, comprising AAV pharmaceutical, processing and storage formulations of the present disclosure.

In certain embodiments, one or more ion exchange (IEX) chromatography steps may be used to isolate viral particles. The ion exchange step can comprise anion exchange (AEX) chromatography, cation exchange (CEX) chromatography, or a combination thereof. In certain embodiments, ion exchange chromatography is used in a bind/elute mode. Bind/elute IEX can be used by binding viral particles to a stationary phase based on charge-charge interactions between capsid proteins (or other charged components) of the viral particles and charged sites present on the stationary phase. This process can comprise the use of a column through which viral preparations (e.g., clarified lysates) are passed. After application of viral preparations to the charged stationary phase (e.g., column), bound viral particles may then be eluted from the stationary phase by applying an elution solution to disrupt the charge-charge interactions. Elution solutions may be optimized by adjusting salt concentration and/or pH to enhance recovery of bound viral particles. In certain embodiments, the elution solution can comprise a nuclease such as Benzonase nuclease. Depending on the charge of viral capsids being isolated, cation or anion exchange chromatography methods may be selected. In certain embodiments, ion exchange chromatography is used in a flow-through mode. Flow-through IEX can be used by binding non-viral impurities or unwanted viral particles to a stationary phase (based on charge-charge interactions) and allowing the target viral particles in the viral preparation to “flow through” the IEX system into a collection pool.

Methods or systems of ion exchange chromatography may comprise, but are not limited to any of those taught in U.S. Pat. Nos. 7,419,817, 6,143,548, 7,094,604, 6,593,123, 7,015,026 and 8,137,948, the contents of each of which are herein incorporated by reference in their entirety.

In certain embodiments, the IEX process uses an AEX chromatography system such as a Sartorius Sartobind Q membrane, a Sartorius Sartobind STIC membrane, a Millipore Fractogel TMAE HiCap(m) Flow-Through membrane, a GE Q Sepharose HP membrane, Poros XQ or Poros HQ. In certain embodiments, the IEX process uses a CEX system such as a Poros XS membrane. In certain embodiments, the AEX system comprises a stationary phase which comprises a trimethylammoniumethyl (TMAE) functional group. In certain embodiments, the IEX process uses a Multimodal Chromatography (MMC) system such as a Nuvia aPrime 4A membrane.

In certain embodiments, one or more affinity chromatography steps, such as immunoaffinity chromatography, may be used to isolate viral particles Immunoaffinity chromatography is a form of chromatography that utilizes one or more immune compounds (e.g., antibodies or antibody-related structures) to retain viral particles Immune compounds may bind specifically to one or more structures on viral particle surfaces, comprising, but not limited to one or more viral coat protein. In certain embodiments, immune compounds may be specific for a particular viral variant. In certain embodiments, immune compounds may bind to multiple viral variants. In certain embodiments, immune compounds may comprise recombinant single-chain antibodies. Such recombinant single chain antibodies may comprise those described in Smith, R. H. et al., 2009. Mol. Ther. 17(11):1888-96, the contents of which are herein incorporated by reference in their entirety. Such immune compounds (e.g., recombinant protein ligands) are capable of binding to several AAV capsid variants, comprising, but not limited to AAV1, AAV2, AAV3, AAV5, AAV6 and/or AAV8 or any of those taught herein. In some embodiments, such immune compounds (e.g., recombinant protein ligands) are capable of binding to at least AAV2. In certain embodiments, the AFC process uses a GE AVB Sepharose HP column resin, Poros CaptureSelect AAV8 resins (ThermoFisher), Poros CaptureSelect AAV9 resins (ThermoFisher) and Poros CaptureSelect AAVX resins (ThermoFisher).

In certain embodiments, one or more affinity chromatography steps precedes one or more anion exchange chromatography steps. In certain embodiments, one or more anion exchange chromatography steps precedes one or more affinity chromatography steps.

In certain embodiments, one or more size-exclusion chromatography (SEC) steps may be used to isolate viral particles. SEC may comprise the use of a gel to separate particles according to size. In viral particle purification, SEC filtration is sometimes referred to as “polishing.” In certain embodiments, SEC may be carried out to generate a final product that is near-homogenous. Such final products may in certain embodiments be used in pre-clinical studies and/or clinical studies (Kotin, R. M. 2011. Human Molecular Genetics. 20(1):R2-R6, the contents of which are herein incorporated by reference in their entirety.) In certain embodiments, SEC may be carried out according to any of the methods taught in U.S. Pat. Nos. 6,143,548, 7,015,026, 8,476,418, 6,410,300, 8,476,418, 7,419,817, 7,094,604, 6,593,123, and 8,137,948, the contents of each of which are herein incorporated by reference in their entirety.

In some embodiments, purification of recombinant AAV produces a total rAAV process yield of 30-50%.

### III. DEFINITIONS

At various places in the present disclosure, substituents, or properties of compounds of the present disclosure are disclosed in groups or in ranges. It is specifically intended that the present disclosure comprise each and every individual or sub-combination of the members of such groups and ranges.

Unless stated otherwise, the following terms and phrases have the meanings described below. The definitions are not meant to be limiting in nature and serve to provide a clearer understanding of certain aspects of the present disclosure.

Adeno-associated virus: The term “adeno-associated virus” or “AAV” as used herein refers to members of the dependovirus genus comprising any particle, sequence, gene, protein, or component derived therefrom.

AAV Particle: As used herein, an “AAV particle” refers to a particle or a virion comprising an AAV capsid, e.g., an AAV capsid variant, and a polynucleotide, e.g., a viral genome or a vector genome. In some embodiments, the viral genome of the AAV particle comprises at least one payload region and at least one ITR. In some embodiments, an AAV particle of the disclosure is an AAV particle comprising an AAV variant. In some embodiments, the AAV particle is capable of delivering a nucleic acid, e.g., a payload region, encoding a payload to cells, typically, mammalian, e.g., human, cells. In some embodiments, an AAV particle of the present disclosure may be produced recombinantly. In some embodiments, an AAV particle may be derived from any serotype, described herein or known in the art, including combinations of serotypes (e.g., “pseudotyped” AAV) or from various genomes (e.g., single stranded or self-complementary). In some embodiments, the AAV particle may be replication defective and/or targeted. It is to be understood that reference to the AAV particle of the disclosure also includes pharmaceutical compositions thereof, even if not explicitly recited.

Administering: As used herein, the term “administering” refers to providing a pharmaceutical agent or composition to a subject.

Amelioration: As used herein, the term “amelioration” or “ameliorating” refers to a lessening of severity of at least one indicator of a condition or disease. For example, in the context of neurodegeneration disorder, amelioration comprises the reduction of neuron loss.

Animal: As used herein, the term “animal” refers to any member of the animal kingdom. In certain embodiments, “animal” refers to humans at any stage of development. In certain embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In certain embodiments, animals comprise, but are not limited to, mammals, birds, reptiles, amphibians, fish, and worms. In certain embodiments, the animal is a transgenic animal, genetically-engineered animal, or a clone.

Antisense strand: As used herein, the term “the antisense strand” or “the first strand” or “the guide strand” of a siRNA molecule refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process.

Approximately: As used herein, the term “approximately” or “about,” as applied to one or more values of interest, refers to a value that is similar to a stated reference value. As used herein, the term “about” means+/−10% of the recited value. In certain embodiments, the term “approximately” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization-based connectivity sufficiently stable such that the “associated” entities remain physically associated.

Baculoviral expression vector (BEV): As used herein a BEV is a baculoviral expression vector, e.g., a polynucleotide vector of baculoviral origin. A baculovirus expression vector (BEV) is a recombinant baculovirus that has been genetically modified to lead the expression of a foreign gene. Systems using BEVs are known as baculoviral expression vector systems (BEVSs).

mBEV or modified BEV: As used herein, a modified BEV is an expression vector of baculoviral origin which has been altered from a starting BEV (whether wild type or artificial), e.g., by the disruption, addition and/or deletion and/or duplication and/or inversion of one or more: genes; gene fragments; cleavage sites; restriction sites; sequence regions; sequence(s) encoding a payload or gene of interest; or combinations of the foregoing.

Baculovirus genome: As used herein a “baculovirus genome” comprises a wild-type or altered baculovirus or portion thereof. In some embodiments, the wild-type or altered baculovirus comprises a Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV). In some embodiments, a variant baculovirus genome is an altered baculovirus genome or a portion thereof.

BIIC: As used herein a BIIC is a baculoviral infected insect cell.

Capsid: As used herein, the term “capsid” refers to the exterior, e.g., a protein shell, of a virus particle, e.g., an AAV particle, that is substantially (e.g., >50%, >60%, >70%, >80%, >90%, >95%, >99%, or 100%) protein. In some embodiments, the capsid is an AAV capsid comprising an AAV capsid protein described herein, e.g., a VP1, VP2, and/or VP3 polypeptide. The AAV capsid protein can be a wild-type AAV capsid protein or a variant, e.g., a structural and/or functional variant from a wild-type or a reference capsid protein, referred to herein as an “AAV capsid variant.” In some embodiments, the AAV capsid variant described herein has the ability to enclose, e.g., encapsulate, a viral genome and/or is capable of entry into a cell, e.g., a mammalian cell. In some embodiments, the AAV capsid variant described herein may have modified tropism compared to that of a wild-type AAV capsid, e.g., the corresponding wild-type capsid.

Codon optimized: As used herein, the terms “codon optimized” or “codon optimization” refers to a modified nucleic acid sequence which encodes the same amino acid sequence as a parent/reference sequence, but which has been altered such that the codons of the modified nucleic acid sequence are optimized or improved for expression in a particular system (such as a particular species or group of species). As a non-limiting example, a nucleic acid sequence which comprises an AAV capsid protein can be codon optimized for expression in insect cells or in a particular insect cell such Spodoptera frugiperda cells.

Complementary and substantially complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can form base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present disclosure, the ability to substitute a T is implied, unless otherwise stated. Perfect complementarity or 100% complementarity refers to the situation in which each nucleotide unit of one polynucleotide strand can form hydrogen bond with a nucleotide unit of a second polynucleotide strand. Less than perfect complementarity refers to the situation in which some, but not all, nucleotide units of two strands can form hydrogen bond with each other. For example, for two 20-mers, if only two base pairs on each strand can form hydrogen bond with each other, the polynucleotide strands exhibit 10% complementarity. In the same example, if 18 base pairs on each strand can form hydrogen bonds with each other, the polynucleotide strands exhibit 90% complementarity.

Conserved: As used herein, the term “conserved” refers to nucleotides or amino acid residues of a polynucleotide sequence or polypeptide sequence, respectively, that are those that occur unaltered in the same position of two or more sequences being compared. Nucleotides or amino acids that are relatively conserved are those that are conserved amongst more related sequences than nucleotides or amino acids appearing elsewhere in the sequences.

In certain embodiments, two or more sequences are said to be “completely conserved” if they are 100% identical to one another. In certain embodiments, two or more sequences are said to be “highly conserved” if they are at least 70% identical, at least 80% identical, at least 90% identical, or at least 95% identical to one another. In certain embodiments, two or more sequences are said to be “highly conserved” if they are about 70% identical, about 80% identical, about 90% identical, about 95%, about 98%, or about 99% identical to one another. In certain embodiments, two or more sequences are said to be “conserved” if they are at least 30% identical, at least 40% identical, at least 50% identical, at least 60% identical, at least 70% identical, at least 80% identical, at least 90% identical, or at least 95% identical to one another. In certain embodiments, two or more sequences are said to be “conserved” if they are about 30% identical, about 40% identical, about 50% identical, about 60% identical, about 70% identical, about 80% identical, about 90% identical, about 95% identical, about 98% identical, or about 99% identical to one another. Conservation of sequence may apply to the entire length of a polynucleotide or polypeptide or may apply to a portion, region or feature thereof.

Conservative amino acid substitution: As used herein, a “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

Control Elements: As used herein, “control elements”, “regulatory control elements” or “regulatory sequences” refers to promoter regions, polyadenylation signals, transcription termination sequences, upstream regulatory domains, origins of replication, internal ribosome entry sites (“IRES”), enhancers, and the like, which provide for the replication, transcription, and translation of a coding sequence in a recipient cell. Not all of these control elements need always be present as long as the selected coding sequence is capable of being replicated, transcribed and/or translated in an appropriate host cell.

Delivery: As used herein, “delivery” refers to the act or manner of delivering an AAV particle, a compound, substance, entity, moiety, cargo, or payload.

Encapsulate: As used herein, the term “encapsulate” means to enclose, surround, or encase.

Engineered: As used herein, embodiments of the present disclosure are “engineered” when they are designed to have a feature or property, whether structural or chemical, that varies from a starting point, wild-type or native molecule.

Effective Amount: As used herein, the term “effective amount” of an agent is that amount sufficient to effect beneficial or desired results, for example, clinical results, and, as such, an “effective amount” depends upon the context in which it is being applied. For example, in the context of administering an agent that treats cancer, an effective amount of an agent is, for example, an amount sufficient to achieve treatment, as defined herein, of cancer, as compared to the response obtained without administration of the agent.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

AAV expression construct: As used herein, “AAV expression construct” refers to a polynucleotide comprising nucleotide sequences encoding at least an AAV capsid protein (e.g., a VP1 protein, a VP2 protein, and/or a VP3 protein), and/or an AAV rep protein (e.g., a Rep52, Rep40, Rep68, or Rep78 protein, or a combination thereof). In some embodiments, the AAV expression construct further comprises a nucleotide sequence encoding a payload (e.g., a payload encoding region). In some embodiments, the AAV expression construct comprises at least a portion of a baculovirus genome (e.g. a variant baculovirus genome).

ExpressionBac: As used herein, “expressionBac” or “rep/cap bac” refers to a an AAV expression construct and/or region comprising a baculovirus genome. In some embodiments, the AAV expression construct comprising the expressionBac comprises one or more polynucleotides encoding capsid and/or replication genes for an AAV, such as but not limited to AAV2. For example, the one or more polynucleotides encoding capsid and/or replication genes for an AAV may encode VP1, VP2, VP3, Rep52, and/or Rep78, and these polynucleotides may be present in the construct in one or more open reading frames, e.g., in two open reading frames.

Expression BIIC: As used herein, “expression BIIC” or “rep/cap BIIC” refers to an insect cell comprising an AAV expression construct comprising a baculovirus genome (e.g., expressionBac). In some embodiments, the insect cell is an Sf9 cell.

Formulation: As used herein, a “formulation” comprises at least one AAV particle and a delivery agent or excipient.

Fragment: A “fragment,” as used herein, refers to a portion. For example, fragments of proteins may comprise polypeptides obtained by digesting full-length protein isolated from cultured cells.

Functional: As used herein, a “functional” biological molecule is a biological molecule in a form in which it exhibits a property and/or activity by which it is characterized.

Gene expression: The term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Homology: As used herein, the term “homology” refers to the overall relatedness between polymeric molecules, e.g., between polynucleotide molecules (e.g., DNA molecules and/or RNA molecules) and/or between polypeptide molecules. In certain embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the present disclosure, two polynucleotide sequences are considered to be homologous if the polypeptides they encode are at least about 50%, 60%, 70%, 80%, 90%, 95%, or even 99% for at least one stretch of at least about 20 amino acids. In certain embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the present disclosure, two protein sequences are considered to be homologous if the proteins are at least about 50%, 60%, 70%, 80%, or 90% identical for at least one stretch of at least about 20 amino acids.

Identity: As used herein, the term “identity” refers to the overall relatedness between polymeric molecules, e.g., between polynucleotide molecules (e.g., DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; the contents of which are each incorporated herein by reference in their entireties, insofar as they do not conflict with the present disclosure. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences comprise, but are not limited to those disclosed in Carillo, H., and Lipman, D., SIAM J Applied Math., 48:1073 (1988); the contents of which are each incorporated herein by reference in their entireties, insofar as they does not conflict with the present disclosure. Techniques for determining identity are codified in publicly available computer programs. Exemplary computer software to determine homology between two sequences comprise, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically, a reduction in the level of an mRNA results in a reduction in the level of a polypeptide translated therefrom. The level of expression may be determined using standard techniques for measuring mRNA or protein.

Isolated: As used herein, the term “isolated” refers to a substance or entity that is altered or removed from the natural state, e.g., altered or removed from at least some of component with which it is associated in the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell. Such polynucleotides could be part of a vector and/or such polynucleotides or polypeptides could be part of a composition, and still be isolated in that such vector or composition is not part of the environment in which it is found in nature. In some embodiments, an isolated nucleic acid is recombinant, e.g., incorporated into a vector.

Linker: As used herein “linker” refers to a molecule or group of molecules which connects two molecules. A linker may be a nucleic acid sequence connecting two nucleic acid sequences encoding two different polypeptides. The linker may or may not be translated. The linker may be a cleavable linker.

MicroRNA (miRNA) binding site: As used herein, a microRNA (miRNA) binding site represents a nucleotide location or region of a nucleic acid transcript to which at least the “seed” region of a miRNA binds.

Modified: As used herein “modified” refers to a changed state or structure of a molecule of the present disclosure. Molecules may be modified in many ways comprising chemically, structurally, and functionally. As used herein, embodiments of the disclosure are “modified” when they have or possess a feature or property, whether structural or chemical, that varies from a starting point, wild type or native molecule.

Mutation: As used herein, the term “mutation” refers to any changing of the structure of a gene, resulting in a variant (also called “mutant”) form. In some embodiments, mutations in a gene may be caused by the alternation of single base in DNA, or the deletion, insertion, or rearrangement of larger sections of genes or chromosomes.

Naturally Occurring: As used herein, “naturally occurring” or “wild-type” means existing in nature without artificial aid, or involvement of the hand of man.

Non-human vertebrate: As used herein, a “non-human vertebrate” comprises all vertebrates except Homo sapiens, comprising wild and domesticated species. Examples of non-human vertebrates comprise, but are not limited to, mammals, such as alpaca, banteng, bison, camel, cat, cattle, deer, dog, donkey, gayal, goat, guinea pig, horse, llama, mule, pig, rabbit, reindeer, sheep water buffalo, and yak.

Nucleic Acid: As used herein, the term “nucleic acid”, “polynucleotide” and ‘oligonucleotide” refer to any nucleic acid polymers composed of either polydeoxyribonucleotides (containing 2-deoxy-D-ribose), or polyribonucleotides (containing D-ribose), or any other type of polynucleotide which is an N glycoside of a purine or pyrimidine base, or modified purine or pyrimidine bases. There is no intended distinction in length between the term “nucleic acid”, “polynucleotide” and “oligonucleotide”, and these terms will be used interchangeably. These terms refer only to the primary structure of the molecule. Thus, these terms comprise double- and single-stranded DNA, as well as double- and single stranded RNA.

Open reading frame: As used herein, “open reading frame” or “ORF” refers to a sequence which does not contain a stop codon within the given reading frame, other than at the end of the reading frame.

Operably linked: As used herein, the phrase “operably linked” refers to a functional connection between two or more molecules, constructs, transcripts, entities, moieties, or the like. As a non-limiting example, a promoter is “operably linked” to a nucleotide sequence when the promoter sequence controls and/or regulates the transcription of the nucleotide sequence.

Payload: As used herein, “payload” or “payload region” refers to one or more polynucleotides or polynucleotide regions encoded by or within a viral genome or an expression product of such polynucleotide or polynucleotide region, e.g., a transgene, a polynucleotide encoding a polypeptide or multi-polypeptide, or a modulatory nucleic acid or regulatory nucleic acid.

PayloadBac: As used herein, “payloadBac” refers to a baculovirus comprising a payload construct and/or region. In some embodiments, the payload construct and/or region of the payloadBac comprises a polynucleotide encoding the payload.

Payload BIIC: As used herein, “payloadBIIC” refers to an insect cell comprising one or more baculovirus (e.g., payloadBac) comprising a payload construct and/or region. In some embodiments, the payload construct and/or region comprises a polynucleotide encoding the payload. In some embodiments, the insect cell is an Sf9 cell.

Payload construct: As used herein, “payload construct” is one or more vector construct which comprises a polynucleotide region encoding or comprising a payload that is flanked on one or both sides by an inverted terminal repeat (ITR) sequence. The payload construct may present a template that is replicated in a viral production cell to produce a therapeutic viral genome.

Payload construct vector: As used herein, “payload construct vector” is a vector encoding or comprising a payload construct, and regulatory regions for replication and expression of the payload construct in bacterial cells.

Payload construct expression vector: As used herein, a “payload construct expression vector” is a vector encoding or comprising a payload construct and which further comprises one or more polynucleotide regions encoding or comprising components for viral expression in a viral replication cell.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient.

Pharmaceutically acceptable salts: The present disclosure also comprises pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid).

Pharmaceutically acceptable solvate: The term “pharmaceutically acceptable solvate,” as used herein, means a compound of the present disclosure wherein molecules of a suitable solvent are incorporated in the crystal lattice.

Preventing: As used herein, the term “preventing” or “prevention” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Purified: As used herein, “purify,” “purified,” “purification” means to make substantially pure or clear from unwanted components, material defilement, admixture or imperfection. “Purified” refers to the state of being pure. “Purification” refers to the process of making pure.

Region: As used herein, the term “region” refers to a zone or general area. In certain embodiments, when referring to a protein or protein module, a region may comprise a linear sequence of amino acids along the protein or protein module or may comprise a three-dimensional area, an epitope and/or a cluster of epitopes. In certain embodiments, regions comprise terminal regions. As used herein, the term “terminal region” refers to regions located at the ends or termini of a given agent. When referring to proteins, terminal regions may comprise N- and/or C-termini N-termini refer to the end of a protein comprising an amino acid with a free amino group. C-termini refer to the end of a protein comprising an amino acid with a free carboxyl group. N- and/or C-terminal regions may there for comprise the N- and/or C-termini as well as surrounding amino acids. In certain embodiments, N- and/or C-terminal regions comprise from about 3 amino acid to about 30 amino acids, from about 5 amino acids to about 40 amino acids, from about 10 amino acids to about 50 amino acids, from about 20 amino acids to about 100 amino acids and/or at least 100 amino acids. In certain embodiments, N-terminal regions may comprise any length of amino acids that comprises the N-terminus but does not comprise the C-terminus. In certain embodiments, C-terminal regions may comprise any length of amino acids, which comprise the C-terminus, but do not comprise the N-terminus.

In certain embodiments, when referring to a polynucleotide, a region may comprise a linear sequence of nucleic acids along the polynucleotide or may comprise a three-dimensional area, secondary structure, or tertiary structure. In certain embodiments, regions comprise terminal regions. As used herein, the term “terminal region” refers to regions located at the ends or termini of a given agent. When referring to polynucleotides, terminal regions may comprise 5′ and 3′ termini 5′ termini refer to the end of a polynucleotide comprising a nucleic acid with a free phosphate group. 3′ termini refer to the end of a polynucleotide comprising a nucleic acid with a free hydroxyl group. 5′ and 3′ regions may there for comprise the 5′ and 3′ termini as well as surrounding nucleic acids. In certain embodiments, 5′ and 3′ terminal regions comprise from about 9 nucleic acids to about 90 nucleic acids, from about 15 nucleic acids to about 120 nucleic acids, from about 30 nucleic acids to about 150 nucleic acids, from about 60 nucleic acids to about 300 nucleic acids and/or at least 300 nucleic acids. In certain embodiments, 5′ regions may comprise any length of nucleic acids that comprises the 5′ terminus but does not comprise the 3′ terminus. In certain embodiments, 3′ regions may comprise any length of nucleic acids, which comprise the 3′ terminus, but does not comprise the 5′ terminus.

RNA or RNA molecule: As used herein, the term “RNA” or “RNA molecule” or “ribonucleic acid molecule” refers to a polymer of ribonucleotides; the term “DNA” or “DNA molecule” or “deoxyribonucleic acid molecule” refers to a polymer of deoxyribonucleotides. DNA and RNA can be synthesized naturally, e.g., by DNA replication and transcription of DNA, respectively; or be chemically synthesized. DNA and RNA can be single-stranded (i.e., ssRNA or ssDNA, respectively) or multi-stranded (e.g., double stranded, i.e., dsRNA and dsDNA, respectively). The term “mRNA” or “messenger RNA”, as used herein, refers to a single stranded RNA that encodes the amino acid sequence of one or more polypeptide chains.

RNA interfering or RNAi: As used herein, the term “RNA interfering” or “RNAi” refers to a sequence specific regulatory mechanism mediated by RNA molecules which results in the inhibition or interfering or “silencing” of the expression of a corresponding protein-coding gene. RNAi has been observed in many types of organisms, comprising plants, animals and fungi. RNAi occurs in cells naturally to remove foreign RNAs (e.g., viral RNAs). Natural RNAi proceeds via fragments cleaved from free dsRNA which direct the degradative mechanism to other similar RNA sequences. RNAi is controlled by the RNA-induced silencing complex (RISC) and is initiated by short/small dsRNA molecules in cell cytoplasm, where they interact with the catalytic RISC component argonaute. The dsRNA molecules can be introduced into cells exogenously. Exogenous dsRNA initiates RNAi by activating the ribonuclease protein Dicer, which binds and cleaves dsRNAs to produce double-stranded fragments of 21-25 base pairs with a few unpaired overhang bases on each end. These short double stranded fragments are called small interfering RNAs (siRNAs).

Self-complementary viral particle: As used herein, a “self-complementary viral particle” is a particle comprised of at least two components, a protein capsid and a polynucleotide sequence encoding a self-complementary genome enclosed within the capsid.

Sense Strand: As used herein, the term “the sense strand” or “the second strand” or “the passenger strand” of a siRNA molecule refers to a strand that is complementary to the antisense strand or first strand. The antisense and sense strands of a siRNA molecule are hybridized to form a duplex structure. As used herein, a “siRNA duplex” comprises a siRNA strand having sufficient complementarity to a section of about 10-50 nucleotides of the mRNA of the gene targeted for silencing and a siRNA strand having sufficient complementarity to form a duplex with the other siRNA strand.

Short interfering RNA or siRNA: As used herein, the terms “short interfering RNA,” “small interfering RNA” or “siRNA” refer to an RNA molecule (or RNA analog) comprising between about 5-60 nucleotides (or nucleotide analogs) which is capable of directing or mediating RNAi. In certain embodiments, a siRNA molecule comprises between about 15-30 nucleotides or nucleotide analogs, such as between about 16-25 nucleotides (or nucleotide analogs), between about 18-23 nucleotides (or nucleotide analogs), between about 19-22 nucleotides (or nucleotide analogs) (e.g., 19, 20, 21 or 22 nucleotides or nucleotide analogs), between about 19-25 nucleotides (or nucleotide analogs), and between about 19-24 nucleotides (or nucleotide analogs). The term “short” siRNA refers to a siRNA comprising 5-23 nucleotides, such as 21 nucleotides (or nucleotide analogs), for example, 19, 20, 21 or 22 nucleotides. The term “long” siRNA refers to a siRNA comprising 24-60 nucleotides, such as about 24-25 nucleotides, for example, 23, 24, 25 or 26 nucleotides. Short siRNAs may, in some instances, comprise fewer than 19 nucleotides, e.g., 16, 17 or 18 nucleotides, or as few as 5 nucleotides, provided that the shorter siRNA retains the ability to mediate RNAi. Likewise, long siRNAs may, in some instances, comprise more than 26 nucleotides, e.g., 27, 28, 29, 30, 35, 40, 45, 50, 55, or even 60 nucleotides, provided that the longer siRNA retains the ability to mediate RNAi or translational repression absent further processing, e.g., enzymatic processing, to a short siRNA. siRNAs can be single stranded RNA molecules (ss-siRNAs) or double stranded RNA molecules (ds-siRNAs) comprising a sense strand and an antisense strand which hybridized to form a duplex structure called siRNA duplex.

Signal Sequences: As used herein, the phrase “signal sequences” refers to a sequence which can direct the transport or localization of a protein.

Similarity: As used herein, the term “similarity” refers to the overall relatedness between polymeric molecules, e.g., between polynucleotide molecules (e.g., DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Subject: As used herein, the term “subject” or “patient” refers to any organism to which a composition in accordance with the present disclosure may be administered, e.g., for experimental, diagnostic, prophylactic, and/or therapeutic purposes. Typical subjects comprise animals (e.g., mammals such as mice, rats, rabbits, non-human primates, and humans) and/or plants. The subject or patient may seek or need treatment, require treatment, is receiving treatment, will receive treatment, or is under care by a trained professional for a particular disease or condition.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Synthetic: As used herein, the term “synthetic” or “chemically synthesized” in the context of a nucleic acid sequence refers to a nucleic acid molecule that is, at least in part, formed through a chemical process, as opposed to molecules of natural origin, or molecules derived via template-based amplification of molecules of natural origin. In some embodiments, chemically-synthesized DNA is non-templated (e.g., the sequence is arbitrarily decided and does not physically depend on a parental sequence), unlike natural DNA replication or in vitro polymerase reactions like PCR.

Targeting: As used herein, “targeting” means the process of design and selection of nucleic acid sequence that will hybridize to a target nucleic acid and induce a desired effect.

Targeted Cells: As used herein, “targeted cells” refers to any one or more cells of interest. The cells may be found in vitro, in vivo, in situ or in the tissue or organ of an organism. The organism may be an animal, such as a mammal, a human, or a human patient.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition. In certain embodiments, a therapeutically effective amount is provided in a single dose. In certain embodiments, a therapeutically effective amount is administered in a dosage regimen comprising a plurality of doses. Those skilled in the art will appreciate that in certain embodiments, a unit dosage form may be considered to comprise a therapeutically effective amount of a particular agent or entity if it comprises an amount that is effective when administered as part of such a dosage regimen.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

Variant: As used herein, the term “variant” refers to a polypeptide or polynucleotide that has an amino acid or a nucleotide sequence that is substantially identical, e.g., having at least 70%, 75%, 80%, 85%, 90%, 95% or 99% sequence identity to a reference sequence. In some embodiments, the variant is a functional variant.

Functional Variant: As used herein, the term “functional variant” refers to a polypeptide variant or a polynucleotide variant that has at least one activity of the reference sequence.

Vector: As used herein, the term “vector” refers to any molecule or moiety which transports, transduces or otherwise acts as a carrier of a heterologous molecule. In some embodiments, vectors may be plasmids. In some embodiments, vectors may be viruses. An AAV particle is an example of a vector. Vectors of the present disclosure may be produced recombinantly and may be based on and/or may comprise adeno-associated virus (AAV) parent or reference sequences. The heterologous molecule may be a polynucleotide and/or a polypeptide.

Viral genome: As used herein, the term “viral genome” refers to the nucleic acid sequence(s) encapsulated in an AAV particle. A viral genome comprises a nucleic acid sequence with at least one payload region encoding a payload and at least one ITR.

Those skilled in the art will recognize or be able to ascertain using no more than routine experimentation, many equivalents to the specific embodiments in accordance with the present disclosure described herein. The scope of the present disclosure is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The present disclosure includes embodiments in which exactly one member of the group is present in, employed in, or otherwise relevant to a given product or process. The present disclosure includes embodiments in which more than one, or the entire group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the present disclosure, to the tenth of the unit of the lower limit of the range, unless the context clearly dictates otherwise.

In addition, it is to be understood that any particular embodiment of the present disclosure that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the present disclosure (e.g., any antibiotic, therapeutic or active ingredient; any method of production; any method of use; etc.) can be excluded from any one or more claims, for any reason, whether or not related to the existence of prior art.

It is to be understood that the words which have been used are words of description rather than limitation, and that changes may be made within the purview of the appended claims without departing from the true scope and spirit of the present disclosure in its broader aspects.

While the present disclosure has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the present disclosure.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

### Examples

### Example 1. Polh-Rep78-Only and Polh-Rep52-Only Polynucleotides

### Polh-Rep78-Only

Polynucleotide cassettes were engineered to include a polh promoter (very late) and a Rep78-only coding sequence (i.e., very little translation of Rep52 protein from Rep78 ORF). The polynucleotide was engineered to be inserted/cloned into suitable baculovirus plasmids or vectors using Gibson Assembly methods. A polh-Rep78-only polynucleotide (with Gibson Assembly sequences) is presented in SEQ ID NO: 9.

### Polh-Rep52-Only

Polynucleotide cassettes were engineered to include a polh promoter (very late) and a Rep52-only coding sequence (i.e., includes the entire coding sequence of the Rep52 protein from wild-type AAV2, but does not include the normally upstream coding sequence of the Rep78 protein). The polh promoter was included to drive the expression of the Rep52 coding sequence. The polynucleotide was engineered to be inserted/cloned into suitable baculovirus plasmids or vectors using Gibson Assembly methods. A polh-Rep52 polynucleotide (with Gibson Assembly sequences) is presented in SEQ ID NO: 10.

### Example 2. Yield of AAV Produced Using Rep78/Rep52-Separated Bacmids

Studies were completed to investigate the yield of AAV particle production using viral expression constructs which included a range of configurations for Rep78-coding regions and Rep52-coding regions within the production bacmids, including standard bicistronic ORFs (i.e., sequences encoding both Rep78 and Rep52) and split ORFs (i.e., Rep78-only coding regions in a first location and Rep52-only coding regions in a second location). expressionBacs were prepared according to Table 1 (N/A—Indicates that the specific coding region was not included in expressionBac construct).

TABLE 1
expressionBac Designs

| Cul- | v-cath | Rep78-only | | Rep52-only | |
|---|---|---|---|---|---|
| ture | deletion | coding | | coding | |
| Num- | in | region | Rep78/Rep52 | region | VP |
| ber | bacmid | location | location | location | capsid |
| 1 | YES | N/A | N/A | N/A | N/A |
| 2 | YES | N/A | N/A | egt | N/A |
| 3 | YES | Tn7 | N/A | N/A | AAV1 |
| 4 | YES | N/A | Tn7 (bicistronic) | N/A | AAV1 |
| 5 | YES | Tn7 | N/A | egt | AAV1 |
| 6 | NO | N/A | Tn7 (bicistronic) | N/A | AAV1 |
| 7 | YES | N/A | Tn7 (bicistronic) | N/A | AAV2 |
| 8 | YES | Tn7 | N/A | egt | AAV2 |
| 9 | NO | N/A | Tn7 (bicistronic) | N/A | AAV2 |

Sf9 cells were seeded into 50 mL conical flasks and expanded in 1 mL cultures up to a VPC density of about 2×10⁶ vc/mL. 5 MOI of payloadBac material (cmvGFP transgene payload) and 5 MOI of each expressionBac sample were combined in individual flasks, and incubated for about 30 hours. Each flask was topped to 50 mL with sterile PBS containing pluronic F-68. Cells were pelleted by centrifugation at 300 g for 8 min, followed by aspiration of the supernatant. Pellets were resuspended in 50 mL with sterile PBS containing pluronic F-68 (by mixture and shaking), and again centrifuged at 300 g for 8 min. Isolated cell pellets were then resuspended in 6 mL of ESF media.

Each test culture flask was then split into 5 replicate flasks (1.1, 1.2, 1.3, 1.4, 1.5, etc.) to provide 45 replicate test culture flasks in total. The flasks were shaken at 250 RPM and 27° C. for about for about 90 hours. Cells were then lysed using 58.8 μL of 10% TritonX-100 (0.5% final) and 117.6 μL of 2.0 M arginine (0.2 M final), followed by 0.047 μL of Benzonase (250 U per uL) (10 U per mL final), with agitated incubation (220 RPM and 37° C.) for about 30 hours.

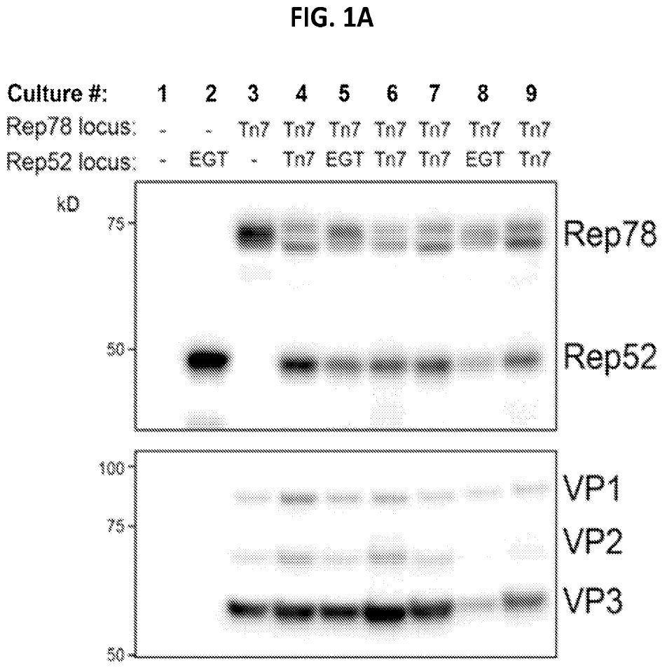

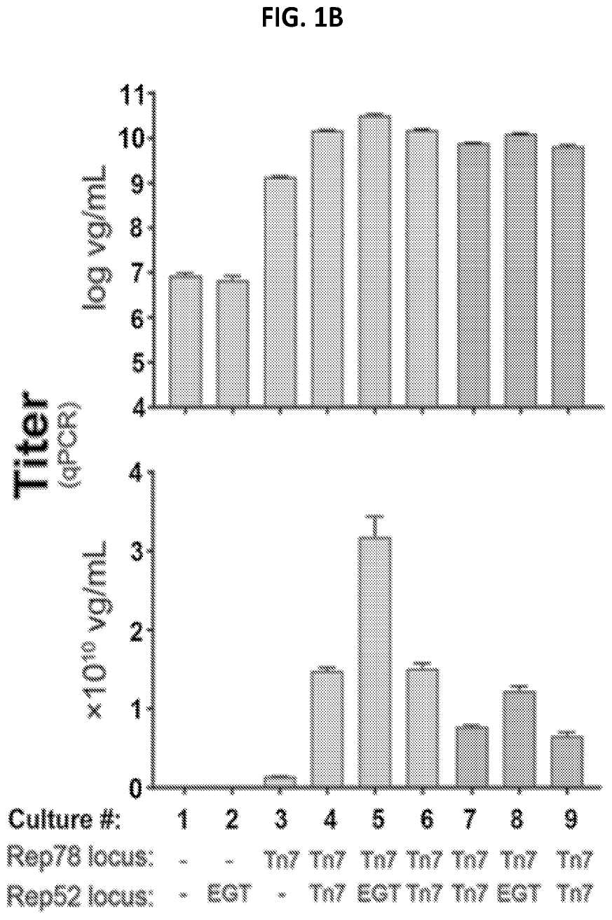

65 μL samples from each test culture replicate flask were extracted and combined with 10 μL 10× NuPage reducing agent+25 μL 4× NuPage sample loading buffer to provide 100 μL protein samples. Each test culture protein sample was centrifuged at 4000 g for 10 minutes, and resulting pellets were analyzed by Western blot analysis for AAV Rep and AAV Cap proteins. Results of the Western blot analysis are shown in FIG. 1A. Western blot analysis confirmed Rep protein and VP protein production from each test culture, though none of the expressionBac designs provided for strong production of both Rep78 and Rep52 proteins.

Each test culture was also analyzed for AAV titer using qPCR (assuming self-complementary vector genomes per mL). Results of the AAV titer qPCR analysis are shown in FIG. 1B. Results showed that split Rep protein ORFs provided for improved AAV vector genome titer when compared to standard bicistronic Rep protein ORFs, both for AAV1 and AAV2 capsid particles.

### Example 3. Expression-Modifying Minicistron Inserts

Studies were completed to investigate the effect of using expression-modifying minicistron inserts with Rep78/Rep52 bicistronic expression cassettes in Rep protein production.

Polynucleotide cassettes were engineered to include a polh promoter (very late), a minicistron insert (SEQ ID NO: 4) and a Rep78/Rep52 bicistronic coding sequence. Five different constructs were prepared with minicistron inserts having varying proximity (i.e., intermediate nucleotides) from the Rep78 start codon, ATG. A sixth construct was prepared with an inactive minicistron insert (SEQ ID NO: 11). A Control cassette was also prepared with Rep78/Rep52 bicistronic coding sequence, a CTG start codon for Rep78, and no minicistron insert. Each of the six polh_MC_atgRep78 cassettes and the Control cassette were then incorporated into an expressionBac for further study. Table 2 presents further details of the polh_MC_atgRep78 and Control constructs designed for study (“Proximity” in Table 2 represents the separation of the minicistron insert from the ATG start codon, i.e., number of intermediate nucleotide base pairs).

TABLE 2
polh_MC_atgRep78 Designs

| Construct | minicistron insert | minicistron insert | | polh_MC_atgRep78 |
|---|---|---|---|---|
| Number | (SEQ ID NO) | proximity (bp) | | (SEQ ID NO) |
| MC1 | 4 | 3 | bp | 12 |
| MC2 | 4 | 20 | bp | 13 |
| MC3 | 4 | 37 | bp | 14 |
| MC4 | 4 | 54 | bp | 15 |
| MC5 | 4 | 71 | bp | 16 |
| MCX | 11 | 3 | bp | 17 |

| | |
|---|---|
| CON- | Rep78/Rep52 bicistronic coding sequence; |
| TROL | ctg start codon; No minicistron insert |

Sf9 cells were seeded into 50 mL conical flasks and expanded in 2 mL cultures up to a VPC density of about 3×10⁶ vc/mL. 5 MOI of payloadBac material (cmvGFP transgene payload) and 5 MOI of each of the seven expressionBac samples were combined in individual flasks, and incubated for about 96 hours. Cells were harvested from each cell and lysed, followed by protein pelleting and Western blot analysis for VP and Rep78/Rep52 protein production.

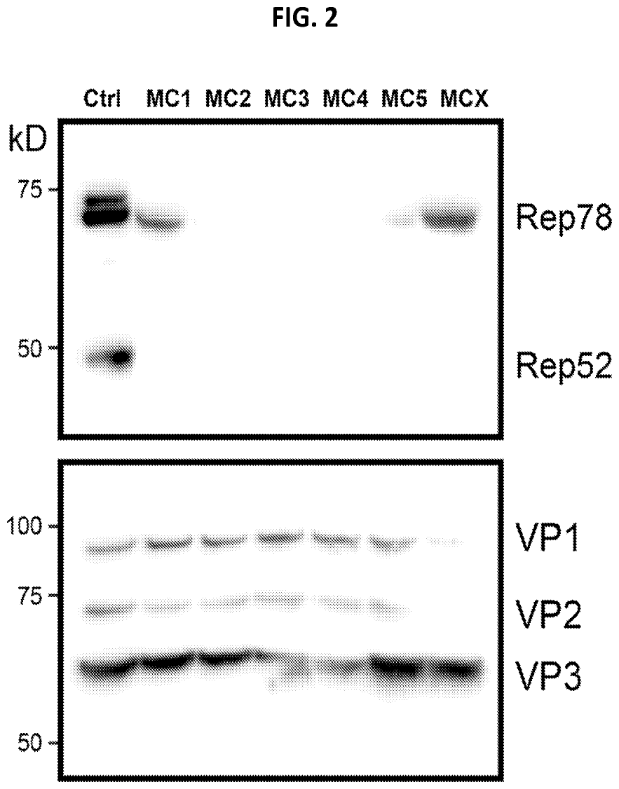

Results of the Western blot analysis are shown in FIG. 2. Western blot analysis confirmed Rep78 and Rep52 protein production from the Control cassette only. MC1 and MCX cassettes produced observable amounts of Rep78, but no measurable Rep52. MC5 cassette produced small amounts of Rep78 and no measurable Rep52. MC2, MC3, and MC4 cassettes produced no measurable Rep78 or Rep52.

### Example 4. Split Polh-MC-Rep78/Rep52-Only ORFs

Studies were completed to investigate AAV particle production using viral expression constructs which included a range of configurations for Rep78-coding regions and Rep52-coding regions within the production bacmids, including: (i) a prototype split ORF construct, which included a polh-atgRep78-only cassette in the Tn7 locus of the bacmid and a polh-atgRep52-only cassette in the egt locus of the bacmid; (ii) an MC split ORF construct, which included a polh-MC-atgRep78-only cassette (SEQ ID NO: 12) in the Tn7 locus of the bacmid and a polh-atgRep52-only cassette in the egt locus of the bacmid; and (iii) an Opie1 split ORF construct, which included a Opie1-atgRep78-only cassette in the Tn7 loci of the bacmid and a polh-atgRep52-only cassette in the egt locus of the bacmid. An FIH Control bacmid was also prepared with a polh-ctgRep78/Rep52 bicistronic coding sequence and no minicistron insert.

### Rep and Cap Expression

sf9 cells were seeded into flasks and expanded in 200 mL cultures up to a VPC density of about 2.8×10⁶ vc/mL. 0.002 MOI (TCID50 per cell) of payloadBac material (HTT-targeting siRNA payload) and 0.002 MOI (TCID50 per cell) of each of the four expressionBacs were combined in individual flasks, and incubated for about 6 days. Samples from each flask were collected 3 days, 4 days, 5 days, and 6 days post infection.

Cell samples spun down at 4000 g, then lysed in 200 μL of the following mix: 650 μL H₂O+250 μL 4× NuPage sample buffer+100 μL 10× NuPage reducing agent. Resulting samples were boiled at 80° C. for 10 min and then vortexed heavily. Samples were then process through Western blot analysis for VP and Rep78/Rep52 protein production.

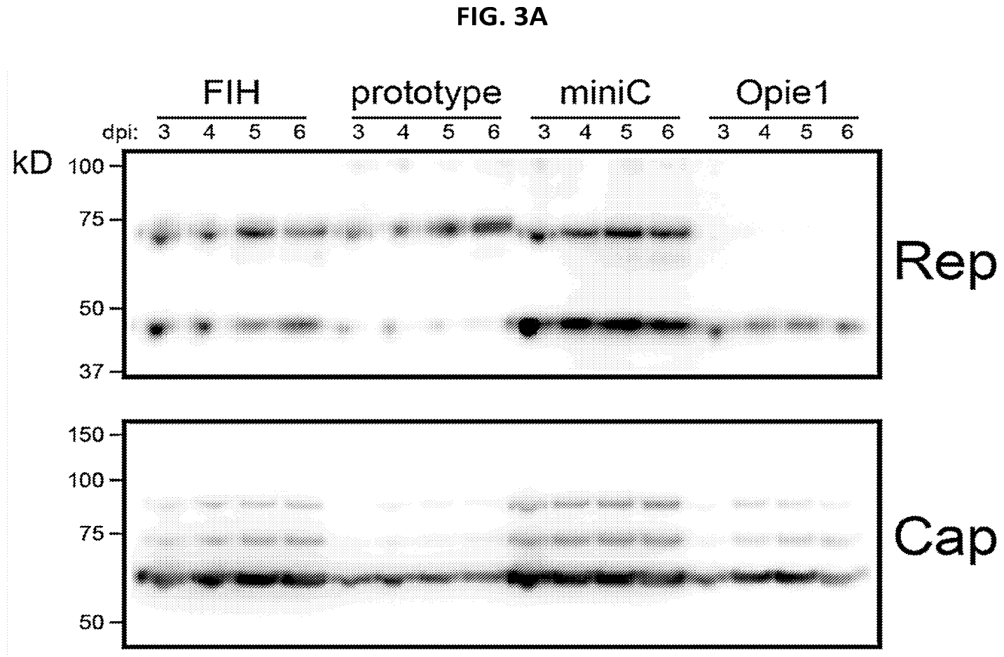

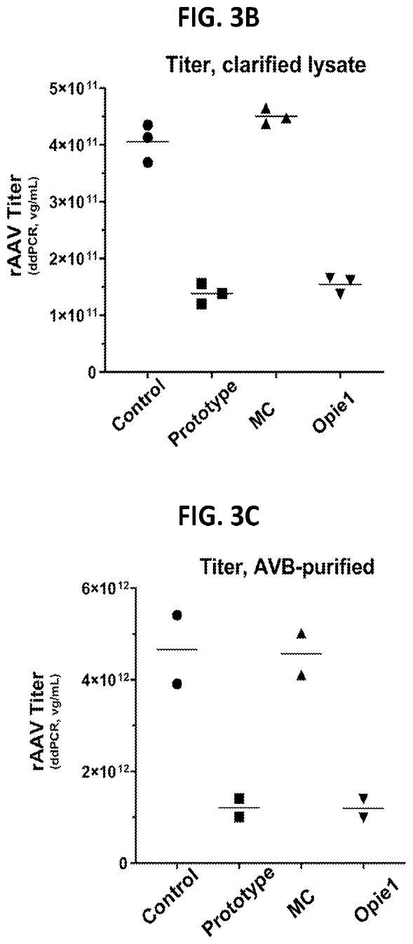

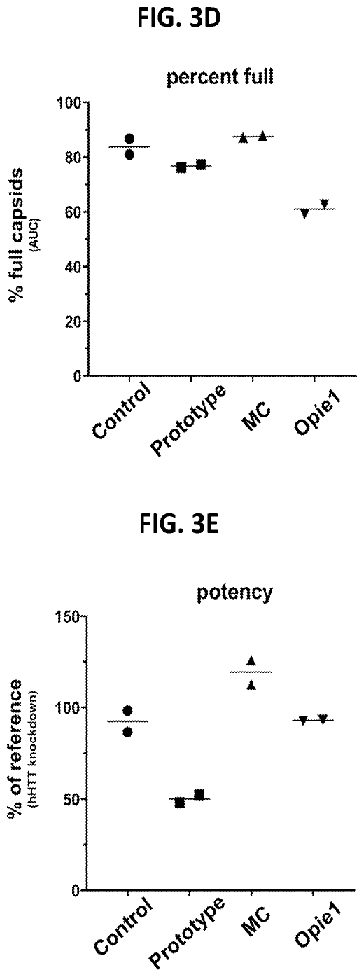

Results of the Western blot analysis are shown in FIG. 3A. Western blot analysis confirmed Rep and Cap protein production from all four expressionBacs. The MC split ORF construct showed Rep and Cap protein production comparable to the FIH control bacmid. The MC split ORF construct also showed clear increased Rep and Cap protein production compared to the prototype split ORF construct (which showed reduced Rep52 production and Cap production) and the Opie1 split ORF construct (which showed minimal Rep78 production and reduced Cap production).

### AAV Particle Production

sf9 cells were seeded into flasks and expanded in 200 mL cultures up to a VPC density of about 2.8×10⁶ vc/mL. 0.002 MOI (TCID50 per cell) of payloadBac material (HTT-targeting siRNA payload) and 0.002 MOI (TCID50 per cell) of each of the four expressionBacs (3× samples of each expressionBac in triplicate) were combined in individual flasks, and incubated for about 6 days. Cells were then lysed using 11.7 mL of 10% TritonX-100 (0.5% final) and 22.0 mL of 2.0 M arginine (0.2 M final), followed by 10 μL of Benzonase (250 U per uL) (10 U per mL final), with agitated incubation (220 RPM and 37° C.) for about 12 hours.

Each test culture sample was centrifuged at 4000 g for 15 minutes, and then filtered through 0.22 μm PES membranes. 400 μL of each clarified lysate sample was collected for ddPCR titer analysis (FIG. 3B). Each clarified lysate sample was then processed through AKTA affinity chromatography purification (with one replicate sample from each expressionBac begin discarded for AFB processing errors). Each AFB-purified sample was then sampled again for ddPCR titer analysis (FIG. 3C), % full capsid analysis by analytical ultracentrifugation (AUC) (FIG. 3D), potency by HTT knockdown (FIG. 3E), and VP protein ratios by CE-SDS.

ddPCR titer analysis from clarified lysate samples (FIG. 3B) and AFB-purified samples (FIG. 3C) showed that MC split ORF constructs (Tn7:polh-MC-atgRep78-only/egt:polh-atgRep52-only) produced AAV vector genome titers comparable (or slightly higher) than the FIH Control constructs (Tn7:polh-ctgRep78/Rep52 bicistronic), with both AFB-purified samples showing mean ddPCR titers between 4.5-5.0×10¹² vg/mL. Prototype split ORF constructs (Tn7:polh-atgRep78-only/egt: polh-atgRep52-only) and Opie1 split ORF constructs (Tn7:Opie1-atgRep78-only/egt:polh-atgRep52-only) both showed lower ddPCR titer for both clarified lysate and AFB-purified samples, with AFB-purified samples showing mean ddPCR titers between 1.0-1.5×10¹² vg/mL.

AUC % full capsid analysis of AFB-purified samples (FIG. 3D) showed that MC split ORF constructs and FIH Control constructs both provided mean %-full measurements between 85-90%. Prototype split ORF constructs provided a mean %-full measurement between 75-80%, and Opie1 split ORF constructs provided a mean %-full measurements between 60-65%.

rAAV potency analysis by HTT knockdown relative to a reference (FIG. 3E) showed that MC split ORF constructs provided a mean relative rAAV potency of between about 120-125%. FIH Control constructs and Opie1 split ORF constructs both provided a mean relative rAAV potency of between about 90-95%. Prototype split ORF provided a mean relative rAAV potency of between about 50-55%.

VP protein ratio analysis by CE-SDS are provided in Table 3.

TABLE 3
VP protein ratios

| | | Relative |
|---|---|---|
| Construct | VP3:VP2:VP1 | Purity (%) |
| FIH (1) | 17:01:01 | 98 |
| FIH (2) | 20:02:01 | 99 |
| Prototype (1) | 18:02:01 | 98 |
| Prototype (2) | 19:02:01 | 99 |
| MC (1) | 14:02:01 | 98 |
| MC (2) | 15:02:01 | 99 |
| Opei1 (1) | 19:03:01 | 99 |
| Opei1 (2) | 17:02:01 | 100 |

### Example 5. Production of Bacmid AB189 and AB191

### Production of Bacmid AA965

A baculovirus genome Bacmid AA862, an engineered variant of the bacmid bMON14272 (described in Luckow et al. Efficient generation of infectious recombinant baculoviruses by site-specific transposon-mediated insertion of foreign genes into a baculovirus genome propagated in Escherichia coli. J Virol. 1993 August 67(8):4566-79; the contents of which are hereby incorporated by reference in its entirety), was provided which included the following alterations: (i) v-cath proteinase and chiA chitinase genes were deleted by homologous recombination in Sf9 insect cell culture, with the homology regions mapping to the chiA C terminus and gp64 C terminus being derived from AcMNPV strain C6, rather than parental strain E2.; and (ii) p26, p10, and p74 genes were deleted and replaced with an I-SceI-flanked chloramphenicol-resistance cassette. Plasmids were also provided which included polynucleotide insert cassettes including a polh-atgRep52-only sequence (with Gibson Assembly sequences) is presented in SEQ ID NO: 10.

Bacmid DNA AA862 was digested with AvrII enzyme at 37° C. for 1.5 hours. The polh-atgRep52-only insert fragments were then incorporated into the AvrII-digested AA862 bacmid genomes by Gibson assembly reaction by combining 1.5 μL of digested AA862 bacmid, 3.5 μL of the polh-atgRep52-only product and 5 μL of NEB Hi-Fi Master, followed by mixing for 1 hour at 50° C. Samples were desalted, electroporated, and then plated for colony selection on KanR plates. Bacmid colony AA965 (with a the polh-atgRep52-only expression cassette in the bacmid egt locus) was selected for further engineering work.

### Production of Bacmid AB189

Bacmid AA965 was first processed to remove existing AscI sites and LacZa cassettes in the starting bacmids. 5 μg of Bacmid AA965 was digested with AscI for 1 hour at 37° C. in 10 μL reactions (1× Cutsmart). Bacmids were then re-ligated using the primer of SEQ ID NO: 18, in the following reaction: 4 μL AscI-digseted AA965+1 μL ligation primer (450 nM; SEQ ID NO: 18)+5 μL 2×Hifi Assembly Mix. The mixture was incubated for 1 hour at 50° C., then dialyzed, electroporated, and then plated for colony selection on KAN+IPTG+X-GAL plates. Colony clones AB162 and AB163 were selected for further engineering work.

Midiprep bacmid DNA of clones AB162 and AB163 were prepared using the Macherey-Nagel Xtra Midi kit, yielding about 100 μg of bacmid DNA for each. Both bacmid DNA samples were confirmed to be infectious by transfection into Sf9 GFP-reporter cells. Polynucleotide insert cassettes were also provided which included a polh-MC1-atgRep78-p10-LacZa cassette insert as presented in SEQ ID NO: 19 (with Gibson Assembly sequences), and which included the polh_MC1_atgRep78 sequence of SEQ ID NO: 12.

5 μg of clones AB162 bacmid midiprep was digested with 2 μL I-CeuI enzyme in a 20 μL reaction (1× Cutsmart) for 5 hours at 37° C. The I-CeuI-cut AB162 bacmid was then ligated with the polh-MC1-atgRep78-p10-LacZa cassette insert using the following Gibson Assembly reaction: 5 μL I-CeuI-cut AB162+1 μL polh-MC1-atgRep78-p10-LacZa product (˜40 ng)+6 μL 2× Hifi Assembly Mastermix. The mixture was incubated for 1 hour at 50° C., then dialyzed, electroporated, and then plated for colony selection on KAN+IPTG+X-GAL plates. Colony clones AB178 and AB179 were selected for further analysis and engineering work.

Midiprep bacmid DNA of clones AB178 and AB179 were prepared using the Macherey-Nagel Xtra Midi kit, yielding about 100-130 μg of bacmid DNA for each. Both bacmid DNA samples were confirmed to be infectious by transfection into Sf9 GFP-reporter cells. Insertion of the polh-MC1-atgRep78-p10-LacZa cassette into the v-cath locus of AB178 was also confirmed through PCR extraction and DNA sequencing.

Bacmid AB178 was then processed to remove existing chloramphenicol-resistance cassettes. 5 tig of Bacmid AB178 was digested with I-SceI enzyme for 1 hour at 37° C. in 10 μL reactions (1× Cutsmart), followed by heat inactivation at 65° C. for 20 minutes. 5 μL of the I-SceI-cut AB178 was then re-ligated in a 20 μL T4 ligase reaction (1×T4 ligase buffer). The mixture was incubated for 30 minutes at 20° C., then dialyzed, electroporated, and plated for colony selection on KAN plates. Colony clones AB189 and AB190 were selected for further analysis.

Midiprep bacmid DNA of clones AB189 and AB190 were prepared using the Macherey-Nagel Xtra Midi kit, yielding about 100 μg of bacmid DNA for each. Both bacmid DNA samples were confirmed to be infectious by transfection into Sf9 GFP-reporter cells. Deletion of the CAM cassette was confirmed using I-SceI digestion, followed by gel analysis comparison with Bacmid AB178.

### Testing of Bacmid AB189

Bacmid AB189 was tested using AAV capsids sequence inserts from 13 different AAV Capsid serotypes, including AAV1, AAV2, AAV8, AAV9, and several other AAV9 variants.

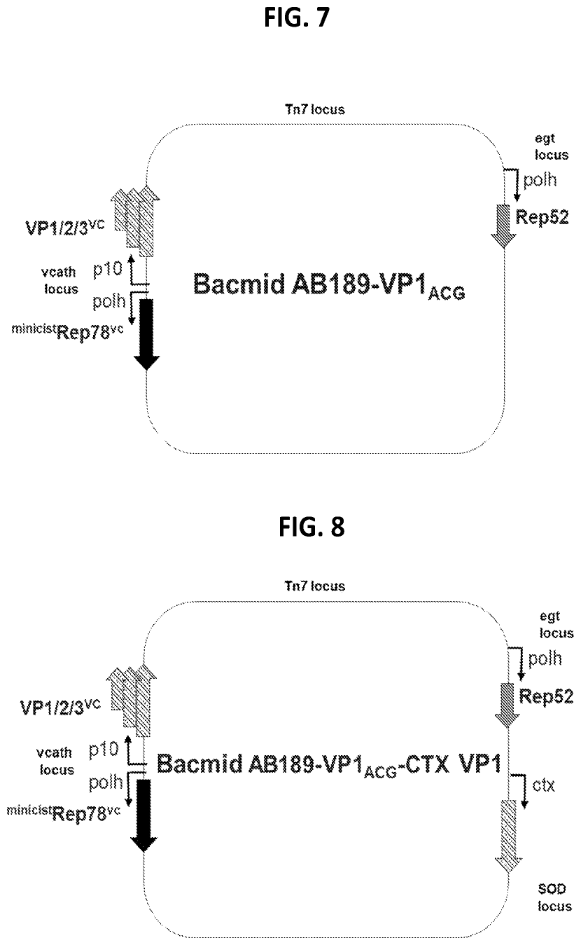

The resulting Bacmid AB189 comprising the capsid sequences, Bacmid AB189-VP1_{ACG}, as shown in FIG. 7, comprised Rep78 and Rep52 coding regions in two separate loci (e.g., distinct transcriptional cassettes) in the bacmid as well as overlapping VP 1, 2, and 3 coding regions (single polycistronic ORF) present in a single loci. The Rep78 and overlapping VP coding regions were both inserted into the v-cath locus, where the Rep78 coding region was under the control of a polyhedrin (polh) promoter and the overlapping VP regions were under the control of a p10 promoter. A minicistron was inserted upstream of the Rep78 ORF to further modulate expression of the Rep78 protein. In order to control expression of the VP proteins, the start codons for the VP1 and the VP2 coding region were an ACG start codon, and the start codon for the VP3 coding region was an ATG start codon. The Rep52 coding region was inserted into the egt locus under the control of a polh promoter. The chiA, p26, p10, and p74 genes were also deleted from the genome of Bacmid AB189.

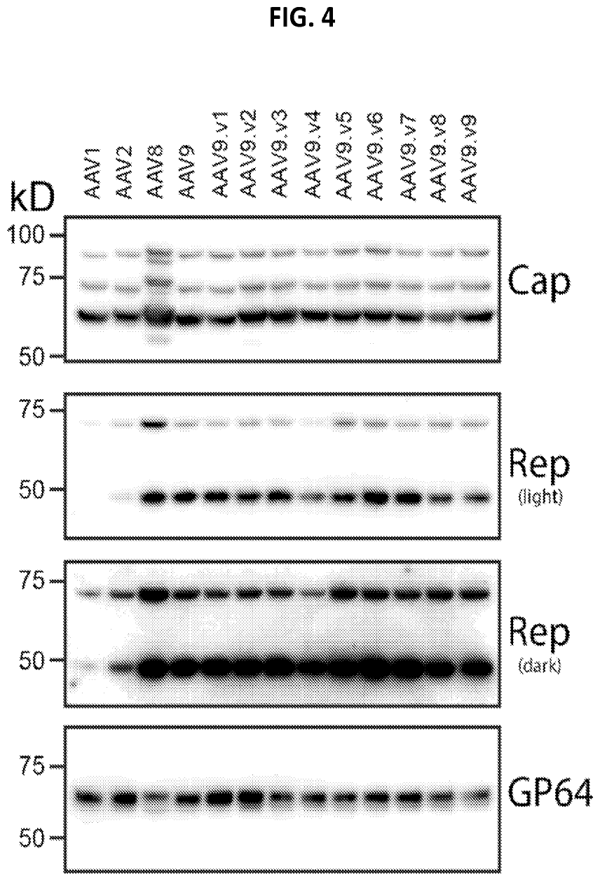

20 μg of Bacmid AB189 was digested with AscI enzyme for 2 hours at 37° C. in 20 μL reactions (1× Cutsmart). Capsid sequence insert cassettes were prepared for each of the 13 AAV serotypes using PCR extraction from source bacmids. The AscI-cut AB189 bacmid was then ligated with each of the 13 capsid sequence insert cassettes using the following Gibson Assembly reaction: 1.98 μL AscI-cut AB189+1.06 μL capsid insert product+3.04 μL 2× Hifi Assembly Mastermix. Each mixture was incubated for 1 hour at 50° C., then dialyzed, electroporated, and then plated for colony selection on KAN+IPTG+X-GAL plates. Each colony was subjected to colony selection and subsequent Midiprep bacmid DNA extraction. Bacmid DNA samples were confirmed to be infectious by transfection into Sf9 GFP-reporter cells and through REP/CAP western blot analysis, and insertion of cap sequence cassettes into AB189 was also confirmed through PCR extraction and DNA sequencing. Results of AB189 testing are shown in FIG. 4. All tested capsid constructs showed measurable CAP, REP, and GP64 production.

Taken together these data demonstrate that Bacmid AB189 comprising Rep78 and Rep52 coding regions in two separate loci (e.g., distinct transcriptional cassettes) in the bacmid as well overlapping VP 1, 2, and 3 coding regions that express all three from a polyscistronic ORF, present in the same loci, was capable of producing AAV capsid and replication proteins for a variety of serotypes for production of AAV particles.

### Production of Bacmid AB191

Bacmid AA862 was first processed to remove existing AscI sites and LacZa cassettes in the starting bacmids. 5 μg of Bacmid AA862 was digested with AscI for 1 hour at 37° C. in 10 μL reactions (1× Cutsmart). Bacmids were then re-ligated using the primer of SEQ ID NO: 18, in the following reaction: 4 μL AscI-digseted AA862+1 μL ligation primer (450 nM; SEQ ID NO: 18)+5 μL 2× Hifi Assembly Mix. The mixture was incubated for 1 hour at 50° C., then dialyzed, electroporated, and then plated for colony selection on KAN+IPTG+X-GAL plates. Colony clones AB164 and AB165 were selected for further engineering work.

Midiprep bacmid DNA of clones AB164 and AB165 were prepared using the Macherey-Nagel Xtra Midi kit, yielding about 100 μg of bacmid DNA for each. Both bacmid DNA samples were confirmed to be infectious by transfection into Sf9 GFP-reporter cells. Polynucleotide insert cassettes were also provided which included a TgDest-LacZa cassette insert as presented in SEQ ID NO: 20 (with Gibson Assembly sequences).

5 μg of clones AB164 bacmid midiprep was digested with 2 μL I-CeuI enzyme in a 20 μL reaction (1× Cutsmart) for 5 hours at 37° C. The I-CeuI-cut AB164 bacmid was then ligated with the TgDest-LacZa cassette insert using the following Gibson Assembly reaction: 5 μL I-CeuI-cut AB164+1 μL TgDest-LacZa product (˜40 ng)+6 μL 2× Hifi Assembly Mastermix. The mixture was incubated for 1 hour at 50° C., then dialyzed, electroporated, and then plated for colony selection on KAN+IPTG+X−GAL plates. Colony clones AB180 and AB181 were selected for further analysis and engineering work.

Midiprep bacmid DNA of clones AB180 and AB181 were prepared using the Macherey-Nagel Xtra Midi kit, yielding about 100-130 μg of bacmid DNA for each. Both bacmid DNA samples were confirmed to be infectious by transfection into Sf9 GFP-reporter cells. Insertion of the TgDest-LacZa cassette into the v-cath locus of AB180 was also confirmed through PCR extraction and DNA sequencing.

Bacmid AB180 was then processed to remove existing chloramphenicol-resistance cassettes. 5 tig of Bacmid AB180 was digested with I-SceI enzyme for 1 hour at 37° C. in 10 μL reactions (1× Cutsmart), followed by heat inactivation at 65° C. for 20 minutes. 5 μL of the I-SceI-cut AB180 was then re-ligated in a 20 μL T4 ligase reaction (1×T4 ligase buffer). The mixture was incubated for 30 minutes at 20° C., then dialyzed, electroporated, and plated for colony selection on KAN plates. Colony clones AB191 and AB192 were selected for further analysis

Midiprep bacmid DNA of clones AB191 and AB192 were prepared using the Macherey-Nagel Xtra Midi kit, yielding about 100 μg of bacmid DNA for each. Both bacmid DNA samples were confirmed to be infectious by transfection into Sf9 GFP-reporter cells. Deletion of the CAM cassette was confirmed using I-SceI digestion, followed by gel analysis comparison with Bacmid AB178.

### Example 6. Bacmid Passage Stability Study

Bacmid AA965 and Bacmid AB189 were analyzed for BIIC passage stability using two AAV9-variant capsids.

AAV capsids sequence inserts for each of the two AAV9-variant capsids were cloned into Bacmid AA965 and Bacmid AB189 (confirmed by sequencing), followed by transfection of each resulting bacmid into Sf9 cells to provide corresponding p0 BIIC banks: BIIC 965_801, BIIC 965_804, BIIC 189_801, and BIIC 189_804. Each p0 BIIC batch was titered, and then infected into 50 mL of Sf9 cells (2×10⁶ cell/mL) at 0.01 MOI. Each culture then went through 5 passages, roughly 3 days apart for each passage, using a 1:10000 fold dilution (3 μl of the previous culture went into 30 mL of fresh Sf9 cells at 2×10⁶ cell/mL). TCID50 titers were then determined (Spearman-Karber analysis) for each resulting passage for each BIIC batch. Resulting Sf9 cell count, viability, cell diameter, and BIIC TCID50 titer are presented in Table 4

TABLE 4
Bacmid Passage Stability Results - TCID50

| | | Cell | Viability | Diameter | TCID50 |
|---|---|---|---|---|---|
| Culture | Passage | Count | % | (μM) | (Titer/mL) |
| 965_801 | p0 | — | — | — | 1.33 × 10⁷ |
| | p1 | 3.5 × 10⁶ | 39.5 | 18.8 | 2.37 × 10⁸ |
| | p2 | 3.94 × 10⁶ | 81.6 | 20.4 | 1.78 × 10⁸ |
| | p3 | 3.84 × 10⁶ | 92.6 | 20.0 | 4.22 × 10⁸ |
| | p4 | 3.12 × 10⁶ | 92.1 | 20.3 | 7.5 × 10⁸ |
| | p5 | 4.84 × 10⁶ | 92.4 | 20.1 | 1.78 × 10⁸ |
| 965_804 | p0 | — | — | — | 7.5 × 10⁶ |
| | p1 | 3.09 × 10⁶ | 34 | 18.5 | 5.62 × 10⁷ |
| | p2 | 4.17 × 10⁶ | 74.4 | 20.4 | 4.22 × 10⁸ |
| | p3 | 3.08 × 10⁶ | 86.9 | 17.7 | 5.62 × 10⁷ |
| | p4 | 3.41 × 10⁶ | 93.3 | 20.4 | 1.33 × 10⁸ |
| | p5 | 5.0 × 10⁶ | 92.5 | 20.2 | 1.78 × 10⁸ |
| 189_801 | p0 | — | — | — | 5.62 × 10⁷ |
| | p1 | 3.28 × 10⁶ | 21.6 | 17.9 | 1.0 × 10⁸ |
| | p2 | 3.62 × 10⁶ | 91.9 | 17.4 | 1.0 × 10⁷ |
| | p3 | 3.79 × 10⁶ | 80.7 | 20.2 | 4.22 × 10⁸ |
| | p4 | 3.04 × 10⁶ | 9039 | 20.1 | 7.5 × 10⁸ |
| | p5 | 5.44 × 10⁶ | 82.2 | 20.5 | 1.78 × 10⁸ |
| 189_804 | p0 | — | — | — | 1.0 × 10⁸ |
| | p1 | 3.47 × 10⁶ | 22 | 18.0 | 4.22 × 10⁷ |
| | p2 | 4.06 × 10⁶ | 69.5 | 20.5 | 1.78 × 10⁸ |
| | p3 | 3.46 × 10⁶ | 69 | 19.9 | 5.62 × 10⁸ |
| | p4 | 2.86 × 10⁶ | 83.7 | 20.4 | 4.22 × 10⁸ |
| | p5 | 4.62 × 10⁶ | 79.3 | 20.6 | 4.22 × 10⁸ |

Samples from each resulting passage for each BIIC batch were then tested for AAV production in a small-scale bioreactor with GFP Transgene BIICS. 22 bioreactor tubes were seeded with 25 mL each of Sf9 cells at 6.7×10⁶ viable cells/mL (95.5% viability). Each bioreactor was then infected with 0.03 MOI GFP-Transgene BIIC and 0.01 MOI Capsid BIIC (3:1 ratio). After 3 days, viral production cells were collected, with: (i) a portion boiled in SDS loading buffer and then frozen for western blot analysis, and (ii) the remaining portion lysed using 3.6 mL of 10% Triton X-100 and 2.4 mL of 2M Arginine (for final concentration of 1.2% Triton X-100 and 164 mM Arginine), followed by 10 μL of benzonase mixture (33 μL benzonase in 217 μL PBS), and then allowed to shake overnight.

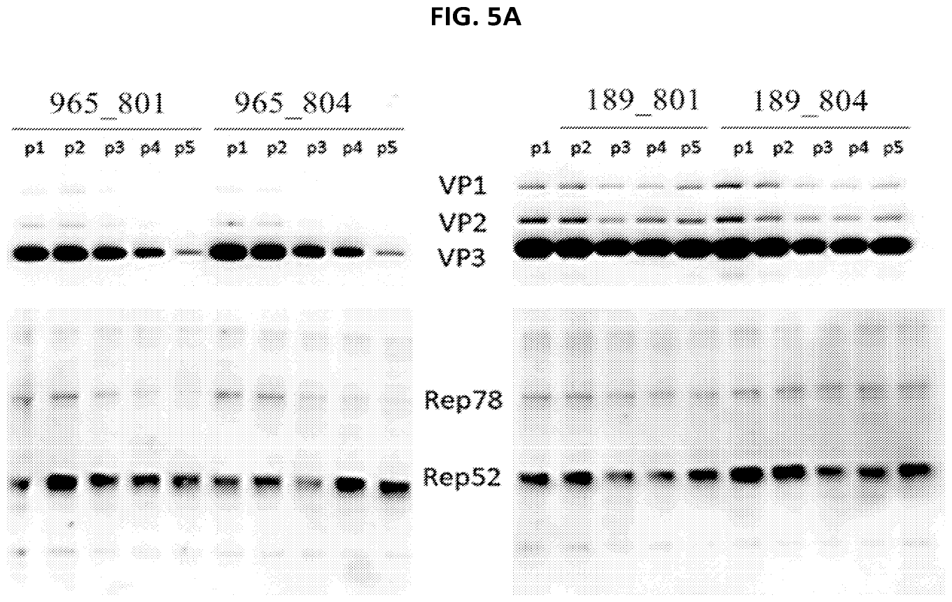

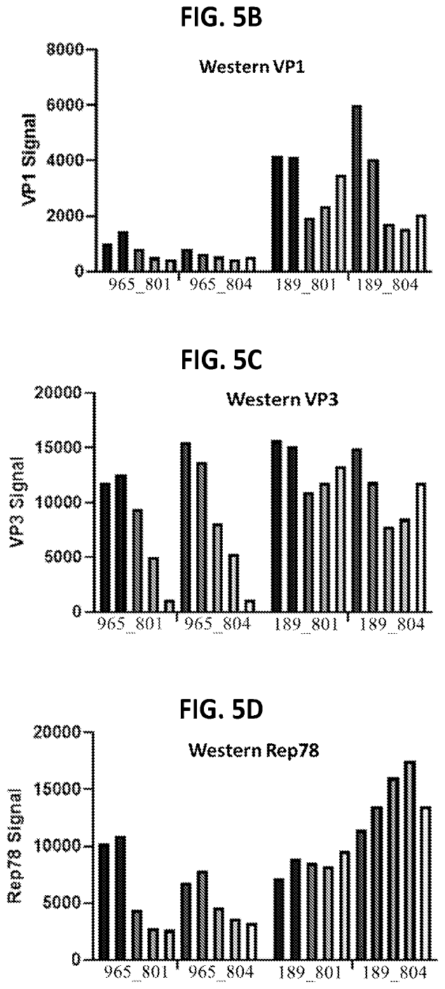

For the first portion, a 10,000-cell equivalent of each production run was loaded on an SDS-PAGE gel for a western blot analysis for AAV REP and CAP proteins. Results are shown in FIG. 5A. Resulting dosimetry of the bands was measured using ImageJ software and graphed, as shown in FIG. 5B, FIG. 5C, and FIG. 5D. Western blot analysis showed a clear decline VP1, VP3, and Rep78 production for Bacmid AA965 after the first 2 passages, while Bacmid AB189 maintains VP1, VP3, and Rep78 production through at least the first 5 passages.

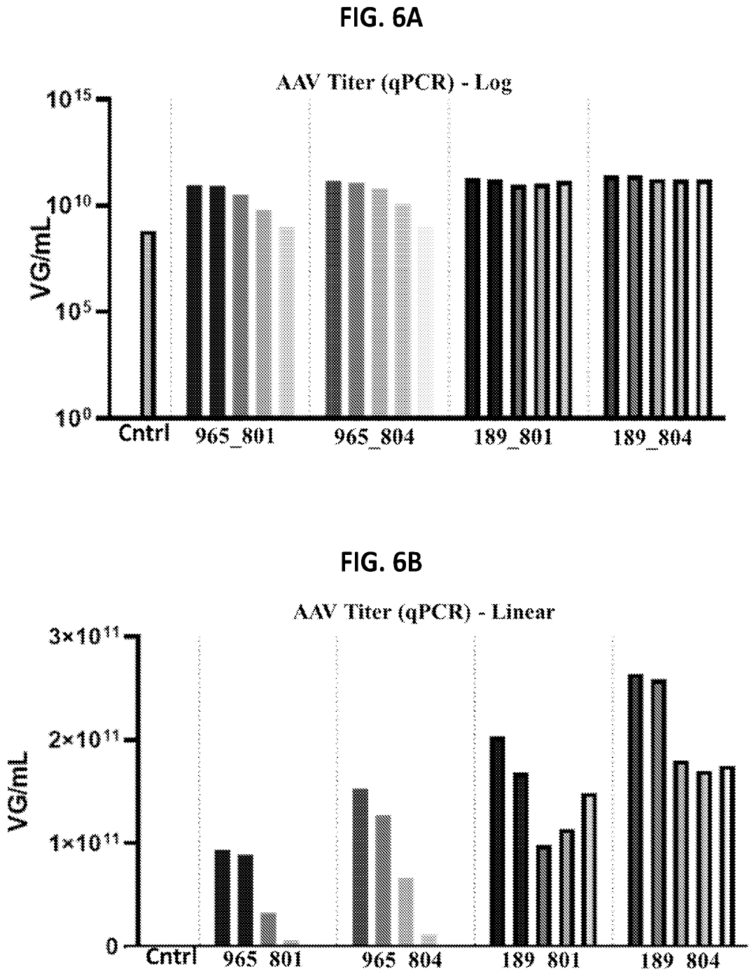

For the remaining portion, crude lysates from each batch were titered via qPCR. Results are presented in Table 5, and in FIG. 6A (log graph) and FIG. 6B (linear graph).

TABLE 5
Bacmid Passage Stability Results - Titer

| Culture | Passage | qPCR Titer (vg/mL) |
|---|---|---|
| 965_801 | p1 | 9.35 × 10¹⁰ |
| | p2 | 8.91 × 10¹⁰ |
| | p3 | 3.28 × 10¹⁰ |
| | p4 | 6.23 × 10⁹ |
| | p5 | 1.02 × 10⁹ |
| 965_804 | p1 | 1.53 × 10¹¹ |
| | p2 | 1.27 × 10¹¹ |
| | p3 | 6.68 × 10¹⁰ |
| | p4 | 1.20 × 10¹⁰ |
| | p5 | 9.69 × 10⁸ |
| 189_801 | p1 | 2.03 × 10¹¹ |
| | p2 | 1.68 × 10¹¹ |
| | p3 | 9.80 × 10¹⁰ |
| | p4 | 1.14 × 10¹¹ |
| | p5 | 1.49 × 10¹¹ |
| 189_804 | p1 | 2.64 × 10¹¹ |
| | p2 | 2.58 × 10¹¹ |
| | p3 | 1.80 × 10¹¹ |
| | p4 | 1.70 × 10¹¹ |
| | p5 | 1.75 × 10¹¹ |

qPCR titer results showed a clear decline AAV titer for Bacmid AA965 after the first 2 passages, while Bacmid AB189 maintains stead AAV titer through at least the first 5 passages.

### Example 7: Modulation of VP1 Expression

The Example describes the generation of expression systems for AAV production (e.g., in Sf9 cell), designed to modulate and increase expression of the VP1 protein to further improve capsid protein ratios, using Bacmid AB189 and Bacmid AB189-VP1_{ACG} as described in Examples 5 and 6.

### Modulation of VP1 Expression Using a CTX Promoter

In order to increase VP1 expression, a second copy of the VP1 gene was added to Bacmid AB189-VP1_{ACG}, in addition to the copy of VP1 present in the overlapping VP 1, 2, and 3 coding region previously inserted into the v-cath baculovirus gene locus. This second copy of the VP1 gene was engineered to be under the control of the CTX baculovirus promoter (SEQ ID NO: 164). The CTX promoter was selected as it is activated in the very late stage of baculovirus transcription and was predicted to produce 23% of the RNA transcripts produced by p10 promoter at the same time. The CTX promoter used comprises the CTX promoter as well as the 5′ UTR typically present upstream of the CTX promoter in the CTX gene (AcORF3) (SEQ ID NO: 164).

Using Gibson Assembly, the VP1 gene under the control of the CTX promoter was inserted into the SOD baculovirus gene locus at the AbsI restriction endonuclease (REN) cleavage site, which is present in the middle of the SOD ORF. The resulting bacmid, Bacmid AB189-VP1_{ACG}-CTX VP1 is shown in FIG. 8. As shown in FIG. 8, Bacmid AB189-VP_{ACG}-CTX VP1 comprised a Rep78 gene with a minicistron upstream under the control of the polh promoter and overlapping VP1, VP2, and VP3 coding regions under the control of the p10 promoter in the v-cath locus; a Rep52 gene under the control of the polh promoter in the egt locus; and a second VP1 gene under the control of the CTX promoter in the SOD locus. In order to further control expression of the VP proteins in the overlapping VP coding regions in the v-cath locus, the start codons for the VP1 and the VP2 coding regions were an ACG start codon, and the start codon for the VP3 coding region was an ATG start codon. The chiA, p26, p10, and p74 genes were also deleted from the genome of Bacmid AB189-VP_{ACG}-CTX VP1.

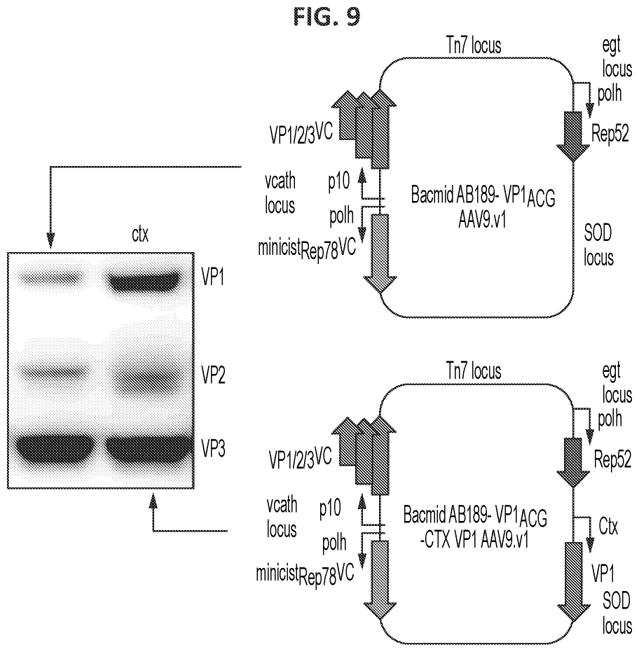

Bacmid AB189-VP1_{ACG}-CTX VP1 was then tested for its ability to produce AAV capsids and increased VP1 protein levels, relative to Bacmid AB189, only containing 1 copy of VP1. These bacmids were engineered to express the VP1, VP2, and VP3 proteins of the AAV9 capsid variant, AAV9.v1. Bacmid AB189-VP1_{ACG}-CTX VP1 encoding the AAV9.v1 capsid variant and Bacmid AB189-VP1 ACG also encoding the AAV9v.1 capsid variant were transfected into Sf9 GFP-reporter cells. These Sf9 viral production cells were later collected (˜4 days post transfection) and subjected to SDS-PAGE and Western blot analysis to determine relative levels of the different VP capsid proteins produced by these two bacmids. As shown in FIG. 9, Bacmid AB189-VP1_{ACG}-CTX VP1 encoding AAV9.vl with two copies of VP1, including one under the control of the CTX, promoter resulted in increased levels of VP1 (right lane of gel) relative to Bacmid AB189-VP1_{ACG} only encoding a single copy of VP1 (left lane of gel). These results demonstrate that AB189-VP1_{ACG}-CTX VP1 comprising two copies of VP1, including one under the control of the CTX promoter, was capable of producing higher quantities of VP1, than a bacmid comprising only a single copy of the VP1 gene.

### Modulation of VP1 Expression Using Modified Kozak Sequences

In order to increase VP1 expression, a modified Kozak translation initiation sequence was inserted upstream of the VP1 gene, in the overlapping VP1, 2, and 3 coding regions present in the v-cath baculovirus gene locus. Without wishing to be bound by theory, this modified Kozak approach was selected such that a weak initiation context would be present before the AUG strong start codon of the VP1 gene, and this would lead to increased leaky ribosome scanning, altered translation initiation, and modification of the stoichiometry of VP1, VP2, and VP3.

The modified Kozak sequences in Table 6 were designed and VPlaug13 and VPlaug14 were selected for further characterization (also provided in Table 9).

TABLE 6
Exemplary Modified Kozak Sequences

| | | | | | Amino |
|---|---|---|---|---|---|
| | | SEQ | | SEQ | acid |
| | Sequence | ID | Sequence | ID | at posi- |
| Name | (RNA) | NO: | (DNA) | NO: | tion 2 |
| VP1aug13 | cgguguAUGgc | 21 | cggtgtATGgc | 32 | Alanine |
| VP1aug14 | cgguguAUGag | 22 | cggtgtATGag | 33 | Serine |
| VP1aug28 | uaguggAUGag | 23 | tagtggATGag | 34 | Serine |
| VP1aug29 | uauuggAUGag | 24 | tattggATGag | 35 | Serine |
| VP1aug30 | caguggAUGag | 25 | cagtggATGag | 36 | Serine |
| VP1aug31 | cauuggAUGag | 26 | cattggATGag | 37 | Serine |
| VP1aug32 | ugguggAUGag | 27 | tggtggATGag | 38 | Serine |
| VP1aug33 | aauuggAUGag | 28 | aattggATGag | 39 | Serine |
| VP1aug34 | aaguggAUGag | 29 | aagtggATGag | 40 | Serine |
| VP1aug35 | ccguggAUGag | 30 | ccgtggATGag | 41 | Serine |
| VP1aug36 | ggguggAUGag | 31 | gggtggATGag | 42 | Serine |

VPlaug13 (SEQ ID NO: 21 (RNA) or 32 (DNA)) and VPlaug14 (SEQ ID NO: 22 (RNA) or SEQ ID NO: 33 (DNA)) were inserted immediately upstream (e.g., at the 5′ end) of the VP1 gene encoding the AAV9.v2 capsid variant and the AAV9.v5 capsid variant in Bacmid AB189. The nucleotide sequence of the AAV9.v2 VP1 coding region comprising the VPlaug13 modified Kozak sequence and the encoded VP1 amino acid sequence are provided as SEQ ID NOs: 50 and 52, respectively. The nucleotide sequence of the AAV9.v2 VP1 coding region comprising the VPlaug14 modified Kozak sequence and the encoded VP1 amino acid sequence are provided as SEQ ID NOs: 51 and 53, respectively. The nucleotide sequence of the AAV9.v5 VP1 coding region comprising the VPlaug13 modified Kozak sequence and the encoded VP1 amino acid sequence are provided as SEQ ID NOs: 58 and 60, respectively. The nucleotide sequence of the AAV9.v2 VP1 coding region comprising the VPlaug14 modified Kozak sequence and the encoded VP1 amino acid sequence are provided as SEQ ID NOs: 59 and 61, respectively. A nucleotide sequence encoding an AAP protein was also inserted into the p10, p26, and p74 gene loci, which were previously deleted from Bacmid AB189. The nucleotide sequence encoding the AAP protein was under the control of the gp64 promoter (SEQ ID NO: 217).

TABLE 18
Exemplary AAP protein sequences

| | SEQ ID | |
|---|---|---|
| Description | NO: | Sequences |
| AAP (amino | 218 | METQTQYLTPSLSDSHQQPPLVWELIRWLQAVAHQWQTITRAPTEWVIPREIGIAI |
| acid) | | PHGWATESSPPAPEPGPCPPTTTTSTNKFPANQEPRITITTLATAPLGGILTSTDS |
| | | TATFHHVTGKDSSTTTGDSDPRDSTSSSLTFKSKRSRRMTVRRRLPITLPARFRCL |
| | | LTRSTSSRISSARRIKDASRRSQQTSSWCHSMDTSP |
| AAP (DNA) | 219 | ATGGAGACACAGACGCAGTACCTGACGCCATCATTGTCTGATAGCCATCAGCAGCC |
| | | TCCGCTTGTATGGGAACTTATACGTTGGCTCCAGGCCGTCGCCCATCAATGGCAGA |
| | | CGATCACTAGGGCTCCTACCGAATGGGTAATCCCCAGAGAGATTGGTATCGCCATC |
| | | CCCCATGGCTGGGCTACCGAATCATCTCCTCCCGCCCCTGAGCCAGGTCCTTGTCC |
| | | TCCGACAACTACAACTTCGACGAATAAATTCCCGGCTAACCAAGAGCCACGTACTA |
| | | CCATCACAACTTTGGCAACAGCTCCACTGGGTGGAATACTCACAAGCACAGACTCG |
| | | ACTGCTACGTTCCACCATGTGACAGGTAAGGATAGTAGTACAACGACCGGCGACTC |
| | | TGATCCAAGGGACTCTACGTCATCGTCTTTGACTTTTAAGTCGAAAAGAAGCCGTC |
| | | GTATGACTGTTCGTCGCAGGCTTCCCATTACCCTTCCCGCTAGGTTCAGATGCCTC |
| | | CTTACACGCAGTACCAGTTCCAGAACTTCATCGGCTAGGAGAATAAAGGATGCTTC |
| | | TAGACGCAGCCAACAAACTAGTTCATGGTGCCATTCCATGGACACTAGCCCAAGCG |
| | | GAGACTATAAGGATGATGACGACAAGTAATAA |

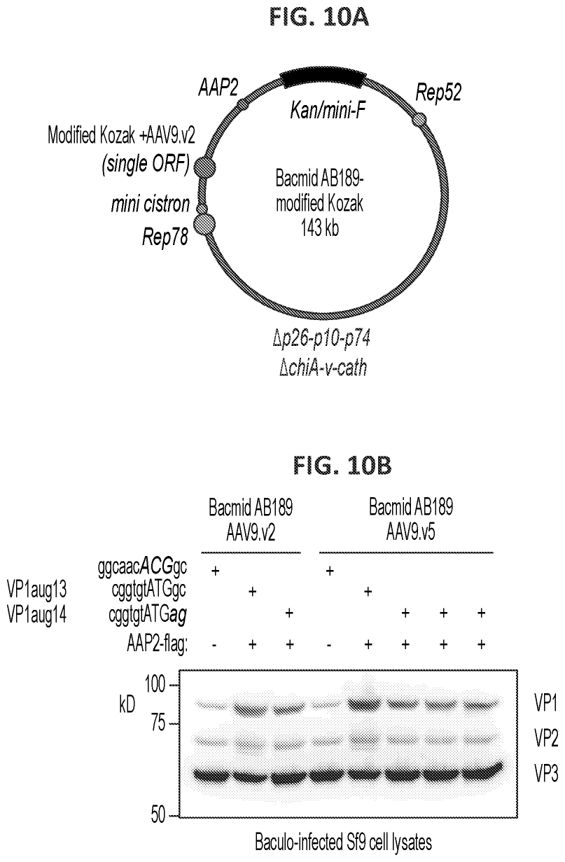

The resulting bacmid, Bacmid AB189-modified Kozak, comprised overlapping VP1, VP2, and VP3 coding regions (expressed all 3 from a single polycistronic ORF), where VP1 comprised a modified Kozak sequence with an ATG initiation codon (either VPlaug13 or VPlaug14) at the 5′ end of the VP1 gene, the VP2 gene comprised an ACG start codon, and the VP3 gene comprised an ATG start codon. The overlapping VP coding region and modified Kozak sequence were inserted into the v-cath baculovirus gene locus under the control of the of the p10 promoter. Bacmid AB189-modified Kozak further comprised a Rep78 gene with a minicistron upstream under the control of the polh promoter also in the v-cath baculovirus gene locus, a Rep52 gene under the control of the polh promoter in the egt locus, and the gene encoding the AAP protein in p26-p10-p74 baculovirus gene loci under the control of a gp64 promoter. The chiA, p26, p10, and p74 genes were also deleted from the genome of Bacmid AB189-modified Kozak. An exemplary Bacmid-modified Kozak encoding AAV9.v2 VP1 is provided in FIG. 10A.

Bacmid AB189-modified Kozak comprising the VPlaug13 modified Kozak sequence and Bacmid AB189-modified Kozak comprising the VPlaug14 modified Kozak sequence upstream of VP1 were then tested for their ability to produce AAV capsids and increased VP1 protein levels relative to the Bacmid AB189 control (comprising ggcaacACGgc (SEQ ID NO: 163) comprising an ACG start codon for initiating translation of the VP1 gene. Bacmid AB189-modified Kozak comprising the VPlaug13 modified Kozak sequence and Bacmid AB189-modified Kozak comprising the VPlaug14 modified Kozak sequence upstream of VP1 encoding the AAV9.v2 or AAV9.v5 capsid variants along with the Bacmid AB189 control encoding the AAV9.v2 or AAV9.v5 capsid variants were transfected into Sf9 cells. These Sf9 viral production cells were later collected (˜4 days post transfection) and subjected to SDS-PAGE and Western blot analysis to determine relative levels of the different VP capsid proteins produced by these two bacmids. As shown in FIG. 10B, the VPlaug13 and VPlaug14 designs supported robust VP1 expression levels for both the AAV9.v2 and AAV9.v5 capsid proteins, compared to the control Bacmid AB189 comprising the sequence ggcaacACGgc (SEQ ID NO: 163) for initiating translation of VP1.

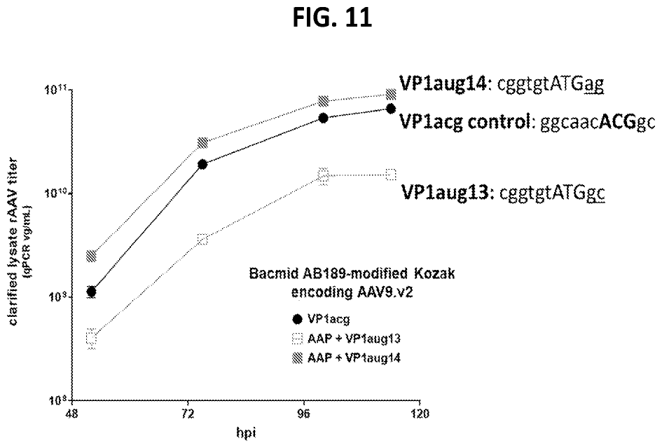

AAV productivity and titer was also measured for Bacmid AB189-modified Kozak comprising the VPlaug13 modified Kozak sequence and Bacmid AB189-modified Kozak comprising the VPlaug14 modified Kozak sequence upstream of VP1 gene encoding the AAV9.v2 capsid variant (Bacmid AB189-modified Kozak-VPlaug13-AAV9.v2 and Bacmid AB189-modified Kozak-VPlaug14-AAV9.v2, respectively) compared to the Bacmid AB189 control encoding the AAV.v2 capsid variant with an ACG start codon to initiate VP1 translation (Bacmid AB189-AAV9.v2_{ACG}). Bacmid AB189-modified Kozak-VPlaug13-AAV9.v2 and Bacmid AB189-modified Kozak-VPlaug14-AAV9.v2 were transfected into Sf9 cells and samples of these transfected cells were taken at 52, 75, 100, and 114 hours post transfection. These samples were analyzed by qPCR to quantify the rAAV titer in the clarified cellular lysates in vg/mL (FIG. 11). As shown in FIG. 11, the VPlaug14 design (e.g., Bacmid AB189-modified Kozak-VPlaug14-AAV9.v2) exhibited superior productivity compared to the VPlaug13 design (e.g., Bacmid AB189-modified Kozak-VPlaug13-AAV9.v2) and the Bacmid AB189-AAV9.v2_{ACG} control. Additionally, the VP ratios (ratio of VP3:VP2:VP1) were calculated for Bacmid AB189-modified Kozak-VPlaug13-AAV9.v2, Bacmid AB189-modified Kozak-VPlaug14-AAV9.v2, and Bacmid AB189-AAV9.v2_{ACG} control. As shown in Table 11, the VPlaug13 and VPlaug14 designs significantly improved VP ratios in purified AAV9.v2 capsid variants, with incorporation of the VPlaug14 modified Kozak sequence resulting in 10:1:1 ratios for VP3:VP2:VP1.

TABLE 11
Quantification of VP ratios for AAV9.v2 capsid proteins produced
by Bacmid AB189 without or without modified Kozak sequences

| | VP3:VP2:VP1 |
|---|---|
| | ratio for |
| AAV Expression Construct | AAV9.v2 |
| Bacmid AB189-modified Kozak-VP1aug13-AAV9.v2 | 6:1:1 |
| Bacmid AB189-modified Kozak-VP1aug14-AAV9.v2 | 10:1:1 |
| Bacmid AB189-AAV9.v2ACG control | 37:3:1 |

These results demonstrate the Bacmid AB189-modified Kozak comprising either the VPlaug13 (SEQ ID NO: 21 (RNA) or 32 (DNA)) or VPlaug14 (SEQ ID NO: 22 (RNA) or SEQ ID NO: 33 (DNA)) modified Kozak sequences at the 5′ end of the VP1 gene for translation initiation of the VP1 protein were capable of robust expression of VP1 for two AAV9 capsid variants, increased AAV productivity and titer, as well as improved VP ratios, as compared to Bacmid AB189-VP1_{ACG} comprising VP1 with an ACG start codon for initiating translation. Therefore, these modified Kozak sequences are able to successfully modify VP1 expression for regulation of VP capsid protein levels during AAV production in insect cells.

### Comparison of AB189-VP_{ACG}-CTX VP1 and Bacmid AB189-Modified Kozak

The AAV expression constructs Bacmid AB189-VP1_{ACG}-CTX VP1 and Bacmid AB189-modified Kozak were compared with respect to the potency of the encoded AAV capsid and VP capsid ratios produced for AAV9 and variants thereof.

In order to evaluate AAV potency, Sf9 cells were co-transfected with SEAP-GFP payload BIIC material (Bacmid AB191 encoding a SEAP-GFP transgene which is inserted into the chiA/v-cath locus of Bacmid AB191) and either AB189-VP1_{ACG}-CTX VP1 encoding AAV9.v1 (Bacmid AB189-AAV9.v1_{ACG}-CTX VP1), Bacmid AB189-modified Kozak-VPlaug13 encoding AAV9 (Bacmid AB189-modified Kozak-VPlaug13-AAV9), Bacmid AB189-modified Kozak-VPlaug14 encoding AAV9 (Bacmid AB189-modified Kozak-VPlaug14-AAV9) or a Bacmid AB189-VP1_{ACG} control encoding AAV9.v1 (Bacmid AB189-AAV9.v1_{ACG}) The nucleotide sequence of the VP1 coding region comprising the VP1aug13 modified Kozak sequence and encoded VP1 amino acid sequence are provided as SEQ ID NOs: 44 and 46, respectively, and the nucleotide sequence of the VP1 coding region comprising the VP1aug14 modified Kozak sequence and encoded VP1 amino acid sequence are provided as SEQ ID NOs: 45 and 47, respectively. The Bacmid AB189-AAV9.v1_{ACG}-CTX VP1 and Bacmid AB189-AAV9.v1_{ACG} control encode the VP1 amino acid sequence of SEQ ID NO: 48.

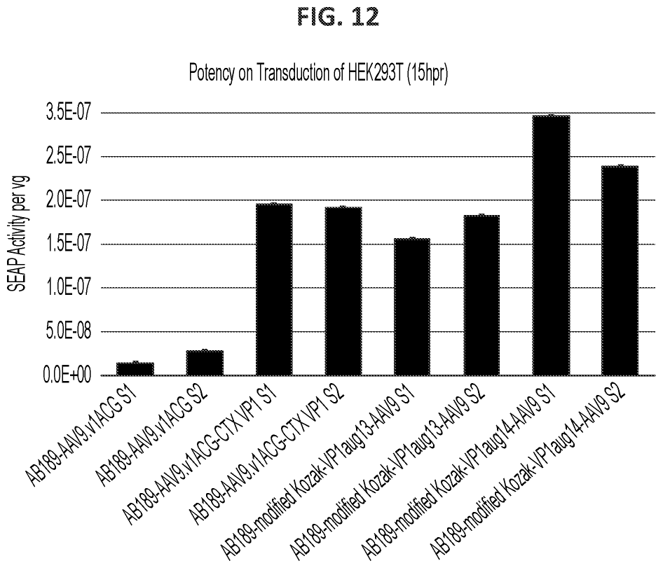

Cells were collected post-Sf9 cellular transfection and lysed. Cellular lysate comprising the AAV samples were then affinity purified and used to transduce HEK293T cells Alkaline phosphatase activity (e.g., potency) of the transduced HEK293T cells was measured at 15 hours post infection at an OD405. The SEAP activity per viral genome (vg) was determined for each AAV expression construct (Table 12 and FIG. 12).

TABLE 12
SEAP activity as a measure of AAV potency
on transduction of HEK293T cells

| AAV Expression Construct | Sample | SEAP activity/vg |
|---|---|---|
| Bacmid AB189-AAV9.v1_{ACG} | 1 | 1.38E−08 |
| | 2 | 2.76E−08 |
| Bacmid AB189-AAV9.v1_{ACG}-CTX VP1 | 1 | 1.95E−07 |
| | 2 | 1.91E−07 |
| Bacmid AB189-modified Kozak- | 1 | 1.56E−07 |
| VP1aug13-AAV9 | 2 | 1.83E−07 |
| Bacmid AB189-modified Kozak- | 1 | 2.97E−07 |
| VP1aug14-AAV9 | 2 | 2.38E−07 |

Both the CTX (Bacmid AB189-AAV9.v1_{ACG}-CTX VP1) and modified Kozak Bacmid designs (Bacmid AB189-modified Kozak-VPlaug13-AAV9 and Bacmid AB189-modified Kozak-VPlaug14-AAV9) resulted in significantly improved transduction efficiency and potency of AAV9 capsid variants on HEK293T cells compared to the Bacmid AB189-AAV9.v1_{ACG} control (FIG. 12 and Table 12).

Additionally, SDS-PAGE gel analysis, coomassie staining, and ImageJ quantification was used to quantify the relative amounts of VP1, VP2, and VP3 proteins produced by the Bacmid AB189-AAV9.v1_{ACG}-CTX VP1, Bacmid AB189-modified Kozak-VPlaug13-AAV9, Bacmid AB189-modified Kozak-VPlaug14-AAV9, compared to the Bacmid AB189-AAV9.v1_{ACG} control (n=2 samples tested), which are provided in Table 13.

TABLE 13
Quantification of VP proteins produced by Bacmid AB189-CTX VP1
or Bacmid AB189-modified Kozak compared to Bacmid AB189 Control

| | | VP3 | | VP2 | | VP1 | | Total | |
|---|---|---|---|---|---|---|---|---|---|
| Bacmid | Sample | (ng/μL) | | (ng/μL) | | (ng/μL) | | (ng/μL) | |
| Bacmid AB189- | Sample 1 | 60 | ng/μL | 6 | ng/μL | 10 | ng/μL | 76 | ng/μL |
| AAV9.v1_{ACG}-CTX VP1 | Sample 2 | 59 | ng/μL | 7 | ng/μL | 11 | ng/μL | 77 | ng/μL |
| | Average | 59.5 | ng/μL | 6.5 | ng/μL | 10.5 | ng/μL | 76.5 | ng/μL |
| AB189-modified Kozak- | Sample 1 | 38 | ng/μL | 4 | ng/μL | 8 | ng/μL | 50 | ng/μL |
| VP1aug13-AAV9 | Sample 2 | 48 | ng/μL | 4 | ng/μL | 10 | ng/μL | 62 | ng/μL |
| | Average | 43 | ng/μL | 4 | ng/μL | 9 | ng/μL | 56 | ng/μL |
| AB189-modified Kozak- | Sample 1 | 54 | ng/μL | 3 | ng/μL | 5 | ng/μL | 61 | ng/μL |
| VP1aug14-AAV9 | Sample 2 | 83 | ng/μL | 4 | ng/μL | 6 | ng/μL | 92 | ng/μL |
| | Average | 68.5 | ng/μL | 3.5 | ng/μL | 5.5 | ng/μL | 76.5 | ng/μL |
| AB189-AAV9.v1_{ACG} | Sample 1 | 93 | ng/μL | 4 | ng/μL | 3 | ng/μL | 99 | ng/μL |
| | Sample 2 | 87 | ng/μL | 3 | ng/μL | 3 | ng/μL | 93 | ng/μL |
| | Average | 90 | ng/μL | 3.5 | ng/μL | 3 | ng/μL | 96 | ng/μL |

As provided in Table 14, the VP ratios for the VP proteins produced by the Bacmid AB189-AAV9.v1ACG-CTX VP1, Bacmid AB189-modified Kozak-VPlaug13-AAV9, and Bacmid AB189-modified Kozak-VPlaug14-AAV9 were quantified and compared to the Bacmid AB189-AAV9.v1 ACG control (n=2 samples tested).

TABLE 14
Quantification of VP ratios of Bacmid AB189-
AAV9.v1ACG-CTX VP1or Bacmid AB189-modified
Kozak compared to Bacmid AB189 Control

| | | Ratio |
|---|---|---|
| Bacmid | Sample | (VP3:VP2:VP1) |
| Bacmid AB189-AAV9.v1_{ACG}-CTX VP1 | 1 | 6:01:01 |
| | 2 | 5:01:01 |
| AB189-modified Kozak- | 1 | 5:01:01 |
| VP1aug13-AAV9 | 2 | 5:01:01 |
| AB189-modified Kozak- | 1 | 12:01:01 |
| VP1aug14-AAV9 | 2 | 12:01:01 |
| AB189-AAV9.v1_{ACG} | 1 | 32:02:01 |
| | 2 | 40:02:01 |

These data demonstrate that both the use of a CTX promoter driving expression of a second copy of the VP1 gene, as well as use of modified Kozak sequences (e.g., VPlaug13 (SEQ ID NO: 21 (RNA) or 32 (DNA)) and VPlaug14 (SEQ ID NO: 22 (RNA) or SEQ ID NO: 33 (DNA))) to initiate translation of the VP1 protein from overlapping VP1, VP2, and VP3 coding regions (a single polycistronic ORF) can each successfully modulate and increase VP1 production to produce desired ratios of VP proteins for improved AAV production.

### Example 8: Passage Stability of Bacmid AB189 Encoding an AAV1 Capsid Protein

This example investigates the passage stability of the Bacmid AB189-VP1_{ACG} (see Examples 5 and 6) compared to the bMON14272 bac-to-bac construct (described in Luckow et al. Efficient generation of infectious recombinant baculoviruses by site-specific transposon-mediated insertion of foreign genes into a baculovirus genome propagated in Escherichia coli. J Virol. 1993 August 67(8):4566-79; the contents of which are hereby incorporated by reference in its entirety).

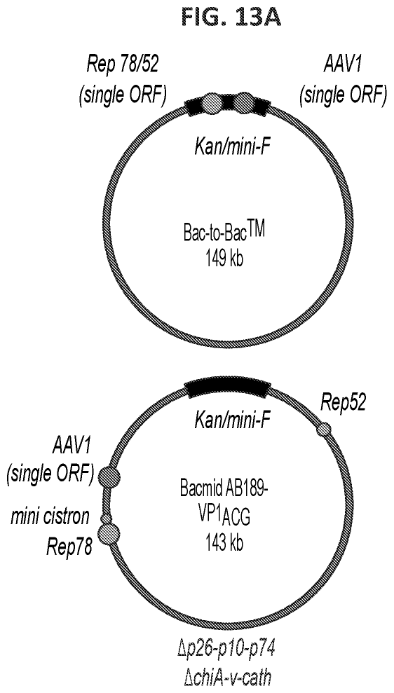

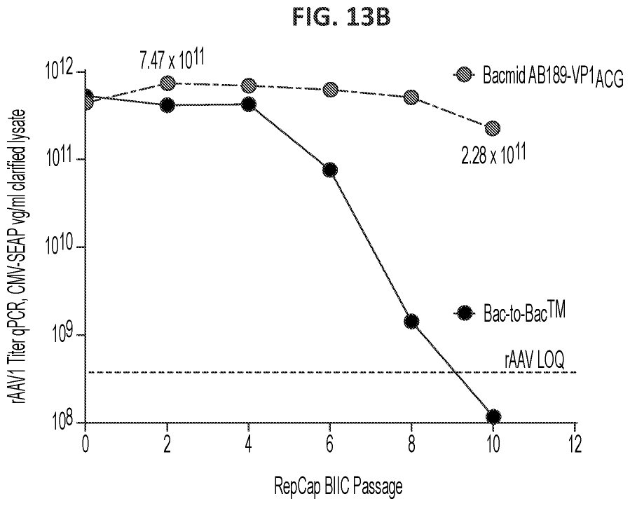

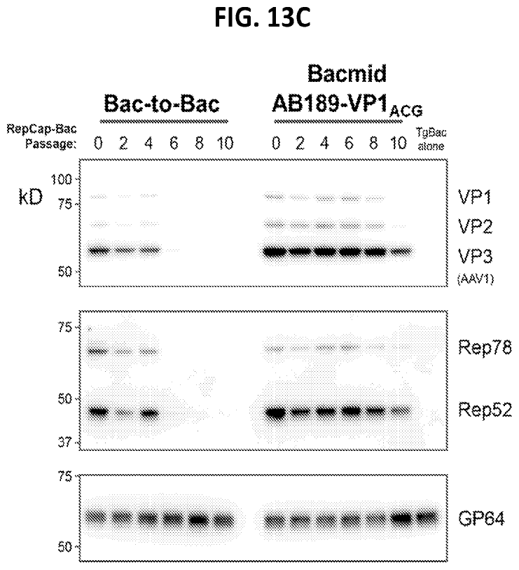

As shown in FIG. 13A (bottom construct), Bacmid AB189-VP1_{ACG} expression construct comprises Rep78 and Rep52 coding regions in two separate loci (e.g., distinct transcriptional cassettes) in the bacmid as well overlapping VP 1, 2, and 3 coding regions (single polycistronic ORF) present in a single loci. The Rep78 and overlapping VP coding regions were both inserted into the v-cath locus, where the Rep78 coding region was under the control of a polyhedrin (polh) promoter and the overlapping VP regions were under the control of a p10 promoter. A minicistron was inserted upstream of the Rep78 ORF to further modulate expression of the Rep78 protein. The Rep52 coding region was inserted into the egt locus under the control of a polh promoter. The chiA, p26, p10, and p74 genes were also deleted from the genome of Bacmid AB189. As shown in FIG. 13A (top construct), the bMON14272 bac-to-bac control expression construct harbors overlapping VP coding regions, with a ACG start codon for expressing VP1, as well as a bicistronic Rep78/52 coding region, also comprising a CTG start codon. Additionally, in the bac-to-bac Bacmid, the Rep and Cap expression cassettes are inserted into the KAN/Mini-F region. Both the bMON14272 bac-to-bac and the Bacmid AB189 were engineered to express AAV1 capsid proteins.

Bacmid AB189-VP1_{ACG} and the control bac-to-bac Bacmid were used to transfect Sf9 cells. Samples were collected over time for 10 passages and subjected to qPCR analysis to quantify AAV1 titers, and Western blot analysis to measure Rep/Cap protein levels produced by both AAV expression constructs. Additionally, AAV potency was measured by co-transfecting Sf9 cells with SEAP-GFP transgene payloadBIIC material (Bacmid AB191 encoding a SEAP transgene which is inserted into the chiA/v-cath locus of Bacmid AB191) and either the Bacmid AB189 or bac-to-bac expression construct encoding the AAV1 capsid protein. Cells were collected over time for 10 passages post-transfection and lysed. Cellular lysate comprising the AAV samples were then affinity purified and used to transduce HEK293T cells. Alkaline phosphatase activity (e.g., potency) of the transduced HEK293T cells was measured at various time points post infection at an OD405. The SEAP activity measured as transducing units per μL was determined for each AAV expression construct.

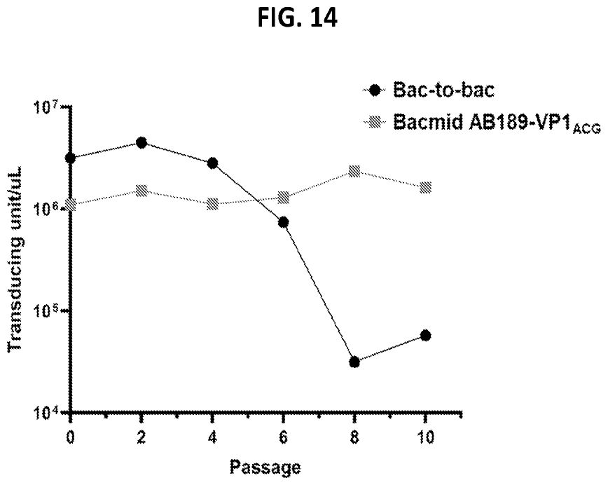

As shown in FIG. 13B, Bacmid AB189-VP1_{ACG} showed superior stability and continuously high levels of AAV1 production/titers over at least 10 passages, whereas the bac-to-bac control expression construct maintained production levels for about 4 passages but demonstrated a decrease in stability from passages 5 to 10. The Western blot analysis in FIG. 13C confirmed the qPCR results in FIG. 13B. As demonstrated in FIG. 13C, Bacmid AB189-VP1_{ACG} (right side of Western blots) not only demonstrated increased protein levels for the AAV1 capsid and Rep protein relative to the bac-to-bac construct (left side of Western blots), it also resulted in stable protein levels for both the Rep and the Cap proteins over 10 passages, However, for the bac-to-bac construct, no Rep and Cap proteins expression was detected by Western blot after 4-5 passages. Additionally, as shown in FIG. 14 and Table 15, Bacmid AB189-VP1_{ACG} also showed increased stability and potency compared to bac-to-bac, when expressed as transducing units per μL over 10 passages as calculated by the SEAP activity assay, providing further confirmation of the qPCR and Western blot results shown in FIGS. 13B-13C.

TABLE 15
Quantification of transducing units/μL for Bacmid AB189-
VP1_{ACG} and Bac-to-Bac control construct expressing AAV1

| | Bacmid AB189-VP1_{ACG} | Bac-to-Bac |
|---|---|---|
| Passage | transducing units/μL | transducing units/μL |
| 0 | 3.16E+06 | 1.10E+06 |
| 2 | 4.47E+06 | 1.51E+06 |
| 4 | 2.82E+06 | 1.12E+06 |
| 6 | 7.41E+05 | 1.29E+06 |
| 8 | 3.16E+04 | 2.34E+06 |
| 10 | 5.75E+04 | 1.62E+06 |

Taken together, these data demonstrate the superior passage stability of Bacmid AB189-VP1_{ACG} with separated Rep coding regions in different locations in the baculovirus genome as compared to bac-to-bac with single bicistronic Rep78/52 coding region located in the mini-F region of the baculovirus genome.

We claim:

1. An AAV expression construct comprising:
(i) at least two Rep-coding regions, each comprising a nucleotide sequence encoding a Rep protein independently chosen from Rep52, Rep40, Rep68, or Rep78 protein, e.g., a Rep52 protein and a Rep78 protein; and
(ii) a VP-coding region comprising a nucleotide sequence encoding at least one, two, or three VP proteins, chosen from a VP1 protein, a VP2 protein, a VP3 protein, or a combination thereof,
wherein the at least two Rep-coding regions each comprise a different nucleotide sequence and is present in different location;
wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94;
optionally wherein the AAV expression construct is stably maintained for at least 5-10 passages, e.g., at least 5, 6, 7, 8, 9, or 10 passages in an insect cell.

2. The AAV expression construct of claim 1, wherein the variant baculovirus genome comprises a nucleotide sequence or a portion thereof from a baculovirus genome selected from Autographa californica multiple nucleopolyhedrovirus (AcMNPV) (e.g., an AcMNPV strain E2, C6, or HR3), Bombyx mori nucleopolyhedrovirus (BmNPV), Anticarsia gemmatalis nucleopolyhedrovirus (AgMNPV), Orgyia pseudotsugata nucleopolyhedrovirus (OpMNPV), or Thysanoplusia orichalcea nucleopolyhedrovirus (ThorMNPV).

3. The AAV expression construct of claim 1 or 2, wherein the variant baculovirus genome comprises a nucleotide sequence or a portion thereof from the AcMNPV (e.g., AcMNPV E2) baculovirus genome.

4. The AAV expression construct of any one of claims 1-3, wherein the disruption results in inactivation of the non-essential gene (e.g., auxiliary and/or per os infectivity factor gene) or the regulatory region of the non-essential gene (e.g., promoter modification or insertion of heterologous DNA adjacent to non-essential gene).

5. The AAV expression construct of any one of claims 1-4, wherein the disruption in the at least two non-essential gene comprises an insertion, deletion, substitution, or mutation (e.g., frame-shift mutation).

6. The AAV expression construct of any one of claims 1-4, wherein the disruption of one or both of the at least two non-essential genes is present in the regulatory region of the non-essential gene (e.g., a promoter modification or insertion of heterologous DNA adjacent to non-essential gene).

7. The AAV expression construct of any one of claims 1-6, wherein the variant baculovirus genome comprises a disruption of at least three, four, five, six, seven, eight, nine, or ten non-essential genes (e.g., auxiliary and/or per os infectivity factor genes), wherein the at least three, four, five, six, seven, eight, nine, or ten non-essential genes are independently chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.

8. The AAV expression construct of any one of claims 1-7, wherein the at least two non-essential genes comprise:
(i) v-cath and egt;
(ii) v-cath, egt, and SOD;
(iii) chiA, v-cath, egt, p26, p10, and p74;
(iv) chiA, v-cath, egt, p26, p10, p74, and SOD; or
(v) chiA, v-cath, egt, p26, p10, p74, SOD, AcORF-91, and AcORF-108.

9. The AAV expression construct of any one of claims 1-8, wherein the disruption comprises a deletion of a chiA gene, a v-cath gene, a p26 gene, a p10 gene, and/or a p74 gene, or a portion thereof.

10. The AAV expression construct of any one of claims 1-9, wherein the at least two Rep-coding regions comprise a first Rep-coding region and a second Rep-coding region.

11. The AAV expression construct of claim 10, wherein the first Rep-coding region comprises a first a first open reading frame (ORF) comprising a start codon and a nucleotide sequence encoding a Rep78 protein and the second Rep-coding region comprises a second ORF comprising a start codon and a nucleotide sequence encoding a Rep52 protein.

12. The AAV expression construct of claim 10 or 11, wherein the first Rep-coding region, the second Rep-coding region or both comprises an ATG start codon (e.g., a canonical start codon).

13. The AAV expression construct of claim 10 or 11, wherein the first Rep-coding region, the second Rep-coding region or both comprises an ACG start codon, a CTG start codon, a TTG start codon, or a GTG start codon (e.g., a non-canonical start codon).

14. The AAV expression construct of any one of claims 10-13, wherein the first Rep-coding region comprises a nucleotide sequence encoding primarily a Rep78 protein, e.g., at least 50%, 60%, 70%, 80%, 90% or more Rep78 protein relative to a Rep52 protein.

15. The AAV expression construct of any one of claims 10-14, wherein the second Rep-coding comprises a nucleotide sequence encoding a Rep52 protein but not a Rep78 protein.

16. The AAV expression construct of any one of claims 10-15, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) the second Rep-coding region comprises a nucleotide sequence encoding a Rep52 protein but not a Rep78 protein.

17. The AAV expression construct of any one of claims 10-16, wherein the first Rep-coding region:
(i) comprises the nucleotide sequence of SEQ ID NO: 201, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 201; or a nucleotide sequence having at least 10, 20, 50, 100, 150, 200, 250, 300, 350, 400, or 450 but no more than 500 modifications (e.g., substitutions) relative to SEQ ID NO: 201; and/or
(ii) encodes the amino acid sequence of SEQ ID NO: 202; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 202; or an amino acid sequence comprising at least 1, 2, 3, 4, 5, 10, 15, or 20 but no more than 30 modifications (e.g., substitutions (e.g., conservative substitutions), insertions, or deletions) relative to the amino acid sequence of SEQ ID NO: 202.

18. The AAV expression construct of any one of claims 10-17, wherein the second Rep-coding region:
(i) comprises the nucleotide sequence of SEQ ID NO: 203, or a nucleotide sequence at least 70%, 75%, 80%, 85%, 90%, 95%, 96%, 97%, 98%, or 99% identical thereto; a nucleotide sequence having at least 10, 20, 50, 100, 150, 200, 250, 300, 350, 400, or 450 but no more than 500 different nucleotides relative to SEQ ID NO: 203; or a nucleotide sequence having at least 10, 20, 50, 100, 150, 200, 250, 300, 350, 400, or 450 but no more than 500 modifications (e.g., substitutions) relative to SEQ ID NO: 203; and/or
(ii) encodes the amino acid sequence of SEQ ID NO: 204; an amino acid sequence at least 70%, 75%, 80%, 85%, 90%, 95%, 96%, 97%, 98%, or 99% identical thereto; an amino acid sequence comprising at least 1, 2, 3, 4, 5, 10, 15, or 20 but no more than 30 different amino acids relative to SEQ ID NO: 204; or an amino acid sequence comprising at least 1, 2, 3, 4, 5, 10, 15, or 20 but no more than 30 modifications (e.g., substitutions (e.g., conservative substitutions), insertions, or deletions) relative to SEQ ID NO: 204.

19. The AAV expression construct of any one of claims 10-18, wherein the nucleotide sequence of the first Rep-coding region is operably linked to a first promoter.

20. The AAV expression construct of any one of claims 10-19, wherein the nucleotide sequence of the second Rep-coding region is operably linked to a second promoter.

21. The AAV expression construct of claim 19 or 20, wherein the first promoter, the second promoter, or both the first promoter and the second promoter is chosen from a polyhedrin (polh) promoter, a p10 promoter, a conotoxin (ctx) promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

22. The AAV expression construct of claim 19-21, the first promoter and the second promoter are each a polh promoter, optionally wherein the polh promoter comprises the nucleotide sequence of SEQ ID NO: 167; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 167; or a nucleotide sequence comprising at least one, two, three, four, five, six, or seven, but no more than ten modifications (e.g., substitutions) relative to SEQ ID NO: 167.

23. The AAV expression construct of any one of claims 10-22, the first Rep-coding region comprises a minicistron sequence, optionally wherein the minicistron sequence is present at the 5′ end of the first Rep-coding region, optionally wherein the minicistron sequence comprises SEQ ID NO: 4 or SEQ ID NO: 5; a nucleotide sequence at least 70%, 75%, 80%, 85%, 90%, 95%, 96%, 97%, 98%, or 99% identical to SEQ ID NO: 4 or 5; a nucleotide sequence comprising one, two, or three modifications (e.g., substitutions), but no more than four modifications (e.g., substitutions) relative to SEQ ID NO: 4 or 5; or a nucleotide sequence comprising one, two, or three, but no more than four different nucleotides relative to SEQ ID NO: 4 or 5.

24. The AAV expression construct of any one of claims 10-23, which comprises:
(i) in 5′ to 3′ order: a polh promoter, a minicistron sequence, and the first Rep-coding region comprising a nucleotide sequence encoding primarily a Rep78 protein, e.g., at least 50%, 60%, 70%, 80%, 90% or more Rep78 protein relative to a Rep52 protein (e.g., but not a Rep52 protein); and
(ii) in 5′ to 3′ order: a polh promoter and the second Rep-coding region comprising a nucleotide sequence encoding a Rep52 protein but not a Rep78 protein.

25. The AAV expression construct of any one of claims 10-24, wherein:
(i) the first Rep-coding region is present in first location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94; and
(ii) the second Rep-coding region is present in a second location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94;
wherein the first locus and the second locus are different.

26. The AAV expression construct of claim 10-25, wherein the first Rep-coding region is present in v-cath locus and the second Rep-coding region is present in the egt locus.

27. The AAV expression construct of any one of claims 10-26, wherein the first Rep-coding region is present in the v-cath locus of the variant baculovirus genome and is operably linked to a polh promoter, and the second Rep-coding region is present in the egt locus of the variant baculovirus genome and is operably linked to a polh promoter.

28. The AAV expression construct of any one of claims 10-27, wherein:
(i) the first Rep-coding region comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is present in the v-cath locus of the variant baculovirus genome; and
(ii) the second Rep-coding region comprises a nucleotide sequence encoding a Rep52 protein but not a Rep78 protein, wherein the second Rep-coding region is present in the egt locus of the variant baculovirus genome.

29. The AAV expression construct of any one of claims 1-28, wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

30. The AAV expression construct of any one of claims 1-29, wherein the VP-coding region comprises a single polycistronic ORF encoding a VP1 protein, a VP2 protein, and a VP3 protein.

31. The AAV expression construct of any one of claims 1-30, wherein the ORF encoding the VP1 protein comprises an ACG start codon, the ORF encoding the VP2 protein comprises an ACG start codon, and the ORF encoding the VP3 protein comprises an ATG start codon.

32. The AAV expression construct of any one of claims 1-31, wherein the ORF encoding the VP1 protein comprises an ATG start codon, the ORF encoding the VP2 protein comprises an ACG start codon, and the ORF encoding the VP3 protein comprises an ATG start codon.

33. The AAV expression construct of any one of claims 1-32, wherein the VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins (e.g., an AAV5 capsid protein or variant thereof, or an AAV9 capsid protein or variant thereof).

34. The AAV expression construct of any one of claims 1-33, wherein the VP-coding region encodes:
(i) a VP1 protein comprising the amino acid sequence of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences;
(ii) a VP2 protein e.g., a fragment or a portion, of any of SEQ ID NOs: 46-48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences, optionally wherein the VP2 protein comprises amino acids 138-736 or SEQ ID NOs: 71 or 46-48; amino acids 138-743 of SEQ ID NOs: 52, 53, 54, 56, 60, 61, 64, 66, 68; or amino acids 137-724 of SEQ ID NO: 168; and/or
(iii) a VP3 protein e.g., a fragment or a portion, of any of SEQ ID NOs: 46, 47, 48, 52, 53, 54, 56, 60, 61, 64, 66, 68, 70, 71, or 168, or an amino acid sequence at least 70%, 75%, 80%, 85%, 90%, 95%, 96%, 97%, 98%, or 99% identical to any of the aforesaid amino acid sequences, optionally wherein the VP3 protein comprises amino acids 203-736 of SEQ ID NOs: 71 or 46-48; amino acids 203-743 of SEQ ID NOs: 52, 53, 54, 56, 60, 61, 64, 66, 68; or amino acids 193-724 of SEQ ID NO: 168.

35. The AAV expression construct of any one of claims 1-34, wherein nucleotide sequence of the VP-coding region is operably linked to a promoter.

36. The AAV expression construct of claim 35, wherein the promoter is chosen from a polh promoter, a p10 promoter, a ctx promoter, a gp64 promoter, an IE promoter, an IE-1 promoter, a p6.9 promoter, a Dmhsp70 promoter, a Hsp70 promoter, a p5 promoter, a p19 promoter, a p35 promoter, a p40 promoter, or a variant, e.g., functional fragment, thereof.

37. The AAV expression of claim 35 or 36, wherein the promoter is a p10 promoter, optionally wherein the p10 promoter comprises the nucleotide sequence of SEQ ID NO: 200; a nucleotide sequence at least 70%, 75%, 80%, 85%, 90%, 95%, 96%, 97%, 98%, or 99% identical thereto; a nucleotide sequence comprising at least one, two, three, four, five, six, or seven, but no more than ten different nucleotides relative to SEQ ID NO: 200; or a nucleotide sequence comprising at least one, two, three, four, five, six, or seven, but no more than ten modifications (e.g., substitutions) relative to SEQ ID NO: 200.

38. The AAV expression construct of any one of claims 1-37, wherein the VP-coding region is present in a location in variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.

39. The AAV expression construct of any one of claims 1-38, wherein the VP-coding region is present in the v-cath gene locus of the variant baculovirus genome.

40. The AAV expression construct of any one of claims 1-39, wherein the VP-coding region is present in the v-cath gene locus of the variant baculovirus genome and wherein the VP-coding region comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

41. The AAV expression construct of any one of claims 1-40, wherein the VP-coding region is present in the v-cath gene locus of the variant baculovirus genome and wherein the VP-coding region:
(i) comprises a single polycistronic ORF encoding a VP1 protein, a VP2 protein, and a VP3 protein, wherein the ORF encoding the VP1 protein comprises an ACG or ATG start codon, the ORF encoding the VP2 protein comprises an ACG start codon, and the ORF encoding the VP3 protein comprises an ATG start codon; and
(ii) is operably linked to a p10 promoter.

42. The AAV expression construct of any one of claims 1-41, which further comprises a second VP-coding region, wherein the second VP-coding region comprises a nucleotide sequence encoding primarily a VP1 protein, e.g., at least 50%, 60%, 70%, 80%, 90% or more VP1 protein relative to a VP2 protein and/or a VP3 protein.

43. The AAV expression construct of claim 42, wherein the second VP-coding region encodes an AAV1 capsid protein, an AAV2 capsid protein, an AAV3 capsid protein, an AAV4 capsid protein, an AAV5 capsid protein, an AAV6 capsid protein, an AAV8 capsid protein, an AAV9 capsid protein, an AAVrh10 capsid protein or a variant of any of the aforesaid capsid proteins (e.g., an AAV5 capsid protein or variant thereof, or an AAV9 capsid protein or variant thereof).

44. The AAV expression construct of claim 42 or 43, wherein the second VP-coding region is present in the SOD gene locus of the variant baculovirus genome and is operably linked to a ctx promoter, optionally wherein the ctx promoter comprises the nucleotide sequence of SEQ ID NO: 164; a nucleotide sequence at least 70%, 75%, 80%, 85%, 90%, 95%, 96%, 97%, 98%, or 99% identical to SEQ ID NOs: 164; a nucleotide sequence comprising at least one, two, three, four, five, six, or seven, but no more than ten different nucleotides relative to SEQ ID NO: 164; or a nucleotide sequence comprising at least one, two, three, four, five, six, or seven, but no more than ten modifications (e.g., substitutions) relative to SEQ ID NOs: 164.

45. The AAV expression construct of any one of claims 1-44, which further comprises a modified Kozak sequence.

46. The AAV expression construct of claim 45, wherein the modified Kozak sequence:
(i) is capable of modulating expression, e.g., increasing expression, of a protein encoded by a gene that is immediately downstream of the modified Kozak sequence; and/or
(ii) comprises a start codon for the translation of a protein encoded by a gene that is immediately downstream of the modified Kozak sequence.

47. The AAV expression construct of claim 45 or 46, wherein:
(i) the modified Kozak sequence comprises the nucleotide sequence of sequence of any one of SEQ ID NOs: 32-42, or a nucleotide sequence comprising no more than one, two, or three different nucleotides relative to SEQ ID NOs: 32-42; and/or
(ii) the nucleotide sequence encoding the modified Kozak sequence comprises the nucleotide sequence of any one of SEQ ID NOs: 21-31, or a nucleotide sequence comprising no more than one, two, or three different nucleotides relative to SEQ ID NOs: 21-31.

48. The AAV expression construct of any one of claims 45-47, which comprises in 5′ to 3′ order, a p10 promoter, and a VP-coding region comprising a modified Kozak sequence and a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein;
wherein the modified Kozak sequence is present at the 5′ end of the VP-coding region, e.g., at the start of the VP-coding region encoding the VP1 protein (e.g., the ORF encoding the VP1 protein);
optionally wherein, the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.

49. The AAV expression construct of any one of claims 10-48, wherein:
(i) the first Rep-coding region is present in the v-cath locus of the variant baculovirus genome;
(ii) the second Rep-coding region is present in the egt locus of the variant baculovirus genome; and
(iii) the VP-coding region is present in the v-cath locus of the variant baculovirus genome.

50. The AAV expression construct of any one of claims 10-49, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

51. The AAV expression construct of any one of claims 10-50, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter; and
(iii) the VP-coding region is present in the v-cath locus of the variant baculovirus genome and is operably linked to a p10 promoter.

52. The AAV expression construct of any one of claims 10-51, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, wherein the second Rep-coding region is operably linked to a polh promoter; and
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter;
optionally wherein the VP-coding region is present in the reverse orientation relative to the first Rep-coding region.

53. The AAV expression construct of any one of claims 42-52, wherein:
(i) the first Rep-coding region is present in the v-cath locus of the variant baculovirus genome;
(ii) the second Rep-coding region is present in the egt locus of the variant baculovirus genome;
(iii) the VP-coding region is present in the v-cath locus of the variant baculovirus genome; and
(iv) the second VP-coding region is present in the SOD locus of the variant baculovirus genome.

54. The AAV expression construct of any one of claims 42-53, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein; and
(iv) the second VP-coding region is present in the SOD locus of the variant baculovirus genome and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein).

55. The AAV expression construct of any one of claims 42-54, wherein:
(i) the first Rep-coding region is present in the v-cath locus of the variant baculovirus genome and is operably linked to a polh promoter;
(ii) the second Rep-coding region is present in the egt locus of the variant baculovirus genome and is operably linked to a polh promoter;
(iii) the VP-coding region is present in the v-cath locus of the variant baculovirus genome and is operably linked to a p10 promoter; and
(iv) the second VP-coding region is present in the SOD locus of the variant and is operably linked to a ctx promoter.

56. The AAV expression construct of any one of claims 42-55, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and comprises a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein, and wherein the VP-coding region is operably linked to a p10 promoter; and
(iv) the second VP-coding region is present in the SOD locus of the variant baculovirus genome, and comprises a nucleotide sequence encoding primarily a VP1 protein, e.g., at least 50%, 60%, 70%, 80%, 90% or more VP1 protein relative to a VP2 protein or a VP3 protein (e.g., but not a VP2 or a VP3 protein), and wherein the second VP-coding region is operably linked to a ctx promoter;
optionally wherein, the VP-coding region is present in the reverse orientation relative to the first Rep-coding region.

57. The AAV expression construct of any one of claims 45-56, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.

58. The AAV expression construct of any one of claims 45-57, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein);
(ii) the second Rep-coding region is present in the egt locus of the variant baculovirus genome and comprises a nucleotide sequence encoding a Rep52 protein but not a Rep78 protein; and
(iii) the VP-coding region is present in the v-cath locus of the variant baculovirus genome, wherein the VP-coding region comprises a modified Kozak sequence, which is present at the 5′ end of the VP-coding region, e.g., at the start of the VP-coding region encoding the VP1 protein (e.g., the ORF encoding the VP1 protein), optionally wherein the modified Kozak sequence comprises the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33.

59. The AAV expression construct of any one of claims 45-58, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises:
(a) a modified Kozak sequence (e.g., comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33), which is present at the 5′ end of the VP-coding region (e.g., at the start of the VP-coding region); and
(b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

60. The AAV expression construct of any one of claims 45-59, wherein:
(i) the first Rep-coding region is present in the v-cath locus of the variant baculovirus genome and comprises a nucleotide sequence encoding primarily a Rep78 protein, e.g., at least 50%, 60%, 70%, 80%, 90% or more Rep78 protein relative to a Rep52 protein (e.g., but not a Rep52 protein), and wherein the first Rep-coding region is operably linked to a polh promoter;
(ii) the second Rep-coding region is present in the egt locus of the variant baculovirus genome and comprises a nucleotide sequence encoding a Rep52 protein but not a Rep78 protein, and wherein the second Rep-coding region is operably linked to a polh promoter;
(iii) the VP-coding region is present in the v-cath locus of the variant baculovirus genome and is operably linked to a p10 promoter, wherein the VP region comprises in 5′ to 3′ order:
(a) a modified Kozak sequence, optionally comprising the nucleotide sequence of SEQ ID NO: 32 or SEQ ID NO: 33, or a nucleotide sequence comprising no more than one, two, or three different nucleotides relative to SEQ ID NO: 32 or SEQ ID NO: 33; and
(b) a nucleotide sequence encoding a VP1 protein, a VP2 protein, and a VP3 protein, wherein the nucleotide sequence encoding the VP2 protein and the nucleotide sequence encoding the VP3 protein are comprised within the nucleotide sequence encoding the VP1 protein.

61. The AAV expression construct of any one of claims 1-60, which further comprises a payload coding region comprising a nucleotide sequence encoding a payload.

62. The AAV expression construct of any one of the preceding claims, which is stably maintained for at least 5-10 passages, e.g., at least 5, 6, 7, 8, 9, or 10 passages, in a host cell (e.g., an insect cell), e.g., when measured by an assay described herein, e.g., Western blot assay, a qPCR assay, or a SEAP assay, e.g., as described in Examples 5-8.

63. The AAV expression construct of any one of claims 1-62, which is capable of producing higher AAV titers relative to a reference, e.g., an AAV expression construct comprising overlapping VP coding regions and a bicistronic Rep78/52 coding region (e.g., Bac-to-Bac expression construct as described in Example 8), when measured by an assay, e.g., a SEAP assay or qPCR assay, e.g., as described in Example 7 or 8.

64. The AAV expression construct of any one of claims 42-63, which is capable of producing improved VP ratios (e.g., ratios of VP3:VP2:VP1), relative to a reference, e.g., an AAV expression construct comprising a single VP-coding region having an ACG start codon, when measured by an assay, e.g., a Western blot assay or qPCR assay, e.g., as described in Example 7.

65. The AAV expression construct of any one of claims 42-64, which is capable of producing more potent AAV capsid (e.g., AAV capsid with increased transduction efficiency), relative to a reference, e.g., an AAV expression construct comprising a single VP-coding region having an ACG start codon, when measured by an assay, e.g., a Western blot assay or qPCR assay, e.g., as described in Example 7.

66. An AAV payload expression construct comprising a payload coding region comprising a nucleotide sequence encoding a payload wherein the AAV expression construct comprises at least a portion of a baculovirus genome, e.g., a variant baculovirus genome, comprising a disruption of at least two non-essential genes (e.g., auxiliary and/or per os infectivity factor genes), wherein the at least two non-essential genes are independently chosen from egt, p74 (PIF0), p26, SOD, ChiA, v-cath, p10, polyhedrin, ctx, odv-e56, PIF1, PIF2, PIF3, PIF4, PIF5, Tn7, AcORF-91, AcORF-108, AcORF-52, v-ubi, or p94.

67. The AAV payload construct of claim 66, wherein the payload coding region is present in a location in the variant baculovirus genome chosen from ChiA, v-cath, p10, egt, polyhedrin, SOD, ctx, p26, odv-e56, p74 (PIF0), PIF1, PIF2, PIF3, PIF4, PIF5, Tn7, AcORF-91, AcORF-108, AcORF-52, v-ubi, or p94.

68. The AAV expression construct of any one of claims 61-65, or the AAV payload construct of claim 66 or 67, wherein the payload coding region comprises a start codon and a nucleotide sequence encoding the payload.

69. The AAV expression construct of any one of claim 61-65 or 68, or the AAV payload construct of any one of claims 66-68, wherein the payload coding region is present in the v-cath locus of the variant baculovirus genome.

70. The AAV expression construct of any one of claim 61-65, 68, or 69, or the AAV payload construct of any one of claims 66-69, wherein the encoded payload comprises a therapeutic protein or functional variant thereof; an antibody or antibody fragment; an enzyme; a component of a gene editing system; an RNAi agent (e.g., a dsRNA, siRNA, shRNA, pre-miRNA, pri-miRNA, miRNA, stRNA, lncRNA, piRNA, or snoRNA); or a combination thereof.

71. A cell comprising the AAV expression construct of any one of claim 61-65 or 68-70, and/or the AAV payload construct of any one of claims 66-70, optionally wherein the cell is an insect cell (e.g., an Sf9 cell or an Sf21 cell).

72. An AAV viral production system comprising the AAV expression construct of any one of claims 1-65, and the AAV payload expression construct of claim 66-70.

73. The AAV viral production system of claim 72, which further comprises a viral production cell, which comprises the AAV expression construct and the AAV payload expression construct, optionally wherein the viral production cell is an insect cell (e.g., an Sf9 cell or an Sf21 cell).

74. A method of producing one, two, three, four, or all of a Rep78 protein, a Rep52 protein, a VP1 protein, a VP protein, and/or a VP3 protein, the method comprising:
(i) providing a cell comprising the AAV expression construct of any one of claims 1-65;
(ii) incubating the cell under conditions suitable to produce the one, two, three, four, or all of the Rep78 protein, the Rep52 protein, the VP1 protein, the VP protein, and/or the VP3 protein.

75. A method of producing an AAV particle, the method comprising:
(i) providing a cell comprising the AAV expression construct of any one of claims 1-65 and the AAV payload construct of any one of claims 66-70, or a cell comprising the AAV production system of claims 197-199;
(ii) incubating the cell under conditions suitable to produce the AAV particle;
thereby producing the AAV particle.

76. The method of claim 74 or 75, wherein the cell is an insect cell, e.g., an Sf9 cell or an Sf21 cell.

77. An AAV particle made by the method of any one of claims 74-76.

78. A nucleic acid comprising a nucleotide sequence comprising a modified Kozak sequence and a VP-coding region, wherein the modified Kozak sequence comprises the nucleotide sequence of any one of SEQ ID NOs: 32-42, or a nucleotide sequence comprising no more than one, two, or three different nucleotides relative to SEQ ID NOs: 32-42.
